(19) United States
(12) Patent Application Publication
(10) Pub. No.: US 2025/0320204 A1
(43) Pub. Date: Oct. 16, 2025

(54) 7-AZA BICYCLIC HETEROARYL DERIVATIVES AS ECTONUCLEOTIDE PYROPHOSPHATASE PHOSPHODIESTERASE 1 INHIBITORS

(71) Applicant: RIBOSCIENCE LLC, Palo Alto, CA (US)

(72) Inventor: Ronald HAWLEY, Palo Alto, CA (US)

(21) Appl. No.: 18/962,188

(22) Filed: Nov. 27, 2024

Related U.S. Application Data
(60) Provisional application No. 63/603,798, filed on Nov. 29, 2023.

(51) Int. Cl. C07D 471/04; A61K 31/4375; A61K 31/438; A61K 31/4545; A61K 31/4725; A61K 31/519; C07D 519/00

(52) U.S. Cl. CPC C07D 471/04; A61K 31/4375; A61K 31/438; A61K 31/4545; A61K 31/4725; A61K 31/519; C07D 519/00

(57) ABSTRACT

The present disclosure provides certain 7-aza bicyclic heteroaryl compounds of Formula (Id)

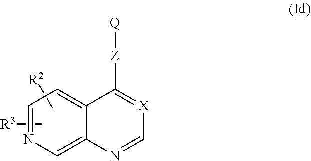

that inhibit ectonucleotide pyrophosphatase/phosphodiesterase 1 (ENPP1) enzymatic activity and are therefore useful for the treatment of diseases treatable by inhibition of ENPP1. Also provided are pharmaceutical compositions containing such compounds and processes for preparing such compounds.

### CROSS-REFERENCES TO RELATED APPLICATIONS

This application claims the benefit of priority under 35 U.S.C δ 119 (e) to U.S. Provisional Application No. 63/603,798 filed Nov. 29, 2023, the contents of which is herein incorporated by reference in its entirety for all purposes.

### FIELD OF THE DISCLOSURE

The present disclosure provides certain 7-aza bicyclic heteroaryl compounds that inhibit ectonucleotide pyrophosphatase/phosphodiesterase 1 (ENPP1) enzymatic activity and are therefore useful for the treatment of diseases treatable by inhibition of ENPP1. Also provided are pharmaceutical compositions containing such compounds and processes for preparing such compounds.

### BACKGROUND

ENPP1 enzyme is present in a wide range of tissues and cell types, such as lymphocytes, macrophages, liver, brain, heart, kidney, vascular smooth muscle cells, and chondrocytes. ENPP1 hydrolyzes ATP and other nucleoside triphosphates and releases AMP or other nucleoside monophosphates as well as pyrophosphate (PPi) (Kato K et al. 2012 PNAS 109:16876-16881; Hessle L et al. 2002 PNAS 99:9445-9449). The enzyme can also hydrolyze other nucleoside monophosphate esters (Kato K et al. 2012 PNAS 109:16876-16881). ENPP1 has been identified as the dominant 2′-3′-cGAMP hydrolase in cultured cells, tissue extracts and blood (Li L et al. 2014 Nat Chem Biol 10:1043-1048). Tissues and blood from ENPP1 knockout mice lack 2′-3′-cGAMP hydrolase activity. Elevated levels of ENPP1 have been associated with calcific aortic valve disease (CAVD) and calcium pyrophosphate dihydrate (CPPD) disease, an inflammatory disease resulting from calcium pyrophosphate dihydrate crystal deposits in the joint and surrounding tissues (Cote N et al. 2012 Eur J Pharmacol 689:139-146; Johnson K et al. 2001 Arthritis Rheum 44:1071). ENPP1 expression is upregulated in certain hepatocellular carcinomas, glioblastomas, melanomas, testicular, pancreatic and thyroid and breast cancers and has been associated with resistance to chemotherapy (see Lau W M et al. 2013 PLOS One 8:5; Bageritz J et al. 2014 Mol Cell Oncology 1:3; Bageritz J et al. 2014 Cell Death, Differentiation 21:929-940; Umar A et al. 2009 Mol Cell Proteomics 8:1278-1294). ENPP1 upregulation and variants of ENPP1 are also associated with insulin resistance and type 2 diabetes (Meyre D et al. 2005 Nat Genet 37:863-867; Maddux B A et al. 1995 Nature 373:448-451; Rey D et al. 2012 Mol Biol Rep 39:7687-7693) and enzyme activity of ENPP1 was reported to be required for the inhibition of insulin receptor signaling (Chin C N et al. 2009 Eur J Pharmacol 606:17-24).

Cyclic GMP-AMP synthase (cGAS) is a pattern recognition receptor that synthesizes the endogenous messenger molecule cGAMP from ATP and GTP in response to the presence of DNA derived from viruses, bacteria, damaged mitochondria, or cancer cells. The cGAMP molecule then binds to the stimulator of interferon genes (STING) protein, which initiates a signaling response that activates innate immunity and results in the production of type I interferon, antiviral and immune-stimulatory cytokines (Sun L et al. 2013 Science 339:786-791; Wu J et al. 2013 Science 339:826-830; Gao D et al. 2013 Science 341:903-906; Li X et al. 2013 Science 341:1390-1394; Schoggins J W et al. 2014 Nature 505:691-695; Wassermann R et al. 2015 Cell Host Microbe 17:799-810; Watson R O et al. 2015 Cell Host Microbe 17:811-819; Collins A et al. 2015 Cell Host Microbe 17:820-828; West A et al. 2015 Nature 520:533-557; Woo S R et al. 2014 Immunity 41:830-842; Deng L et al. 2014 Immunity 41:843-852; Chen Q et al. 2016 Nat Immunol 17:1142-1148). The cGAS enzyme, cGAMP messenger and STING is also involved in host defense against RNA viruses and the immune control of tumor development (Aguirre S et al. 2012 PLOS Pathog 8: e1002934; Barber GN 2015 Nat Rev Immunol 15:760-770). ENPP1 has been identified as the enzyme that naturally hydrolyzes cGAMP and therefore counteracts the innate immune response against infectious agents, damaged cells, and cancer cells (Li L et al. 2014 Nat Chem Biol 10:1043-1048). The efficacy of non-hydrolyzable cGAMP analogs in inducing functional immune responses is higher than that of natural, hydrolysable cGAMP (Li L et al. 2014 Nat Chem Biol 10:1043-1048; Corrales L et al. 2015 Cell Rep 11:1018-1030). Virus infection has been demonstrated to be facilitated by ENPP1 overexpression and is attenuated by silencing of ENPP1 (Wang J et al. 2018 Mol Immunol 95:56-63).

Inhibitors of cGAMP hydrolysis, including by inhibition of ENPP1, may therefore be used to increase the effectiveness of immune responses against cancer cells and tumors and against infections by RNA or DNA viruses or bacteria. Inhibitors of ENPP1 and of cGAMP or nucleoside triphosphate hydrolysis may also be used for the treatment of inflammatory diseases that are associated with elevated nucleotidase levels, reduced nucleoside triphosphate, reduced cGAMP or reduced nucleoside monophosphate ester levels or diseases associated with elevated nucleoside or nucleoside monophosphate levels. For these reasons, ENPP1 is an attractive therapeutic target for the treatment of diseases.

The present disclosure addresses these needs and provides related advantages as well.

### SUMMARY

In a first aspect, provided is a compound of Formula (I):

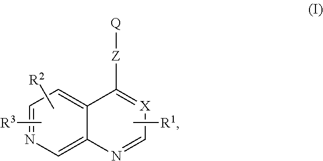

wherein:
X is N, CH, or C when attached to R¹;
R¹ is alkyl, alkoxy, halo, haloalkyl, haloalkoxy, amino, alkylamino, dialkylamino, hydroxyalkyl, alkoxyalkyl, hydroxyalkoxy, alkoxyalkoxy, hydroxyalkylamino, alkoxyalkylamino, aminoalkyl, aminoalkoxy, aminoalkylamino, diaminoalkyl, diaminoalkoxy, diaminoalkylamino, or cyano;
R² and R³ are independently absent, alkyl, hydroxy, alkoxy, halo, haloalkyl, haloalkoxy, cyano, amino, alkylamino, dialkylamino, aminocarbonyl, alkylaminocarbonyl, dialkylaminocarbonyl, hydroxyalkyl, alkoxyalkyl, hydroxyalkoxy, alkoxyalkoxy, hydroxyalkylamino, alkoxyalkylamino, aminoalkyl, aminoalkoxy, aminoalkylamino, heterocyclyl, heterocyclyloxy, heterocyclylamino (wherein heterocyclyl either alone or part of heterocyclyloxy and heterocyclylamino is optionally substituted with one, two, or three substituents independently selected from alkyl, halo, hydroxy, alkoxy, hydroxyalkyl, alkoxyalkyl, and aminoalkyl), heterocyclylalkyl, heterocyclylalkyloxy, heterocyclylalkylamino (wherein the heterocyclyl ring in heterocyclylalkyl, heterocyclylalkyloxy, and heterocyclylalkylamino is optionally substituted with one, two, or three substituents independently selected from alkyl, halo, hydroxy, alkoxy, hydroxyalkyl, alkoxyalkyl, and aminoalkyl), cycloalkyloxy, phenyloxy, or heteroaryloxy (where phenyl in phenyloxy and heteroaryl in heteroaryloxy are optionally substituted with one, two, or three substituents independently selected from alkyl, hydroxy, alkoxy, halo, haloalkyl, haloalkoxy, and cyano);
Z is cyclylaminylene, spiro cyclylaminylene, fused cyclylaminylene, NH, N (alkyl), O, S, SO, or SO₂, wherein:
(a) when Z is cyclylaminylene, spiro cyclylaminylene, or fused cyclylaminylene, then Q is -(alk)ₘ-W, wherein each Z moiety of (a) is substituted with R⁴ and R⁵, and R⁴ and R⁵ are each independently absent, alkyl, alkoxy, halo, haloalkyl, haloalkoxy, hydroxy, or cyano; and
(b) when Z is NH, N (alkyl), O, S, SO, or SO₂, then Q is -(alk₁)ₙ-Ar—W, wherein Ar is arylene or 5 or 6-membered heteroarylene, each Ar is substituted with R⁶ and R⁷, and R⁶ and R⁷ are independently absent, alkyl, alkoxy, halo, haloalkyl, haloalkoxy, hydroxy, or cyano;
alk and alk₁ are independently alkylene optionally substituted with one, two, or three halo;
m and n are independently 0 or 1; and
W is a group consisting of formula (i) or (ii):

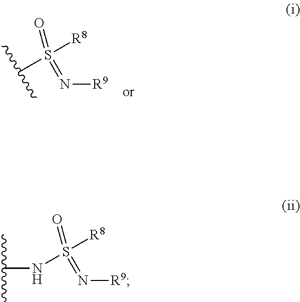

each R⁸ and R⁹ are independently hydrogen, alkyl, substituted alkyl, haloalkyl, substituted haloalkyl, cycloalkyl, substituted cycloalkyl, cycloalkylalkyl, heterocyclyl, or substituted heterocyclyl; or
a pharmaceutically acceptable salt thereof.

In some embodiments, provided are compounds of Formula (Id):

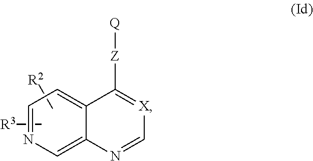

wherein:
X is:
   (a) CH or CR¹; or
   (b) N;
R¹ is alkyl, alkoxy, halo, haloalkyl, haloalkoxy, amino, alkylamino, dialkylamino, hydroxyalkyl, alkoxyalkyl, hydroxyalkoxy, alkoxyalkoxy, hydroxyalkylamino, alkoxyalkylamino, aminoalkyl, aminoalkoxy, aminoalkylamino, diaminoalkyl, diaminoalkoxy, diaminoalkylamino, or cyano;
R² and R³ are independently absent, alkyl, hydroxy, alkoxy, halo, haloalkyl, haloalkoxy, cyano, amino, alkylamino, dialkylamino, aminocarbonyl, alkylaminocarbonyl, dialkylaminocarbonyl, hydroxyalkyl, alkoxyalkyl, hydroxyalkoxy, alkoxyalkoxy, hydroxyalkylamino, alkoxyalkylamino, aminoalkyl, aminoalkoxy, aminoalkylamino, heterocyclyl, heterocyclyloxy, heterocyclylamino (wherein heterocyclyl either alone or part of heterocyclyloxy and heterocyclylamino is optionally substituted with one, two, or three substituents independently selected from alkyl, halo, hydroxy, alkoxy, hydroxyalkyl, alkoxyalkyl, and aminoalkyl), heterocyclylalkyl, heterocyclylalkyloxy, heterocyclylalkylamino (wherein the heterocyclyl ring in heterocyclylalkyl, heterocyclylalkyloxy, and heterocyclylalkylamino is optionally substituted with one, two, or three substituents independently selected from alkyl, halo, hydroxy, alkoxy, hydroxyalkyl, alkoxyalkyl, and aminoalkyl), cycloalkyloxy, phenyloxy, or heteroaryloxy (where phenyl in phenyloxy and heteroaryl in heteroaryloxy are optionally substituted with one, two, or three substituents independently selected from alkyl, hydroxy, alkoxy, halo, haloalkyl, haloalkoxy, and cyano);
Z is cyclylaminylene, spiro cyclylaminylene, fused cyclylaminylene, NH, N (alkyl), O, S, SO, or SO₂, wherein:
(a) when Z is cyclylaminylene, spiro cyclylaminylene, fused cyclylaminylene, then Q is -(alk) m-W, wherein each Z moiety of (a) is substituted with R⁴ and R⁵, and R⁴ and R⁵ are independently absent, alkyl, alkoxy, halo, haloalkyl, haloalkoxy, hydroxy, or cyano; or
(b) when Z is NH, N (alkyl), O, S, SO, or SO₂, then Q is -(alk₁)ₙ-Ar—W, wherein Ar is arylene or 5 or 6-membered heteroarylene, each Ar is substituted with R⁶ and R⁷, and R⁶ and R⁷ are independently absent, alkyl, alkoxy, halo, haloalkyl, haloalkoxy, hydroxy, or cyano;
alk and alk₁ are independently alkylene optionally substituted with one, two, or three halo;
m and n are independently 0 or 1; and
W is a group consisting of formula (i) or (ii):

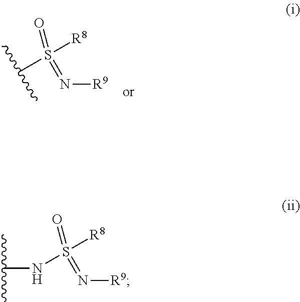

each R⁸ and R⁹ are independently hydrogen, alkyl, substituted alkyl, haloalkyl, substituted haloalkyl, cycloalkyl, substituted cycloalkyl, cycloalkylalkyl, heterocyclyl, or substituted heterocyclyl; or
a pharmaceutically acceptable salt thereof.

In a second aspect, provided is a pharmaceutical composition comprising a compound of (I) (or any of the embodiments thereof described herein) or a pharmaceutically acceptable salt thereof and a pharmaceutically acceptable excipient.

In a third aspect, provided are methods of treating a disease or condition treatable by inhibition of ENPP1 in a patient, preferably in a patient recognized as needing such a treatment, comprising administering to the patient a compound of Formula (I) (or any of the embodiments thereof described herein) or a pharmaceutically acceptable salt thereof in a therapeutically effective amount. In one embodiment, the disease is cancer. In a second embodiment, the disease is cancer selected from hepatocellular carcinomas, glioblastomas, melanomas, testicular cancer, pancreatic cancer, thyroid cancer, cervical cancer, ovarian cancer, bladder cancer, colon cancer, lung cancer, breast cancer, multiple myeloma, acute lymphocytic leukemia, acute myeloid leukemia, chronic lymphocytic leukemia, chronic myeloid leukemia, hairy cell leukemia, large granular lymphocytic leukemia, T-cell prolymphocytic leukemia, prolymphocytic leukemia, Hodgkin lymphoma, non-Hodgkin lymphoma, diffuse large B cell lymphoma, low grade glioma, colorectal cancer, gastric and gastrointestinal cancer, esophageal cancer, anal cancer, cancer of the appendix, kidney cancer, skin cancer, uterine cancer, brain cancer, adrenal cancer, bile duct cancer, bone cancer, fallopian tube cancer, sarcomas, germ cell tumors, head and neck cancer, neuroblastoma, pheochromocytoma and paraganglioma, cholangiocarcinoma, peritoneal cancer, retinoblastoma, liver cancer, thymoma, urethral cancer, prostate cancer, uveal melanoma, adenoid cystic carcinoma, and vaginal and vulvar cancer. In a third embodiment, the disease is cancer selected from hepatocellular carcinomas, glioblastomas, melanomas, testicular cancer, pancreatic cancer, thyroid cancer, cervical cancer, ovarian cancer, bladder cancer, colon cancer, and lung cancer.

In another embodiment, the disease is an inflammatory disease e.g., calcific aortic valve disease, osteoarthritis, and calcium pyrophosphate dihydrate disease. In yet another embodiment the disease metabolic disease e.g., type 2 diabetes, or a viral infection such as DNA virus infections, HIV, Herpes virus infections, Papilloma virus infections, RNA virus infections, and HBV.

In a fourth aspect, provided is a compound of Formula (I) (or any embodiments thereof described herein) or a pharmaceutically acceptable salt thereof for use as a medicament. In one embodiment, the medicament is for use in the treatment of cancer. In a second embodiment, the disease is cancer selected from hepatocellular carcinomas, glioblastomas, melanomas, testicular cancer, pancreatic cancer, thyroid cancer, cervical cancer, ovarian cancer, bladder cancer, colon cancer, lung cancer, breast cancer, multiple myeloma, acute lymphocytic leukemia, acute myeloid leukemia, chronic lymphocytic leukemia, chronic myeloid leukemia, hairy cell leukemia, large granular lymphocytic leukemia, T-cell prolymphocytic leukemia, prolymphocytic leukemia, Hodgkin lymphoma, non-Hodgkin lymphoma, diffuse large B cell lymphoma, low grade glioma, colorectal cancer, gastric and gastrointestinal cancer, esophageal cancer, anal cancer, cancer of the appendix, kidney cancer, skin cancer, uterine cancer, brain cancer, adrenal cancer, bile duct cancer, bone cancer, fallopian tube cancer, sarcomas, germ cell tumors, head and neck cancer, neuroblastoma, pheochromocytoma and paraganglioma, cholangiocarcinoma, peritoneal cancer, retinoblastoma, liver cancer, thymoma, urethral cancer, prostate cancer, uveal melanoma, adenoid cystic carcinoma, and vaginal and vulvar cancer. In a third embodiment, the disease is cancer selected from hepatocellular carcinomas, glioblastomas, melanomas, testicular cancer, pancreatic cancer, thyroid cancer, cervical cancer, ovarian cancer, bladder cancer, colon cancer, and lung cancer.

In another embodiment, the disease is an inflammatory disease e.g., calcific aortic valve disease, osteoarthritis, and calcium pyrophosphate dihydrate disease. In yet another embodiment the disease metabolic disease e.g., type 2 diabetes, or a viral infection such as DNA virus infections, HIV, Herpes virus infections, Papilloma virus infections, RNA virus infections, and HBV.

In a fifth aspect provided is use of a compound of Formula (I) or a pharmaceutically acceptable salt thereof (and any embodiments thereof disclosed herein) in the manufacture of a medicament for treating a disease in a patient in which the activity of ENPP1 contributes to the pathology and/or symptoms of the disease. In one embodiment, the disease is cancer.

In a second embodiment, the disease is cancer selected from hepatocellular carcinomas, glioblastomas, melanomas, testicular cancer, pancreatic cancer, thyroid cancer, cervical cancer, ovarian cancer, bladder cancer, colon cancer, lung cancer, breast cancer, multiple myeloma, acute lymphocytic leukemia, acute myeloid leukemia, chronic lymphocytic leukemia, chronic myeloid leukemia, hairy cell leukemia, large granular lymphocytic leukemia, T-cell prolymphocytic leukemia, prolymphocytic leukemia, Hodgkin lymphoma, non-Hodgkin lymphoma, diffuse large B cell lymphoma, low grade glioma, colorectal cancer, gastric and gastrointestinal cancer, esophageal cancer, anal cancer, cancer of the appendix, kidney cancer, skin cancer, uterine cancer, brain cancer, adrenal cancer, bile duct cancer, bone cancer, fallopian tube cancer, sarcomas, germ cell tumors, head and neck cancer, neuroblastoma, pheochromocytoma and paraganglioma, cholangiocarcinoma, peritoneal cancer, retinoblastoma, liver cancer, thymoma, urethral cancer, prostate cancer, uveal melanoma, adenoid cystic carcinoma, and vaginal and vulvar cancer. In a third embodiment, the disease is cancer selected from hepatocellular carcinomas, glioblastomas, melanomas, testicular cancer, pancreatic, thyroid cancer, cervical cancer, ovarian cancer, bladder cancer, colon cancer, and lung cancer.

In another embodiment, the disease is an inflammatory disease e.g., calcific aortic valve disease, osteoarthritis, and calcium pyrophosphate dihydrate disease. In yet another embodiment the disease metabolic disease e.g., type 2 diabetes, or a viral infection such as DNA virus infections, HIV, Herpes virus infections, Papilloma virus infections, RNA virus infections, and HBV.

In a sixth aspect, provided is a method of increasing the activity of an immune cell comprising contacting the immune cell with a compound of Formula (I) or a pharmaceutically acceptable salt thereof (and any embodiments thereof disclosed herein).

In a seventh aspect, provided is a method of increasing the activity of an immune cell in a subject comprising administering to a subject in need thereof a therapeutically effective amount of a compound of Formula (I) or a pharmaceutically acceptable salt thereof (and any embodiments thereof disclosed herein). In one embodiment of the seventh aspect, the activity of the immune cell is increased in a subject suffering from cancer or a viral disease.

In a second embodiment, the disease is cancer selected from hepatocellular carcinomas, glioblastomas, melanomas, testicular cancer, pancreatic cancer, thyroid cancer, cervical cancer, ovarian cancer, bladder cancer, colon cancer, lung cancer, breast cancer, multiple myeloma, acute lymphocytic leukemia, acute myeloid leukemia, chronic lymphocytic leukemia, chronic myeloid leukemia, hairy cell leukemia, large granular lymphocytic leukemia, T-cell prolymphocytic leukemia, prolymphocytic leukemia, Hodgkin lymphoma, non-Hodgkin lymphoma, diffuse large B cell lymphoma, low grade glioma, colorectal cancer, gastric and gastrointestinal cancer, esophageal cancer, anal cancer, cancer of the appendix, kidney cancer, skin cancer, uterine cancer, brain cancer, adrenal cancer, bile duct cancer, bone cancer, fallopian tube cancer, sarcomas, germ cell tumors, head and neck cancer, neuroblastoma, pheochromocytoma and paraganglioma, cholangiocarcinoma, peritoneal cancer, retinoblastoma, liver cancer, thymoma, urethral cancer, prostate cancer, uveal melanoma, adenoid cystic carcinoma, and vaginal and vulvar cancer. In a third embodiment, the disease is cancer selected from hepatocellular carcinomas, glioblastomas, melanomas, testicular cancer, pancreatic cancer, thyroid cancer, cervical cancer, ovarian cancer, bladder cancer, colon cancer, and lung cancer.

In yet another embodiment the disease is a viral infection such as DNA virus infections, HIV, Herpes virus infections, Papilloma virus infections, RNA virus infections, and HBV.

In an eighth aspect, provided is a compound of Formula (I) or a pharmaceutically acceptable salt thereof (and any embodiments thereof disclosed herein) for use in the treatment of:
1, cancer;
2. an inflammatory disease;
3. a metabolic disease; or
4. a viral disease.

In a first embodiment, the disease is cancer. In a second embodiment, the disease is cancer selected from hepatocellular carcinomas, glioblastomas, melanomas, testicular cancer, pancreatic cancer, thyroid cancer, cervical cancer, ovarian cancer, bladder cancer, colon cancer, lung cancer, breast cancer, multiple myeloma, acute lymphocytic leukemia, acute myeloid leukemia, chronic lymphocytic leukemia, chronic myeloid leukemia, hairy cell leukemia, large granular lymphocytic leukemia, T-cell prolymphocytic leukemia, prolymphocytic leukemia, Hodgkin lymphoma, non-Hodgkin lymphoma, diffuse large B cell lymphoma, low grade glioma, colorectal cancer, gastric and gastrointestinal cancer, esophageal cancer, anal cancer, cancer of the appendix, kidney cancer, skin cancer, uterine cancer, brain cancer, adrenal cancer, bile duct cancer, bone cancer, fallopian tube cancer, sarcomas, germ cell tumors, head and neck cancer, neuroblastoma, pheochromocytoma and paraganglioma, cholangiocarcinoma, peritoneal cancer, retinoblastoma, liver cancer, thymoma, urethral cancer, prostate cancer, uveal melanoma, adenoid cystic carcinoma, and vaginal and vulvar cancer. In a third embodiment, the disease is cancer selected from hepatocellular carcinomas, glioblastomas, melanomas, testicular cancer, pancreatic cancer, thyroid cancer, cervical cancer, ovarian cancer, bladder cancer, colon cancer, and lung cancer.

In another embodiment, the disease is an inflammatory disease e.g., calcific aortic valve disease, osteoarthritis, and calcium pyrophosphate dihydrate disease. In yet another embodiment the disease metabolic disease e.g., type 2 diabetes, or a viral infection such as DNA virus infections, HIV, Herpes virus infections, Papilloma virus infections, RNA virus infections, and HBV.

In any of the aforementioned aspects involving the treatment of cancer, are further embodiments comprising administering the compound of Formula (I) or a pharmaceutically acceptable salt thereof (or any embodiments thereof disclosed herein) in combination with at least one additional anticancer. When combination therapy is used, the agents can be administered simultaneously or sequentially.

### DETAILED DESCRIPTION

### Definitions:

Unless otherwise stated, the following terms used in the specification and claims are defined for the purposes of this Application and have the following meaning:

“Alkyl” means a linear or branched saturated monovalent hydrocarbon radical of one to six carbon atoms, e.g., methyl, ethyl, propyl, 2-propyl, butyl, pentyl, and the like.

“Alkylene” means a linear saturated divalent hydrocarbon radical of one to six carbon atoms or a branched saturated divalent hydrocarbon radical of three to six carbon atoms unless otherwise stated e.g., methylene, ethylene, propylene, 1-methylpropylene, 2-methylpropylene, butylene, pentylene, and the like.

“Amino” means a —NH₂.

“Alkylamino” means a —NHR radical where R is alkyl as defined above, e.g., methylamino, ethylamino, propylamino, or 2-propylamino, and the like.

“Aminoalkyl” means a linear monovalent hydrocarbon radical of one to six carbon atoms or a branched monovalent hydrocarbon radical of three to six carbons substituted with —NR′R″ where R′ and R″ are independently hydrogen or alkyl as defined above, e.g., aminomethyl, aminoethyl, methylaminomethyl, and the like.

“Aminoalkylamino” means a —NRR radical where R is hydrogen or alkyl and R is aminoalkyl as defined above, e.g., aminoethylamino, dimethylaminoethylamino, diethylaminoethylamino, dimethylaminopropylamino, diethylaminopropylamino, and the like.

“Aminoalkyloxy” or “aminoalkoxy” means a —OR radical where Ris aminoalkyl as defined above, e.g., aminoethyloxy, dimethylaminoethyloxy, diethylaminoethyloxy, dimethylaminopropyloxy, diethylaminopropyloxy, and the like.

“Aminocarbonyl” means —CONH₂ radical.

“Alkylaminocarbonyl” means —CONHR where R is alkyl as defined above radical, e.g., methylaminocarbonyl, propylaminocarbonyl.

“Alkoxy” means a —OR radical where R is alkyl as defined above, e.g., methoxy, ethoxy, propoxy, or 2-propoxy, n-, iso-, or tert-butoxy, and the like.

“Alkoxyalkyl” means a linear monovalent hydrocarbon radical of one to six carbon atoms or a branched monovalent hydrocarbon radical of three to six carbons substituted with at least one alkoxy group, such as one or two alkoxy groups, as defined above, e.g., 2-methoxyethyl, 1-, 2-, or 3-methoxypropyl, 2-ethoxyethyl, and the like.

“Alkoxyalkyloxy” or “alkoxyalkoxy” means a —O—R radical where R is alkoxyalkyl as defined above, e.g., methoxyethoxy, ethoxyethoxy, and the like.

“Alkoxyalkylamino” means a —NRR′ radical where R is hydrogen or alkyl and R′ is alkoxyalkyl as defined above, e.g., methoxyethylamino, ethoxyethylamino, propoxypropylamino, ethoxypropylamino, and the like.

“Alkoxycarbonyl” means a —C(O) OR radical where R is alkyl as defined above, e.g., methoxycarbonyl, ethoxycarbonly, propoxycarbonyl, and the like.

“Aryl” means a monovalent monocyclic or bicyclic aromatic hydrocarbon radical of 6 to 10 ring atoms e.g., phenyl or naphthyl.

“Arylene” means a divalent monocyclic or bicyclic aromatic hydrocarbon radical of 6 to 10 ring atoms e.g., 1,3- or 1,4-phenylene or 1,4-naphthylene, and the like.

“Phenyloxy” means a —OR radical where R is phenyl.

“Phenylalkyl” means a -(alkylene)-R radical where R is phenyl e.g., benzyl.

“Cycloalkyl” means a cyclic saturated monovalent hydrocarbon radical of three to ten carbon atoms, e.g., cyclopropyl, cyclobutyl, cyclopentyl, or cyclohexyl, and the like.

“Cycloalkylalkyl” means a -(alkylene)-R radical where R is cycloalkyl as defined above e.g., cyclopropylmethyl, and the like.

“Cycloalkyloxy” means a —OR radical where R is cycloalkyl (including specific cycloalkyl rings) as defined above e.g., cyclopropyloxy, and the like.

“Carboxy” means —COOH.

“Cyclylaminyl” means a saturated monovalent monocyclic ring of 4 to 8 ring atoms in which one ring atom is nitrogen, an additional ring atom can be nitrogen, O, or S(O) n (where n is 0, 1 or 2), and the remaining ring atoms are C. Representative examples of cyclylaminyl include, but is not limited to, piperidinyl, piperazinyl, azetidinyl, morpholinyl, and the like.

“Cyclylaminylene” means a saturated divalent monocyclic ring of 4 to 8 ring atoms in which one ring atom is nitrogen, an additional ring atom can be nitrogen, O, or S(O) n (where n is 0, 1 or 2), and the remaining ring atoms are C. Representative examples of cyclylaminylene include, but is not limited to, 1,3- or 1,4-piperidindiyl, 1,4-piperazindiyl, 1,3-azetidindiyl, 1,3-morpholindiyl, and the like.

“Dialkylamino” means a —NRR′ radical where R and R′ are alkyl as defined above, e.g., dimethylamino, methylethylamino, and the like.

“Dialkylaminocarbonyl” means —CONRR′ where R and R′ are alkyl as defined above radical, e.g., dimethylaminocarbonyl, diethylaminocarbonyl.

“Diaminoalkyl” means a linear monovalent hydrocarbon radical of one to six carbon atoms or a branched monovalent hydrocarbon radical of three to six carbons substituted with two-NR′R″ where R′ and R″ are independently hydrogen or alkyl as defined above, e.g., diaminoethyl, 1,3-diaminopropyl, 2-amino-3-methylaminopropyl, and the like.

“Diaminoalkylamino” means a —NR^{a}R^{b} radical where R^{a} is hydrogen or alkyl and R^{b} is diaminoalkyl as defined above, e.g., diaminoethylamino, 1,3-diaminopropylamino, 2-amino-3-methylaminopropylamino, and the like.

“Diaminoalkyloxy” means a —OR^{a} radical where R^{a} is diaminoalkyl as defined above, e.g., 2-diaminoethyloxy, 1,3-diaminopropyloxy, 2-amino-3-methylaminopropyloxy, and the like.

“Fused cyclylaminylene” means a fused divalent bicyclic ring in which a first ring is a saturated ring having 4 to 8 ring atoms in which one ring atom is nitrogen, an additional ring atom can be nitrogen, O, or S(O) n (where n is 0, 1 or 2), and the remaining ring atoms are C. Two adjacent ring atoms of the first ring are fused to two adjacent ring atoms of a phenyl or a five or six membered heteroaryl, each as defined herein. Any two suitable ring atoms of the fused cyclcylaminylene can be points of attachment. Non limiting examples of the fused cyclylaminylene include indolin-2-one-1-yl, indolinyl, isoindolinyl, and the like.

“Halo” means fluoro, chloro, bromo, or iodo, preferably fluoro or chloro.

“Haloalkyl” means alkyl radical as defined above, which is substituted with one or more halogen atoms, such as one to five halogen atoms, such as fluorine or chlorine, including those substituted with different halogens, e.g., —CH₂Cl, —CF₃, —CHF₂, —CH₂CF₃, —CF₂CF₃, —CF(CH₃) 2, and the like. When the alkyl is substituted with only fluoro, it can be referred to in this Application as fluoroalkyl.

“Haloalkoxy” means a —OR radical where R is haloalkyl as defined above e.g., —OCF₃, —OCHF₂, and the like. When R is haloalkyl where the alkyl is substituted with only fluoro, it is referred to in this Application as fluoroalkoxy.

“Hydroxyalkyl” means a linear monovalent hydrocarbon radical of one to six carbon atoms or a branched monovalent hydrocarbon radical of three to six carbons substituted with one or two hydroxy groups, provided that if two hydroxy groups are present they are not both on the same carbon atom. Representative examples include, but are not limited to, hydroxymethyl, 2-hydroxy-ethyl, 2-hydroxypropyl, 3-hydroxypropyl, 1-(hydroxymethyl)-2-methylpropyl, 2-hydroxybutyl, 3-hydroxybutyl, 4-hydroxybutyl, 2,3-dihydroxypropyl, 1-(hydroxymethyl)-2-hydroxyethyl, 2,3-dihydroxybutyl, 3,4-dihydroxybutyl and 2-(hydroxymethyl)-3-hydroxypropyl, preferably 2-hydroxyethyl, 2,3-dihydroxypropyl, and 1-(hydroxymethyl)-2-hydroxyethyl.

“Hydroxyalkylamino” means a —NRR radical where R is hydrogen or alkyl and R is hydroxyalkyl as defined above, e.g., hydroxyethylamino, hydroxypropylamino, and the like.

“Hydroxyalkyloxy” or “hydroxyalkoxy” means a —OR radical where Ris hydroxyoalkyl as defined above, e.g., hydroxyethyloxy, hydroxypropyloxy, and the like.

“Heterocyclyl” means a saturated or unsaturated monovalent monocyclic group of 4 to 8 ring atoms in which one or two ring atoms are heteroatom selected from N, O, or S(O) n, where n is an integer from 0 to 2, the remaining ring atoms being C. Additionally, one or two ring carbon atoms in the heterocyclyl ring can optionally be replaced by a —CO-group. More specifically the term heterocyclyl includes, but is not limited to, pyrrolidino, piperidino, homopiperidino, 2-oxopyrrolidinyl, 2-oxopiperidinyl, morpholino, piperazino, tetrahydro-pyranyl, thiomorpholino, and the like. When the heterocyclyl ring is unsaturated it can contain one or two ring double bonds provided that the ring is not aromatic. When the heterocyclyl group contains at least one nitrogen atom, it is also referred to herein as heterocycloamino and is a subset of the heterocyclyl group.

“Heterocyclylalkyl” or “heterocycloalkyl” means a -(alkylene)-R radical where R is heterocyclyl ring (including specific heterocyclyl rings) as defined above e.g., tetraydrofuranylmethyl, piperazinylmethyl, morpholinylethyl, and the like.

“Heterocyclylamino” means a —NRR′ radical where R is hydrogen or alkyl and R′ is heterocyclyl (including specific heterocyclyl rings) as defined above.

“Heterocyclylalkylamino” or “heterocycloalkylamino” means a —NRR′ radical where R is hydrogen or alkyl and R′ is heterocyclylalkyl ring (including specific heterocyclyl rings) as defined above e.g., tetraydrofuranylmethylamino, piperazinylethylamino, morpholinylethylamino, piperidinylmethylamino, and the like.

“Heterocyclyloxy” means a —OR radical where R is heterocyclyl (including specific heterocyclyl rings) as defined above.

“Heterocyclylalkyloxy” or “heterocycloalkyloxy” means a —OR radical where R is heterocyclylalkyl ring (including specific heterocyclyl rings) as defined above e.g., tetraydrofuranylmethyloxy, piperazinylethyloxy, morpholinylethyloxy, piperidinylmethyloxy, and the like.

“Heteroaryl” means a monovalent monocyclic or bicyclic aromatic radical of 5 to 10 ring atoms, unless otherwise stated, where one or more, (in one embodiment, one, two, or three), ring atoms are heteroatom selected from N, O, or S, the remaining ring atoms being carbon. Representative examples include, but are not limited to, pyrrolyl, thienyl, thiazolyl, imidazolyl, furanyl, indolyl, isoindolyl, oxazolyl, isoxazolyl, benzothiazolyl, benzoxazolyl, quinolinyl, isoquinolinyl, pyridinyl, pyrimidinyl, pyrazinyl, pyridazinyl, triazolyl, tetrazolyl, and the like. As defined herein, the terms “heteroaryl” and “aryl” are mutually exclusive. When the heteroaryl ring contains 5- or 6 ring atoms it is also referred to herein as 5- or 6-membered heteroaryl.

“Heteroarylene” means a divalent monocyclic or fused bicyclic aromatic radical of 5 to 10 ring atoms, unless otherwise stated, where one or more, (in one embodiment, one, two, or three), ring atoms are heteroatom selected from N, O, or S, the remaining ring atoms being carbon. Representative examples include, but are not limited to, pyrroldiyl, thiendiyl, thiazoldiyl, imidazoldiyl, indoldiyl, oxazoldiyl, quinolindiyl, pyridindiyl, pyrimidindiyl, pyrazindiyl, pyridazindiyl, and the like. When the heteroarylene ring contains 5- or 6 ring atoms it is also referred to herein as 5- or 6-membered heteroarylene.

“Heteroaryloxy” means a —OR radical where R is heteroaryl (including specific heteroaryl rings) as defined above.

The present disclosure also includes protected derivatives of compounds of the present disclosure (I). For example, when compounds of the present disclosure contain groups such as hydroxy, carboxy, thiol or any group containing a nitrogen atom(s), these groups can be protected with a suitable protecting groups. A comprehensive list of suitable protective groups can be found in T.W. Greene, Protective Groups in Organic Synthesis, John Wiley & Sons, Inc. (1999), the disclosure of which is incorporated herein by reference in its entirety. The protected derivatives of compounds of the present disclosure can be prepared by methods well known in the art.

The present disclosure also includes polymorphic forms and deuterated forms of the compound of the present disclosure and/or a pharmaceutically acceptable salt thereof.

A “pharmaceutically acceptable salt” of a compound means a salt that is pharmaceutically acceptable and that possesses the desired pharmacological activity of the parent compound. Such salts include: acid addition salts, formed with inorganic acids such as hydrochloric acid, hydrobromic acid, sulfuric acid, nitric acid, phosphoric acid, and the like; or formed with organic acids such as formic acid, acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, glucoheptonic acid, 4,4′-methylenebis -(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like. It is understood that the pharmaceutically acceptable salts are non-toxic. Additional information on suitable pharmaceutically acceptable salts can be found in Remington's Pharmaceutical Sciences, 17th ed., Mack Publishing Company, Easton, PA, 1985, which is incorporated herein by reference in its entirety.

The compounds of the present disclosure may have asymmetric centers. Compounds of the present disclosure containing an asymmetrically substituted atom may be isolated in optically active or racemic forms. It is well known in the art how to prepare optically active forms, such as by resolution of materials. All chiral, diastereomeric, all mixtures of chiral or diasteromeric forms, and racemic forms are within the scope of this disclosure, unless the specific stereochemistry or isomeric form is specifically indicated. It will also be understood by a person of ordinary skill in the art that when a compound is denoted as (R) stereoisomer, it may contain the corresponding(S) stereoisomer as an impurity i.e., the(S) stereoisomer in less than about 5%, preferably 2% by wt and then it is denoted as a mixture of R and S isomers, the amounts of R or S isomer in the mixture is greater than about 5%, preferably 2% w/w.

Certain compounds of the present disclosure can exist as tautomers and/or geometric isomers. All possible tautomers and cis and trans isomers, as individual forms and mixtures thereof are within the scope of this disclosure. Additionally, as used herein the term alkyl includes all the possible isomeric forms of said alkyl group albeit only a few examples are set forth. Furthermore, when the cyclic groups such as aryl, heteroaryl, heterocyclyl are substituted, they include all the positional isomers albeit only a few examples are set forth. Furthermore, all hydrates of a compound of the present disclosure are within the scope of this disclosure.

The compounds of the present disclosure may also contain unnatural amounts of isotopes at one or more of the atoms that constitute such compounds. Unnatural amounts of an isotope may be defined as ranging from the amount found in nature to an amount 100% of the atom in question, that differ only in the presence of one or more isotopically enriched atoms. Exemplary isotopes that can be incorporated into compounds of the present invention, such as a compound of Formula (I) (and any embodiment thereof disclosed herein including specific compounds) include isotopes of hydrogen, carbon, nitrogen, oxygen, phosphorus, sulfur, fluorine, chlorine, and iodine, such as ²H, ³H, ¹¹C, ¹³C, ¹⁴C, ¹³N, ¹⁵N, ¹⁵O, ¹⁷O, ¹⁸O, ³²P, ³³P, ³⁵S, ¹⁸F, ³⁶Cl, ¹²³I, and ¹²⁵I, respectively. Isotopically labeled compounds (e.g., those labeled with ³H and ¹⁴C) can be useful in compound or substrate tissue distribution assays. Tritiated (i.e., ³H) and carbon-14 (i.e., ¹⁴C) isotopes can be useful for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements). In some embodiments, in compounds disclosed herein, including in Table 1 below one or more hydrogen atoms are replaced by ²H or ³H, or one or more carbon atoms are replaced by ¹³C- or ¹⁴C-enriched carbon. Positron emitting isotopes such as ¹⁵O, ¹³N, ¹¹C, and ¹⁵F are useful for positron emission tomography (PET) studies to examine substrate receptor occupancy. Isotopically labeled compounds can generally be prepared by following procedures analogous to those disclosed in the Schemes or in the Examples herein, by substituting an isotopically labeled reagent for a non-isotopically labeled reagent.

“Oxo” or “carbonyl” means ═(O) group.

“Optional” or “optionally” means that the subsequently described event or circumstance may but need not occur, and that the description includes instances where the event or circumstance occurs and instances in which it does not. For example, “heterocyclyl group optionally substituted with an alkyl group” means that the alkyl may but need not be present, and the description includes situations where the heterocyclyl group is substituted with an alkyl group and situations where the heterocyclyl group is not substituted with alkyl.

A “pharmaceutically acceptable carrier or excipient” means a carrier or an excipient that is useful in preparing a pharmaceutical composition that is generally safe, non-toxic and neither biologically nor otherwise undesirable, and includes a carrier or an excipient that is acceptable for veterinary use as well as human pharmaceutical use. “A pharmaceutically acceptable carrier/excipient” as used in the specification and claims includes both one and more than one such excipient.

“Spiro cyclylaminylene” means a saturated bicyclic divalent ring having 6 to 10 ring atoms in which one ring atom is N and an additional ring atom can be a heteroatom selected from N, O, and S(O) n, where n is an integer selected from 0 to 2, the remaining ring atoms being C and the rings are connected through only one atom, the connecting atom is also called the spiroatom, most often a quaternary carbon (“spiro carbon”). Spiro cyclylaminylene is optionally substituted with one or two substituents independently selected from alkyl, halo, alkoxy, hydroxy, and cyano, unless stated otherwise. Representative examples include, but are not limited to,

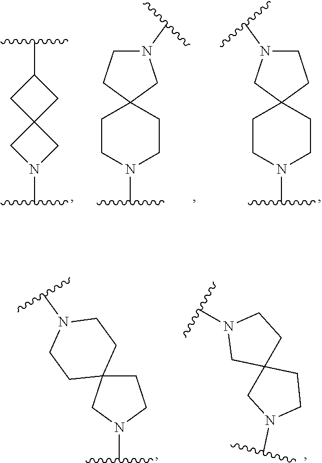

and the like.

“Substituted alkyl” means alkyl as defined above, that is substituted with one or two substituents independently selected from hydroxy, alkoxy, cyano, amino, alkylamino, and dialkylamino, each as defined herein.

“Substituted cycloalkyl” means cycloalkyl as defined above, that is substituted with one or two substituents independently selected from alkyl, hydroxy, alkoxy, halo, cyano, and haloalkyl, each as defined herein.

“Substituted haloalkyl” means haloalkyl as defined above, that is substituted with one or two substituents independently selected from hydroxy, alkoxy, cyano, amino, alkylamino, and dialkylamino, each as defined herein.

“Substituted heterocyclyl” means heterocyclyl as defined above, that is substituted with one, two, or three substituents independently selected from hydroxy, alkoxy, cyano, amino, alkylamino, dialkylamino, —COR (where R is alkyl), alkoxycarbonyl, each as defined herein.

Certain structures provided herein are drawn with one or more floating substituents. Unless provided otherwise or otherwise clear from the context (see, compounds in Table 1), the substituent(s) may be present on any atom of the ring through which the substituent is drawn, where chemically feasible and valency rules permitting. Therefore, based on the teachings of this application, including the specific compounds of Compound Table 1, in the structure:

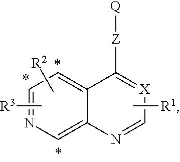

R² and R³ are absent when the *C are denoted as CH.

The term “about,” as used herein, is intended to qualify the numerical values which it modifies, denoting such a value as variable within a margin of error. When no particular margin of error, such as a standard deviation to a mean value given in a chart or table of data, is recited, the term “about” should be understood to mean that range which would encompass ±10%, preferably ±5%, the recited value and the range is included.

The term “disease” as used herein is intended to be generally synonymous, and is used interchangeably with, the terms “disorder,” “syndrome,” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

The term “patient” is generally synonymous with the term “subject” and includes all mammals including humans. Examples of patients include humans, livestock such as cows, goats, sheep, pigs, and rabbits, and companion animals such as dogs, cats, and horses. Preferably, the patient is a human.

The terms “inhibiting” and “reducing,” or any variation of these terms in relation of ENPP1, includes any measurable decrease or complete inhibition to achieve a desired result. For example, there may be a decrease of about, at most about, or at least about 5%, 10%, 15%, 20%, 25%, 30%, 35%, 40%, 45%, 50%, 55%, 60%, 65%, 70%, 75%, 80%, 85%, 90%, 95%, 99%, or more, or any range derivable therein, reduction of ENPP1 activity compared to normal.

“Treating” or “treatment” of a disease includes:
(1) preventing the disease, i.e. causing the clinical symptoms of the disease not to develop in a mammal that may be exposed to or predisposed to the disease but does not yet experience or display symptoms of the disease;
(2) inhibiting the disease, i.e., arresting or reducing (stabilizing) the development of the disease or its clinical symptoms; or
(3) relieving the disease, i.e., causing regression of the disease or its clinical symptoms.

A “therapeutically effective amount” means the amount of a compound of the present disclosure and/or a pharmaceutically acceptable salt thereof that, when administered to a patient for treating a disease, is sufficient to effect such treatment for the disease. The “therapeutically effective amount” will vary depending on the compound, the disease and its severity and the age, weight, etc., of the mammal to be treated.

Representative compound of Formula (I) are disclosed in Table 1 below:

| Cpd | | |
|---|---|---|
| No. | Structure | Name |
| 1 | 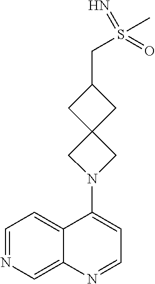 [SMILES: CS(=N)(=O)CC1CC2(C1)CN(c1ccnc3cnccc13)C2] | Imino(methyl)[[2-(1,7-naphthyridin-4-yl)-2- azaspiro[3.3]heptan-6-yl]methyl]-lambda6-sulfanone |
| 2a | 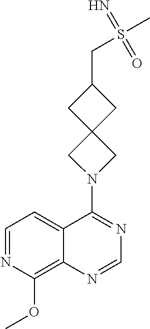 [SMILES: COc1nccc2c(N3CC4(CC(CS(C)(=N)=O)C4)C3)ncnc12] | Imino((2-(8-methoxypyrido[3,4-d]pyrimidin-4-yl)-2- azaspiro[3.3]heptan-6-yl)methyl)(methyl)-lambda6- sulfanone |
| 2b | 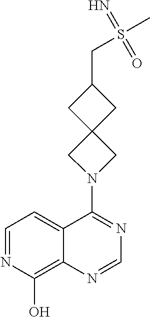 [SMILES: CS(=N)(=O)CC1CC2(C1)CN(c1ncnc3c(O)nccc13)C2] | ((2-(8-Hydroxypyrido[3,4-d]pyrimidin-4-y1)-2- azaspiro[3.3]heptan-6-yl)methyl)(imino)(methyl)- lambda6-sulfanone |
| 3 | 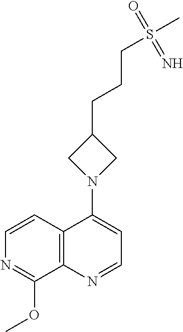 [SMILES: COc1nccc2c(N3CC(CCCS(C)(=N)=O)C3)ccnc12] | Imino(3-(1-(8-methoxy-1,7-naphthyridin-4-yl)azetidin- 3-yl)propyl)(methyl)-lambda6-sulfanone |
| 4 | 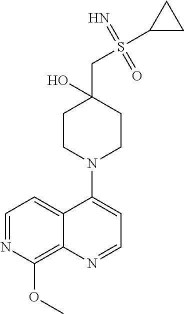 [SMILES: COc1nccc2c(N3CCC(O)(CS(=N)(=O)C4CC4)CC3)ccnc12] | Cyclopropyl((4-hydroxy-1-(8-methoxy-1,7- naphthyridin-4-yl)piperidin-4-yl)methyl)(imino)- lambda6-sulfanone |
| 5 | 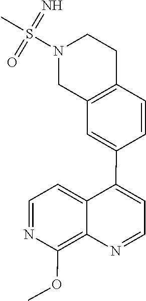 [SMILES: COc1nccc2c(-c3ccc4c(c3)CN(S(C)(=N)=O)CC4)ccnc12] | 8-Methoxy-4-(2-(S-methylsulfonimidoyl)-1,2,3,4- tetrahydro-isoquinolin-7-yl)-1,7-naphthyridine |
| 6 | 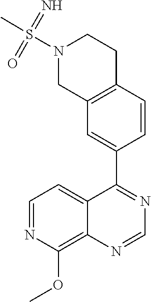 [SMILES: COc1nccc2c(-c3ccc4c(c3)CN(S(C)(=N)=O)CC4)ncnc12] | 8-Methoxy-4-(2-(S-methylsulfonimidoyl)-1,2,3,4- tetrahydro-isoquinolin-7-yl)pyrido[3,4-d]pyrimidine |
| 7 | 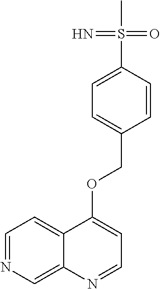 [SMILES: CS(=N)(=O)c1ccc(COc2ccnc3cnccc23)cc1] | (4-(((1,7-Naphthyridin-4- yl)oxy)methyl)phenyl)(imino)(methyl)-lambda6- sulfanone |
| 8 | 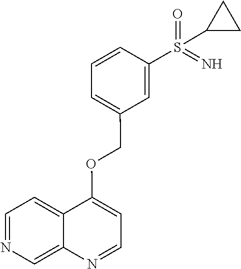 [SMILES: N=S(=O)(c1cccc(COc2ccnc3cnccc23)c1)C1CC1] | (3-(((1,7-Naphthyridin-4- yl)oxy)methyl)phenyl)(cyclopropyl)(imino)-lambda6- sulfanone |
| 9 | 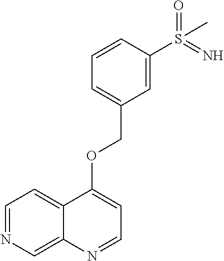 [SMILES: CS(=N)(=O)c1cccc(COc2ccnc3cnccc23)c1] | (3-(((1,7-Naphthyridin-4- yl)oxy)methyl)phenyl)(imino)(methyl)-lambda6- sulfanone |
| 10 | 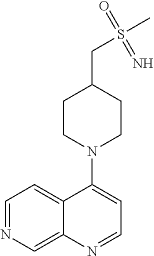 [SMILES: CS(=N)(=O)CC1CCN(c2ccnc3cnccc23)CC1] | ((1-(1,7-Naphthyridin-4-yl)piperidin-4- yl)methyl)(imino)(methyl)-lambda6-sulfanone |
| 11 | 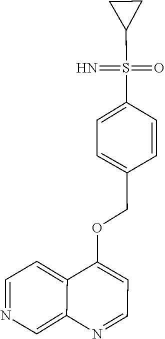 [SMILES: N=S(=O)(c1ccc(COc2ccnc3cnccc23)cc1)C1CC1] | (4-(((1,7-Naphthyridin-4- yl)oxy)methyl)phenyl)(cyclopropyl)(imino)-lambda6- sulfanone |
| 12 | 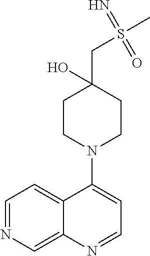 [SMILES: CS(=N)(=O)CC1(O)CCN(c2ccnc3cnccc23)CC1] | ((4-Hydroxy-1-(1,7-naphthyridin-4-yl)piperidin-4- yl)methyl)-(imino)(methyl)-lambda6-sulfanone |
| 13 | 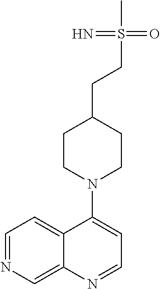 [SMILES: CS(=N)(=O)CCC1CCN(c2ccnc3cnccc23)CC1] | (2-(1-(1,7-Naphthyridin-4-yl)piperidin-4- yl)ethyl)(imino)(methyl)-lambda6-sulfanone |
| 14 | 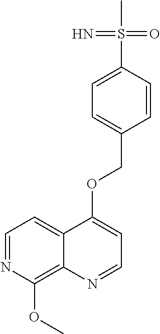 [SMILES: COc1nccc2c(OCc3ccc(S(C)(=N)=O)cc3)ccnc12] | Imino(4-(((8-methoxy-1,7-naphthyridin-4- yl)oxy)methyl)-phenyl)(methyl)-lambda6-sulfanone |
| 15 | 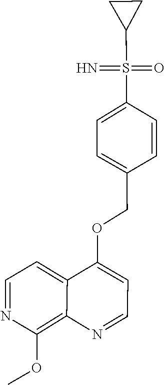 [SMILES: COc1nccc2c(OCc3ccc(S(=N)(=O)C4CC4)cc3)ccnc12] | Cyclopropyl(imino)(4-(((8-methoxy-1,7-naphthyridin- 4-yl)oxy)-methyl)phenyl)-lambda6-sulfanone |
| 16 | 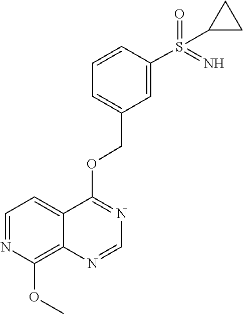 [SMILES: COc1nccc2c(OCc3cccc(S(=N)(=O)C4CC4)c3)ncnc12] | Cyclopropyl(imino)(3-(((8-methoxypyrido[3,4- d]pyrimidin-4-yl)oxy)methyl)phenyl)-lambda6- sulfanone |
| 17 | 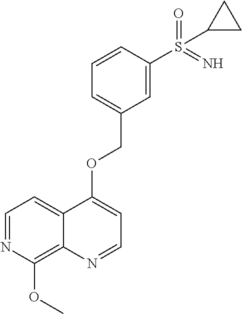 [SMILES: COc1nccc2c(OCc3cccc(S(=N)(=O)C4CC4)c3)ccnc12] | Cyclopropyl(imino)(3-(((8-methoxy-1,7-naphthyridin- 4-yl)oxy)-methyl)phenyl)-lambda6-sulfanone |
| 17a | 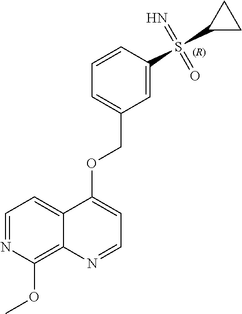 | (R)-Cyclopropyl(imino)(3-(((8-methoxy-1,7- naphthyridin-4-yl)oxy)methyl)phenyl)-lambda6- sulfanone |
| 17b | 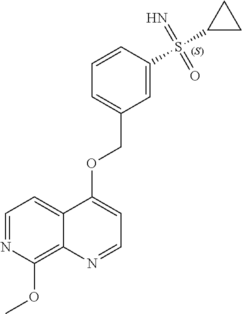 | (S)-Cyclopropyl(imino)(3-(((8-methoxy-1,7- naphthyridin-4-yl)oxy)methyl)phenyl)-lamba6- sulfanone |
| 18 | 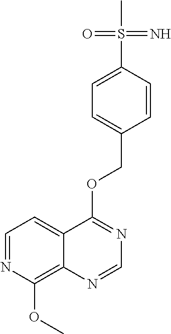 [SMILES: COc1nccc2c(OCc3ccc(S(C)(=N)=O)cc3)ncnc12] | Imino(4-(((8-methoxypyrido[3,4-d]pyrimidin-4- yl)oxy)methyl)-phenyl)(methyl)-lambda6-sulfanone |
| 19 | 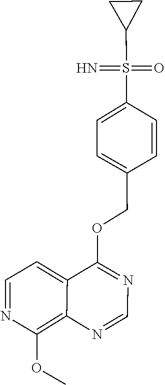 [SMILES: COc1nccc2c(OCc3ccc(S(=N)(=O)C4CC4)cc3)ncnc12] | Cyclopropyl(imino)(4-(((8-methoxypyrido[3,4- d]pyrimidin-4-yl)oxy)methyl)phenyl)-lambda6- sulfanone |
| 20 | 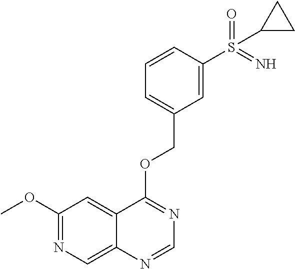 [SMILES: COc1cc2c(OCc3cccc(S(=N)(=O)C4CC4)c3)ncnc2cn1] | Cyclopropyl(imino)(3-(((6-methoxypyrido[3,4- d]pyrimidin-4-yl)oxy)methyl)phenyl)-lambda6- sulfanone |
| 21 | 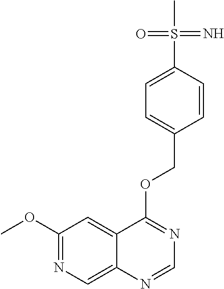 [SMILES: COc1cc2c(OCc3ccc(S(C)(=N)=O)cc3)ncnc2cn1] | Imino(4-(((6-methoxypyrido[3,4-d]pyrimidin-4- yl)oxy)methyl)-phenyl)(methyl)-lambda6-sulfanone |
| 22 | 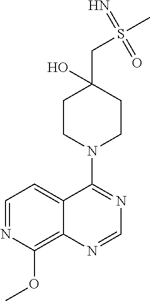 [SMILES: COc1nccc2c(N3CCC(O)(CS(C)(=N)=O)CC3)ncnc12] | ((4-Hydroxy-1-(8-methoxypyrido[3,4-d]pyrimidin-4- yl)piperidin-4-yl)methyl)(imino)(methyl)-lambda6- sulfanone |
| 23 | 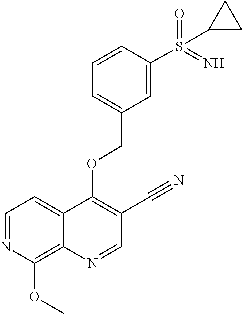 [SMILES: COc1nccc2c(OCc3cccc(S(=N)(=O)C4CC4)c3)c(C#N)cnc12] | 4-((3-(Cyclopropanesulfonimidoyl)benzyl)oxy)-8- methoxy-1,7-naphthyridine-3-carbonitrile |
| 24 | 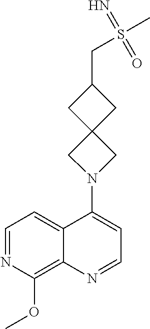 [SMILES: COc1nccc2c(N3CC4(CC(CS(C)(=N)=O)C4)C3)ccnc12] | Imino((2-(8-methoxy-1,7-naphthyridin-4-yl)-2- azaspiro[3.3]heptan-6-yl)methyl)(methyl)-lambda6- sulfanone |
| 25 | 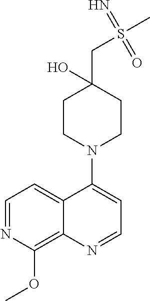 [SMILES: COc1nccc2c(N3CCC(O)(CS(C)(=N)=O)CC3)ccnc12] | ((4-Hydroxy-1-(8-methoxy-1,7-naphthyridin-4- yl)piperidin-4-yl)methyl)(imino)(methyl)-lambda6- sulfanone |
| 26 | 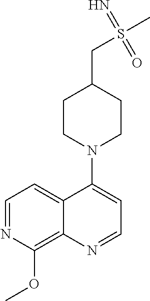 [SMILES: COc1nccc2c(N3CCC(CS(C)(=N)=O)CC3)ccnc12] | Imino((1-(8-methoxy-1,7-naphthyridin-4-yl)piperidin- 4-yl)methyl)-(methyl)-lambda6-sulfanone. |
| 27 | 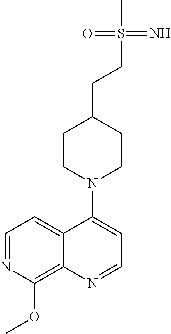 [SMILES: COc1nccc2c(N3CCC(CCS(C)(=N)=O)CC3)ccnc12] | Imino(2-(1-(8-methoxy-1,7-naphthyridin-4- yl)piperidin-4-yl)ethyl)-(methyl)-lambda6-sulfanone |
| 28 | 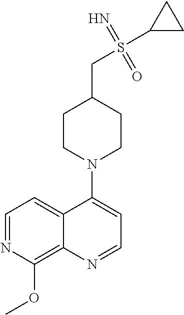 [SMILES: COc1nccc2c(N3CCC(CS(=N)(=O)C4CC4)CC3)ccnc12] | Cyclopropyl(imino)((1-(8-methoxy-1,7-naphthyridin- 4-y1)piperidin-4-yl)methyl)-lambda6-sulfanone |
| 29 | 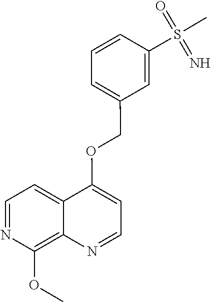 [SMILES: COc1nccc2c(OCc3cccc(S(C)(=N)=O)c3)ccnc12] | Imino(3-(((8-methoxy-1,7-naphthyridin-4- yl)oxy)methyl)-phenyl)(methyl)-lambda6-sulfanone |
| 30 | 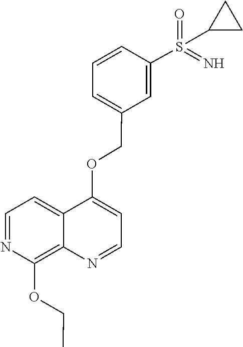 [SMILES: CCOc1nccc2c(OCc3cccc(S(=N)(=O)C4CC4)c3)ccnc12] | Cyclopropyl(3-(((8-ethoxy-1,7-naphthyridin-4- yl)oxy)methyl)-phenyl)(imino)-lambda6-sulfanone |
| 31 | 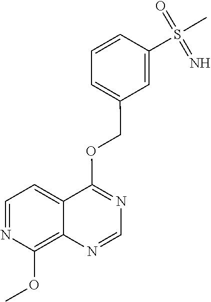 | Imino(3-(((8-methoxypyrido[3,4-d]pyrimidin-4- yl)oxy)methyl)-phenyl)(methyl)-lambda6-sulfanone |
| 32 | 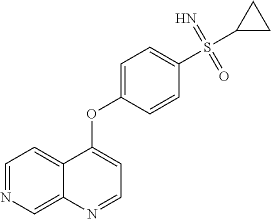 [SMILES: N=S(=O)(c1ccc(Oc2ccnc3cnccc23)cc1)C1CC1] | (4-((1,7-Naphthyridin-4- yl)oxy)phenyl)(cyclopropyl)(imino)-lambda6- sulfanone |
| 33 | 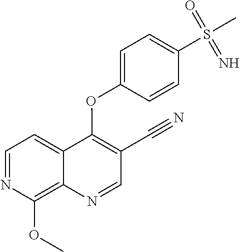 [SMILES: COc1nccc2c(Oc3ccc(S(C)(=N)=O)cc3)c(C#N)cnc12] | 4-[4-[Imino(methyl)oxo-lambda6-sulfanyl]phenoxy]-8- methoxy-1,7-naphthyridine-3-carbonitrile |
| 34 | 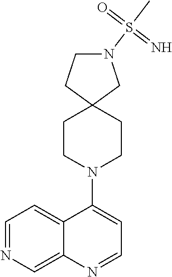 [SMILES: CS(=N)(=O)N1CCC2(CCN(c3ccnc4cnccc34)CC2)C1] | 2-(S-Methylsulfonimidoyl)-8-(1,7-naphthyridin-4-yl)- 2,8-diazaspiro[4.5]decane |
| 35 | 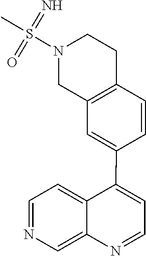 [SMILES: CS(=N)(=O)N1CCc2ccc(-c3ccnc4cnccc34)cc2C1] | 4-(2-(S-Methylsulfonimidoyl)-1,2,3,4- tetrahydroisoquinolin-7-y1)-1,7-naphthyridine |
| 36 | 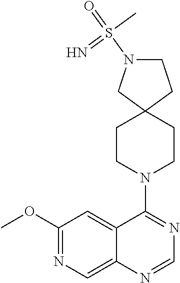 [SMILES: COc1cc2c(N3CCC4(CC3)CCN(S(C)(=N)=O)C4)ncnc2cn1] | 6-Methoxy-4-(2-(S-methylsulfonimidoyl)-2,8- diazaspiro[4.5]decan-8-yl)pyrido[3,4-d]pyrimidine |
| 37 | 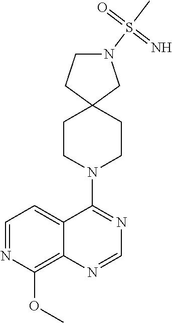 [SMILES: COc1nccc2c(N3CCC4(CC3)CCN(S(C)(=N)=O)C4)ncnc12] | 8-Methoxy-4-(2-(S-methylsulfonimidoyl)-2,8- diazaspiro[4.5]decan-8-yl)pyrido[3,4-d]pyrimidine |
| 38 | 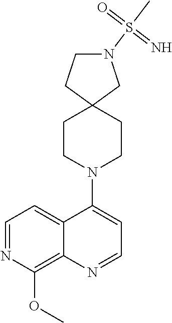 [SMILES: COc1nccc2c(N3CCC4(CC3)CCN(S(C)(=N)=O)C4)ccnc12] | 8-(8-Methoxy-1,7-naphthyridin-4-yl)-2-(S- methylsulfonimidoyl)-2,8-diazaspiro[4.5]decane |
| 39 | 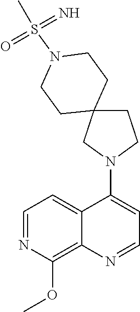 [SMILES: COc1nccc2c(N3CCC4(CCN(S(C)(=N)=O)CC4)C3)ccnc12] | 2-(8-Methoxy-1,7-naphthyridin-4-yl)-8-(S- methylsulfonimidoyl)-2,8-diazaspiro[4.5]decane |
| 40 | 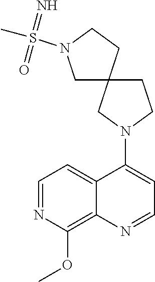 [SMILES: COc1nccc2c(N3CCC4(CCN(S(C)(=N)=O)C4)C3)ccnc12] | 8-Methoxy-4-(7-(S-methylsulfonimidoyl)-2,7- diazaspiro[4.4]nonan-2-yl)-1,7-naphthyridine |
| 41 | 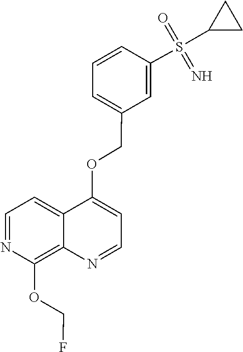 [SMILES: N=S(=O)(c1cccc(COc2ccnc3c(OCF)nccc23)c1)C1CC1] | Cyclopropyl(3-(((8-(fluoromethoxy)-1,7-naphthyridin- 4-yl)oxy)-methyl)phenyl)(imino)-lambda6-sulfanone |
| 42 | 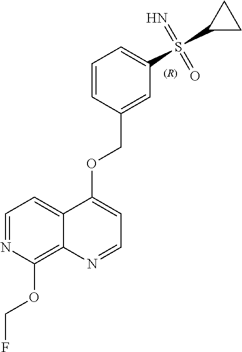 | (R)-Cyclopropyl(3-(((8-(fluoromethoxy)-1,7- naphthyridin-4-yl)oxy)-methyl)phenyl)(imino)- lambda6-sulfanone |
| 43 | 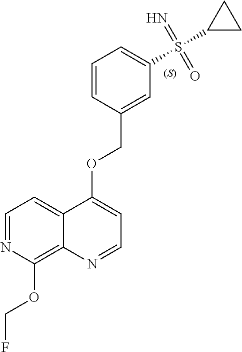 | (S)-Cyclopropyl(3-(((8-(fluoromethoxy)-1,7- naphthyridin-4-yl)oxy)-methyl)phenyl)(imino)- lambda6-sulfanone |
| 44 | 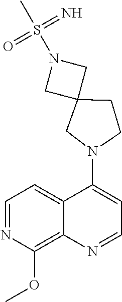 [SMILES: COc1nccc2c(N3CCC4(C3)CN(S(C)(=N)=O)C4)ccnc12] | 8-Methoxy-4-(2-(S-methylsulfonimidoyl)-2,6- diazaspiro[3.4]octan-6-yl)-1,7-naphthyridine |
| 45 | 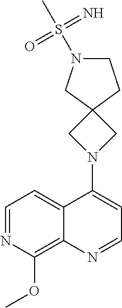 [SMILES: COc1nccc2c(N3CC4(CCN(S(C)(=N)=O)C4)C3)ccnc12] | 8-Methoxy-4-(6-(S-methylsulfonimidoyl)-2,6- diazaspiro[3.4]octan-2-yl)-1,7-naphthyridine |
| 46 | 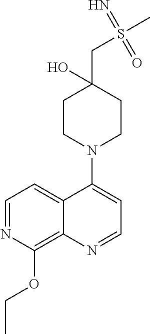 [SMILES: CCOc1nccc2c(N3CCC(O)(CS(C)(=N)=O)CC3)ccnc12] | ((1-(8-Ethoxy-1,7-naphthyridin-4-yl)-4- hydroxypiperidin-4-yl)-methyl)(imino)(methyl)- lambda6-sulfanone |
| 47 | 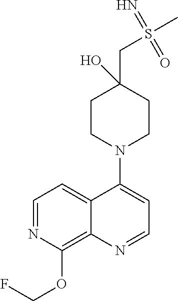 [SMILES: CS(=N)(=O)CC1(O)CCN(c2ccnc3c(OCF)nccc23)CC1] | ((1-(8-(Fluoromethoxy)-1,7-naphthyridin-4-yl)-4- hydroxypiperidin-4-yl)methyl)(imino)(methyl)- lambda6-sulfanone |
| 48 | 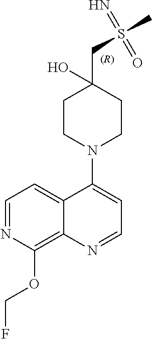 | (R)-((1-(8-(Fluoromethoxy)-1,7-naphthyridin-4-yl)-4- hydroxy-piperidin-4-yl)methyl)(imino)(methyl)- lambda6-sulfanone |
| 49 | 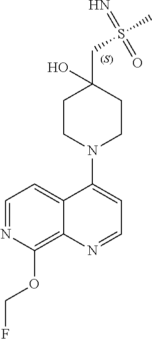 | (S)-((1-(8-(Fluoromethoxy)-1,7-naphthyridin-4-yl)-4- hydroxy-piperidin-4-yl)methyl)(imino)(methyl)- lambda6-sulfanone |
| 50 | 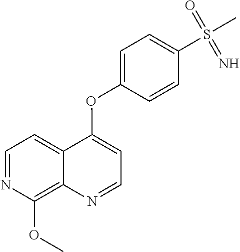 [SMILES: COc1nccc2c(Oc3ccc(S(C)(=N)=O)cc3)ccnc12] | Imino(4-((8-methoxy-1,7-naphthyridin-4- yl)oxy)phenyl)-(methyl)-lambda6-sulfanone |
| 51 | 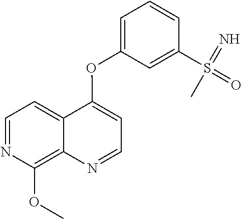 [SMILES: COc1nccc2c(Oc3cccc(S(C)(=N)=O)c3)ccnc12] | Imino(3-((8-methoxy-1,7-naphthyridin-4- yl)oxy)phenyl)(methyl)-lambda6-sulfanone |
| 52 | 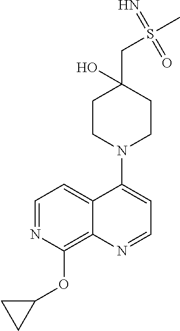 [SMILES: CS(=N)(=O)CC1(O)CCN(c2ccnc3c(OC4CC4)nccc23)CC1] | ((1-(8-Cyclopropoxy-1,7-naphthyridin-4-yl)-4- hydroxypiperidin-4-yl)methyl)(imino)(methyl)- lambda6-sulfanone |
| 53 | 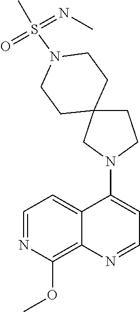 [SMILES: CN=S(C)(=O)N1CCC2(CCN(c3ccnc4c(OC)nccc34)C2)CC1] | ((2-(8-Methoxy-1,7-naphthyridin-4-yl)-2,8- diazaspiro[4.5]decan-8-yl)(methyl)oxo-lambda6- sulfanylidene)(methyl)amine |
| 54 | 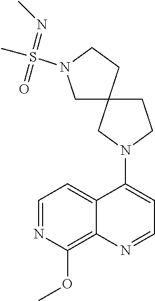 [SMILES: CN=S(C)(=O)N1CCC2(CCN(c3ccnc4c(OC)nccc34)C2)C1] | ((7-(8-Methoxy-1,7-naphthyridin-4-yl)-2,7- diazaspiro(4.4)nonan-2-yl)(methyl)oxo-lambda6- sulfanylidene)(methyl)amine |
| 55 | 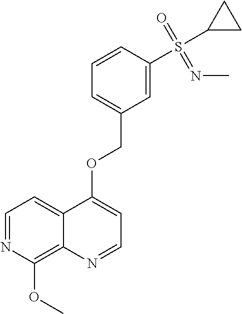 [SMILES: CN=S(=O)(c1cccc(COc2ccnc3c(OC)nccc23)c1)C1CC1] | Cyclopropyl(3-(((8-methoxy-1,7-naphthyridin-4- yl)oxy)methyl)-phenyl)(methylimino)-lambda6- sulfanone |
| 56 | 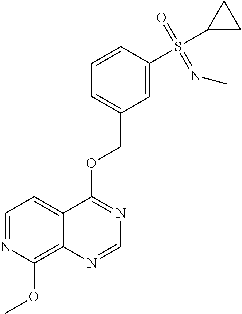 [SMILES: CN=S(=O)(c1cccc(COc2ncnc3c(OC)nccc23)c1)C1CC1] | Cyclopropyl(3-(((8-methoxypyrido[3,4-d]pyrimidin-4- yl)oxy)-methyl)phenyl)(methylimino)-lambda6- sulfanone |
| 57 | 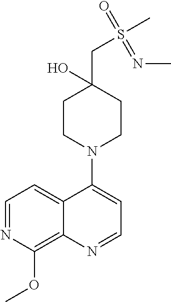 [SMILES: CN=S(C)(=O)CC1(O)CCN(c2ccnc3c(OC)nccc23)CC1] | ((4-hydroxy-1-(8-methoxy-1,7-naphthyridin-4- yl)piperidin-4-yl)-methyl)(methyl)(methylimino)- lambda6-sulfanone |
| 58 | 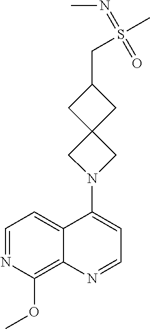 [SMILES: CN=S(C)(=O)CC1CC2(C1)CN(c1ccnc3c(OC)nccc13)C2] | ((2-(8-Methoxy-1,7-naphthyridin-4-yl)-2- azaspiro[3.3]heptan-6- yl)methyl)(methyl)(methylimino)-lambda6-sulfanone |
| 59 | 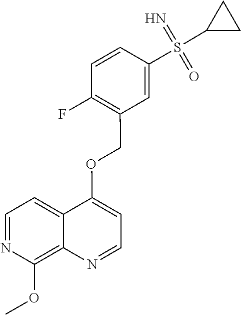 [SMILES: COc1nccc2c(OCc3cc(S(=N)(=O)C4CC4)ccc3F)ccnc12] | Cyclopropyl(4-fluoro-3-(((8-methoxy-1,7- naphthyridin-4-yl)oxy)-methyl)phenyl)(imino)- lambda6-sulfanone |
| 60 | 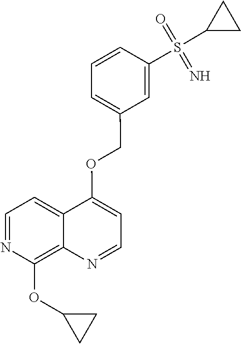 [SMILES: N=S(=O)(c1cccc(COc2ccnc3c(OC4CC4)nccc23)c1)C1CC1] | (3-(((8-Cyclopropoxy-1,7-naphthyridin-4- yl)oxy)methyl)-phenyl)(cyclopropyl)(imino)-lambda6- sulfanone |
| 61 | 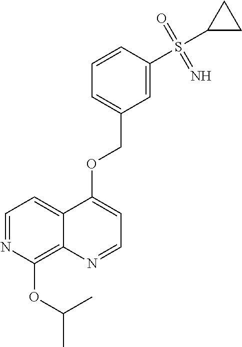 [SMILES: CC(C)Oc1nccc2c(OCc3cccc(S(=N)(=O)C4CC4)c3)ccnc12] | Cyclopropyl(imino)(3-(((8-isopropoxy-1,7- naphthyridin-4-yl)oxy)-methyl)phenyl)-lambda6- sulfanone |
| 62 | 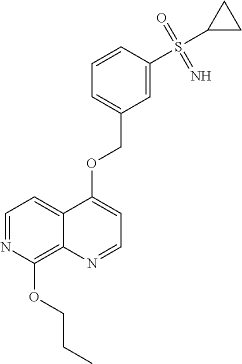 [SMILES: CCCOc1nccc2c(OCc3cccc(S(=N)(=O)C4CC4)c3)ccnc12] | Cyclopropyl(imino)(3-(((8-propoxy-1,7-naphthyridin- 4-yl)oxy)-methyl)phenyl)-lambda6-sulfanone |
| 63 | 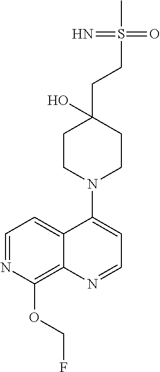 [SMILES: CS(=N)(=O)CCC1(O)CCN(c2ccnc3c(OCF)nccc23)CC1] | (2-(1-(8-(Fluoromethoxy)-1,7-naphthyridin-4-yl)-4- hydroxypiperidin-4-yl)ethyl)(imino)(methyl)-lambda6- sulfanone |
| 64 | 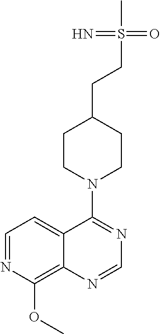 [SMILES: COc1nccc2c(N3CCC(CCS(C)(=N)=O)CC3)ncnc12] | Imino(2-(1-(8-methoxypyrido[3,4-d]pyrimidin-4- yl)piperidin-4-yl)ethyl)(methyl)-lambda6-sulfanone |
| 65 | 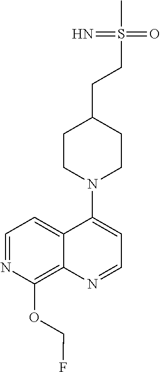 [SMILES: CS(=N)(=O)CCC1CCN(c2ccnc3c(OCF)nccc23)CC1] | (2-(1-(8-(Fluoromethoxy)-1,7-naphthyridin-4- yl)piperidin-4-yl)-ethyl)(imino)(methyl)-lambda6- sulfanone |
| 66 | 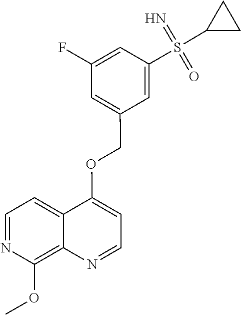 [SMILES: COc1nccc2c(OCc3cc(F)cc(S(=N)(=O)C4CC4)c3)ccnc12] | Cyclopropyl(3-fluoro-5-(((8-methoxy-1,7- naphthyridin-4-yl)oxy)-methyl)phenyl)(imino)- lambda6-sulfanone |
| 67 | 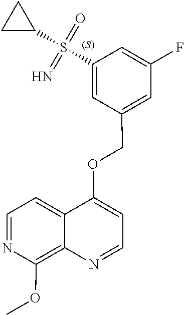 | (S)-Cyclopropyl(3-fluoro-5-(((8-methoxy-1,7- naphthyridin-4-yl)oxy)-methyl)phenyl)(imino)- lambda6-sulfanone |
| 68 | 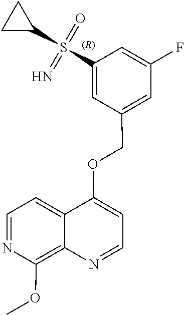 | (R)-Cyclopropyl(3-fluoro-5-(((8-methoxy-1,7- naphthyridin-4-yl)oxy)-methyl)phenyl)(imino)- lambda6-sulfanone |
| 69 | 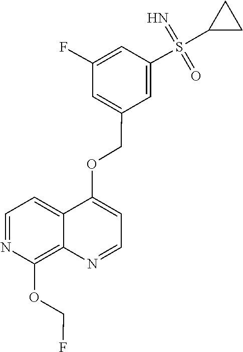 [SMILES: N=S(=O)(c1cc(F)cc(COc2ccnc3c(OCF)nccc23)c1)C1CC1] | Cyclopropyl(3-fluoro-5-(((8-(fluoromethoxy)-1,7- naphthyridin-4-yl)oxy)methyl)phenyl)(imino)- lambda6-sulfanone |
| 70 | 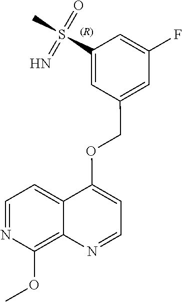 | (R)-(3-Fluoro-5-(((8-methoxy-1,7-naphthyridin-4- yl)oxy)methyl)-phenyl)(imino)(methyl)-lambda6- sulfanone |
| 71 | 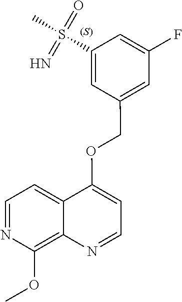 | (S)-(3-Fluoro-5-(((8-methoxy-1,7-naphthyridin-4- yl)oxy)methyl)-phenyl)(imino)(methyl)-lambda6- sulfanone |
| 72 | 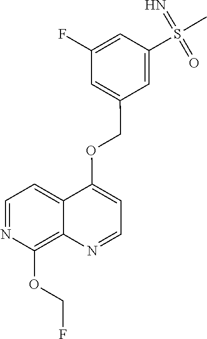 [SMILES: CS(=N)(=O)c1cc(F)cc(COc2ccnc3c(OCF)nccc23)c1] | 3-Fluoro-5-(((8-(fluoromethoxy)-1,7-naphthyridin-4- yl)oxy)methyl)-phenyl)(imino)(methyl)-lambda6- sulfanone |
| 73 | 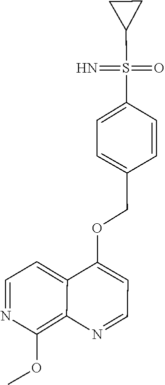 [SMILES: COc1nccc2c(OCc3ccc(S(=N)(=O)C4CC4)cc3)ccnc12] | Cyclopropyl(imino)(4-(((8-methoxy-1,7-naphthyridin- 4-yl)oxy)-methyl)phenyl)-lambda6-sulfanone |
| 74 | 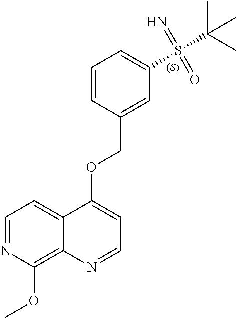 | (S)-tert-Butyl(imino)(3-(((8-methoxy-1,7-naphthyridin- 4-yl)oxy)-methyl)phenyl)-lambda6-sulfanone |
| 75 | 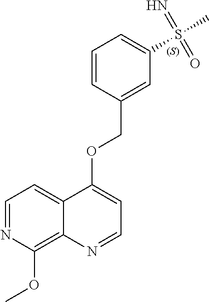 | (S)-Imino(3-(((8-methoxy-1,7-naphthyridin-4- yl)oxy)methyl)-phenyl)(methyl)-lambda6-sulfanone |
| 76 | 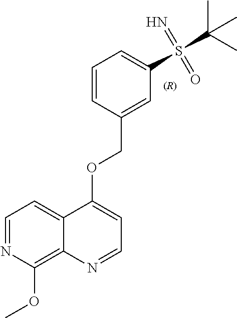 | (R)-tert-Butyl(imino)(3-(((8-methoxy-1,7- naphthyridin-4-yl)oxy)-methyl)phenyl)-lambda6- sulfanone |
| 77 | 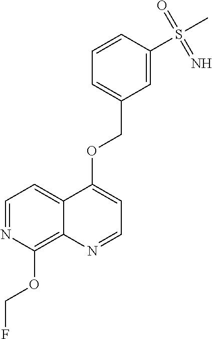 [SMILES: CS(=N)(=O)c1cccc(COc2ccnc3c(OCF)nccc23)c1] | (3-(((8-(Fluoromethoxy)-1,7-naphthyridin-4- yl)oxy)methyl)-phenyl)(imino)(methyl)-lambda6- sulfanone |
| 78 | 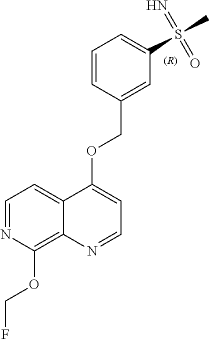 | (R)-(3-(((8-(Fluoromethoxy)-1,7-naphthyridin-4- yl)oxy)methyl)-phenyl)(imino)(methyl)-lambda6- sulfanone |
| 79 | 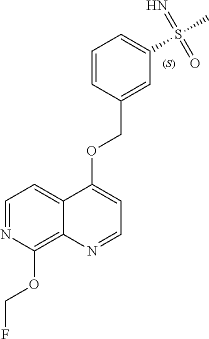 | (S)-(3-(((8-(Fluoromethoxy)-1,7-naphthyridin-4- yl)oxy)methyl)-phenyl)(imino)(methyl)-lambda6- sulfanone |
| 80 | 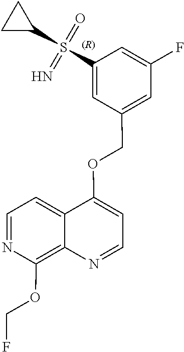 | (R)-cyclopropyl(3-fluoro-5-(((8-(fluoromethoxy)-1,7- naphthyridin-4-yl)oxy)methyl)phenyl)(imino)- lambda6-sulfanone |
| 81 | 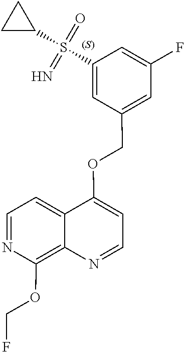 | (S)-cyclopropyl(3-fluoro-5-(((8-(fluoromethoxy)-1,7- naphthyridin-4-yl)oxy)methyl)phenyl)(imino)- lambda6-sulfanone |
| 82 | 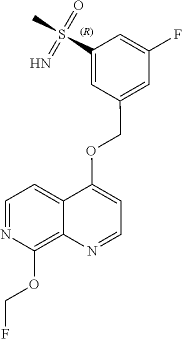 | (R)-(3-fluoro-5-(((8-(fluoromethoxy)-1,7-naphthyridin- 4-yl)oxy)methyl)phenyl)(imino)(methyl)-lambda6- sulfanone |
| 83 | 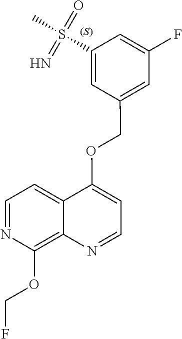 | (S)-(3-fluoro-5-(((8-(fluoromethoxy)-1,7-naphthyridin- 4-yl)oxy)methyl)phenyl)(imino)(methyl)-lambda6- sulfanone |
| 84 | 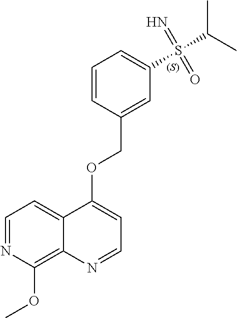 | (S)-imino(isopropyl)(3-(((8-methoxy-1,7-naphthyridin- 4-yl)oxy)methyl)phenyl)-lambda6-sulfanone |
| | M H+ = 372.15 | |

### EMBODIMENTS

### Embodiment A

In embodiment A, the compounds of Formula (I) or a pharmaceutically acceptable salt thereof is as defined in the Summary above.

### Embodiment B

(B) In embodiment B, the compounds of embodiment A, or a pharmaceutically acceptable salt thereof, are where Z is cyclylaminylene, spiro cyclylaminylene, or fused cyclylaminylene, each Z moiety is substituted with R⁴ and R⁵.

(Bi) In embodiment (Bi), the compounds of embodiments A and B, or a pharmaceutically acceptable salt thereof, are wherein Z is cyclylaminylene substituted with R⁴ and R⁵.

(Bii) In embodiment (Bii) of embodiment B, the compounds of embodiments A and B, or a pharmaceutically acceptable salt thereof, are wherein Z is spiro cyclylaminylene substituted with R⁴ and R⁵.

(Biii) In embodiment (Biii) of embodiment B, the compounds of embodiments A and B, or a pharmaceutically acceptable salt thereof, are wherein Z is fused cyclylaminylene substituted with R⁴ and R⁵.

(Biv) In embodiment (Biv) of embodiment B, the compounds of embodiments A, B, (Bi), (Bii), or (Biii), or a pharmaceutically acceptable salt thereof, are wherein the cyclylaminylene and spiro cyclylaminylene are attached to

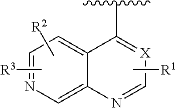

via nitrogen ring atom, wherein the wavy line is the point of attachment.

(Bv) In embodiment (Bv) of embodiment B, the compounds of embodiments A, B, (Bi), (Bii), (Biii), or (Biv), or a pharmaceutically acceptable salt thereof, are wherein the cyclylaminylene, spiro cyclylaminylene, or fused cyclylaminylene of Z are selected from:

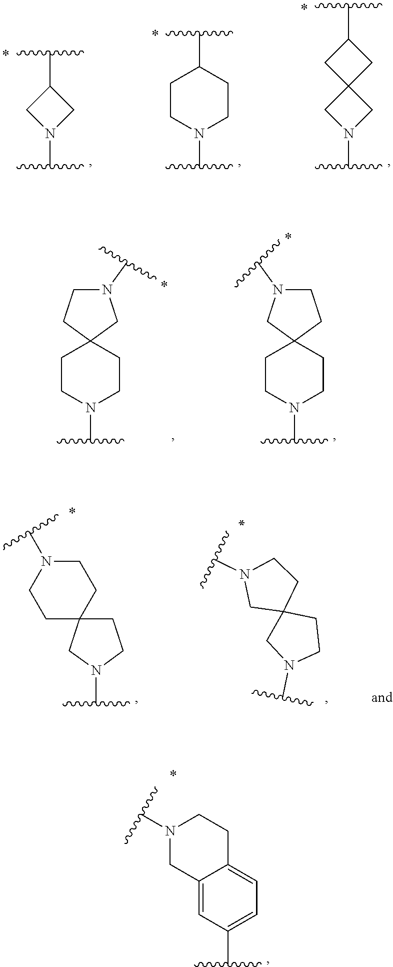

wherein each ring substituted with R⁴ and R⁵ and wherein

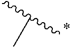

is the point of attachment to -(alk)ₘ-W and

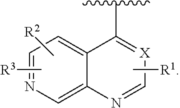

is the attachment to

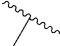

### Embodiment C

(C) In embodiment C, the compounds of embodiments A, or a pharmaceutically acceptable salt thereof, are those wherein Z is O, N (alkyl), or NH.

(Ci) In embodiment (Ci), the compounds of embodiments A and C, or a pharmaceutically acceptable salt thereof, are where Z is O.

(Cii) In embodiment (Cii), the compounds of embodiments A and C, or a pharmaceutically acceptable salt thereof, are wherein Z is NH or N (alkyl), preferably NH.

### Embodiment D

In embodiment D, the compounds of Embodiment A, or a pharmaceutically acceptable salt thereof, are wherein Z is S or SO₂.

### Embodiment E

In embodiment E, the compounds of any one of embodiments A, B, (Bi), (Bii), (Biii), (Biv), (Bv), C, (Ci), (Cii), and D, or a pharmaceutically acceptable salt thereof, are wherein X is N.

### Embodiment F

In embodiment F, the compound of any one of embodiments, A, B, (Bi), (Bii), (Biii), (Biv), (Bv), C, (Ci), (Cii), and D, or a pharmaceutically acceptable salt thereof, are wherein X is CH or CR¹.

It is understood that embodiment F, defining X is CH or CR¹, specifies that the R¹ moiety is at the X position and the carbon adjacent to X is CH. As such, the resulting chemical structure of the compound of embodiment F, or a pharmaceutically acceptable salt thereof, is according to Formula (Id)

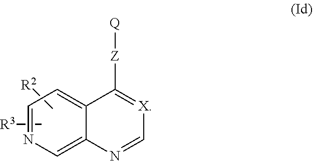

(Fi) In embodiment (Fi), the compound of Formula (Id) of embodiment F, or a pharmaceutically acceptable salt thereof, is wherein:
R¹ is cyano;
R² is absent, alkoxy, hydroxy, cycloalkoxy, or haloalkoxy; R³ is absent or alkoxy;
Z is cyclylaminylene, spiro cyclylaminylene, fused cyclylaminylene, NH, or O, wherein:
(a) when Z is cyclylaminylene, spiro cyclylaminylene, fused cyclylaminylene, then Q is -(alk)ₘ-W, wherein each Z moiety of (a) is substituted with R⁴ and R⁵ where R⁴ is absent and R⁵ is absent or hydroxy; and
(b) when Z is NH or O, then Q is -(alk₁)ₙ-Ar—W, wherein Ar is arylene substituted R⁶ and R⁷, wherein R₇ is absent; and R⁶ is absent or halo;
alk and alk¹ are independently alkylene;
m and n are independently 0 or 1;
W is formula (i):

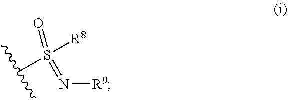

R⁸ is hydrogen, alkyl, or cycloalkyl; and
R⁹ is hydrogen or alkyl.

(Fii) In embodiment (Fii), the compound of Formula (Id) of embodiment F and Fi, or a pharmaceutically acceptable salt thereof, is wherein is wherein X is CH.

(Fiii) In embodiment (Fiii), the compound of Formula (Id) of embodiment F, Fi and Fii, or a pharmaceutically acceptable salt thereof, is wherein X is CR¹.

(Fiv) In embodiment (Fiv), the compound of Formula (Id) of embodiment F, Fi, Fii, and Fiii, or a pharmaceutically acceptable salt thereof, is wherein the compound of Formula (Id) is according to Formula (Id1)

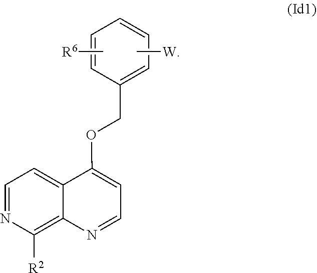

(Fv) In embodiment (Fv), the compound of Formula (Id) of embodiment F, Fi, Fii, and Fiii, or a pharmaceutically acceptable salt thereof, is wherein the compound of Formula (Id) is according to Formula (Id2)

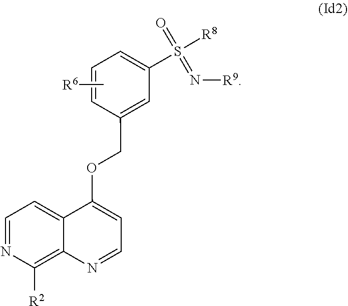

### Embodiment G

In embodiment G, the compounds of any one of embodiments A, B, (Bi), (Bii), (Biii), (Biv), (Bv), C, (Ci), (Cii), D, E, F, (Fi), (Fii), and (Fiii), or a pharmaceutically acceptable salt thereof, are wherein m is 0 and n is 0.

### Embodiment H

In embodiment H, the compounds of any one of embodiments A, B, (Bi), (Bii), (Biii), (Biv), (Bv), C, (Ci), (Cii), D, E, F, (Fi), (Fii), and (Fiii), or a pharmaceutically acceptable salt thereof, are wherein m is 1 and n is 1.

### Embodiment I

(Ii) In embodiment (Ii), the compounds of any one of Embodiments, A, B, (Bi), (Bii), (Biii), (Biv), (Bv), C, (Ci), (Cii), D, E, F, (Fi), (Fii), (Fiii), and H, or a pharmaceutically acceptable salt thereof, are wherein alk and alk¹ are independently methylene, ethylene, or propylene.

(Iii) In embodiment (Iii), the compounds of any one of Embodiments, A, B, (Bi), (Bii), (Biii), (Biv), (Bv), C, (Ci), (Cii), D, E, F, (Fi), (Fii), (Fiii), H, and (Ii), or a pharmaceutically acceptable salt thereof, are wherein alk and alk¹ are methylene.

### Embodiment J

In embodiment J, the compounds of any one of embodiments, A, C, (Ci), (Cii), D, E, F, (Fi), (Fii), (Fiii), G, H, (Ii) and (Iii), or a pharmaceutically acceptable salt thereof, are wherein Ar is phenylene or 5- or 6-membered heteroarylene substituted with R⁶ and R⁷.

(Ji). In embodiment (Ji), the compounds of any one of embodiments, A, C, (Ci), (Cii), D, E, F, (Fi), (Fii), (Fiii), G, H, (Ii), (Iii), and (J), or a pharmaceutically acceptable salt thereof, are wherein Ar is phenylene substituted with R⁶ and R⁷.

(Jii). In embodiment (Jii), the compounds of any one of embodiments, A, C, (Ci), (Cii), D, E, F, (Fi), (Fii), (Fiii), (Fiv), G, H, (Ii), (Iii), (J), and (Ji), or a pharmaceutically acceptable salt thereof, are wherein Ar is phenylene and W is attached to carbon of the phenylene ring that is meta to the carbon of the phenylene ring that is attached to -(alk¹)ₙ- of -Z-(alk¹)ₙ-.

(Jiii). In embodiment (Jiii), the compounds of any one of embodiments, A, C, (Ci), (Cii), D, E, F, (Fi), (Fii), (Fiii), (Fiv), G, H, (Ii), (Iii), (J), and (Ji), or a pharmaceutically acceptable salt thereof, are wherein Ar is phenylene and W is attached to carbon of the phenylene ring that is para to the carbon of the phenylene ring that is attached to -(alk¹)ₙ- of -Z-(alk¹)ₙ-.

(Jiv). In embodiment (Jiv), the compounds of any one of embodiments, A, C, (Ci), (Cii), D, E, F, (Fii), (Fiii), G, H, (Ii), (Iii), and (J), or a pharmaceutically acceptable salt thereof, are wherein Ar is heteroarylene.

(Jv). In embodiment (Jv), the compounds of any one of embodiments, A, C, (Ci), (Cii), D, E, F, (Fii), (Fiii), G, H, (Ii), (Iii), (J), and (Jiv), or a pharmaceutically acceptable salt thereof, are wherein are wherein the 5- or 6-membered heteroarylene of Ar is selected from divalent pyridinyl, pyrimidinyl, pyridazinyl, thienyl, furanyl, thiazolyl, oxazolyl, isoxazolyl, pyrazolyl, triazolyl, oxadiazolyl, and imidazolyl.

(Jvi). In embodiment (Jvi), the compounds of any one of embodiments, A, C, (Ci), (Cii), D, E, F, (Fii), (Fiii), G, H, (Ii), (Iii), (J), (Jiv), and (Jv), or a pharmaceutically acceptable salt thereof, are wherein are wherein the heteroarylene of Ar is a 6-membered ring such as divalent pyridinyl, pyrimidinyl, or pyridazinyl wherein W is attached to carbon on the divalent pyridinyl, pyrimidinyl, or pyridazinyl ring that is meta to the carbon attaching the divalent pyridinyl, pyrimidinyl, or pyridazinyl ring to -(alk¹)ₙ- of -Z-(alk¹)ₙ-.

(Jvii). In embodiment (Jvii), the compounds of any one of embodiments, A, C, (Ci), (Cii), D, E, F, (Fii), (Fiii), G, H, (Ii), (Iii), (J), (Jiv), and (Jv), or a pharmaceutically acceptable salt thereof, are wherein are wherein the heteroarylene of Ar is a 6-membered ring such as divalent pyridinyl, pyrimidinyl, or pyridazinyl wherein W is attached to carbon on the divalent pyridinyl, pyrimidinyl, or pyridazinyl ring that is para to the carbon attaching the divalent pyridinyl, pyrimidinyl, or pyridazinyl ring to -(alk¹)ₙ- of -Z-(alk¹)ₙ-.

### Embodiment K

(Ki) In embodiment K, the compounds of any one of embodiments A, B, (Bi), (Bii), (Biii), (Biv), (Bv), C, (Ci), (Cii), D, E, F, (Fi), (Fii), (Fiii), F (iv), G, H, (Ii), (Iii), (Ji), (Jii), (Jiii), (Jiv), (Jv), (Jvi), and (Jvii), or a pharmaceutically acceptable salt thereof, are wherein W is:

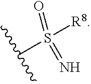

In a first subembodiment, of embodiment (Ki), W is:

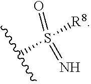

In a second subembodiment, of embodiment (Ki), W is:

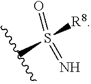

(Kii) In embodiment K, the compounds of any one of embodiments A, B, (Bi), (Bii), (Biii), (Biv), (Bv), C, (Ci), (Cii), D, E, F, (Fii), (Fiii), F (iv), G, H, (Ii), (Iii), (Ji), (Jii), (Jiii), (Jiv), (Jv), (Jvi), and (Jvii), or a pharmaceutically acceptable salt thereof, are wherein W is:

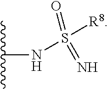

In a first subembodiment, of embodiment (Kii), W is:

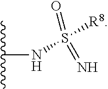

In a second subembodiment, of embodiment (Kii), W is:

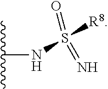

### Embodiment L

(Li) In embodiment (Li), the compounds of any one of embodiments A, B, (Bi), (Bii), (Biii), (Biv), (Bv), C, (Ci), (Cii), D, E, F, (Fiii), G, H, (Ii), (Iii), (Ji), (Jii), (Jiii), (Jiv), (Jv), (Jvi), (Jvii), (Ki) and (Kii), or a pharmaceutically acceptable thereof, are wherein R¹ is alkyl, halo, haloalkyl, haloalkoxy, or cyano. Reference of Ki and Kii here and embodiments below include subembodiments thereof.

(Lii) In embodiment (Lii), the compounds of any one of embodiments A, B, (Bi), (Bii), (Biii), (Biv), (Bv), C, (Ci), (Cii), D, E, F, (Fiii), G, H, (Ii), (Iii), (Ji), (Jii), (Jiii), (Jiv), (Jv), (Jvi), (Jvii), (Ki) and (Kii), or a pharmaceutically acceptable thereof, are wherein R¹ is methyl, ethyl, isopropyl, cyano, fluoro, chloro, difluoromethyl, trifluoromethyl, difluoromethoxy, or trifluoromethoxy.

(Liii) In embodiment (Liii), the compounds of any one of embodiments A, B, (Bi), (Bii), (Biii), (Biv), (Bv), C, (Ci), (Cii), D, E, F, (Fiii), G, H, (Ii), (Iii), (Ji), (Jii), (Jiii), (Jiv), (Jv), (Jvi), (Jvii), (Ki) and (Kii), or a pharmaceutically acceptable thereof, are wherein R¹ is amino, alkylamino, or dialkylamino, or R¹ is amino, methylamino or dimethylamino.

(Liv) In embodiment (Liv), the compounds of any one of embodiments A, B, (Bi), (Bii), (Biii), (Biv), (Bv), C, (Ci), (Cii), D, E, F, (Fiii), G, H, (Ii), (Iii), (Ji), (Jii), (Jiii), (Jiv), (Jv), (Jvi), (Jvii), (Ki), and (Kii), or a pharmaceutically acceptable thereof, are wherein R¹ is hydroxyalkyl, alkoxyalkyl, hydroxyalkoxy, alkoxyalkoxy, hydroxyalkylamino, alkoxyalkylamino, aminoalkyl, aminoalkoxy, aminoalkylamino, diaminoalkyl, diaminoalkoxy, or diaminoalkylamino.

(Lv) In embodiment (Lv), the compounds of any one of embodiments A, B, (Bi), (Bii), (Biii), (Biv), (Bv), C, (Ci), (Cii), D, E, F, (Fiii), G, H, (Ii), (Iii), (Ji), (Jii), (Jiii), (Jiv), (Jv), (Jvi), (Jvii), (Ki), and (Kii), or a pharmaceutically acceptable thereof, are wherein R¹ is cyano.

### Embodiment M

In embodiment M, the compounds of any one of embodiments A, B, (Bi), (Bii), (Biii), (Biv), (Bv), C, (Ci), (Cii), D, E, F, (Fi), (Fii), (Fiii), (Fiv), F (iv), G, H, (Ii), (Iii), (Ji), (Jii), (Jiii), (Jiv), (Jv), (Jv), (Jvi), (Jvii), (Ki), (Kii), (Li), (Lii), (Liii), (Liv), and (Lv), or a pharmaceutically acceptable thereof, are wherein R⁴, R⁵, R⁶, and R⁷ are independently absent, methyl, ethyl, methoxy, fluoro, trifluoromethyl, trifluoromethoxy, hydroxy, or cyano; or (a) R⁴ and R⁵ and R⁶, and R⁷ are independently absent, methyl, ethyl, hydroxy, fluoro, or chloro, unless stated otherwise. In a subembodiment of embodiment M, R⁴ and R⁵ and R⁶, and R⁷ are independently absent or fluoro unless stated otherwise. In a subembodiment of embodiment M, R⁴ and R⁵ and R⁶, and R⁷ are independently absent or hydroxy unless stated otherwise. In a subembodiment of embodiment M, R⁴ and R⁵ and R⁶, and R⁷ are absent.

### Embodiment N

(Ni) In embodiment (Ni), the compounds of any one of embodiments A, B, (Bi), (Bii), (Biii), (Biv), (Bv), C, (Ci), (Cii), D, E, F, (Fi), (Fii), (Fiii), (Fiv), F (iv), G, H, (Ii), (Iii), (Ji), (Jii), (Jiii), (Jiv), (Jv), (Jvi), (Jvii), (Ki), (Kii), (Li), (Lii), (Liii), (Liv), and (M), or a pharmaceutically acceptable thereof, are wherein R² is absent, alkyl, hydroxy, alkoxy, halo, haloalkyl, haloalkoxy, cyano, aminocarbonyl, alkylaminocarbonyl, or dialkylaminocarbonyl unless stated otherwise.

(Nii) In subembodiment (Nii), the compounds of any one of embodiments A, B, (Bi), (Bii), (Biii), (Biv), (Bv), C, (Ci), (Cii), D, E, F, (Fi), (Fii), (Fiii), (Fiv), F (iv), G, H, (Ii), (Iii), (Ji), (Jii), (Jiii), (Jiv), (Jv), (Jvi), (Jvii), (Ki), (Kii), (Li), (Lii), (Liii), (Liv), (M), and (Ni), or a pharmaceutically acceptable salt thereof, are wherein R² is absent, methyl, hydroxy, methoxy, ethoxy, fluoro, chloro, trifluoromethyl, cyano, trifluoromethyl, aminocarbonyl, methylaminocarbonyl, or dimethylaminocarbonyl unless stated otherwise.

(Niii) In subembodiment (Niii), the compounds of any one of embodiments A, B, (Bi), (Bii), (Biii), (Biv), (Bv), C, (Ci), (Cii), D, E, F, (Fi), (Fii), (Fiii), (Fiv), F (iv), G, H, (Ii), (Iii), (Ji), (Jii), (Jiii), (Jiv), (Jv), (Jvi), (Jvii), (Ki), (Kii), (Li), (Lii), (Liii), (Liv), (M), (Ni), and (Nii), or a pharmaceutically acceptable salt thereof, wherein R² is absent, methoxy, ethoxy, aminocarbonyl, or cyano unless stated otherwise, preferably methoxy. In a further subembodiment of embodiment (Niii), R² is absent, methoxy, or ethoxy.

(Niv) In subembodiment (Niv), the compounds of any one of embodiments A, B, (Bi), (Bii), (Biii), (Biv), (Bv), C, (Ci), (Cii), D, E, F, (Fi), (Fii), (Fiii), (Fiv), F (iv), G, H, (Ii), (Iii), (Ji), (Jii), (Jiii), (Jiv), (Jv), (Jvi), (Jvii), (Ki), (Kii), (Li), (Lii), (Liii), (Liv), and (M), or a pharmaceutically acceptable salt thereof, are wherein R² is absent, alkoxy, haloalkoxy, or cycloalkyloxy.

(Nv) In subembodiment (Nv), the compounds of any one of embodiments A, B, (Bi), (Bii), (Biii), (Biv), (Bv), C, (Ci), (Cii), D, E, F, (Fi), (Fii), (Fiii), (Fiv), F (iv), G, H, (Ii), (Iii), (Ji), (Jii), (Jiii), (Jiv), (Jv), (Jvi), (Jvii), (Ki), (Kii), (Li), (Lii), (Liii), (Liv), and (M), or a pharmaceutically acceptable salt thereof, are wherein R² is absent, methoxy, fluoromethoxy, ethoxy, isopropyloxy, n-propyloxy, aminocarbonyl, cyano, or cyclopropyloxy, unless stated otherwise.

(Nvi) In embodiment (Nvi), the compounds of any one of embodiments A, B, (Bi), (Bii), (Biii), (Biv), (Bv), C, (Ci), (Cii), D, E, F, (Fi), (Fii), (Fiii), (Fiv), F (iv), G, H, (Ii), (Iii), (Ji), (Jii), (Jiii), (Jiv), (Jv), (Jvi), (Jvii), (Ki), (Kii), (Li), (Lii), (Liii), (Liv), and (M), or a pharmaceutically acceptable thereof, are wherein R² is absent, methoxy, fluoromethoxy, ethoxy, isopropyloxy, n-propyloxy, or cyclopropyloxy.

(Nvii) In subembodiment (Nvii), the compounds of any one of embodiments A, B, (Bi), (Bii), (Biii), (Biv), (Bv), C, (Ci), (Cii), D, E, F, (Fi), (Fii), (Fiii), G, H, (Ii), (Iii), (Ji), (Jii), (Jiii), (Jiv), (Jv), (Jvi), (Jvii), (Ki), (Kii), (Li), (Lii), (Liii), (Liv), (M), (Ni), (Nii), and (Niii), or a pharmaceutically acceptable salt thereof, are wherein the compound of Formula (I) is a compound of Formula (Ia), (Ib), or (Ib1):

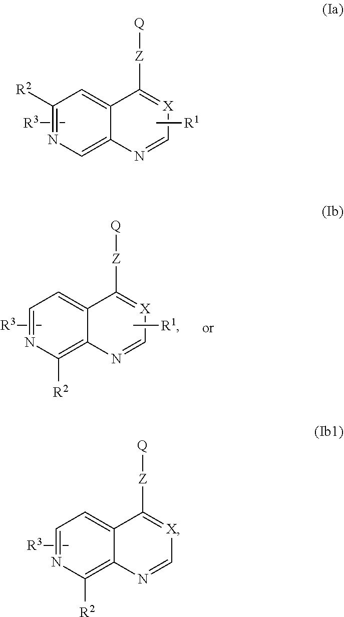

where R², R³, Z, Q, and R¹ are as defined therein.

(Nviii) In subembodiment (Nviii), the compounds of any one of embodiments A, B, (Bi), (Bii), (Biii), (Biv), (Bv), C, (Ci), (Cii), D, E, F, (Fi), (Fii), (Fiii), F (iv), F (v), G, H, (Ii), (Iii), (Ji), (Jii), (Jiii), (Jiv), (Jv), (Jvi), (Jvii), (Ki), (Kii), (Li), (Lii), (Liii), (Liv), (M), (Ni), (Nii), (Niii), (Niv), (Nv), (Nvi), and (Nvii), or a pharmaceutically acceptable salt thereof, are wherein R³ is absent.

(Nix) In embodiment (Nix), the compounds of any one of embodiments A, B, (Bi), (Bii), (Biii), (Biv), (Bv), C, (Ci), (Cii), D, E, F, (Fi), (Fii), (Fiii), F (iv), F (v), G, H, (Ii), (Iii), (Ji), (Jii), (Jiii), (Jiv), (Jv), (Jvi), (Jvii), (Ki), (Kii), (Li), (Lii), (Liii), (Liv), (M), N (vii), and N (viii), or a pharmaceutically acceptable thereof, are wherein R² is absent, haloalkoxy, or cycloalkyloxy.

(Nx) In embodiment (Nx), the compounds of any one of embodiments A, B, (Bi), (Bii), (Biii), (Biv), (Bv), C, (Ci), (Cii), D, E, F, (Fi), (Fii), (Fiii), F (iv), F (v), G, H, (Ii), (Iii), (Ji), (Jii), (Jiii), (Jiv), (Jv), (Jvi), (Jvii), (Ki), (Kii), (Li), (Lii), (Liii), (Liv), (M), N (vii), N (viii), and (Nix), or a pharmaceutically acceptable thereof, are wherein R² is absent, fluoromethoxy, difluoromethoxy, or cycloalkyloxy.

### Embodiment O

(Oi) In embodiment (Oi), the compounds of any one of embodiments A, B, (Bi), (Bii), (Biii), (Biv), (Bv), C, (Ci), (Cii), D, E, F, (Fi), (Fii), (Fiii), F (iv), F (v), G, H, (Ii), (Iii), (Ji), (Jii), (Jiii), (Jiv), (Jv), (Jvi), (Jvii), (Ki), (Kii), (Li), (Lii), (Liii), (Liv), and (M), or a pharmaceutically acceptable thereof, are wherein R² and R³ are independently absent, alkyl, alkoxy, hydroxy, halo, haloalkyl, or haloalkoxy unless stated otherwise.

(Oii). In embodiment (Oii), the compounds of any one of embodiments A, B, (Bi), (Bii), (Biii), (Biv), (Bv), C, (Ci), (Cii), D, E, F, (Fi), (Fii), (Fiii), F (iv), F (v), G, H, (Ii), (Iii), (Ji), (Jii), (Jiii), (Jiv), (Jv), (Jvi), (Jvii), (Ki), (Kii), (Li), (Lii), (Liii), (Liv), (M), and (Oi) are those wherein R² and R³ are independently, alkoxy or halo, preferably R² and R³ are independently methoxy, ethoxy, fluoro, and chloro unless stated otherwise.

(Oiii). In embodiment (Oiii), the compounds of any one of embodiments A, B, (Bi), (Bii), (Biii), (Biv), (Bv), C, (Ci), (Cii), D, E, F, (Fi), (Fii), (Fiii), F (iv), F (v), G, H, (Ii), (Iii), (Ji), (Jii), (Jiii), (Jiv), (Jv), (Jvi), (Jvii), (Ki), (Kii), (Li), (Lii), (Liii), (Liv), and (M), or a pharmaceutically acceptable thereof, are wherein:

R² is absent, alkyl, alkoxy, hydroxy, halo, haloalkyl, or haloalkoxy; and

R³ is hydroxyalkyl, alkoxyalkyl, hydroxyalkoxy, alkoxyalkoxy, hydroxyalkylamino, alkoxyalkylamino, aminoalkyl, aminoalkoxy, aminoalkylamino, heterocyclyl, heterocyclyloxy, heterocyclylamino (wherein heterocyclyl either alone or part of heterocyclyloxy and heterocyclylamino is optionally substituted with one, two, or three substituents independently selected from alkyl, halo, hydroxy, alkoxy, hydroxyalkyl, alkoxyalkyl, and aminoalkyl), heterocyclylalkyl, heterocyclylalkyloxy, heterocyclylalkylamino (wherein the heterocyclyl ring in heterocyclylalkyl, heterocyclylalkyloxy, and heterocyclylalkylamino is optionally substituted with one, two, or three substituents independently selected from alkyl, halo, hydroxy, alkoxy, hydroxyalkyl, alkoxyalkyl, and aminoalkyl), cycloalkyloxy, phenyloxy, or heteroaryloxy (where phenyl in phenyloxy and heteroaryl in heteroaryloxy are optionally substituted with one, two, or three substituents where two of the optional substituents are independently selected from alkyl, hydroxy, alkoxy, halo, haloalkyl, haloalkoxy, and cyano) unless stated otherwise.

(Oiv). In embodiment (Oiv), the compounds of any one of embodiments A, B, (Bi), (Bii), (Biii), (Biv), (Bv), C, (Ci), (Cii), D, E, F, (Fi), (Fii), (Fiii), F (iv), F (v), G, H, (Ii), (Iii), (Ji), (Jii), (Jiii), (Jiv), (Jv), (Jv), (Jvi), (Jvii), (Ki), (Kii), (Li), (Lii), (Liii), (Liv), (M), and (Oii), or a pharmaceutically acceptable salt thereof is wherein R² is absent, methoxy, ethoxy, or hydroxy, preferably R² is methoxy or ethoxy; and R³ is 2-hydroxyethyloxy, 3-hydroxypropyloxy, 2-methoxyethyloxy, 2-ethoxyethyloxy, 3-methoxypropyloxy, 3-ethoxypropyloxy, 2-aminoethyloxy, 2-methylaminoethyloxy, 2-dimethylaminoethyloxy, 2-diethylaminoethyloxy, 3-aminopropyloxy, 3-methylaminopropyloxy, 3-dimethylaminopropyloxy, 3-diethylaminopropyloxy, pyrrolidinyloxy, piperidinyloxy, pyrrolidinylmethyloxy, piperidinylmethyloxy, pyrrolidinylethyloxy, piperidinylethyloxy, 2-hydroxyethylamino, 3-hydroxypropylamino, 2-methoxyethylamino, 2-ethoxyethylamino, 3-methoxypropylamino, 3-ethoxypropylamino, 2-aminoethylamino, 2-methylaminoethylamino, 2-dimethylaminoethylamino, 2-diethylaminoethylamino, 3-aminopropylamino, 3-methylaminopropylamino, 3-dimethylaminopropylamino, 3-diethylaminopropylamino, pyrrolidinylamino, piperidinylamino, pyrrolidinylmethylamino, piperidinylmethylamino, pyrrolidinylethylamino, or piperidinylethylamino (wherein pyrrolidinyl and piperidinyl in each of aforementioned groups, alone or part of another group is optionally substituted with one or two substituents independently selected from methyl, fluoro, hydroxy, or methoxy) unless stated otherwise.

(Ov). In embodiment (Ov), the compounds of any one of embodiments A, B, (Bi), (Bii), (Biii), (Biv), (Bv), C, (Ci), (Cii), D, E, F, (Fi), (Fii), (Fiii), F (iv), F (v), G, H, (Ii), (Iii), (Ji), (Jii), (Jiii), (Jiv), (Jv), (Jvi), (Jvii), (Ki), (Kii), (Li), (Lii), (Liii), (Liv), and (M) or a pharmaceutically acceptable thereof are those wherein R² and R³ are independently hydroxyalkyl, alkoxyalkyl, hydroxyalkoxy, alkoxyalkoxy, hydroxyalkylamino, alkoxyalkylamino, aminoalkyl, aminoalkoxy, aminoalkylamino, heterocyclyl, heterocyclyloxy, heterocyclylamino (wherein heterocyclyl either alone or part of heterocyclyloxy and heterocyclylamino is optionally substituted with one, two, or three substituents independently selected from alkyl, halo, hydroxy, alkoxy, hydroxyalkyl, alkoxyalkyl, and aminoalkyl), heterocyclylalkyl, heterocyclylalkyloxy, heterocyclylalkylamino (wherein the heterocyclyl ring in heterocyclylalkyl, heterocyclylalkyloxy, and heterocyclylalkylamino is optionally substituted with one, two, or three substituents independently selected from alkyl, halo, hydroxy, alkoxy, hydroxyalkyl, alkoxyalkyl, and aminoalkyl), cycloalkyloxy, phenyloxy, or heteroaryloxy (where phenyl of phenyloxy and heteroaryl of heteroaryloxy are optionally substituted with one, two, or three substituents independently selected from alkyl, hydroxy, alkoxy, halo, haloalkyl, haloalkoxy, and cyano) unless stated otherwise

In a first subembodiment of embodiment (Ov), R² and R³ are independently 2-hydroxyethyloxy, 3-hydroxypropyloxy, 2-methoxyethyloxy, 2-ethoxyethyloxy, 3-methoxypropyloxy, 3-ethoxypropyloxy, 2-aminoethyloxy, 2-methylaminoethyloxy, 2-dimethylaminoethyloxy, 2-diethylaminoethyloxy, 3-aminopropyloxy, 3-methylaminopropyloxy, 3-dimethylaminopropyloxy, 3-diethylaminopropyloxy, pyrrolidinyloxy, piperidinyloxy, pyrrolidinylmethyloxy, piperidinylmethyloxy, pyrrolidinylethyloxy, piperidinylethyloxy, 2-hydroxyethylamino, 3-hydroxypropylamino, 2-methoxyethylamino, 2-ethoxyethylamino, 3-methoxypropylamino, 3-ethoxypropylamino, 2-aminoethylamino, 2-methylaminoethylamino, 2-dimethylaminoethylamino, 2-diethylaminoethylamino, 3-aminopropylamino, 3-methylaminopropylamino, 3-dimethylaminopropylamino, 3-diethylaminopropylamino, pyrrolidinylamino, piperidinylamino, pyrrolidinylmethylamino, piperidinylmethylamino, pyrrolidinylethylamino, or piperidinylethylamino (wherein pyrrolidinyl and piperidinyl in each of aforementioned groups, alone or part of another group is optionally substituted with one or two substituents independently selected from methyl, fluoro, hydroxy, or methoxy).

(Ovi) In subembodiment (Ovi), the compounds of any one of embodiments A, B, (Bi), (Bii), (Biii), (Biv), (Bv), C, (Ci), (Cii), D, E, F, (Fi), (Fii), (Fiii), (Fiv), G, H, (Ii), (Iii), (Ji), (Jii), (Jiii), (Jiv), (Jv), (Jv), (Jvi), (Jvii), (Ki), (Kii), (Li), (Lii), (Liii), (Liv), (M), (Oi), (Oii), (Oiii), (Oiv), and (Ov), or a pharmaceutically acceptable salt thereof, are wherein the compound of Formula (I) is a compound of Formula (Ic) or (Ic1):

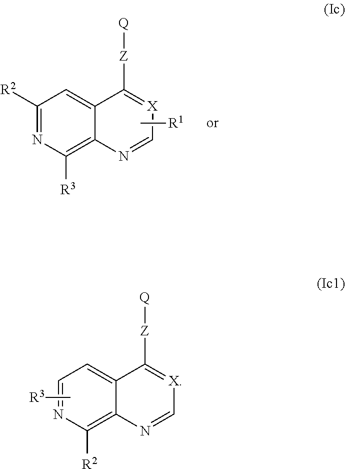

### Embodiment P

(Pi) In subembodiment (Pi), the compounds of any one of embodiments A, B, (Bi), (Bii), (Biii), (Biv), (Bv), C, (Ci), (Cii), D, E, F, (Fi), (Fii), (Fiii), (Fiv), F (v), G, H, (Ii), (Iii), (Ji), (Jii), (Jiii), (Jiv), (Jv), (Jvi), (Jvii), (Ki), (Kii), (Li), (Lii), (Liii), (Liv), (M), (Oi), (Oii), (Oiii), (Oiv), (Ov), and (Ovi), or a pharmaceutically acceptable salt thereof, are wherein each R⁸ is alkyl, substituted alkyl, haloalkyl, substituted haloalkyl, cycloalkyl, substituted cycloalkyl, or cycloalkylalkyl.

(Pii) In subembodiment (Pii), the compounds of any one of embodiments A, B, (Bi), (Bii), (Biii), (Biv), (Bv), C, (Ci), (Cii), D, E, F, (Fi), (Fii), (Fiii), (Fiv), F (v), G, H, (Ii), (Iii), (Ji), (Jii), (Jiii), (Jiv), (Jv), (Jvi), (Jvii), (Ki), (Kii), (Li), (Lii), (Liii), (Liv), (M), (Oi), (Oii), (Oiii), (Oiv), (Ov), (Ovi), and (Pi), or a pharmaceutically acceptable salt thereof, are wherein each R⁸ is methyl, ethyl, isopropyl, cyclopropyl, cyclobutyl, cyclopentyl, cyclohexyl, cyclopropylmethyl, or cyclobutylmethyl.

(Piii) In subembodiment (Piii), the compounds of any one of embodiments A, B, (Bi), (Bii), (Biii), (Biv), (Bv), C, (Ci), (Cii), D, E, F, (Fi), (Fii), (Fiii), (Fiv), F (v), G, H, (Ii), (Iii), (Ji), (Jii), (Jiii), (Jiv), (Jv), (Jvi), (Jvii), (Ki), (Kii), (Li), (Lii), (Liii), (Liv), (M), (Oi), (Oii), (Oiii), (Oiv), (Ov), (Ovi), and (Pi), or a pharmaceutically acceptable salt thereof, are wherein R⁸ is alkyl or cycloalkyl.

(Piv) In subembodiment (Piv), the compounds of any one of embodiments A, B, (Bi), (Bii), (Biii), (Biv), (Bv), C, (Ci), (Cii), D, E, F, (Fi), (Fii), (Fiii), (Fiv), F (v), G, H, (Ii), (Iii), (Ji), (Jii), (Jiii), (Jiv), (Jv), (Jvi), (Jvii), (Ki), (Kii), (Li), (Lii), (Liii), (Liv), (M), (Oi), (Oii), (Oiii), (Oiv), (Ov), (Ovi), (Pi), (Pii), and (Piii), or a pharmaceutically acceptable salt thereof, are wherein R⁸ is methyl or cyclopropyl.

(Pv) In subembodiment (Pv), the compounds of any one of embodiments A, B, (Bi), (Bii), (Biii), (Biv), (Bv), C, (Ci), (Cii), D, E, F, (Fi), (Fii), (Fiii), (Fiv), F (v), G, H, (Ii), (Iii), (Ji), (Jii), (Jiii), (Jiv), (Jv), (Jvi), (Jvii), (Li), (Lii), (Liii), (Liv), (M), (Oi), (Oii), (Oiii), (Oiv), (Ov), (Ovi), (Pi), (Pii), (Piii), (Piv) or a pharmaceutically acceptable salt thereof, are wherein R⁹ is hydrogen.

### Embodiment Q

In embodiment (Q), the compounds of Formula (I) are selected from Table 1 or a pharmaceutically acceptable salt thereof.

### General Synthetic Scheme

Compounds of this disclosure can be made by the methods depicted in the reaction schemes shown below.

The starting materials and reagents used in preparing these compounds are either available from commercial suppliers such as Aldrich Chemical Co., (Milwaukee, Wis.), Bachem (Torrance, Calif.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), March's Advanced Organic Chemistry, (John Wiley and Sons, 4th Edition) and Larock's Comprehensive Organic Transformations (VCH Publishers Inc., 1989). These schemes are merely illustrative of some methods by which the compounds of this disclosure can be synthesized, and various modifications to these schemes can be made and will be suggested to one skilled in the art reading this disclosure. The starting materials and the intermediates, and the final products of the reaction may be isolated and purified if desired using conventional techniques, including but not limited to filtration, distillation, crystallization, chromatography and the like. Such materials may be characterized using conventional means, including physical constants and spectral data.

Unless specified to the contrary, the reactions described herein take place at atmospheric pressure over a temperature range from about −78° C. to about 150° C., such as from about 0° C. to about 125° C. and further such as at about room (or ambient) temperature, e.g., about 20° C.

Compounds of Formula (I) or (Id) where Z is cyclylaminylene, spiro cyclylaminylene, or fused cyclylaminylene attached to core ring via ring nitrogen atom, Q is -(alk) m-W where W is a group consisting of formula (i), and other groups are as defined in compound of Formula (I) or (Id) in the Summary can be prepared as illustrated and described in Scheme 1 below (for sake of convenience only Formula (I) is depicted).

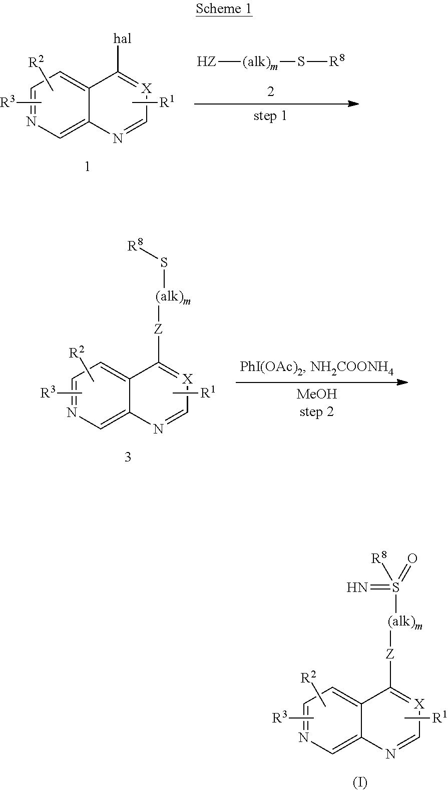

Reaction of a compound of formula 1 where hal is halo such as chloro, X is CH, N, R¹, R⁴, R⁵, and R⁶ as defined in the Summary, with a compound of the formula 2 where Z is, cyclylaminylene, spiro cyclylaminylene, or fused cyclylaminylene attached to ring 1 via ring nitrogen atom and (alk)ₘ, and R⁸ are as defined in the Summary provides a sulfide of compound of formula 3. The reaction is carried out under using an organic base such as DIEA, TEA, and the like, or an inorganic base, in aprotic organic solvents such as NMP, 1,3-dioxolane, TOU ((2,5,7,10-tetraoxaundecane), DMSO, DMPU, HMA, 1,4-dioxane, tetrahydrofuran (THF), dimethylformamide (DMF) and the like, either at room temperature or heating. Compounds of formula 1 such as 4-chloro-1,7-naphthyridine or 4-chloro-8-methoxypyrido[3,4-d]pyrimidine are commercially available. Compounds of formula 1 may also be obtained by treating a hydroxy compound such as 8-methoxy-1,7-naphthyridin-4-ol with a chlorinating agent such as P(O)Cl₃ in aprotic solvents with an organic aprotic base either with heating or room temperature. Compound of formula 3 is converted into a compound of Formula (I) by treatment with ammonium carbamate and (diacetoxyiodo)benzene in methanol.

Compounds of Formula (I) or (Id) where Z is NH, N (alkyl), O, S, SO, or SO₂, Q is -(alk¹)ₙ-Ar—W, wherein W is a group consisting of formula (i), and other groups are as defined in Formula (I) or (Id) in the Summary can be prepared as illustrated and described in Scheme 2 below (for sake of convenience only Formula (I) is depicted) . . .

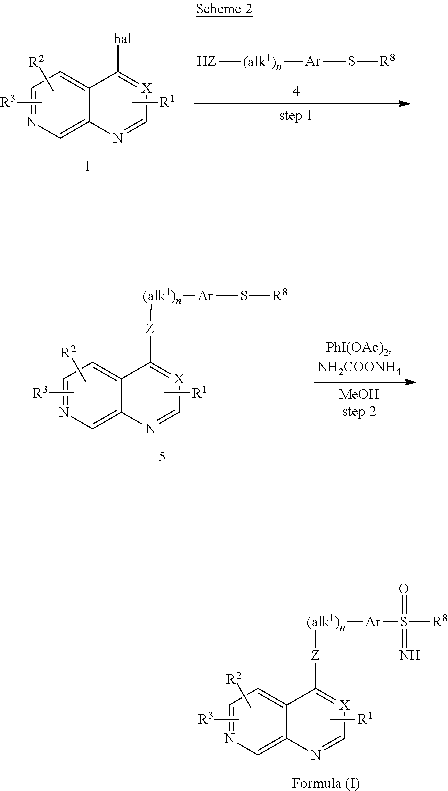

Proceeding as described in Scheme 1 above but replacing compound 2 with compound 4, provides a compound of Formula (I) where Z is NH, N (alkyl), O, S, SO, or SO₂, Q is -(alk)ₙ-Ar—W where W is a group consisting of Formula (I).

Compounds of Formula (I) or (Id) where where Z is cyclylaminylene, spiro cyclylaminylene, or fused cyclylaminylene attached to core ring via ring nitrogen atom, Q is -(alk)ₘ-W where W is a group consisting of formula (ii), and other groups are as defined in Formula (I) or (Id) the Summary can be prepared as illustrated and described in Scheme 3 below (for sake of convenience only Formula (I) is depicted) . . .

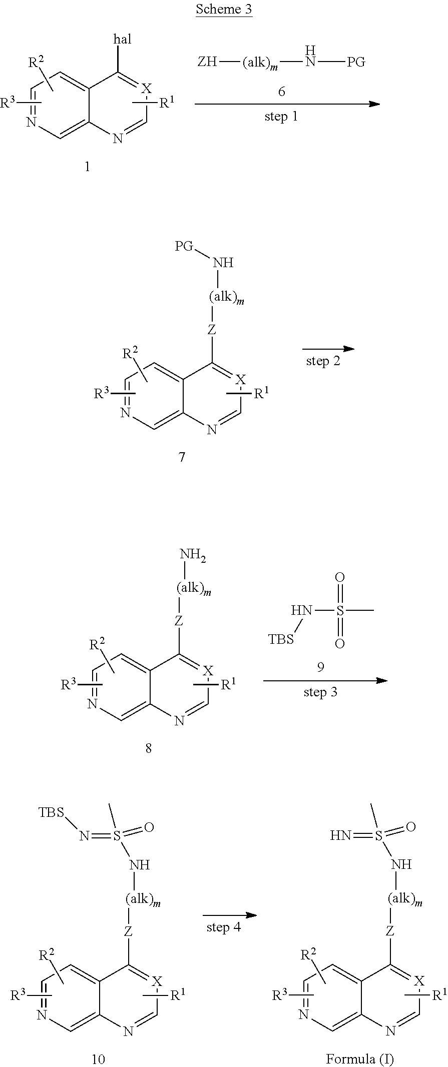

Treatment of a compound of formula 1 with a compound of formula 6, where Z is, cyclylaminylene, spiro cyclylaminylene, or fused cyclylaminylene attached to ring 1 via ring nitrogen atom and (alk)ₘ is as defined in the Summary, and PG is a suitable amino protecting group, in the presence of a base such as DIEA, CsCO₃, or TEA in a suitable organic solvent such as DMF or DMP, either at room temperature or elevated temperature provides a compound of formula 7. Treatment of compound 7 with a mineral or Lewis acid such as TMSOTf, HCl, or TFA in a suitable organic solvent provides an amino compound of formula 8. Compounds of a formula 8 are treated with a preparation of a compound of formula 9 that has been treated with (diphenylphosphoryl)benzene and oxalyl chloride and DIEA or another suitable organic base such as TEA, to provide a compound formula 10. Treatment of compound 10 with TFA or other suitable acid provides compounds of Formula (I).

Chiral sulfoximine compounds of Formula (I) or (Id) where Z is NH, N (alkyl), O, S, SO, or SO₂ (or an embodiment thereof); Q is -(alk¹)-Ar—W, where W is a group consisting of formula (i); X is CH; and other groups are as defined in Formula (I) or (Id) the Summary respectively (or an embodiment thereof) can be prepared as illustrated and described in Scheme 4 below (for sake of convenience only Formula (Id) is depicted in Scheme 4).

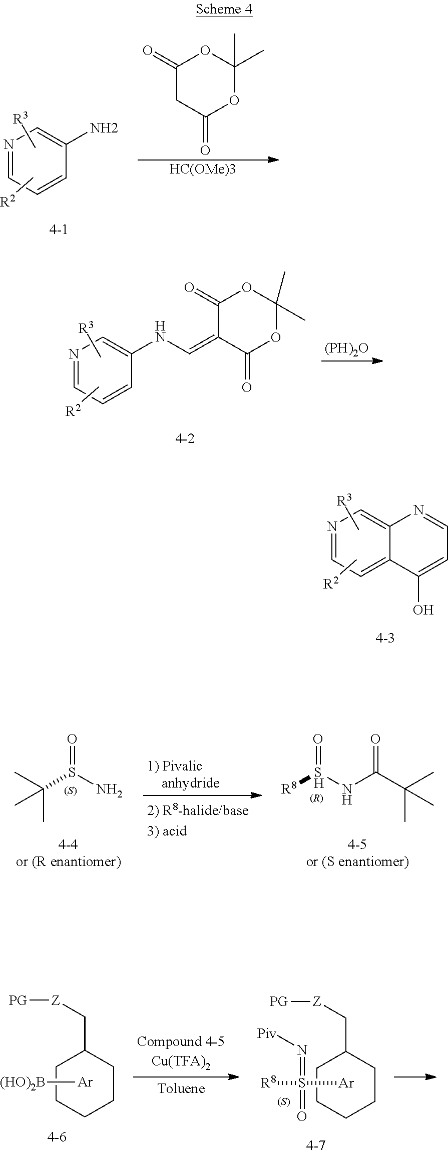

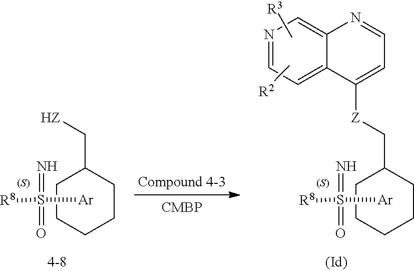

Treatment of a compound of formula 4-1 where R² and R³ are as defined in the Summary (or an embodiment thereof) with 2,2-dimethyl-1,3-dioxane-4,6-dione in the presence of trimethoxymethane provides a compound of formula 4-2. Compound 4-2 is cyclized in diphenyl ether at high temperatures such as about 220° C. to provide a compound of formula 4-3.

Treatment of(S)-2-methylpropane-2-sulfinamide, compound 4-4, with pivalic anhydride, followed by treatment with a compound of formula R⁸-halide where R⁸ is as defined in the Summary (or an embodiment thereof) in the presence of a deprotonating base such as sodium hydride, followed by treatment with an acid such as trifluoroacetic acid, provides a compound of formula 4-5.

Treatment of a boronic acid of compound 4-6, where Ar is as defined in the summary (or an embodiment thereof), Z is NH, N (alkyl), O, S, SO, or SO₂ (or an embodiment thereof), and PG is a suitable amino or oxygen protecting group with a compound of formula 4-5 in the presence of copper triflate in a suitable organic solvent such as toluene provides a compound of formula 4-7. Removal of the pivaloyl and the PG group by methods know in the art provides a benzylmethanol compound of formula 4-8. For example, when the pivaloyl group can be removed upon treatment of compound 4-7 with an inorganic base such as sodium hydroxide, potassium hydroxide, and the like in an organic alcohol solvent such as methanol, ethanol, and the like. For example, if the protecting group is tetrabutyldimethyl silyl, it can be removed by treatment of an acid such as TBAF in a suitable organic solvent such as tetrahydrofuran.

Coupling of a compound of formula 4-8 with a compound of formula 4-3 under Mitsunobu reaction conditions than provides a S sulfoximine compound of Formula (Id). It will be apparent to a person skilled in the art that R-sulfoximine of Formula (Id) can be prepared by using (R)-2-methylpropane-2-sulfinamide instead of(S)-2-methylpropane-2-sulfinamide.

### Testing

The ENPP1 inhibitory activity of the compounds of the present disclosure can be tested using the in vitro and in vivo assays described in Biological Examples 1 and 2 below.

### Administration and Pharmaceutical Composition

In general, the compounds of this disclosure will be administered in a therapeutically effective amount by any of the accepted modes of administration for agents that serve similar utilities. Therapeutically effective amounts of compounds this disclosure may range from about 0.01 to about 500 mg per kg patient body weight per day, which can be administered in single or multiple doses. A suitable dosage level may be from about 0.1 to about 250 mg/kg per day; about 0.5 to about 100 mg/kg per day. A suitable dosage level may be about 0.01 to about 250 mg/kg per day, about 0.05 to about 100 mg/kg per day, or about 0.1 to about 50 mg/kg per day. Within this range the dosage can be about 0.05 to about 0.5, about 0.5 to about 5 or about 5 to about 50 mg/kg per day. For oral administration, the compositions can be provided in the form of tablets containing about 1.0 to about 1000 milligrams of the active ingredient, particularly about 1, 5, 10, 15, 20, 25, 50, 75, 100, 150, 200, 250, 300, 400, 500, 600, 750, 800, 900, and 1000 milligrams of the active ingredient. The actual amount of the compound of this disclosure, i.e., the active ingredient, will depend upon numerous factors such as the severity of the disease to be treated, the age and relative health of the patient, the potency of the compound being utilized, the route and form of administration, and other factors.

In general, compounds of this disclosure will be administered as pharmaceutical compositions by any one of the following routes: oral, systemic (e.g., transdermal, intranasal or by suppository), or parenteral (e.g., intramuscular, intravenous or subcutaneous) administration. The preferred manner of administration is oral using a convenient daily dosage regimen, which can be adjusted according to the degree of affliction. Compositions can take the form of tablets, pills, capsules, semisolids, powders, sustained release formulations, solutions, suspensions, elixirs, aerosols, or any other appropriate compositions.

The choice of formulation depends on various factors such as the mode of drug administration (e.g., for oral administration, formulations in the form of tablets, pills or capsules, including enteric coated or delayed release tablets, pills or capsules are preferred) and the bioavailability of the drug substance. Recently, pharmaceutical formulations have been developed especially for drugs that show poor bioavailability based upon the principle that bioavailability can be increased by increasing the surface area i.e., decreasing particle size. For example, U.S. Pat. No. 4,107,288 describes a pharmaceutical formulation having particles in the size range from 10 to 1,000 nm in which the active material is supported on a cross-linked matrix of macromolecules. U.S. Pat. No. 5,145,684 describes the production of a pharmaceutical formulation in which the drug substance is pulverized to nanoparticles (average particle size of 400 nm) in the presence of a surface modifier and then dispersed in a liquid medium to give a pharmaceutical formulation that exhibits remarkably high bioavailability.

The compositions are comprised of in general, a compound of this disclosure in combination with at least one pharmaceutically acceptable excipient. Acceptable excipients are non-toxic, aid administration, and do not adversely affect the therapeutic benefit of the compound of this disclosure. Such excipient may be any solid, liquid, semi-solid or, in the case of an aerosol composition, gaseous excipient that is generally available to one of skill in the art.

Solid pharmaceutical excipients include starch, cellulose, talc, glucose, lactose, sucrose, gelatin, malt, rice, flour, chalk, silica gel, magnesium stearate, sodium stearate, glycerol monostearate, sodium chloride, dried skim milk and the like. Liquid and semisolid excipients may be selected from glycerol, propylene glycol, water, ethanol and various oils, including those of petroleum, animal, vegetable or synthetic origin, e.g., peanut oil, soybean oil, mineral oil, sesame oil, etc. Preferred liquid carriers, particularly for injectable solutions, include water, saline, aqueous dextrose, and glycols.

Compressed gases may be used to disperse a compound of this disclosure in aerosol form. Inert gases suitable for this purpose are nitrogen, carbon dioxide, etc.

Other suitable pharmaceutical excipients and their formulations are described in Remington's Pharmaceutical Sciences, edited by E. W. Martin (Mack Publishing Company, 20th ed., 2000).

The level of the compound in a formulation can vary within the full range employed by those skilled in the art. Typically, the formulation will contain, on a weight percent (wt. %) basis, from about 0.01-99.99 wt. % of a compound of this disclosure based on the total formulation, with the balance being one or more suitable pharmaceutical excipients. For example, the compound is present at a level of about 1-80 wt. %.

The compounds of this disclosure may be used in combination with one or more other drugs in the treatment of diseases or conditions for which compounds of this disclosure or the other drugs may have utility. Such other drug(s) may be administered, by a route and in an amount commonly used therefore, contemporaneously or sequentially with a compound of the present disclosure. When a compound of this disclosure is used contemporaneously with one or more other drugs, a pharmaceutical composition in unit dosage form containing such other drugs and the compound of the present disclosure is preferred. However, the combination therapy may also include therapies in which the compound of this disclosure and one or more other drugs are administered on different overlapping schedules. It is also contemplated that when used in combination with one or more other active ingredients, the compounds of the present disclosure and the other active ingredients may be used in lower doses than when each is used singly. Accordingly, the pharmaceutical compositions of the present disclosure also include those that contain one or more other drugs, in addition to a compound of the present disclosure.

The above combinations include combinations of a compound of this disclosure not only with one other drug, but also with two or more other active drugs. Likewise, a compound of this disclosure may be used in combination with other drugs that are used in the prevention, treatment, control, amelioration, or reduction of risk of the diseases or conditions for which a compound of this disclosure is useful. Such other drugs may be administered, by a route and in an amount commonly used therefore, contemporaneously or sequentially with a compound of the present disclosure. When a compound of this disclosure is used contemporaneously with one or more other drugs, a pharmaceutical composition containing such other drugs in addition to the compound of this disclosure can be used. Accordingly, the pharmaceutical compositions of the present disclosure also include those that also contain one or more other active ingredients, in addition to a compound of this disclosure. The weight ratio of the compound of this disclosure to the second active ingredient may be varied and will depend upon the effective dose of each ingredient. Generally, an effective dose of each will be used.

Where the subject in need is suffering from or at risk of suffering from cancer, the subject can be treated with a compound of this disclosure in any combination with one or more other anti-cancer agents. In some embodiments, one or more of the anti-cancer agents are proapoptotic agents. Examples of anti-cancer agents include, but are not limited to, any of the following: gossyphol, genasense, polyphenol E, Chlorofusin, all trans-retinoic acid (ATRA), bryostatin, tumor necrosis factor-related apoptosis -inducing ligand (TRAIL), 5-aza -2′-deoxycytidine, all trans retinoic acid, doxorubicin, vincristine, etoposide, gemcitabine, imatinib (Gleevec™), geldanamycin, 17-N-Allylamino-17-Demethoxygeldanamycin (17-AAG), flavopiridol, LY294002, bortezomib, trastuzumab, BAY 11-7082, PKC412, or PD184352, Taxol™, also referred to as “paclitaxel”, which is a well-known anti-cancer drug which acts by enhancing and stabilizing microtubule formation, and analogs of Taxol™., such as Taxotere™. Compounds that have the basic taxane skeleton as a common structure feature, have also been shown to have the ability to arrest cells in the G2-M phases due to stabilized microtubules and may be useful for treating cancer in combination with the compounds described herein.

Further examples of anti-cancer agents for use in combination with a compound of this disclosure include inhibitors of mitogen-activated protein kinase signaling, e.g., U0126, PD98059, PD184352, PD0325901, ARRY-142886, SB239063, SP600125, BAY 43-9006, wortmannin, or LY294002; Syk inhibitors; antibodies (e.g., rituxan); MET inhibitor such as foretinib, carbozantinib, or crizotinib; VEGFR inhibitor such as sunitinib, sorafenib, regorafinib, lenvatinib, vandetanib, carbozantinib, axitinib; EGFR inhibitor such as afatinib, brivanib, carbozatinib, erlotinib, gefitinib, neratinib, lapatinib; PI3K inhibitor such as XL147, XL765, BKM120 (buparlisib), GDC-0941, BYL719, IPI145, BAY80-6946. BEX235 (dactolisib), CAL101 (idelalisib), GSK2636771, TG100-115; MTOR inhibitor such as rapamycin (sirolimus), temsirolimus, everolimus, XL388, XL765, AZD2013, PF04691502, PKI-587, BEZ235, GDC0349; MEK inhibitor such as AZD6244, trametinib, PD184352, pimasertinib, GDC-0973, AZD8330; and proteasome inhibitor such as carfilzomib, MLN9708, delanzomib, or bortezomib.

Other anti-cancer agents that can be employed in combination with a compound of this disclosure include Adriamycin, Dactinomycin, Bleomycin, Vinblastine, Cisplatin, acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cladribine; crisnatol mesylate; cyclophosphamide; cytarabine; dacarbazine; daunorubicin hydrochloride; decitabine; dexormaplatin; dezaguanine; dezaguanine mesylate; diaziquone; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; flurocitabine; fosquidone; fostriecin sodium; gemcitabine; gemcitabine hydrochloride; hydroxyurea; idarubicin hydrochloride; ifosfamide; ilmofosine; interleukin II (including recombinant interleukin II, or Ril2), interferon alfa -2a; interferon alfa -2b; interferon alfa -n1; interferon alfa -n3; interferon beta -1a; interferon gamma -1 b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride.

Other anti-cancer agents that can be employed in combination with a compound of the disclosure such as 8-(3-(4-acryloylpiperazin-1-yl) propyl)-6-(2,6-dichloro-3,5-dimethoxyphenyl)-2-(methylamino)pyrido (2,3-d)pyrimidin-7 (8H)-one used to determine the anti-tumor activity in HGS and RT4 tumor models (Example 4 below: In HGS model, vehicle dosed group reached tumor size 645 dosing at day 42 after inoculation whereas for animals treated with 20/kg of compound, the tumor size was 55 mm3 showing significant antitumor activity and induced tumor regression), include: 20-epi-1, 25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara -CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta -alethine; betaclamycin B; betulinic acid; Bfgf inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorlns; chloroquinoxaline sulfonamide; cicaprost; cis -porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; 9-dioxamycin; diphenyl spiromustine; docosanol; dolasetron; doxifluridine; droloxifene; dronabinol; duocarmycin SA; ebselen; ecomustine; edelfosine; edrecolomab; eflomithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; fmasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A; diethylstilbesterol; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; 06-benzylguanine; octreotide; okicenone; oligonucleotides; onapristone; ondansetron; ondansetron; oracin; oral cytokine inducer; ormaplatin; osaterone; oxaliplatin; oxaunomycin; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis -acridone; prostaglandin J2; proteasome inhibitors; protein A-based immune modulator; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylerie conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; R.sub.11 retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived 1; sense oligonucleotides; signal transduction inhibitors; signal transduction modulators; single chain antigen-binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer.

Yet other anticancer agents that can be employed in combination with a compound of this disclosure include alkylating agents, antimetabolites, natural products, or hormones, e.g., nitrogen mustards (e.g., mechloroethamine, cyclophosphamide, chlorambucil, etc.), alkyl sulfonates (e.g., busulfan), nitrosoureas (e.g., carmustine, lomusitne, etc.), or triazenes (decarbazine, etc.). Examples of antimetabolites include but are not limited to folic acid analog (e.g., methotrexate), or pyrimidine analogs (e.g., cytarabine), purine analogs (e.g., mercaptopurine, thioguanine, pentostatin).

Examples of natural products useful in combination with a compound of this disclosure include but are not limited to vinca alkaloids (e.g., vincristine), epipodophyllotoxins (e.g., etoposide), antibiotics (e.g., daunorubicin, doxorubicin, bleomycin), enzymes (e.g., L-asparaginase), or biological response modifiers (e.g., interferon alpha).

Examples of alkylating agents that can be employed in combination a compound of this disclosure) include, but are not limited to, nitrogen mustards (e.g., mechloroethamine, cyclophosphamide, chlorambucil, melphalan, etc.), ethylenimine and methylmelamines (e.g., hexamethlymelamine, thiotepa), alkyl sulfonates (e.g., busulfan), nitrosoureas (e.g., carmustine, lomusitne, semustine, streptozocin, etc.), or triazenes (decarbazine, etc.). Examples of antimetabolites include, but are not limited to folic acid analog (e.g., methotrexate), or pyrimidine analogs (e.g., fluorouracil, floxuridine, cytarabine), purine analogs (e.g., mercaptopurine, thioguanine, pentostatin.

Examples of hormones and antagonists useful in combination a compound of this disclosure include, but are not limited to, adrenocorticosteroids (e.g., prednisone), progestins (e.g., hydroxyprogesterone caproate, megestrol acetate, medroxyprogesterone acetate), estrogens (e.g., diethylstilbestrol, ethinyl estradiol), antiestrogen (e.g., tamoxifen), androgens (e.g., testosterone propionate, fluoxymesterone), antiandrogen (e.g., flutamide), gonadotropin releasing hormone analog (e.g., leuprolide). Other agents that can be used in the methods and compositions described herein for the treatment or prevention of cancer include platinum coordination complexes (e.g., cisplatin, carboblatin), anthracenedione (e.g., mitoxantrone), substituted urea (e.g., hydroxyurea), methyl hydrazine derivative (e.g., procarbazine), adrenocortical suppressant (e.g., mitotane, aminoglutethimide).

Examples of anti-cancer agents which act by arresting cells in the G2-M phases due to stabilized microtubules and which can be used in combination with an irreversible Btk inhibitor compound include without limitation the following marketed drugs and drugs in development: Erbulozole (also known as R-55104), Dolastatin 10 (also known as DLS-10 and NSC-376128), Mivobulin isethionate (also known as CI-980), Vincristine, NSC-639829, Discodermolide (also known as NVP-XX-A-296), ABT-751 (Abbott, also known as E-7010), Altorhyrtins (such as Altorhyrtin A and Altorhyrtin C), Spongistatins (such as Spongistatin 1, Spongistatin 2, Spongistatin 3, Spongistatin 4, Spongistatin 5, Spongistatin 6, Spongistatin 7, Spongistatin 8, and Spongistatin 9), Cemadotin hydrochloride (also known as LU-103793 and NSC-D-669356), Epothilones (such as Epothilone A, Epothilone B, Epothilone C (also known as desoxyepothilone A or dEpoA), Epothilone D (also referred to as KOS-862, dEpoB, and desoxyepothilone B), Epothilone E, Epothilone F, Epothilone B N-oxide, Epothilone A N-oxide, 16-aza -epothilone B, 21-aminoepothilone B (also known as BMS-310705), 21-hydroxyepothilone D (also known as Desoxyepothilone F and dEpoF), 26-fluoroepothilone), Auristatin PE (also known as NSC-654663), Soblidotin (also known as TZT-1027), LS-4559-P (Pharmacia, also known as LS-4577), LS-4578 (Pharmacia, also known as LS-477-P), LS-4477 (Pharmacia), LS-4559 (Pharmacia), RPR-112378 (Aventis), Vincristine sulfate, DZ-3358 (Daiichi), FR-182877 (Fujisawa, also known as WS-9885B), GS-164 (Takeda), GS-198 (Takeda), KAR-2 (Hungarian Academy of Sciences), BSF-223651 (BASF, also known as ILX-651 and LU-223651), SAH-49960 (Lilly/Novartis), SDZ-268970 (Lilly/Novartis), AM-97 (Armad/Kyowa Hakko), AM-132 (Armad), AM-138 (Armad/Kyowa Hakko), IDN-5005 (Indena), Cryptophycin 52 (also known as LY-355703), AC-7739 (Ajinomoto, also known as AVE-8063A and CS-39.HCl), AC-7700 (Ajinomoto, also known as AVE-8062, AVE-8062A, CS-39-L-Ser.HCl, and RPR-258062A), Vitilevuamide, Tubulysin A, Canadensol, Centaureidin (also known as NSC-106969), T-138067 (Tularik, also known as T-67, TL-138067 and TI-138067), COBRA-1 (Parker Hughes Institute, also known as DDE-261 and WHI-261), H10 (Kansas State University), H16 (Kansas State University), Oncocidin A1 (also known as BTO-956 and DIME), DDE-313 (Parker Hughes Institute), Fijianolide B. Laulimalide, SPA-2 (Parker Hughes Institute), SPA-1 (Parker Hughes Institute, also known as SPIKET-P), 3-IAABU (Cytoskeleton/Mt. Sinai School of Medicine, also known as MF-569), Narcosine (also known as NSC-5366), Nascapine, D-24851 (Asta Medica), A-105972 (Abbott), Hemiasterlin, 3-BAABU (Cytoskeleton/Mt. Sinai School of Medicine, also known as MF-191), TMPN (Arizona State University), Vanadocene acetylacetonate, T-138026 (Tularik), Monsatrol, Inanocine (also known as NSC-698666), 3-1AABE (Cytoskeleton/Mt. Sinai School of Medicine), A-204197 (Abbott), T-607 (Tuiarik, also known as T-900607), RPR-115781 (Aventis), Eleutherobins (such as Desmethyleleutherobin, Desaetyleleutherobin, Isoeleutherobin A, and Z-Eleutherobin), Caribaeoside, Caribacolin, Halichondrin B, D-64131 (Asta Medica), D-68144 (Asta Medica), Diazonamide A, A-293620 (Abbott), NPI-2350 (Nereus), Taccalonolide A, TUB-245 (Aventis), A-259754 (Abbott), Diozostatin, (-)-Phenylahistin (also known as NSCL-96F037), D-68838 (Asta Medica), D-68836 (Asta Medica), Myoseverin B, D-43411 (Zentaris, also known as D-81862), A-289099 (Abbott), A-318315 (Abbott), HTI-286 (also known as SPA-110, trifluoroacetate salt) (Wyeth), D-82317 (Zentaris), D-82318 (Zentaris), SC-12983 (NCI), Resverastatin phosphate sodium, BPR-OY-007 (National Health Research Institutes), and SSR-250411 (Sanofi).

Further examples of anti-cancer agents for use in combination with a compound of this disclosure include immune checkpoint inhibitors. Exemplary immune checkpoint inhibitors include inhibitors (smack molecules or biologics) against immune checkpoint molecules such as CD27, CD28, CD40, CD122, CD96, CD73, CD39, CD47, OX40, GITR, CSFIR, JAK, PI3K delta, PI3K gamma, TAM kinase, arginase, CD137 (also known as 4-1BB), ICOS, A2AR, A2BR, HIF-2a, B7-H3, B7-H4, BTLA, CTLA-4, LAG3, TIM3, VISTA, CD96, TIGIT, PD-1, PD-L1 and PD-L2. In some embodiments, the immune checkpoint molecule is a stimulatory checkpoint molecule selected from CD27, CD28, CD40, ICOS, OX40, GITR, CD137 and STING. In some embodiments, the immune checkpoint molecule is an inhibitory checkpoint molecule selected from B7-H3, B7-H4, BTLA, CTLA-4, IDO, TDO, Arginase, KIR, LAG3, PD-1, TIM3, CD96, TIGIT and VISTA. In some embodiments, the immune checkpoint molecule is an inhibitory checkpoint molecule selected from CTLA-4. In some embodiments, the compounds provided herein can be used in combination with one or more agents selected from KIR inhibitors, TIGIT inhibitors, LAIR1 inhibitors, CD160 inhibitors, 2B4 inhibitors and TGFR beta inhibitors.

In some embodiments, the inhibitor of an immune checkpoint molecule is an inhibitor of PD-1, e.g., an anti-PD-1 monoclonal antibody. In some embodiments, the anti-PD-1 monoclonal antibody is nivolumab, pembrolizumab (also known as MK-3475), pidilizumab, SHR-1210, PDR001, or AMP-224. In some embodiments, the anti-PD-1 monoclonal antibody is nivolumab, or pembrolizumab or PDR001. In some embodiments, the anti-PD1 antibody is pembrolizumab.

In some embodiments, the inhibitor of an immune checkpoint molecule is an inhibitor of PD-L1, e.g., an anti-PD-L1 monoclonal antibody. In some embodiments, the anti-PD-L1 monoclonal antibody is BMS-935559, MEDI4736, MPDL3280A (also known as RG7446), or MSB0010718C. In some embodiments, the anti-PD-L1 monoclonal antibody is MPDL3280A (atezolizumab) or MEDI4736 (durvalumab).

In some embodiments, the inhibitor of an immune checkpoint molecule is an inhibitor of CTLA-4, e.g., an anti-CTLA-4 antibody. In some embodiments, the anti-CTLA-4 antibody is ipilimumab or tremelimumab. In some embodiments, the inhibitor of an immune checkpoint molecule is an inhibitor of LAG3, e.g., an anti-LAG3 antibody. In some embodiments, the anti-LAG3 antibody is BMS-986016 or LAG525. In some embodiments, the inhibitor of an immune checkpoint molecule is an inhibitor of GITR, e.g., an anti-GITR antibody. In some embodiments, the anti-GITR antibody is TRX518 or, MK-4166, INCAGN01876 or MK-1248. In some embodiments, the inhibitor of an immune checkpoint molecule is an inhibitor of OX40, e.g., an anti-OX40 antibody or OX40L fusion protein. In some embodiments, the anti-OX40 antibody is MEDI0562 or, INCAGN01949, GSK2831781, GSK-3174998, MOXR-0916, PF-04518600 or LAG525. In some embodiments, the OX40L fusion protein is MEDI6383 General Methods

All solvents used were commercially available and were used without further purification. Reactions were typically run using anhydrous solvents under an inert atmosphere of nitrogen.

1H spectra were recorded at 400 MHz or 300 MHz for proton on a Bruker 400 NMR Spectrometer equipped with a Bruker 400 BBO probe or Bruker BBFO ULTRASHIELD™300 AVANCE III, respectively. All deuterated solvents contained typically 0.03% to 0.05% v/v tetramethylsilane, which was used as the reference signal (set at d 0.00 for both ¹H and ¹³C).

LCMS analyses were performed on a SHIMADZU LCMS consisting of an UFLC 20-AD and LCMS 2020 MS detector. The Diode Array Detector was scanned from 190-400 nm. The mass spectrometer was equipped with an electrospray ion source (ESI) operated in a positive or negative mode. The mass spectrometer was scanned between m/z 90-900 with a scan time from 0.5 to 3.0 s.

HPLC analyses were performed on a SHIMADZU UFLC with two LC20 AD pump and a SPD-M20A Photodiiode Array Detector. The column used was an XBridge C18, 3.5 μm, 4.6×100 mm. A linear gradient was applied, starting at 90% A (A: 0.05% TFA in water) and ending at 95% B (B: 0.05% TFA in MeCN) over 10 min with a total run time of 15 min. The column temperature was at 40° C. with the flow rate of 1.5 mL/min. The Diode Array Detector was scanned from 200-400 nm.

Thin layer chromatography (TLC) was performed on Alugram® (Silica gel 60 F254) from Mancherey-Nagel and UV was typically used to visualize the spots. Additional visualization methods were also employed in some cases. In these cases, the TLC plate was developed with iodine (generated by adding approximately 1 g of I₂ to 10 g silica gel and thoroughly mixing), ninhydrin (available commercially from Aldrich), or Magic Stain (generated by thoroughly mixing 25 g (NH₄)₆Mo₇O₂₄·4H₂O, 5 g (NH₄)₂Ce (IV) (NO₃)₆ in 450 mL water and 50 mL concentrated H₂SO₄) to visualize the compound. Flash chromatography was performed using 40-63 μm (230-400 mesh) silica gel from Silicycle following analogous techniques to those disclosed in Still, W.C.; Kahn, M.; and Mitra, M. Journal of Organic Chemistry, 1978, 43, 2923. Typical solvents used for flash chromatography or thin layer chromatography were mixtures of chloroform/methanol, dichloromethane/methanol, ethyl acetate/methanol and hexanes/ethyl acetate.

### SYNTHETIC EXAMPLES

### Example 1

### Synthesis of imino(methyl) [[2-(1,7-naphthyridin-4-yl)-2-azaspiro[3.3]heptan-6-yl]methyl]-lambda6-sulfanone

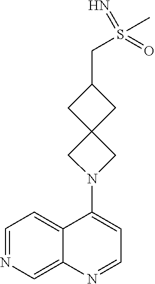 [SMILES: CS(=N)(=O)CC1CC2(C1)CN(c1ccnc3cnccc13)C2]

### Step 1: tert-Butyl 6-[(methanesulfonyloxy)methyl]-2-azaspiro[3.3]heptane-2-carboxylate

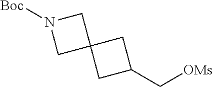 [SMILES: CC(C)(C)OC(=O)N1CC2(CC(COS(C)(=O)=O)C2)C1]

To a stirred solution of tert-butyl 6-(hydroxymethyl)-2-azaspiro[3.3]heptane-2-carboxylate (1.00 g, 4.39 mmol, 1 equiv) in DCM (12 mL) was added TEA (890 mg, 8.79 mmol, 2 equiv) and MsCl (604 mg, 5.27 mmol, 1.2 equiv) at 0° C. After stirring at room temperature for 2 h, the resulting mixture was diluted with DCM and washed with water and brine. The organic layer was dried over anhydrous sodium sulfate, filtered and concentrated under reduced pressure to give tert-butyl 6-[(methanesulfonyloxy)methyl]-2-azaspiro[3.3]heptane-2-carboxylate (1.34 g, 99%) as a yellow solid.

### Step 2: tert-Butyl 6-[(methylsulfanyl)methyl]-2-azaspiro[3.3]heptane-2-carboxylate

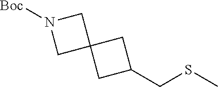 [SMILES: CSCC1CC2(C1)CN(C(=O)OC(C)(C)C)C2]

To a stirred solution of tert-butyl 6-[(methanesulfonyloxy)methyl]-2-azaspiro[3.3]heptane-2-carboxylate (1.34 g, 4.38 mmol, 1 equiv) in EtOH (10 mL) was added sodiummethanethiolate (6.68 mL, 20% in H₂O) at room temperature. After stirring for 2 h, the reaction mixture was concentrated under reduced pressure and purified by silica gel column chromatography, eluted with EA/PE (18:82) to afford tert-butyl 6-[(methylsulfanyl)methyl]-2-azaspiro[3.3]heptane-2-carboxylate (894 mg, 79%) as a yellow solid.

### Step 3:6-[(Methylsulfanyl)methyl]-2-azaspiro[3.3]heptane

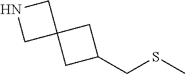

To a solution of tert-butyl 6-[(methylsulfanyl)methyl]-2-azaspiro[3.3]heptane-2-carboxylate (300 mg, 1.16 mmol, 1 equiv) in DCM (10 mL) was added 2,6-lutidine (0.12 g, 1.16 mmol, 1 equiv) and TMSOTf (0.78 g, 3.498 mmol, 3 equiv) at room temperature. After stirring for 2 h, the reaction mixture was concentrated under reduced pressure to give 6-[(methylsulfanyl)methyl]-2-azaspiro[3.3]heptane (380 mg, crude) as a light-yellow oil, which was used for next step directly without further purification.

### Step 4:4-[6-[(Methylsulfanyl)methyl]-2-azaspiro[3.3]heptan-2-yl]-1,7-naphthyridine

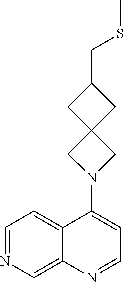 [SMILES: CSCC1CC2(C1)CN(c1ccnc3cnccc13)C2]

To a solution of 4-chloro-1,7-naphthyridine (100 mg, 0.608 mmol, 1 equiv) in NMP (4 mL) was added DIEA (314 mg, 2.43 mmol, 4 equiv) and 6-[(methylsulfanyl)methyl]-2-azaspiro[3.3]heptane (191 mg, crude). The resulting mixture was stirred at 110° C. for 3 h. After cooling to room temperature, the crude product was purified by reversed phase CombiFlash, eluting with ACN/H₂O (45:55) to afford 4-[6-[(methylsulfanyl)methyl]-2-azaspiro[3.3]heptan-2-yl]-1,7-naphthyridine (68 mg, 39%) as a brown solid.

### Step 5: Imino(methyl) [[2-(1,7-naphthyridin-4-yl)-2-azaspiro[3.3]heptan-6-yl]methyl]-lambda6-sulfanone

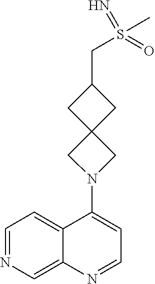 [SMILES: CS(=N)(=O)CC1CC2(C1)CN(c1ccnc3cnccc13)C2]

To a solution of 4-[6-[(methylsulfanyl)methyl]-2-azaspiro[3.3]heptan-2-yl]-1,7-naphthyridine (53 mg, 0.186 mmol, 1 equiv) in MeOH (3 mL) was added (acetyloxy) (phenyl)-lambda3-iodanyl acetate (179 mg, 0.558 mmol, 3 equiv) and ammonium carbamate (58 mg, 0.744 mmol, 4 equiv). The resulting mixture was stirred at room temperature for 1 h and then concentrated under reduce pressure and purified by prep-HPLC with the following conditions (Column: XBridge Prep OBD C18 Column, 30*150 mm, 5 μm; Mobile Phase A: Water (10 nmol/L NH₄HCO₃), Mobile Phase B: ACN; Flow rate: 60 mL/min; Gradient: 2% B to 24% B in 10 min; Wave Length: 254 nm/220 nm nm; RT1 (min): 9.07) to afford imino(methyl) [[2-(1,7-naphthyridin-4-yl)-2-azaspiro[3.3]heptan-6-yl]methyl]-lambda6-sulfanone (20.3 mg, 32%) as an off-white solid. MS (ESI, pos, ion) m/z: 317.1 (M+1). ¹H NMR (300 MHz, DMSO-d₆+D₂O, ppm) δ 9.08 (s, 1H), 8.43 (d, J=5.4 Hz, 1H), 8.34 (d, J=5.8 Hz, 1H), 7.83 (d, J=5.9 Hz, 1H), 6.38 (d, J=5.4 Hz, 1H), 4.43 (s, 2H), 4.28 (s, 2H), 3.21 (d, J=7.2 Hz, 2H), 2.85 (s, 3H), 2.70-2.62 (m, 1H), 2.50-2.41 (m, 2H), 2.18-2.10 (m, 2H).

### Example 2

### Synthesis of imino ((2-(8-methoxypyrido[3,4-d]pyrimidin-4-yl)-2-azaspiro[3.3]heptan-6-yl)methyl)(methyl)-lambda6-sulfanone (2a) and ((2-(8-hydroxypyrido[3,4-d]pyrimidin-4-yl)-2-azaspiro[3.3]heptan-6-yl)methyl) (imino)(methyl)-lambda6-sulfanone (2b)

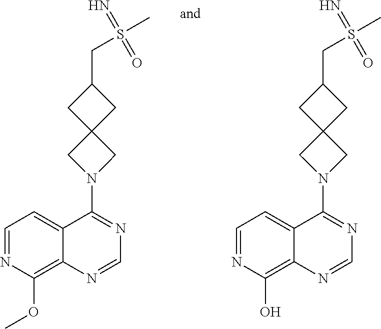 [SMILES: COc1nccc2c(N3CC4(CC(CS(C)(=N)=O)C4)C3)ncnc12 ; CS(=N)(=O)CC1CC2(C1)CN(c1ncnc3c(O)nccc13)C2]

### Step 1:2-[8-Methoxypyrido[3,4-d]pyrimidin-4-yl]-6-[(methylsulfanyl)methyl]-2-azaspiro[3.3]heptane

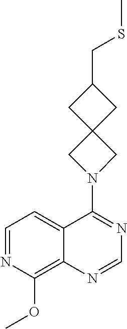 [SMILES: COc1nccc2c(N3CC4(CC(CSC)C4)C3)ncnc12]

The title compound was synthesized by proceeding analogously as described in Example 1, Step 4 except 4-chloro-8-methoxypyrido[3,4-d]pyrimidine (100 mg, 0.51 mmol) was used and stirred at 90° C. for 2 h. 2-[8-Methoxypyrido[3,4-d]pyrimidin-4-yl]-6-[(methylsulfanyl)methyl]-2-azaspiro[3.3]heptane (105 mg, 64%) was obtained as a brown solid.

### Step 2: Imino ((2-(8-methoxypyrido[3,4-d]pyrimidin-4-yl)-2-azaspiro[3.3]heptan-6-yl)methyl)(methyl)-lambda6-sulfanone and ((2-(8-hydroxypyrido[3,4-d]pyrimidin-4-yl)-2-azaspiro[3.3]heptan-6-yl)methyl) (imino)(methyl)-lambda6-sulfanone

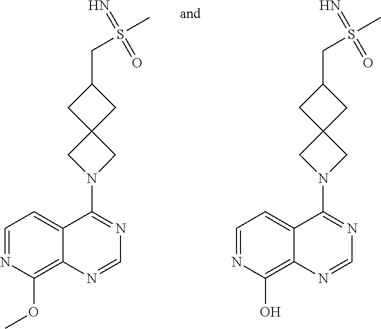 [SMILES: COc1nccc2c(N3CC4(CC(CS(C)(=N)=O)C4)C3)ncnc12 ; CS(=N)(=O)CC1CC2(C1)CN(c1ncnc3c(O)nccc13)C2]

The title compounds were synthesized by proceeding analogously as described in Example 1, Step 5 except 2-[8-methoxypyrido[3,4-d]pyrimidin-4-yl]-6-[(methylsulfanyl)methyl]-2-azaspiro[3.3]heptane (95 mg, 0.30 mmol) was used. The crude product was purified by prep-HPLC with the following conditions: (Column: YMC-Actus Triart C18 ExRS Column, 30*150 mm, 5 μm; Mobile Phase A: Water (10 mmol/L NH₄HCO₃), Mobile Phase B: ACN; Flow rate: 60 mL/min; Gradient: 3% B to 28% B in 8 min; Wave Length: 254 nm/220 nm nm; RT1 (min): 7.12). The fractions containing the desired product were combined and lyophilized to afford:

Fraction 1: Imino ((2-(8-methoxypyrido[3,4-d]pyrimidin-4-yl)-2-azaspiro[3.3]heptan-6-yl)methyl)(methyl)-lambda6-sulfanone (2a, 21.1 mg, 20%) as a white solid. MS (ESI, pos, ion) m/z: 348.1 (M+1). ¹H NMR (400 MHZ, DMSO-d₆, ppm) δ 8.50 (s, 1H), 8.01 (d, J=5.8 Hz, 1H), 7.30 (d, J=5.9 Hz, 1H), 4.45 (s, 4H), 3.99 (s, 3H), 3.56 (s, 1H), 3.24-3.12 (m, 2H), 2.85 (s, 3H), 2.69-2.63 (m, 1H), 2.48-2.45 (m 1H), 2.15 (q, J=11.4, 10.9 Hz, 2H).

Fraction 2: ((2-(8-Hydroxypyrido[3,4-d]pyrimidin-4-yl)-2-azaspiro[3.3]heptan-6-yl)methyl) (imino)(methyl)-lambda6-sulfanone (2b, 12.6 mg, 12%) as a white solid. MS (ESI, pos, ion) m/z: 334.2 (M+1). ¹H NMR (300 MHz, DMSO-d₆, ppm) δ 11.65 (s, 1H), 8.46 (s, 1H), 7.17 (d, J=7.2 Hz, 1H), 6.44 (d, J=7.3 Hz, 1H), 4.47 (s, 2H), 4.31 (s, 2H), 3.55 (s, 1H), 3.21-3.14 (m, 2H), 2.84 (s, 3H), 2.67-2.63 (m, 1H), 2.48-2.43 (m, 2H), 2.12 (q, J=10.7 Hz, 2H).

### Example 3

### Synthesis of imino (3-(1-(8-methoxy-1,7-naphthyridin-4-yl) azetidin-3-yl) propyl)(methyl)-lambda6-sulfanone

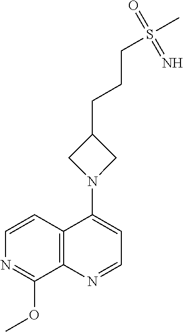 [SMILES: COc1nccc2c(N3CC(CCCS(C)(=N)=O)C3)ccnc12]

### Step 1: tert-Butyl 3-(3-((methylsulfonyl)oxy) propyl) azetidine-1-carboxylate

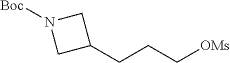 [SMILES: CC(C)(C)OC(=O)N1CC(CCCOS(C)(=O)=O)C1]

The title compound was synthesized byproceeding analogously as described in Example 1, Step 1 except tert-butyl 3-(3-hydroxypropyl) azetidine-1-carboxylate (1.00 g, 4.64 mmol) was used, tert-butyl 3-(3-((methylsulfonyl)oxy) propyl) azetidine-1-carboxylate (1.40 g, crude) was obtained as a light yellow oil, which was used in the next step directly without further purification.

### Step 2: tert-Butyl 3-[3-(methylsulfanyl) propyl]azetidine-1-carboxylate

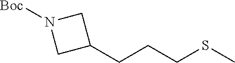 [SMILES: CSCCCC1CN(C(=O)OC(C)(C)C)C1]

The title compound was synthesized by proceeding analogously as described in Example 1, Step 2 except tert-butyl 3-(3-((methylsulfonyl)oxy) propyl) azetidine-1-carboxylate (1.40 g, 4.77 mmol) was used, tert-butyl 3-[3-(methylsulfanyl) propyl]azetidine-1-carboxylate (1.00 g, 85%) was obtained as a colorless oil.

### Step 3:3-(3-(Methylthio) propyl) azetidine

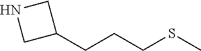

The title compound was synthesized by proceeding analogously as described in Example 1, Step 3 except tert-butyl 3-[3-(methylsulfanyl) propyl]azetidine-1-carboxylate (450 mg, 1.83 mmol) was used. 3-(3-(Methylthio) propyl) azetidine (0.80 g, crude) was obtained as a colorless oil.

### Step 4:4-Chloro-8-methoxy-1,7-naphthyridine

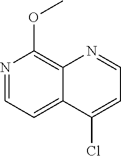

To a stirred solution of 8-methoxy-1,7-naphthyridin-4-ol (1.60 g, 9.08 mmol, 1 equiv) in toluene (32 mL) was added DIEA (2.35 g, 18.16 mmol, 2 equiv) and POCl₃ (1.67 g, 10.89 mmol, 1.2 equiv) at room temperature. After stirring overnight at 70° C., the resulting mixture was concentrated under reduced pressure. The crude product was purified by reversed phase flash eluted with ACN/H₂O (35:65) to give 4-chloro-8-methoxy-1,7-naphthyridine (1.11 g, 62%) as a yellow solid.

### Step 5:8-Methoxy-4-[3-[3-(methylsulfanyl) propyl]azetidin-1-yl]-1,7-naphthyridine

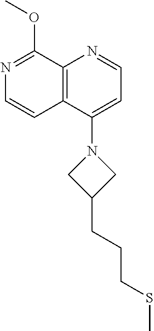 [SMILES: COc1nccc2c(N3CC(CCCSC)C3)ccnc12]

The title compound was synthesized by proceeding analogously as described in Example 1, Step 4 except 4-chloro-8-methoxy-1,7-naphthyridine (150 mg, 0.77 mmol) and 3-[3-(methylsulfanyl)-propyl]azetidine (1.05 g, crude) were used and stirred for 2 h at 130° C. 8-Methoxy-4-[3-[3-(methylsulfanyl) propyl]azetidin-1-yl]-1,7-naphthyridine (42 mg, 18%) was obtained as an off-white solid.

### Step 6: Imino (3-(1-(8-methoxy-1,7-naphthyridin-4-yl) azetidin-3-yl) propyl)(methyl)-lambda6-sulfanone

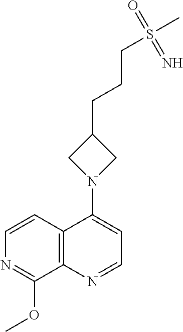 [SMILES: COc1nccc2c(N3CC(CCCS(C)(=N)=O)C3)ccnc12]

The title compound was synthesized by proceeding analogously as described in Example 1, Step 5 except 8-methoxy-4-[3-[3-(methylsulfanyl) propyl]azetidin-1-yl]-1,7-naphthyridine (32 mg, 0.10 mmol) was used. The crude product was purified by prep-HPLC with the following conditions: (Column: XBridge Prep OBD C18 Column, 30*150 mm, 5 μm; Mobile Phase A: Water (10 nmol/LNH₄HCO₃), Mobile Phase B: ACN; Flow rate: 60 mL/min mL/min; Gradient: 2% B to 27% B in 10 min; Wavelength: 254 nm/220 nm nm; RT1 (min): 8.9). The fractions containing the desired product were combined and lyophilized to afford imino (3-(1-(8-methoxy-1,7-naphthyridin-4-yl) azetidin-3-yl) propyl)(methyl)-lambda6-sulfanone (4.1 mg, 10%) as a brown solid. MS (ESI, pos. ion) m/z: 335.2 (M+1). ¹H NMR (400 MHZ, DMSO-d₆, ppm) δ 8.41 (d, J=5.2 Hz, 1H), 7.87 (d, J=6.0 Hz, 1H), 7.39 (d, J=6.1 Hz, 1H), 6.43 (d, J=5.3 Hz, 1H), 4.45 (t, J=8.2 Hz, 2H), 3.99 (s, 5H), 3.64 (s, 1H), 3.07 (t, J=7.5 Hz, 2H), 2.90 (s, 3H), 2.80-2.78 (m, 1H), 1.82-1.66 (m, 4H).

### Example 4

### Synthesis of cyclopropyl((4-hydroxy-1-(8-methoxy-1,7-naphthyridin-4-yl) piperidin-4-yl)-methyl) (imino)-lambda6-sulfanone

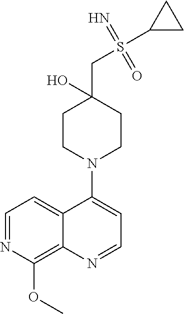 [SMILES: COc1nccc2c(N3CCC(O)(CS(=N)(=O)C4CC4)CC3)ccnc12]

### Step 1: tert-Butyl 4-hydroxy-4-(sulfanylmethyl) piperidine-1-carboxylate

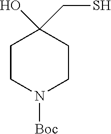 [SMILES: CC(C)(C)OC(=O)N1CCC(O)(CS)CC1]

A mixture of sodium sulfide nonahydrate (5.63 g, 23.44 mmol, 2.5 equiv) and sodium sulfide nonahydrate (5.63 g, 23.44 mmol, 2.5 equiv)) in MeOH (80 mL) was stirred at 0° C. for 15 min under N₂ atmosphere, tert-Butyl 1-oxa -6-azaspiro[2.5]octane-6-carboxylate (2.00 g, 9.37 mmol, 1 equiv) in MeOH (80 mL) was added and the mixture was stirred for 1 h at 0° C. under N₂ atmosphere, then warmed to room temperature over 1 h. Saturated sodium bicarbonate solution was then added slowly at 0° C. The resulting mixture was concentrated to remove MeOH, then extracted with EtOAc. The combined organic layers were washed with water and brine, dried over anhydrous sodium sulfate and filtered. The filtrate was concentrated under reduced pressure. The residue was purified by silica gel column chromatography, eluted with EtOAc/PE (1:8) to give tert-butyl 4-hydroxy-4-(sulfanylmethyl) piperidine-1-carboxylate (1.50 g, 64%) as a colorless oil.

### Step 2: tert-Butyl 4-[(cyclopropylsulfanyl)methyl]-4-hydroxypiperidine-1-carboxylate

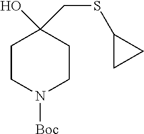 [SMILES: CC(C)(C)OC(=O)N1CCC(O)(CSC2CC2)CC1]

To a mixture of tert-butyl 4-hydroxy-4-(sulfanylmethyl) piperidine-1-carboxylate (600 mg, 2.42 mmol, 1 equiv) and bromocyclopropane (293 mg, 2.42 mmol, 1 equiv) in DMSO (6 mL) was added potassium tert-butoxide (816 mg, 7.27 mmol, 3 equiv). The resulting mixture was stirred overnight at 120° C. under an N₂ atmosphere. After cooling to room temperature, the reaction mixture was quenched with water and extracted with dichloromethane. The combined organic layers were washed with water and brine, dried over anhydrous sodium sulfate and filtered. The filtrate was concentrated under reduced pressure. The residue was purified by silica gel column chromatography, eluted with EA/PE (1:9) to give tert-butyl 4-[(cyclopropylsulfanyl)methyl]-4-hydroxypiperidine-1-carboxylate (410 mg, 58%) as a colorless oil.

### Step 3:4-((Cyclopropylthio)methyl) piperidin-4-ol hydrochloride

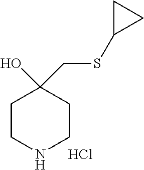 [SMILES: OC1(CSC2CC2)CCNCC1 ; Cl]

A solution of tert-butyl 4-[(cyclopropylsulfanyl)methyl]-4-hydroxypiperidine-1-carboxylate (410 mg, 1.42 mmol) in 3 mL HCl (g, 4 M in dioxane) was stirred for 1 h at room temperature. The resulting mixture was concentrated under reduced pressure to afford 4-((cyclopropylthio)methyl) piperidin-4-ol hydrochloride (380 mg crude), which was used in the next step directly without further purification.

### Step 4:4-[(Cyclopropylsulfanyl)methyl]-1-(8-methoxy-1,7-naphthyridin-4-yl) piperidin-4-ol

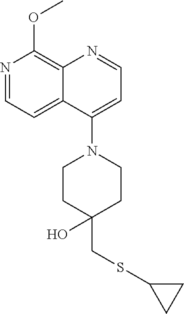 [SMILES: COc1nccc2c(N3CCC(O)(CSC4CC4)CC3)ccnc12]

The title compound was synthesized by proceeding analogously as described in Example 1, Step 4 except 4-chloro-8-methoxy-1,7-naphthyridine (125 mg, 0.64 mmol) and 4-((cyclopropylthio)methyl) piperidin-4-ol hydrochloride (359 mg, 1.60 mmol) were used and stirred overnight at 130° C. 4-[(cyclopropylsulfanyl)methyl]-1-(8-methoxy-1,7-naphthyridin-4-yl) piperidin-4-ol (165 mg, 74%) was obtained as a brown yellow solid.

### Step 5: Cyclopropyl((4-hydroxy-1-(8-methoxy-1,7-naphthyridin-4-yl) piperidin-4-yl)methyl) (imino)-lambda6-sulfanone

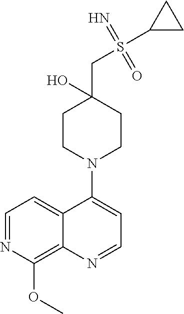 [SMILES: COc1nccc2c(N3CCC(O)(CS(=N)(=O)C4CC4)CC3)ccnc12]

The title compound was synthesized by proceeding analogously as described in Example 1, Step 5 except 4-[(cyclopropylsulfanyl)methyl]-1-(8-methoxy-1,7-naphthyridin-4-yl) piperidin-4-ol (150 mg, 0.43 mmol) was used. The crude product was purified by prep-HPLC with the following conditions: (Column: XBridge Prep OBD C18 Column, 30*150 mm, 5 μm; Mobile Phase A: Water (10 nmol/L NH₄HCO₃), Mobile Phase B: ACN; Flow rate: 60 mL/min Gradient: 9% B to 36% B in 20 min; Wavelength: 254 nm/220 nm RT1 (min): 19.65). The fractions containing the desired product were combined and lyophilized to afford cyclopropyl((4-hydroxy-1-(8-methoxy-1,7-naphthyridin-4-yl) piperidin-4-yl)methyl) (imino)-lambda6-sulfanone (68.3 mg, 41%) as an off-white solid. MS (ESI, pos. ion) m/z: 377.2 (M+1). ¹H NMR (400 MHZ, DMSO-d₆, ppm) δ 8.67 (d, J=5.0 Hz, 1H), 8.02 (d, J=5.9 Hz, 1H), 7.34 (d, J=6.0 Hz, 1H), 7.18 (d, J=5.0 Hz, 1H), 5.89 (s, 1H), 4.03 (s, 3H), 3.99 (t, J=2.7 Hz, 1H), 3.40 (d, J=14.1 Hz, 1H), 3.34 (d, J=1.7 Hz, 1H), 3.31 (s, 2H), 3.24-3.13 (m, 2H), 2.77-2.74 (m, 1H), 2.20-2.06 (m, 2H), 2.06-1.96 (m, 1H), 1.90 (d, J=13.3 Hz, 1H), 1.17-1.04 (m, 1H), 1.02-0.85 (m, 3H).

### Example 5

### Synthesis of (4-(((1,7-naphthyridin-4-yl)oxy)methyl)phenyl) (imino)(methyl)-lambda6-sulfanone

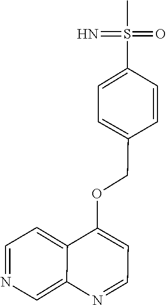 [SMILES: CS(=N)(=O)c1ccc(COc2ccnc3cnccc23)cc1]

### Step 1:1,7-Naphthyridine-2,4-diol

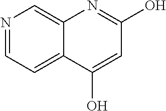 [SMILES: Oc1cc(O)c2ccncc2n1]

To a solution of methyl 3-aminopyridine-4-carboxylate (50.00 g, 328.62 mmol, 1 equiv) in ethyl acetate (400 mL) was added potassium tert-butoxide (77.44 g, 690.10 mmol, 2.1 equiv) under nitrogen atmosphere. The reaction mixture was heated to 75° C. and stirred overnight under nitrogen atmosphere. The reaction mixture was cooled to room temperature and water was added. The organic phase was separated and the aqueous phase was extracted with EtOAc and tert-butyl methyl ether. The aqueous phase was acidified with 2 N HCl to pH=6. The resulting precipitate was collected by filtration, washed with water and dried under vacuum to afford 1,7-naphthyridine-2,4-diol (15.00 g, 28%) as a brown solid.

### Step 2:2,4-Dichloro-1,7-naphthyridine

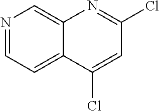 [SMILES: Clc1cc(Cl)c2ccncc2n1]

A solution of 1,7-naphthyridine-2,4-diol (3.80 g, 23.44 mmol, 1 equiv) in POCl₃ (40 mL) was stirred at 110° C. for 40 minutes. The resulting mixture was cooled to room temperature and concentrated under reduced pressure. The residue was diluted with saturated sodium bicarbonate solution, extracted with EtOAc (and brine. The organic layer was dried over anhydrous sodium sulfate, filtered and concentrated under reduced pressure. The residue was purified by silica gel column chromatography, eluted with MeOH/DCM (12/88) to 2,4-dichloro-1,7-naphthyridine (2.80 g, 60%) and concentrated under reduced pressure to give a yellow solid which was used without further purification.

### Step 3:4-Chloro-1,7-naphthyridine

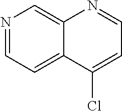 [SMILES: Clc1ccnc2cnccc12]

To a stirred solution of 2,4-dichloro-1,7-naphthyridine (8.30 g, 41.70 mmol, 1 equiv) in DMF (120 mL) and H₂O (60 mL) was added propyl formate (18.37 g, 208.51 mmol, 5 equiv) and PdCl₂ (PPh₃)₂ (585 mg, 0.83 mmol, 0.02 equiv) at room temperature. After stirring for 3 days at 80° C. under nitrogen atmosphere, the resulting mixture was cooled to room temperature. Water was added and extracted with EtOAc and washed with brine. The organic layer was dried over anhydrous sodium sulfate, filtered, and concentrated under reduced pressure. The residue was purified by silica gel column chromatography, eluted with EA/PE (28/72) to give 4-chloro-1,7-naphthyridine (2.66 g, 38%) as a yellow solid.

### Step 4:4-[4-(Methylsulfanyl)phenyl]methoxy]-1,7-naphthyridine

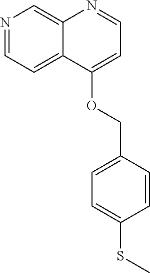 [SMILES: CSc1ccc(COc2ccnc3cnccc23)cc1]

To a solution of [4-(methylsulfanyl)phenyl]methanol (365 mg, 2.37 mmol, 1.5 equiv) in DMF (4 mL) was added NaH (126 mg, 3.16 mmol, 2 equiv, 60%) at 0° C. After stirring for 20 min at 0° C., 4-chloro-1,7-naphthyridine (260 mg, 1.58 mmol, 1 equiv) was added. The resulting mixture was stirred at 80° C. for 2 h, After cooling to 0° C., water was added, then extracted with EtOAc and washed with brine. The organic layer was dried over anhydrous sodium sulfate, filtered and concentrated under reduced pressure. The residue was purified by silica gel column chromatography, eluted with EA/PE (87/13) to afford 4-[[4-(methylsulfanyl)phenyl]methoxy]-1,7-naphthyridine (120 mg, 24%) as an orange solid.

### Step 5: (4-(((1,7-Naphthyridin-4-yl)oxy)methyl)phenyl) (imino)(methyl)-lambda6-sulfanone

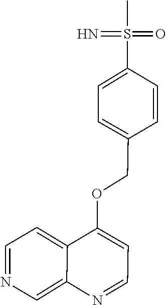 [SMILES: CS(=N)(=O)c1ccc(COc2ccnc3cnccc23)cc1]

The title compound was synthesized by proceeding analgously as described in Example 1, Step 5 except 4-[4-(methylsulfanyl)phenyl]methoxy]-1,7-naphthyridine (100 mg, 0.35 mmol) was used. The crude product was purified by prep-HPLC with the following conditions: (Column: XBridge Prep OBD C18 Column, 30*150 mm, 5 μm; Mobile Phase A: Water (10 nmol/L NH₄HCO₃), Mobile Phase B: ACN; Flow rate: 60 mL/min; Gradient: 2% B to 28% B in 10 min; Wave Length: 254/220 nm; RT1 (min): 8.5). The fractions containing the desired product were combined and lyophilized to afford (4-(((1,7-naphthyridin-4-yl)oxy)methyl)phenyl)-(imino)(methyl)-lambda6-sulfanone (38.3 mg, 33%) as a white solid. MS (ESI, pos. ion) m/z: 314.1 (M+1). ¹H NMR (400 MHZ, DMSO-d₆, ppm) δ 9.35 (d, J=1.0 Hz, 1H), 8.91 (d, J=5.2 Hz, 1H), 8.62 (d, J=5.6 Hz, 1H), 8.09-8.03 (m, 1H), 8.03-7.97 (m, 2H), 7.83-7.76 (m, 2H), 7.36 (d, J=5.2 Hz, 1H), 5.57 (s, 2H), 4.26 (s, 1H), 3.09 (d, J=0.8 Hz, 3H).

### Example 6

### Synthesis of (3-(((1,7-naphthyridin-4-yl)oxy)methyl)phenyl) (cyclopropyl) (imino)-lambda6-sulfanone

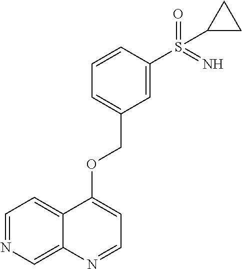 [SMILES: N=S(=O)(c1cccc(COc2ccnc3cnccc23)c1)C1CC1]

### Step 1:4-[3-(Cyclopropylsulfanyl)phenyl]methoxy]-1,7-naphthyridine

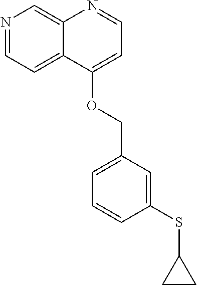 [SMILES: c1cc(COc2ccnc3cnccc23)cc(SC2CC2)c1]

The title compound was synthesized by proceeding analogously as described in Example 5, Step 4 except [3-(cyclopropylsulfanyl)phenyl]methanol (98 mg, 0.55 mmol) was used. 4-[3-(cyclopropylsulfanyl)phenyl]methoxy]-1,7-naphthyridine (133 mg, 65%) was obtained as a yellow oil.

### Step 2: (3-(((1,7-Naphthyridin-4-yl)oxy)methyl)phenyl) (cyclopropyl) (imino)-lambda 6-sulfanone

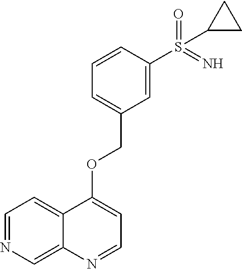 [SMILES: N=S(=O)(c1cccc(COc2ccnc3cnccc23)c1)C1CC1]

The title compound was synthesized by proceeding analogously as described in Example 1, Step 5 except 4-[3-(cyclopropylsulfanyl)phenyl]methoxy]-1,7-naphthyridine (110 mg, 0.36 mmol) was used. The crude product was purified by prep-HPLC with the following conditions: (Column: XBridge Prep OBD C18 Column, 30*150 mm, 5 μm; Mobile Phase A: Water (10 nmol/L NH₄HCO₃), Mobile Phase B: ACN; Flow rate: 60 mL/min; Gradient: 5% B to 35% B in 10 min; Wavelength: 254/220 nm; RT1 (min): 8.5). The fractions containing the desired product were combined and lyophilized to afford (3-(((1,7-naphthyridin-4-yl)oxy)methyl)phenyl)-(cyclopropyl) (imino)-lambda6-sulfanone (44.7 mg, 36%) as a white solid. MS (ESI, pos. ion) m/z: 340.2 (M+1). ¹H NMR (400 MHZ, DMSO-d₆, ppm) δ 9.35 (d, J=1.0 Hz, 1H), 8.92 (d, J=5.2 Hz, 1H), 8.62 (d, J=5.6 Hz, 1H), 8.12-8.07 (m, 1H), 8.05-7.99 (m, 1H), 7.95-7.83 (m, 2H), 7.72-7.64 (m, 1H), 7.38 (d, J=5.2 Hz, 1H), 5.57 (s, 2H), 4.29 (s, 1H), 2.74-2.62 (m, 1H), 1.19-1.06 (m, 1H), 1.04-0.75 (m, 3H).

### Example 7

### Synthesis of (3-(((1,7-naphthyridin-4-yl)oxy)methyl)phenyl) (imino)(methyl)-lambda6-sulfanone

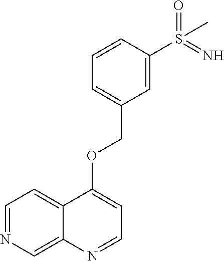 [SMILES: CS(=N)(=O)c1cccc(COc2ccnc3cnccc23)c1]

### Step 1: [3-(Methylsulfanyl)phenyl]methanol

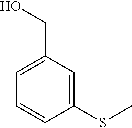

To a solution of 3-(methylsulfanyl)benzoic acid (4.00 g, 23.78 mmol, 1 equiv) in THF (40 mL) was slowly added LiAlH₄ (14.2 mL, 2 M in THF) at 0° C. under nitrogen atmosphere. After stirring at room temperature for 2 h, the mixture was quenched by addition of saturated ammonium chloride solution (100 mL) and concentrated under reduced pressure. The residue was purified by silica gel column chromatography, eluted with EtOAc/PE (43:57) to afford [3-(methylsulfanyl)phenyl]methanol (3.08 g, 84%) as a colorless oil.

### Step 2:4-[[3-(Methylsulfanyl)phenyl]methoxy]-1,7-naphthyridine

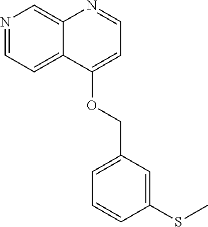 [SMILES: CSc1cccc(COc2ccnc3cnccc23)c1]

The title compound was synthesized by proceeding analogously as described in Example 5 Step 4 except [3-(methylsulfanyl)phenyl]methanol (187 mg, 1.215 mmol) was used. 4-[3-(methylsulfanyl)phenyl]methoxy]-1,7-naphthyridine (300 mg, 87%) was obtained as a yellow oil.

### Step 3: (3-(((1,7-Naphthyridin-4-yl)oxy)methyl)phenyl) (imino)(methyl)-lambda6-sulfanone

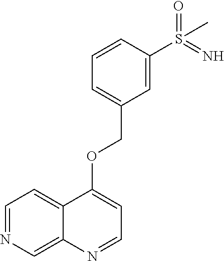 [SMILES: CS(=N)(=O)c1cccc(COc2ccnc3cnccc23)c1]

The title compound was synthesized by proceeding analogously as described in Example 1, Step 5 except 4-[3-(methylsulfanyl)phenyl]methoxy]-1,7-naphthyridine (150 mg, 0.531 mmol) was used. The crude product was purified by prep-HPLC with the following conditions: (Column: XselectCSH Prep OBD C18 Column, 30*150 mm, 5 μm; Mobile Phase A: Water (0.1% FA), Mobile Phase B: ACN; Flow rate: 60 mL/min; Gradient: 2% B to 25% B in 8 min; Wavelength: 254/220 nm; RT1 (min): 7.65). The fractions containing the desired product were combined and lyophilized to afford (3-(((1,7-naphthyridin-4-yl)oxy)methyl)-phenyl) (imino)(methyl)-lambda6-sulfanone (17.5 mg, 10%) as a white solid. MS (ESI, pos. ion) m/z: 314.1 (M+1). ¹H NMR (400 MHZ, DMSO-d₆, ppm) δ 9.35 (s, 1H), 8.92 (d, J=5.2 Hz, 1H), 8.61 (d, J=5.6 Hz, 1H), 8.15 (d, J=1.9 Hz, 1H), 8.03 (d, J=5.6 Hz, 1H), 7.96 (d, J=7.8 Hz, 1H), 7.87 (d, J=7.6 Hz, 1H), 7.69 (t, J=7.7 Hz, 1H), 7.39 (d, J=5.2 Hz, 1H), 5.56 (s, 2H), 4.30 (s, 1H), 3.10 (s, 3H).

### Example 8

### Synthesis of ((1-(1,7-naphthyridin-4-yl) piperidin-4-yl)methyl) (imino)(methyl)-lambda6-sulfanone

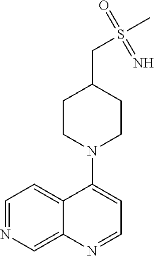 [SMILES: CS(=N)(=O)CC1CCN(c2ccnc3cnccc23)CC1]

### Step 1: (1-(1,7-Naphthyridin-4-yl) piperidin-4-yl) methanol

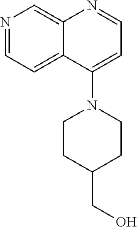 [SMILES: OCC1CCN(c2ccnc3cnccc23)CC1]

A mixture of 4-chloro-1,7-naphthyridine (480 mg, 2.93 mmol, 1 equiv), piperidin-4-ylmethanol (336 mg, 2.92 mmol, 1.50 equiv), X-Phos (279 mg, 0.58 mmol, 0.2 equiv), Pd₂ (dba)₃ (268 mg, 0.29 mmol, 0.1 equiv) and Cs₂CO₃ (2.86 g, 8.7 mmol, 3 equiv) in 1,4-dioxane (10 ml) was stirred at 100° C. for 2 h under nitrogen atmosphere. After cooling to room temperature, the resulting mixture was diluted with DCM and washed with water and brine. The organic layer was dried over anhydrous sodium sulfate, filtered and concentrated under reduced pressure. The residue was purified by reverse phase flash chromatography chromatography, eluted with ACN/H₂O (60:40) to afford (1-(1,7-naphthyridin-4-yl) piperidin-4-yl) methanol (180 mg, 22%) as a yellow solid.

### Step 2: (1-(1,7-Naphthyridin-4-yl) piperidin-4-yl)methyl methanesulfonate

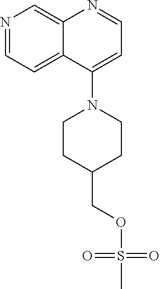 [SMILES: CS(=O)(=O)OCC1CCN(c2ccnc3cnccc23)CC1]

To a mixture of (1-(1,7-naphthyridin-4-yl) piperidin-4-yl) methanol (160 mg, 0.57 mmol, 1 equiv) and TEA (174 mg, 1.72 mmol, 3 equiv) in DCM (10 mL) was added MsCl (78.7 mg, 0.69 mmol, 1.2 equiv) at 0° C. The resulting mixture was stirred for 1 h at room temperature. The reaction mixture was concentrated under reduced pressure. The residue was purified by reverse phase flash chromatography, eluted with ACN/H₂O (70:30) to afford (1-(1,7-naphthyridin-4-yl) piperidin-4-yl)methyl methanesulfonate (100 mg, 54%) as a yellow solid.

### Step 3:4-(4-((Methylthio)methyl) piperidin-1-yl)-1,7-naphthyridine

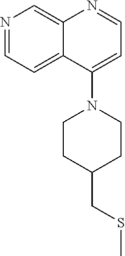 [SMILES: CSCC1CCN(c2ccnc3cnccc23)CC1]

To a solution of (1-(1,7-naphthyridin-4-yl) piperidin-4-yl)methyl methanesulfonate (90 mg, 0.28 mmol, 1 equiv) in EtOH (3 mL) was added 20% NaSMe in water (590 mg, 1.68 mmol, 6 equiv) at 0° C. The resulting solution was stirred for another 4 h at room temperature and then concentrated under reduced pressure. The residue was purified by reverse phase flash chromatography, eluted with ACN/H₂O (45/55). The fractions were concentrated to afford 4-(4-((methylthio)methyl) piperidin-1-yl)-1,7-naphthyridine (23 mg, 30%) as a white solid.

### Step 4: ((1-(1,7-Naphthyridin-4-yl) piperidin-4-yl)methyl) (imino)(methyl)-lambda6-sulfanone

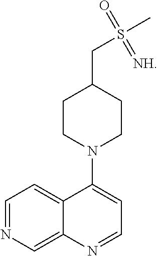 [SMILES: CS(=N)(=O)CC1CCN(c2ccnc3cnccc23)CC1]

The title compound was synthesized by proceeding analogously as described in Example 1, Step 5 except 4-(4-((methylthio)methyl) piperidin-1-yl)-1,7-naphthyridine (23 mg, 0.08 mmol) was used. The crude product was purified by prep-HPLC with the following conditions: (Column: XBridge Prep Phenyl OBD C18 Column, 30*150 mm, 5 μm; Mobile Phase A: Water (10 nmol/L NH₄HCO₃), Mobile Phase B: ACN; Flow rate: 60 mL/min; Gradient: 2% B to 24% B in 13 min; Wavelength: 254/220 nm). The fractions containing the desired product were combined and lyophilized to afford ((1-(1,7-naphthyridin-4-yl) piperidin-4-yl)methyl) (imino)(methyl)-lambda6-sulfanone (3.1 mg, 12%) as a yellow solid. MS (ESI, pos. ion) m/z: 305.0 (M+1). ¹H NMR (400 MHZ, DMSO-d₆, ppm) δ 9.28 (s, 1H), 8.78 (d, J=5.0 Hz, 1H), 8.54 (d, J=5.8 Hz, 1H), 7.80 (d, J=5.8 Hz, 1H), 7.14 (d, J=5.1 Hz, 1H), 3.74 (s, 1H), 3.59 (d, J=12.3 Hz, 2H), 3.15 (d, J=6.3 Hz, 2H), 2.97 (s, 3H), 2.94-2.91 (m, 2H), 2.23 (d, J=12.6 Hz, 1H), 2.13-2.01 (m, 2H), 1.70-1.61 (m, 2H).

### Example 9

### Synthesis of (4-(((1,7-naphthyridin-4-yl)oxy)methyl)phenyl) (cyclopropyl) (imino)-lambda6-sulfanone

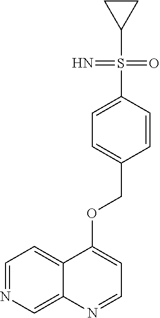 [SMILES: N=S(=O)(c1ccc(COc2ccnc3cnccc23)cc1)C1CC1]

### Step 1:4-(Cyclopropylsulfanyl)benzoic acid

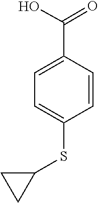 [SMILES: O=C(O)c1ccc(SC2CC2)cc1]

To a stirred solution of 4-mercaptobenzoic acid (2.00 g, 12.97 mmol, 1 equiv) in DMSO (20 mL) was added potassium tert-butoxide (3.64 g, 32.43 mmol, 2.5 equiv) and bromocyclopropane (1.6 mL) at room temperature. After stirring overnight at 120° C., the resulting mixture was dissolved in water and acidified to pH=5 with 1 M HCl (aq). The precipitated solids were collected by filtration, washed with water (150 mL) and dried under reduced pressure to give 4-(cyclopropylsulfanyl)benzoic acid (1.69 g, 67%) as a white solid.

### Step 2: [4-(Cyclopropylsulfanyl)phenyl]methanol

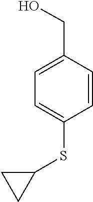 [SMILES: OCc1ccc(SC2CC2)cc1]

The title compound was synthesized by proceeding analogously as described in Example 7, Step 1 except 4-(cyclopropylsulfanyl)benzoic acid (500 mg, 2.57 mmol) was used. [4-(cyclopropylsulfanyl)phenyl]methanol (292 mg, 62%) was obtained as a colorless oil.

### Step 3:4-((4-(Cyclopropylthio)benzyl)oxy)-1,7-naphthyridine

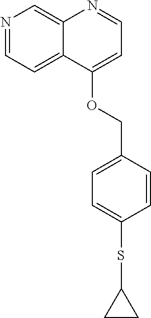 [SMILES: c1cc2c(OCc3ccc(SC4CC4)cc3)ccnc2cn1]

The title compound was synthesized by proceeding analogously as described in Example 5 Step 4 except [4-(cyclopropylsulfanyl)phenyl]methanol (246 mg, 1.36 mmol) was used. 4-((4-(cyclopropylthio)benzyl)oxy)-1,7-naphthyridine (198 mg, 70%) was obtained as a yellow solid.

### Step 4: (4-(((1,7-Naphthyridin-4-yl)oxy)methyl)phenyl) (cyclopropyl) (imino)-lambda6-sulfanone

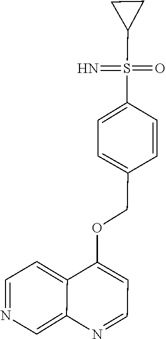 [SMILES: N=S(=O)(c1ccc(COc2ccnc3cnccc23)cc1)C1CC1]

The title compound was synthesized by proceeding analogously as described in Example 1, Step 5 except 4-((4-(cyclopropylthio)benzyl)oxy)-1,7-naphthyridine (178 mg, 0.57 mmol) was used. The crude product was purified by prep-HPLC with the following conditions: (Column: XselectCSH Prep OBD C18 Column, 30*150 mm, 5 μm; Mobile Phase A: Water (0.1% FA), Mobile Phase B: ACN; Flow rate: 60 mL/min; Gradient: 5% B to 35% B in 8 min; Wavelength: 254/220 nm; RT1 (min): 6.97). The fractions containing the desired product were combined and lyophilized to afford (4-(((1,7-naphthyridin-4-yl)oxy)methyl)phenyl)-(cyclopropyl) (imino)-lambda6-sulfanone (70.4 mg, 35%) as a white solid. MS (ESI, pos. ion) m/z: 340.1 (M+1). ¹H NMR (400 MHZ, DMSO-d₆, ppm) δ 9.36 (s, 1H), 8.92 (d, J=5.1 Hz, 1H), 8.62 (d, J=5.7 Hz, 1H), 8.07 (d, J=5.6 Hz, 1H), 8.00-7.93 (m, 2H), 7.79 (d, J=8.1 Hz, 2H), 7.37 (d, J=5.2 Hz, 1H), 5.57 (s, 2H), 4.25 (s, 1H), 2.70-2.67 (m, 1H), 1.18-1.08 (m, 1H), 1.03-0.85 (m, 3H).

### Example 10

### Synthesis of ((4-hydroxy-1-(1,7-naphthyridin-4-yl) piperidin-4-yl)methyl) (imino)(methyl)-lambda6-sulfanone

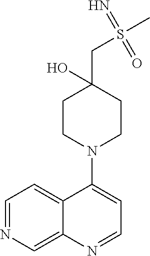 [SMILES: CS(=N)(=O)CC1(O)CCN(c2ccnc3cnccc23)CC1]

### Step 1:4-((Methylthio)methyl)-1-(1,7-naphthyridin-4-yl) piperidin-4-ol

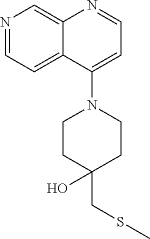 [SMILES: CSCC1(O)CCN(c2ccnc3cnccc23)CC1]

The title compound was synthesized by proceeding analogously as described in Example 1, Step 4 except 4-((methylthio)methyl) piperidin-4-ol hydrochloride (551 mg, 2.80 mmol) was used and stirred overnight at 90° C. 4-((methylthio)methyl)-1-(1,7-naphthyridin-4-yl) piperidin-4-ol (320 mg, 70%) was obtained as a yellow solid.

### Step 2: ((4-Hydroxy-1-(1,7-naphthyridin-4-yl) piperidin-4-yl)methyl) (imino)(methyl)-lambda6-sulfanone

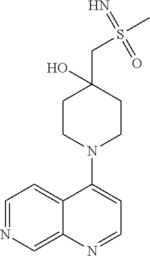 [SMILES: CS(=N)(=O)CC1(O)CCN(c2ccnc3cnccc23)CC1]

The title compound was synthesized by proceeding analogously as described in Example 1, Step 5 except 4-((methylthio)methyl)-1-(1,7-naphthyridin-4-yl) piperidin-4-ol (220 mg, 0.76 mmol) was used. The crude product was purified by prep-HPLC with the following conditions: (Column: XBridge Prep OBD C18 Column, 19*250 mm, 5 μm; Mobile Phase A: Water (10 mmol/L NH₄HCO₃), Mobile Phase B: MEOH; Flow rate: 25 mL/min; Gradient: 3% B to 33% B in 15 min; Wavelength: 254/220 nm; RT1 (min): 14.42). The fractions containing the desired product were combined and lyophilized to afford ((4-hydroxy-1-(1,7-naphthyridin-4-yl) piperidin-4-yl)methyl) (imino)(methyl)-lambda6-sulfanone (23.4 mg, 10%) as a brown semi-solid. MS (ESI, pos. ion) m/z: 321.2 (M+1). ¹H NMR (400 MHz, DMSO-d₆, ppm) δ 9.28 (s, 1H), 8.82 (d, J=4.8 Hz, 1H), 8.52 (d, J=5.6 Hz, 1H), 7.81 (d, J=5.6 Hz, 1H), 7.15 (d, J=4.8 Hz, 1H), 5.64 (s, 1H), 4.03 (s, 1H), 3.47-3.43 (m, 2H), 3.39-3.32 (m, 2H), 3.25-3.19 (m, 2H), 3.06 (s, 3H), 2.08-1.99 (m, 4H).

### Example 11

### Synthesis of (2-(1-(1,7-naphthyridin-4-yl) piperidin-4-yl)ethyl) (imino)(methyl)-lambda6-sulfanone

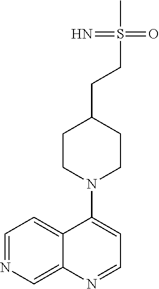 [SMILES: CS(=N)(=O)CCC1CCN(c2ccnc3cnccc23)CC1]

### Step 1:2-(1-(1,7-Naphthyridin-4-yl) piperidin-4-yl) ethan-1-ol

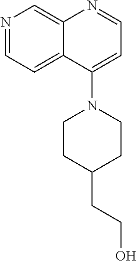 [SMILES: OCCC1CCN(c2ccnc3cnccc23)CC1]

To a stirred solution of 4-chloro-1,7-naphthyridine (500 mg, 3.03 mmol, 1 equiv) in DMF (15 mL) was added Cs₂CO₃ (2.97 g, 9.11 mmol, 3 equiv), 4-piperidineethanol (588 mg, 4.55 mmol, 1.5 equiv), Pd(OAc)₂ (136 mg, 0.61 mmol, 0.2 equiv) and Xantphos (351 mg, 0.61 mmol, 0.2 equiv) at room temperature under nitrogen atmosphere. After stirring for 2 h at 80° C., the resulting mixture was diluted DCM and washed with H₂O and brine. The organic layer was dried over anhydrous sodium sulfate, filtered and concentrated under reduced pressure. The residue was purified by silica gel column chromatography, eluted with MeOH/DCM (10:90) and concentrated under reduced pressure to give 2-(1-(1,7-naphthyridin-4-yl) piperidin-4-yl) ethan-1-ol (302 mg, 38%) as a yellow solid.

### Step 2:2-(1-(1,7-Naphthyridin-4-yl) piperidin-4-yl)ethyl methanesulfonate

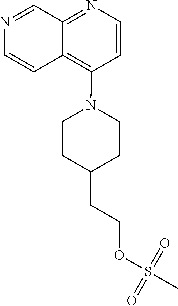 [SMILES: CS(=O)(=O)OCCC1CCN(c2ccnc3cnccc23)CC1]

To a stirred solution of 2-(1-(1,7-naphthyridin-4-yl) piperidin-4-yl) ethan-1-ol (280 mg, 1.08 mmol, 1 equiv) in DCM (6 mL) was added TEA (202 mg, 2.17 mmol, 0.1 equiv) methanesulfonyl methanesulfonate (284 mg, 1.63 mmol, 1.5 equiv) and DMAP (13 mg, 0.11 mmol, 0.1 equiv) at 0° C. After stirring for 1 h at room temperature, the resulting mixture was diluted with DCM and washed with water and brine. The organic layer was dried over anhydrous sodium sulfate, filtered and concentrated under reduced pressure. The residue was purified by silica gel column chromatography, eluted with MeOH/DCM (6:94) to afford 2-(1-(1,7-naphthyridin-4-yl) piperidin-4-yl)ethyl methanesulfonate (200 mg, 54%) as a yellow solid.

### Step 3:4-[4-[2-(methylsulfanyl)ethyl]piperidin-1-yl]-1,7-naphthyridine

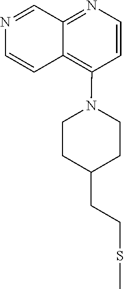 [SMILES: CSCCC1CCN(c2ccnc3cnccc23)CC1]

The title compound was synthesized by proceeding analogously as described in Example 1, Step 2 except 2-(1-(1,7-naphthyridin-4-yl) piperidin-4-yl)ethyl methanesulfonate (180 mg, 0.537 mmol) was used. 4-[4-[2-(methylsulfanyl)ethyl]piperidin-1-yl]-1,7-naphthyridine (111 mg, 72%) was obtained as a yellow solid.

### Step 4: (2-(1-(1,7-Naphthyridin-4-yl) piperidin-4-yl)ethyl) (imino)(methyl)-lambda6-sulfanone

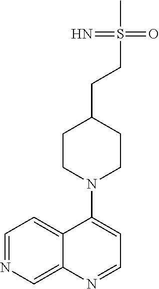 [SMILES: CS(=N)(=O)CCC1CCN(c2ccnc3cnccc23)CC1]

The title compound was synthesized by proceeding analogously as described in Example 1, Step 5 except 4-[4-[2-(methylsulfanyl)ethyl]piperidin-1-yl]-1,7-naphthyridine (95 mg, 0.33 mmol) was used. The crude product was purified by prep-HPLC with the following conditions: (Column: XBridge Prep OBD C18 Column, 30*150 mm, 5 μm; Mobile Phase A: Water (10 nmol/L NH₄HCO₃), Mobile Phase B: ACN; Flow rate: 60 mL/min; Gradient: 2% B to 30% B in 10 min; Wavelength: 254/220 nm; RT1 (min): 8.38). The fractions containing the desired product were combined and lyophilized to afford (2-(1-(1,7-naphthyridin-4-yl) piperidin-4-yl)ethyl) (imino)(methyl)-lambda6-sulfanone (29.4 mg, 27%) as a yellow semi-solid. MS (ESI, pos. ion) m/z: 319.1 (M+1). ¹H NMR (400 MHZ, DMSO-d₆, ppm) δ 9.28 (s, 1H), 8.77 (d, J=5.0 Hz, 1H), 8.53 (d, J=5.8 Hz, 1H), 7.81-7.78 (m, 1H), 7.13 (d, J=5.1 Hz, 1H), 3.61 (d, J=12.6 Hz, 3H), 3.16-3.08 (m, 2H), 2.91 (s, 3H), 2.86-2.83 (m, 2H), 1.89-1.86 (m, 2H), 1.83-1.71 (m, 2H), 1.64-1.61 (m, 1H), 1.53-1.50 (m, 2H).

### Example 12

### Synthesis of imino (4-(((8-methoxy-1,7-naphthyridin-4-yl)oxy)methyl)phenyl)(methyl)-lambda6-sulfanone

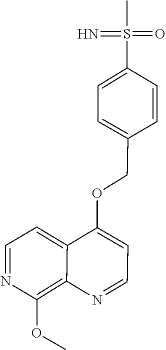 [SMILES: COc1nccc2c(OCc3ccc(S(C)(=N)=O)cc3)ccnc12]

### Step 1:5-(((2-Methoxypyridin-3-yl)amino)methylene)-2,2-dimethyl-1,3-dioxane-4,6-dione

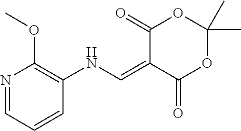 [SMILES: COc1ncccc1NC=C1C(=O)OC(C)(C)OC1=O]

A solution of 2-methoxypyridin-3-amine (3.00 g, 24.2 mmol, 1 equiv), 2,2-dimethyl-1,3-dioxane-4,6-dione (4.50 g, 31.46 mmol, 1.3 equiv) and trimethoxymethane (3.60 g, 33.8 mmol, 1.4 equiv) in acetonitrile (30 mL) was stirred at 80° C. for 2 h under nitrogen atmosphere. After cooling to room temperature, the resulting solution was concentrated under reduced pressure. The residue was purified by silica gel column chromatography, eluted with EA/PE (25:75). The fractions containing the desired product were combined and concentrated under reduced pressure to afford 5-(((2-methoxypyridin-3-yl)amino)methylene)-2,2-dimethyl-1,3-dioxane-4,6-dione (6.50 g, 97%) as a yellow solid.

### Step 2:8-Methoxy-1,7-naphthyridin-4-ol

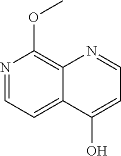

A mixture of 5-(((2-methoxypyridin-3-yl)amino)methylene)-2,2-dimethyl-1,3-dioxane-4,6-dione (1.50 g, 5.4 mmol) in diphenyl ether (38 mL) was stirred at 225° C. for 1.5 h. After cooling to room temperature, 100 ml PE was poured into the reaction mixture, the precipitated solids were collected by filtration and washed with PE (100 mL). The crude product was purified by reverse phase flash chromatography, eluted with ACN/H₂O (23:77) to afford 8-methoxy-1,7-naphthyridin-4-ol (600 mg, 63%) as a white solid.

### Step 3:8-Methoxy-4-((4-(methylthio)benzyl)oxy)-1,7-naphthyridine

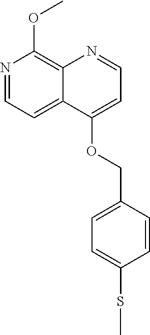 [SMILES: COc1nccc2c(OCc3ccc(SC)cc3)ccnc12]

To a mixture of 8-methoxy-1,7-naphthyridin-4-ol (550 mg, 2.96 mmol, 1 equiv) in toluene (8 mL) was added (4-(methylthio)phenyl) methanol (912 mg, 5.92 mmol, 2 equiv) and 2-(tributylphosphanylidene) acetonitrile (1.40 g, 5.92 mmol, 2 equiv). The resulting mixture was stirred at 130° C. for 2 h. After cooling to room temperature, the resulting mixture was concentrated under reduced pressure. The residue was purified by silica gel chromatography, eluted with MeOH/DCM (9:91) to give 650 mg crude product which was purified by reversedphase flash, eluted with ACN/H₂O (45:55) to afford 8-methoxy-4-((4-(methylthio)benzyl)oxy)-1,7-naphthyridine (470 mg, 44%) as a white solid.

### Step 4: Imino (4-(((8-methoxy-1,7-naphthyridin-4-yl)oxy)methyl)phenyl)(methyl)-lambda6-sulfanone

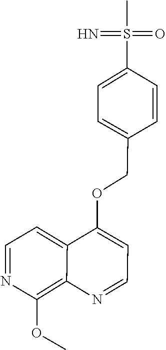 [SMILES: COc1nccc2c(OCc3ccc(S(C)(=N)=O)cc3)ccnc12]

The title compound was synthesized by proceeding analogously as described in Example 1, Step 5 except 8-methoxy-4-((4-(methylthio)benzyl)oxy)-1,7-naphthyridine (110 mg, 0.35 mmol) was used. The crude product was purified by prep-HPLC with the following conditions: (Column: XBridge Prep OBD C18 Column, 30*150 mm, 5 μm; Mobile Phase A: Water (10 nmol/L NH₄HCO₃), Mobile Phase B: ACN; Flow rate: 60 mL/min; Gradient: 10% B to 40% B in 10 min; Wavelength: 254/220 nm; RT1 (min): 8.6). The fractions containing the desired product were combined and lyophilized to afford imino (4-(((8-methoxy-1,7-naphthyridin-4-yl)oxy)methyl)phenyl)(methyl)-lambda6-sulfanone (75.4 mg, 62%) as a white solid. MS (ESI, pos. ion) m/z: 344.0 (M+1). ¹H NMR (400 MHZ, DMSO-d₆, ppm) δ 8.78 (d, J=5.2 Hz, 1H), 8.08 (d, J=6 Hz, 1H), 8.00 (d, J=8.4 Hz, 2H), 7.77 (d, J=8.4 Hz, 2H), 7.58 (d, J=6.0 Hz, 1H), 7.36 (d, J=5.2 Hz, 1H), 5.53 (s, 2H), 4.26 (s, 1H), 4.05 (s, 3H), 3.09 (s, 3H).

### Example 13

### Synthesis of cyclopropyl(imino) (4-(((8-methoxy-1,7-naphthyridin-4-yl)oxy)methyl)phenyl)-lambda6-sulfanone

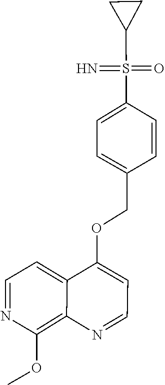 [SMILES: COc1nccc2c(OCc3ccc(S(=N)(=O)C4CC4)cc3)ccnc12]

### Step 1:4-((4-(Cyclopropylthio)benzyl)oxy)-8-methoxy-1,7-naphthyridine

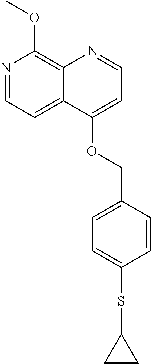 [SMILES: COc1nccc2c(OCc3ccc(SC4CC4)cc3)ccnc12]

The title compound was synthesized by proceeding analogously as described in Example 5, Step 4 except 4-chloro-8-methoxy-1,7-naphthyridine (200 mg, 1.02 mmol) and [4-(cyclopropylsulfanyl)phenyl]methanol (370 mg, 2.05 mmol) were used. 4-((4-(cyclopropylthio)benzyl)oxy)-8-methoxy-1,7-naphthyridine (121 mg, 34%) was obtained as a yellow solid.

### Step 2: Cyclopropyl(imino) (4-(((8-methoxy-1,7-naphthyridin-4-yl)oxy)methyl)phenyl)-lambda6-sulfanone

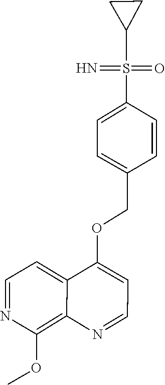 [SMILES: COc1nccc2c(OCc3ccc(S(=N)(=O)C4CC4)cc3)ccnc12]

The title compound was synthesized by proceeding analogously as described in Example 1, Step 5 except 4-((4-(cyclopropylthio)benzyl)oxy)-8-methoxy-1,7-naphthyridine (100 mg, 0.295 mmol) was used. The crude product was purified by prep-HPLC with the following conditions: (Column: XBridge Prep Shield RP C18 Column, 30*150 mm, 5 μm; Mobile Phase A: Water (10 mmol/L NH₄HCO₃), Mobile Phase B: ACN; Flow rate: 60 mL/min; Gradient: 14% B to 35% B in 7 min; Wavelength: 254/220 nm; RT1 (min): 7). The fractions containing the desired product were combined and lyophilized to afford cyclopropyl(imino) (4-(((8-methoxy-1,7-naphthyridin-4-yl)oxy)methyl)phenyl)-lambda6-sulfanone (42.0 mg, 38%) as a white solid. MS (ESI, pos. ion) m/z: 370.1 (M+1). ¹H NMR (400 MHZ, DMSO-d₆, ppm) δ 8.79 (d, J=5.2 Hz, 1H), 8.09 (d, J=5.8 Hz, 1H), 7.96 (d, J=8.2 Hz, 2H), 7.77 (d, J=8.1 Hz, 2H), 7.59 (d, J=5.8 Hz, 1H), 7.37 (d, J=5.3 Hz, 1H), 5.53 (s, 2H), 4.26 (s, 1H), 4.05 (s, 3H), 2.69-2.66 (m, 1H) 1.14-1.11 (m, 1H), 1.03-0.84 (m, 3H).

### Example 14

### Synthesis of cyclopropyl(imino) (3-(((8-methoxypyrido[3,4-d]pyrimidin-4-yl)oxy)methyl)-phenyl)-lambda6-sulfanone

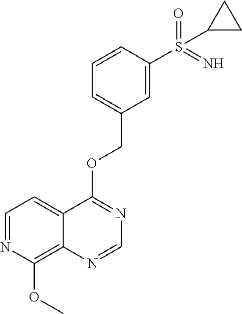 [SMILES: COc1nccc2c(OCc3cccc(S(=N)(=O)C4CC4)c3)ncnc12]

### Step 1:3-(Cyclopropylsulfanyl)benzoic acid

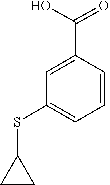 [SMILES: O=C(O)c1cccc(SC2CC2)c1]

The title compound was synthesized by proceeding analogously as described in Example 9, Step 1 except 3-sulfanylbenzoic acid (5.00 g, 30.80 mmol) was used. 3-(cyclopropylsulfanyl)benzoic acid (3.99 g, 61%) was obtained as a colorless oil.

### Step 2: [3-(Cyclopropylsulfanyl)phenyl]methanol

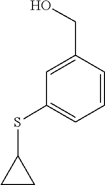 [SMILES: OCc1cccc(SC2CC2)c1]

The title compound was synthesized by proceeding analogously as described in Example 7, Step 1 except 3-(cyclopropylsulfanyl)benzoic acid (24.00 g, 117.37 mmol) was used. [3-(cyclopropylsulfanyl)phenyl]methanol (20.00 g, 86%) was obtained as a yellow oil.

### Step 3:8-Methoxy-3H-pyrido[3,4-d]pyrimidin-4-one

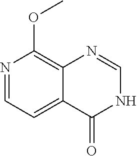

To a stirred solution of 3-amino-2-methoxypyridine-4-carboxylic acid (4.84 g, 28.783 mmol, 1 equiv) in 2-methoxyethan-1-ol (90 mL) was added formamidine acetate (5.99 g, 57.56 mmol, 2 equiv) at room temperature. After stirring at 120° C. for 24 h, the resulting mixture was cooled to room temperature and poured into water. The precipitated solids were collected by filtration, washed with water, and dried under reduced pressure to give 8-methoxy-3H-pyrido[3,4-d]pyrimidin-4-one (4.60 g, 90%) as a grey solid.

### Step 4:4-Chloro-8-methoxypyrido[3,4-d]pyrimidine

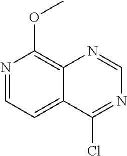

To a stirred solution of 8-methoxy-3H-pyrido[3,4-d]pyrimidin-4-one (2.00 g, 11.28 mmol, 1 equiv) in toluene (40 mL) was added DIEA (2.92 g, 22.57 mmol, 2 equiv) and POCl₃ (8.65 g, 56.44 mmol, 5 equiv) at 0° C. After stirring overnight at 90° C., the resulting mixture was cooled to room temperature, concentrated under reduced pressure, diluted with EtOAc and washed with H₂O and brine. The organic layer was dried over anhydrous sodium sulfate, filtered and concentrated under reduced pressure to give 4-chloro-8-methoxypyrido[3,4-d]pyrimidine (2.08 g, 94%) as a yellow solid.

### Step 5:4-[[3-(Cyclopropylsulfanyl)phenyl]methoxy]-8-methoxypyrido[3,4-d]pyrimidine

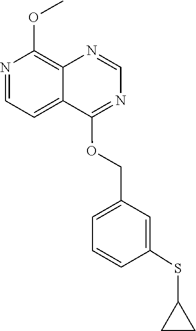 [SMILES: COc1nccc2c(OCc3cccc(SC4CC4)c3)ncnc12]

To a stirred solution of 4-chloro-8-methoxypyrido[3,4-d]pyrimidine (300 mg, 1.53 mmol, 1 equiv) in DMF (5 mL) was added Cs₂CO₃ (999 mg, 3.06 mmol, 2 equiv) and [3-(cyclopropyl-sulfanyl)phenyl]methanol (331 mg, 1.84 mmol, 1.2 equiv) at room temperature. After stirring at 80° C. for 2 h, the resulting mixture was cooled to room temperature, diluted with DCM and washed with H₂O and brine. The organic layer was dried over anhydrous sodium sulfate, filtered and concentrated under reduced pressure. The crude product was purified by silica gel column chromatography, eluted with EA/PE (44:56) to afford 4-[[3-(cyclopropylsulfanyl)phenyl]methoxy]-8-methoxypyrido[3,4-d]pyrimidine (261 mg, 50%) as a yellow solid.

### Step 6: Cyclopropyl(imino) (3-(((8-methoxypyrido[3,4-d]pyrimidin-4-yl)oxy)methyl)phenyl)-lambda6-sulfanone

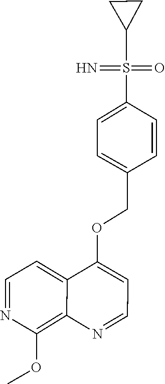 [SMILES: COc1nccc2c(OCc3ccc(S(=N)(=O)C4CC4)cc3)ccnc12]

The title compound was synthesized by proceeding analogously as described in Example 1, Step 5 except 4-[[3-(cyclopropylsulfanyl)phenyl]methoxy]-8-methoxypyrido[3,4-d]pyrimidine (241 mg, 0.71 mmol) was used. The crude product was purified by prep-HPLC with the following conditions: (Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 13% B to 38% B in 10 min; Wavelength: 254/220 nm; RT1 (min): 8.68). The fractions containing the desired product were combined and lyophilized to afford cyclopropyl(imino) (3-(((8-methoxypyrido[3,4-d]pyrimidin-4-yl)oxy)methyl)phenyl)-lambda6-sulfanone (87.7 mg, 33%) as a white solid. MS (ESI, pos. ion) m/z: 371.2 (M+1). ¹H NMR (400 MHZ, DMSO-d₆, ppm) δ 8.93 (s, 1H), 8.24 (d, J=5.7 Hz, 1H), 8.08 (t, J=1.8 Hz, 1H), 7.93-7.86 (m, 1H), 7.85-7.79 (m, 1H), 7.65 (t, J=7.7 Hz, 1H), 7.53 (d, J=5.7 Hz, 1H), 5.76 (s, 2H), 4.27 (s, 1H), 4.07 (s, 3H), 2.82-2.63 (m, 1H), 1.17-1.06 (m, 1H), 1.03-0.82 (m, 3H).

### Example 15

### Synthesis of cyclopropyl(imino) (3-(((8-methoxy-1,7-naphthyridin-4-yl)oxy)methyl)phenyl)-lambda6-sulfanone

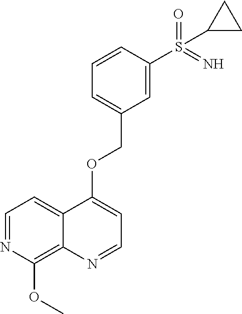 [SMILES: COc1nccc2c(OCc3cccc(S(=N)(=O)C4CC4)c3)ccnc12]

### Step 1:4-((3-(Cyclopropylthio)benzyl)oxy)-8-methoxy-1,7-naphthyridine

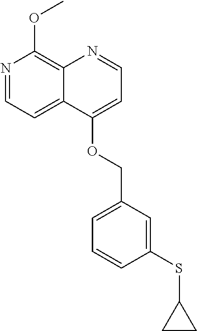 [SMILES: COc1nccc2c(OCc3cccc(SC4CC4)c3)ccnc12]

The title compound was synthesized by proceeding analogously as described in Example 12, Step 3 except (3-(cyclopropylthio)phenyl) methanol (511 mg, 2.84 mmol) was used. 4-((3-(cyclopropylthio)benzyl)oxy)-8-methoxy-1,7-naphthyridine (169 mg, 35%) was obtained as a yellow solid.

### Step 2: Cyclopropyl(imino) (3-(((8-methoxy-1,7-naphthyridin-4-yl)oxy)methyl)phenyl)-lambda6-sulfanone

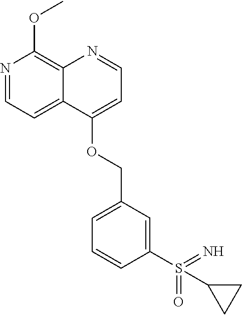 [SMILES: COc1nccc2c(OCc3cccc(S(=N)(=O)C4CC4)c3)ccnc12]

The title compound was synthesized by proceeding analogously as described in Example 1, Step 5 except 4-((3-(cyclopropylthio)benzyl)oxy)-8-methoxy-1,7-naphthyridine (154 mg, 0.46 mmol) was used. The crude product was purified by prep-HPLC with the following conditions: (Column: XBridge Prep OBD C18 Column, 30*150 mm, 5 μm; Mobile Phase A: Water (10 nmol/L NH₄HCO₃), Mobile Phase B: ACN; Flow rate: 60 mL/min; Gradient: 10% B to 40% B in 10 min; Wave Length: 254/220 nm; RT1 (min): 8.6). The fractions containing the desired product were combined and lyophilized to afford cyclopropyl(imino) (3-(((8-methoxy-1,7-naphthyridin-4-yl)oxy)methyl)phenyl)-lambda6-sulfanone (74.4 mg, 44%) as a white solid. MS (ESI, pos. ion) m/z: 370.1 (M+1). ¹H NMR (400 MHZ, DMSO-d₆, ppm) δ 8.79 (d, J=5.2 Hz, 1H), 8.09-8.07 (m, 2H), 7.91 (d, J=8 Hz, 1H), 7.84 (d, J=7.6 Hz, 1H), 7.70-7.67 (m, 1H), 7.55 (d, J=5.6 Hz, 1H), 7.37 (d, J=5.2 Hz, 1H), 5.53 (s, 2H), 4.29 (s, 1H), 4.05 (s, 3H), 2.69-2.66 (m, 1H), 1.15-1.09 (m, 1H), 1.01-0.97 (m, 3H).

Additional racemic cyclopropyl(imino) (3-(((8-methoxy-1,7-naphthyridin-4-yl)oxy)methyl)phenyl)-lambda6-sulfanone was prepared following the procedures in the above paragraphs and purified by prep-chiral-HPLC under following conditions (Column: CHIRALPAK 1H 3*25 cm, 5 μm; Mobile Phase A: CO₂, Mobile Phase B: IPA (1%-2M-NH₃-IPA); Flow rate: 90 mL/min; Gradient: isocratic 46% B; Column Temperature (C): 35; Back Pressure (bar): 100; Wave Length: 220 nm; Sample Solvent: MEOH; Injection Volume: 4 mL; Number Of Runs: 45) to afford (R)-cyclopropyl(imino) (3-(((8-methoxy-1,7-naphthyridin-4-yl)oxy)methyl)phenyl)-lambda6-sulfanone as an off-white solid (RT1:7.03 min) and(S)-cyclopropyl(imino) (3-(((8-methoxy-1,7-naphthyridin-4-yl)oxy)methyl)phenyl)-lambda6-sulfanone as an off-white solid (RT2:10.23 min).

Fraction 1: (R)-cyclopropyl(imino) (3-(((8-methoxy-1,7-naphthyridin-4-yl)oxy)methyl)phenyl)-lambda6-sulfanone. MS (ESI, pos. ion) m/z: 370.0 (M+1). ¹H NMR (400 MHZ, DMSO-d₆, ppm) ¹H NMR (400 MHZ, DMSO-d₆) δ 8.79 (d, J=5.2 Hz, 1H), 8.09-8.07 (m, 2H), 7.92-7.82 (m, 2H), 7.68 (t, J=7.6 Hz, 1H), 7.55 (d, J=5.8 Hz, 1H), 7.38 (d, J=5.3 Hz, 1H), 5.53 (s, 2H), 4.28 (s, 1H), 4.05 (s, 3H), 2.70-2.64 (m, 1H), 1.23-1.11 (m, 1H), 1.09-0.85 (m, 3H).

Fraction 2: (S)-cyclopropyl(imino) (3-(((8-methoxy-1,7-naphthyridin-4-yl)oxy)methyl)phenyl)-lambda6-sulfanone. MS (ESI, pos. ion) m/z: 370.0 (M+1). ¹H NMR (400 MHZ, DMSO-d₆) δ 8.79 (d, J=5.2 Hz, 1H), 8.09-8.07 (m, 2H), 7.92-7.82 (m, 2H), 7.68 (t, J=7.6 Hz, 1H), 7.55 (d, J=5.8 Hz, 1H), 7.38 (d, J=5.3 Hz, 1H), 5.53 (s, 2H), 4.28 (s, 1H), 4.05 (s, 3H), 2.70-2.64 (m, 1H), 1.23-1.11 (m, 1H), 1.09-0.85 (m, 3H).

### Example 16

### Synthesis of imino (4-(((8-methoxypyrido[3,4-d]pyrimidin-4-yl)oxy)methyl)phenyl)(methyl)-lambda6-sulfanone

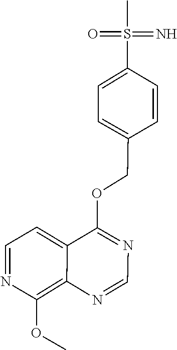 [SMILES: COc1nccc2c(OCc3ccc(S(C)(=N)=O)cc3)ncnc12]

### Step 1:8-Methoxy-4-[4-(methylsulfanyl)phenyl]methoxy]pyrido[3,4-d]pyrimidine

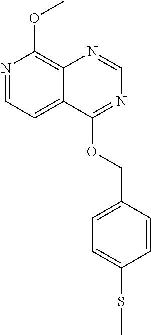 [SMILES: COc1nccc2c(OCc3ccc(SC)cc3)ncnc12]

The title compound was synthesized by proceeding analogously as described in Example 14, Step 5 except [4-(methylsulfanyl)phenyl]methanol (283 mg, 1.84 mmol) was used. 8-methoxy-4-[[4-(methylsulfanyl)phenyl]methoxy]pyrido[3,4-d]pyrimidine (261 mg, 54%) was obtained as a white oil.

### Step 2: Imino (4-(((8-methoxypyrido[3,4-d]pyrimidin-4-yl)oxy)methyl)phenyl)(methyl)-lambda6-sulfanone

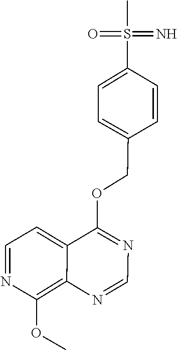 [SMILES: COc1nccc2c(OCc3ccc(S(C)(=N)=O)cc3)ncnc12]

The title compound was synthesized by proceeding analogously as described in Example 1, Step 5 except 8-methoxy-4-[4-(methylsulfanyl)phenyl]methoxy]-pyrido[3,4-d]pyrimidine (180 mg, 0.574 mmol) was used. The crude product was purified by filtration, washed with MeCN and lyophilized to afford imino (4-(((8-methoxypyrido[3,4-d]pyrimidin-4-yl)oxy)methyl)phenyl)(methyl)-lambda6-sulfanone (76.5 mg, 38%) as a white solid. MS (ESI, pos. ion) m/z: 345.2 (M+1). ¹H NMR (400 MHZ, DMSO-d₆, ppm) δ 8.91 (s, 1H), 8.24 (d, J=5.7 Hz, 1H), 7.97 (d, J=8.1 Hz, 2H), 7.77 (d, J=8.1 Hz, 2H), 7.57 (d, J=5.7 Hz, 1H), 5.75 (s, 2H), 4.25 (s, 1H), 4.07 (s, 3H), 3.08 (s, 3H).

### Example 17

### Synthesis of cyclopropyl(imino) (4-(((8-methoxypyrido[3,4-d]pyrimidin-4-yl)oxy)methyl)phenyl)-lambda6-sulfanone

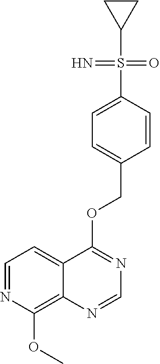 [SMILES: COc1nccc2c(OCc3ccc(S(=N)(=O)C4CC4)cc3)ncnc12]

### Step 1:4-((4-(Cyclopropylthio)benzyl)oxy)-8-methoxypyrido[3,4-d]pyrimidine

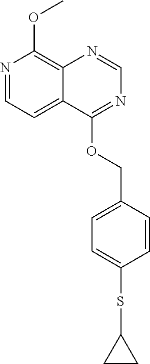 [SMILES: COc1nccc2c(OCc3ccc(SC4CC4)cc3)ncnc12]

The title compound was synthesized by proceeding analogously as described in Example 14, Step 5 except [4-(cyclopropylsulfanyl)phenyl]methanol (332 mg, 1.8 mmol, 1.2 equiv) was used. 4-((4-(cyclopropylthio)benzyl)oxy)-8-methoxypyrido[3,4-d]pyrimidine (60 mg, 11%) was obtained as a yellow oil.

### Step 2: Cyclopropyl(imino) (4-(((8-methoxypyrido[3,4-d]pyrimidin-4-yl)oxy)methyl)phenyl)-lambda6-sulfanone

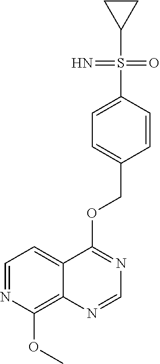 [SMILES: COc1nccc2c(OCc3ccc(S(=N)(=O)C4CC4)cc3)ncnc12]

The title compound was synthesized by proceeding analogously as described in Example 1, Step 5 except 4-((4-(cyclopropylthio)benzyl)oxy)-8-methoxypyrido[3,4-d]-pyrimidine (50 mg, 0.15 mmol) was used. The crude product was purified by prep-HPLC with the following conditions: (Column: YMC-Actus Triart C18 ExRS Column, 30*150 mm, 5 μm; Mobile Phase A: Water (10 mmol/L NH₄HCO₃), Mobile Phase B: ACN; Flow rate: 60 mL/min; Gradient: 7% B to 37% B in 7 min; Wavelength: 254/220 nm; RT1 (min): 6.82). The fractions containing the desired product were combined and lyophilized to afford cyclopropyl(imino) (4-(((8-methoxypyrido[3,4-d]pyrimidin-4-yl)oxy)methyl)phenyl)-lambda6-sulfanone (21.4 mg, 39%) as a white solid. MS (ESI, pos. ion) m/z: 371.1 (M+1). ¹H NMR (400 MHZ, DMSO-d₆, ppm) δ 8.68 (s, 1H), 8.21-8.17 (m, 1H), 7.89-7.82 (m, 2H), 7.57-7.49 (m, 3H), 5.31 (s, 2H), 4.19 (s, 1H), 4.02 (s, 3H), 2.66-2.59 (m, 1H), 1.11-1.05 (m, 1H), 0.99-0.79 (m, 3H).

### Example 18

### Synthesis of cyclopropyl(imino) (3-(((6-methoxypyrido[3,4-d]pyrimidin-4-yl)oxy)methyl)phenyl)-lambda6-sulfanone

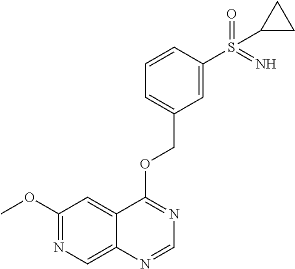 [SMILES: COc1cc2c(OCc3cccc(S(=N)(=O)C4CC4)c3)ncnc2cn1]

### Step 1:4-((3-(Cyclopropylthio)benzyl)oxy)-6-methoxypyrido[3,4-d]pyrimidine

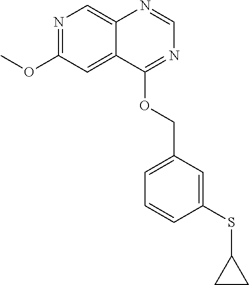 [SMILES: COc1cc2c(OCc3cccc(SC4CC4)c3)ncnc2cn1]

The title compound was synthesized by proceeding analogously as described in Example 14, Step 5 except 4-chloro-6-methoxypyrido[3,4-d]pyrimidine (300 mg, 1.53 mmol) was used. 4-((3-(cyclopropylthio)benzyl)oxy)-6-methoxypyrido[3,4-d]pyrimidine (314 mg, 60%) was obtained as a yellow oil.

### Step 2: Cyclopropyl(imino) (3-(((6-methoxypyrido[3,4-d]pyrimidin-4-yl)oxy)methyl)phenyl)-lambda6-sulfanone

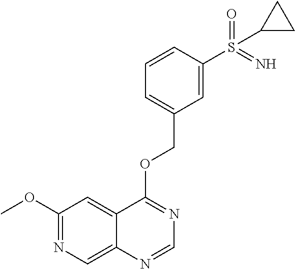 [SMILES: COc1cc2c(OCc3cccc(S(=N)(=O)C4CC4)c3)ncnc2cn1]

The title compound was synthesized by proceeding analogously as described in Example 1, Step 5 except 4-((3-(cyclopropylthio)benzyl)oxy)-6-methoxypyrido[3,4-d]pyrimidine (294 mg, 0.87 mmol) was used. The crude product was purified by prep-HPLC with the following conditions: (Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 15% B to 45% B in 10 min; Wavelength: 254/220 nm; RT1 (min): 8.6). The fractions containing the desired product were combined and lyophilized to afford cyclopropyl(imino) (3-(((6-methoxypyrido[3,4-d]pyrimidin-4-yl)oxy)methyl)phenyl)-lambda6-sulfanone (131.8 mg, 40%) as a white solid. MS (ESI, pos. ion) m/z: 371.1 (M+1). ¹H NMR (400 MHZ, DMSO-d₆, ppm) δ 9.12 (s, 1H), 8.79 (s, 1H), 8.15-8.06 (m, 1H), 7.90-7.83 (m, 2H), 7.67-7.61 (m, 1H), 7.29-7.28 (m, 1H), 5.77 (s, 2H), 4.27 (s, 1H), 4.00 (s, 3H), 2.70-2.64 (m, 1H), 1.17-1.06 (m, 1H), 1.03-0.82 (m, 3H).

### Example 19

### Synthesis of imino (4-(((6-methoxypyrido[3,4-d]pyrimidin-4-yl)oxy)methyl)phenyl)(methyl)-lambda6-sulfanone

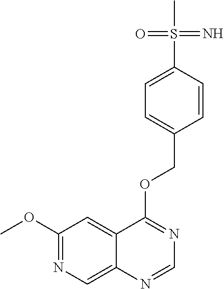 [SMILES: COc1cc2c(OCc3ccc(S(C)(=N)=O)cc3)ncnc2cn1]

### Step 1:6-Methoxy-4-((4-(methylthio)benzyl)oxy) pyrido[3,4-d]pyrimidine

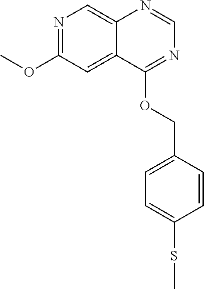 [SMILES: COc1cc2c(OCc3ccc(SC)cc3)ncnc2cn1]

The title compound was synthesized by proceeding analogously as described in Example 14, Step 5 except 4-chloro-6-methoxypyrido[3,4-d]pyrimidine (200 mg, 1.02 mmol) and [4-(methylsulfanyl)phenyl]methanol (189 mg, 1.23 mmol) were used. 6-methoxy-4-((4-(methylthio)benzyl)oxy) pyrido[3,4-d]pyrimidine (184 mg, 57%) was obtained white solid.

### Step 2: Imino (4-(((6-methoxypyrido[3,4-d]pyrimidin-4-yl)oxy)methyl)phenyl)(methyl)-lambda6-sulfanone

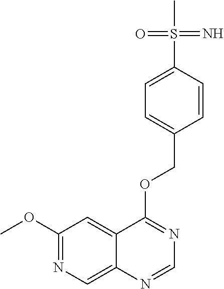 [SMILES: COc1cc2c(OCc3ccc(S(C)(=N)=O)cc3)ncnc2cn1]

The title compound was synthesized by proceeding analogously as described in Example 1, Step 5 except 6-methoxy-4-((4-(methylthio)benzyl)oxy) pyrido[3,4-d]pyrimidine (160 mg, 0.51 mmol) was used. The crude product was purified by prep-HPLC with the following conditions: (Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 10% B to 40% B in 7 min; Wave Length: 254/220 nm; RT1 (min): 6.65). The fractions containing the desired product were combined and lyophilized to afford imino (4-(((6-methoxypyrido[3,4-d]pyrimidin-4-yl)oxy)methyl)phenyl)(methyl)-lambda6-sulfanone (28.1 mg, 15%) as a white solid. MS (ESI, pos. ion) m/z: 345.1 (M+1). ¹H NMR (400 MHZ, DMSO-d₆, ppm) δ 9.12 (d, J=1.0 Hz, 1H), 8.77 (s, 1H), 8.0 (d, J=7.96 Hz, 2H), 7.78 (d, J=8.0 Hz, 2H), 7.33 (d, J=1.0 Hz, 1H), 5.77 (s, 2H), 4.24 (s, 1H), 4.00 (s, 3H), 3.07 (d, J=1.1 Hz, 3H).

### Example 20

### Synthesis of ((4-hydroxy-1-(8-methoxypyrido[3,4-d]pyrimidin-4-yl) piperidin-4-yl)methyl)-(imino)(methyl)-lambda6-sulfanone

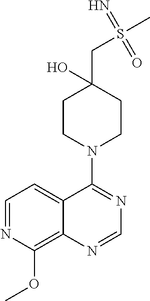 [SMILES: COc1nccc2c(N3CCC(O)(CS(C)(=N)=O)CC3)ncnc12]

### Step 1:1-(8-Methoxypyrido[3,4-d]pyrimidin-4-yl)-4-((methylthio)methyl) piperidin-4-ol

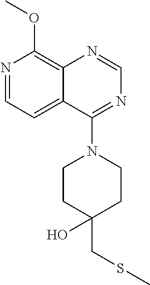 [SMILES: COc1nccc2c(N3CCC(O)(CSC)CC3)ncnc12]

The title compound was synthesized by proceeding analogously as described in Example 1, Step 4 except 4-chloro-8-methoxypyrido[3,4-d]pyrimidine (300 mg, 1.53 mmol) and 4-[(methylsulfanyl)methyl]piperidin-4-ol hydrochloride (364 mg, 1.84 mmol) were used and stirred at 90° C. for 2 h. 1-(8-methoxypyrido[3,4-d]pyrimidin-4-yl)-4-((methylthio)methyl) piperidin-4-ol (366 mg, 74%) was obtained white solid.

### Step 2: ((4-Hydroxy-1-(8-methoxypyrido[3,4-d]pyrimidin-4-yl) piperidin-4-yl)methyl)-(imino)(methyl)-lambda6-sulfanone

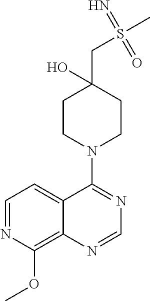 [SMILES: COc1nccc2c(N3CCC(O)(CS(C)(=N)=O)CC3)ncnc12]

The title compound was synthesized by proceeding analogously as described in Example 1, Step 5 except 1-(8-methoxypyrido[3,4-d]pyrimidin-4-yl)-4-((methylthio)methyl) piperidin-4-ol (200 mg, 0.62 mmol) was used. The crude product was purified by prep-HPLC with the following conditions: (Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 3% B to 23% B in 7 min; Wave Length: 254/220 nm; RT1 (min): 6.03). The fractions containing the desired product were combined and lyophilized to give ((4-hydroxy-1-(8-methoxypyrido[3,4-d]pyrimidin-4-yl) piperidin-4-yl)methyl)-(imino)(methyl)-lambda6-sulfanone (130.1 mg, 58%) as a white solid. MS (ESI, pos. ion) m/z: 352.1 (M+1). ¹H NMR (400 MHZ, DMSO-d₆, ppm) δ 8.64 (s, 1H), 8.05 (d, J=6.0 Hz, 1H), 7.34 (d, J=5.6 Hz, 1H), 6.05 (s, 1H), 4.09 (d, J=13.6 Hz, 2H), 4.02 (s, 4H), 3.58-3.52 (m, 2H), 3.42-3.38 (m, 1H), 3.29-3.25 (m, 1H), 3.02 (s, 3H), 2.02-1.80 (m, 4H).

### Example 21

### Synthesis of 4-((3-(cyclopropanesulfonimidoyl)benzyl)oxy)-8-methoxy-1,7-naphthyridine-3-carbonitrile

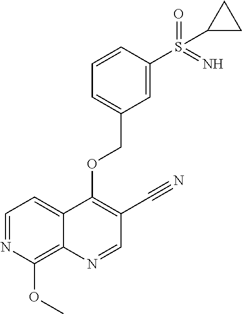 [SMILES: COc1nccc2c(OCc3cccc(S(=N)(=O)C4CC4)c3)c(C#N)cnc12]

### Step 1: Methyl 3-[(E)-[(dimethylamino)methylidene]amino]-2-methoxypyridine-4-carboxylate

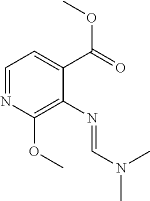 [SMILES: COC(=O)c1ccnc(OC)c1/N=C/N(C)C]

A solution of methyl 3-amino-2-methoxypyridine-4-carboxylate (2.10 g, 11.52 mmol, 1.0 equiv) in DMF-DMA (1.37 g, 11.52 mmol, 1.0 equiv) was stirred at 110° C. for 12 h. The reaction mixture was cooled to room temperature and concentrated under reduced pressure to afford methyl 3-[(E)-[(dimethylamino)methylidene]amino]-2-methoxypyridine-4-carboxylate (1.80 g, 65.8%) as a light-yellow oil.

### Step 2:4-Hydroxy-8-methoxy-1,7-naphthyridine-3-carbonitrile

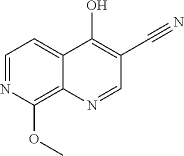

To a solution of MeCN (1.11 g, 26.97 mmol, 2.0 equiv) in THF (30 mL) was added n-BuLi (10.7 mL, 26.97 mmol, 2.0 equiv, 2.5 M in n-hexane) slowly at −70° C. under nitrogen atmosphere. After stirring at −70° C. for 0.5 h, methyl 3-[(E)-[(dimethylamino)methylidene]amino]-2-methoxypyridine-4-carboxylate (3.20 g, 13.48 mmol, 1.0 equiv) in THF (30 mL) was added to the reaction mixture and stirred at −70° C. for another 1.5 h. AcOH (2.4 mL) was added and the reaction mixture was poured into water. The resulting mixture was extracted with EtOAc and the combined organic layer was washed with brine, dried over anhydrous sodium sulfate, filtered, and concentrated under reduced pressure. The crude product was purified by reverse phase flash chromatography, eluted with ACN/H₂O (10:90) to afford 4-hydroxy-8-methoxy-1,7-naphthyridine-3-carbonitrile (1.80 g, 66%) as a light yellow solid.

### Step 3:4-[3-(Cyclopropylsulfanyl)phenyl]methoxy]-8-methoxy-1,7-naphthyridine-3-carbonitrile

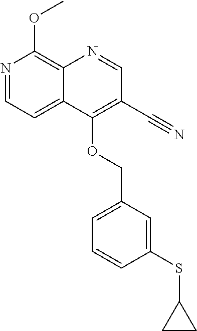 [SMILES: COc1nccc2c(OCc3cccc(SC4CC4)c3)c(C#N)cnc12]

The title compound was synthesized by proceeding analogously as described in Example 12, Step 3 except 4-hydroxy-8-methoxy-1,7-naphthyridine-3-carbonitrile (400 mg, 1.98 mmol) and [3-(cyclopropylsulfanyl)phenyl]methanol (716 mg, 3.97 mmol) were used. 4-[[3-(cyclopropylsulfanyl)phenyl]methoxy]-8-methoxy-1,7-naphthyridine-3-carbonitrile (38 mg, 5%) was obtained as a brown solid.

### Step 4:4-((3-(cyclopropanesulfonimidoyl)benzyl)oxy)-8-methoxy-1,7-naphthyridine-3-carbonitrile

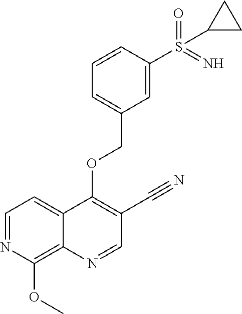 [SMILES: COc1nccc2c(OCc3cccc(S(=N)(=O)C4CC4)c3)c(C#N)cnc12]

The title compound was synthesized by proceeding analogously as described in Example 1, Step 5 except 4-[3-(cyclopropylsulfanyl)phenyl]methoxy]-8-methoxy-1,7-naphthyridine-3-carbonitrile (35 mg, 0.096 mmol) was used. The crude product was purified by prep-HPLC with the following conditions: (Column: XBridge Prep Shield RP C18 Column, 30*150 mm, 5 μm; Mobile Phase A: Water (10 mmol/L NH₄HCO₃), Mobile Phase B: ACN; Flow rate: 60 mL/min mL/min; Gradient: 19% B to 40% B in 7 min; Wavelength: 254 nm/220 nm nm; RT1 (min): 6.57 to afford 4-((3-(cyclopropanesulfonimidoyl)benzyl)oxy)-8-methoxy-1,7-naphthyridine-3-carbonitrile (12.8 mg, 31%) as an off-white solid. MS (ESI, pos. ion) m/z: 395.1 (M+1). ¹H NMR (400 MHZ, DMSO-d₆, ppm) δ 9.03 (d, J=1.6 Hz 1H), 8.24-8.17 (m, 1H), 8.11 (d, J=2.0 Hz, 1H), 7.95 (d, J=7.6 Hz, 1H), 7.87 (d, J=7.6 Hz, 1H), 7.74-7.66 (m, 1H), 7.53-7.51 (m, 1H), 5.99 (s, 2H), 4.29 (s, 1H), 4.08 (s, 3H), 1.15-0.90 (m, 5H).

### Example 22

### Synthesis of imino ((2-(8-methoxy-1,7-naphthyridin-4-yl)-2-azaspiro[3.3]heptan-6-yl)methyl)(methyl)-lambda6-sulfanone

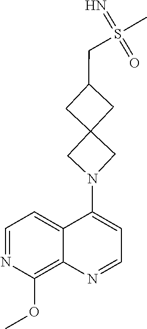 [SMILES: COc1nccc2c(N3CC4(CC(CS(C)(=N)=O)C4)C3)ccnc12]

### Step 1:8-Methoxy-4-[6-[(methylsulfanyl)methyl]-2-azaspiro[3.3]heptan-2-yl]-1,7-naphthyridine

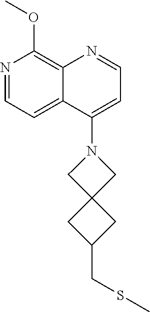 [SMILES: COc1nccc2c(N3CC4(CC(CSC)C4)C3)ccnc12]

The title compound was synthesized by proceeding analogously as described in Example 1, Step 4 except 4-chloro-8-methoxy-1,7-naphthyridine (130 mg, 0.66 mmol) and 6-[(methylsulfanyl)methyl]-2-azaspiro[3.3]heptane (315 mg, 2.00 mmol) were used. 8-methoxy-4-[6-[(methylsulfanyl)methyl]-2-azaspiro[3.3]heptan-2-yl]-1,7-naphthyridine (120 mg, 56%) was obtained as a brown yellow solid.

### Step 2: Imino ((2-(8-methoxy-1,7-naphthyridin-4-yl)-2-azaspiro[3.3]heptan-6-yl)methyl)-(methyl)-lambda6-sulfanone

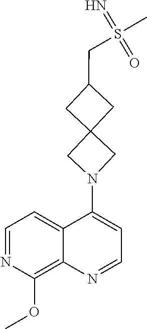 [SMILES: COc1nccc2c(N3CC4(CC(CS(C)(=N)=O)C4)C3)ccnc12]

The title compound was synthesized by proceeding analogously as described in Example 1, Step 5 except 8-methoxy-4-[6-[(methylsulfanyl)methyl]-2-azaspiro[3.3]heptan-2-yl]-1,7-naphthyridine (100 mg, 0.31 mmol) was used. The crude product was purified by prep-HPLC with the following conditions: (Column: XBridge Prep OBD C18 Column, 50*250 mm, 10 μm; Mobile Phase A: Water (10 nmol/L NH₄HCO₃), Mobile Phase B: ACN; Flow rate: 60 mL/min; Gradient: 7% B to 33% B in 20 min; Wavelength: 254/220 nm; RT1 (min): 20.77). The fractions containing the desired product were combined and lyophilized to afford imino ((2-(8-methoxy-1,7-naphthyridin-4-yl)-2-azaspiro[3.3]heptan-6-yl)methyl)(methyl)-lambda6-sulfanone (42.3 mg, 36%) as a light yellow semi-solid. MS (ESI, pos. ion) m/z: 347.1 (M+1). ¹H NMR (400 MHZ, DMSO-d₆ ppm) δ 8.40 (d, J=5.2 Hz, 1H), 7.87 (d, J=6.0 Hz, 1H), 7.35 (d, J=6.0 Hz, 1H), 6.41 (d, J=5.2 Hz, 1H), 4.38 (s, 2H), 4.23 (s, 2H), 3.98 (s, 3H), 3.57 (s, 1H), 3.19-3.18 (m, 2H), 2.84 (s, 3H), 2.68-2.64 (m, 1H), 2.46-2.43 (m, 2H), 2.17-2.09 (m, 2H).

### Example 23

### Synthesis of ((4-hydroxy-1-(8-methoxy-1,7-naphthyridin-4-yl) piperidin-4-yl)methyl)-(imino)(methyl)-lambda6-sulfanone

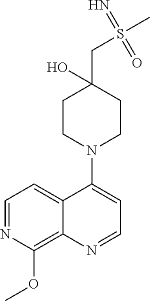 [SMILES: COc1nccc2c(N3CCC(O)(CS(C)(=N)=O)CC3)ccnc12]

### Step 1:1-(8-Methoxy-1,7-naphthyridin-4-yl)-4-[(methylsulfanyl)methyl]piperidin-4-ol

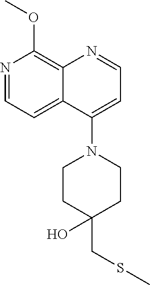 [SMILES: COc1nccc2c(N3CCC(O)(CSC)CC3)ccnc12]

The title compound was synthesized by proceeding analogously as described in Example 1, Step 4 except 4-chloro-8-methoxy-1,7-naphthyridine (150 mg, 0.77 mmol) and 4-[(methylsulfanyl)methyl]piperidin-4-ol hydrochloride (457 mg, 2.31 mmol) were used and stirred overnight at 130° C. 1-(8-methoxy-1,7-naphthyridin-4-yl)-4-[(methylsulfanyl)methyl]piperidin-4-ol (161 mg, 65%) was obtained as a brown oil.

### Step 2: ((4-Hydroxy-1-(8-methoxy-1,7-naphthyridin-4-yl) piperidin-4-yl)methyl) (imino)-(methyl)-lambda6-sulfanone

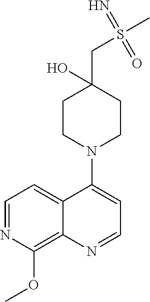 [SMILES: COc1nccc2c(N3CCC(O)(CS(C)(=N)=O)CC3)ccnc12]

The title compound was synthesized by proceeding analogously as described in Example 1, Step 5 except 1-(8-methoxy-1,7-naphthyridin-4-yl)-4-[(methylsulfanyl)methyl]piperidin-4-ol (140 mg, 0.44 mmol) was used. The crude product was purified by prep-HPLC with the following conditions: (Column: Xbridge Prep OBD C18 Column, 19*250 mm, 5 μm; Mobile Phase A: Water (10 mmol/L NH₄HCO₃), Mobile Phase B: MEOH; Flow rate: 25 mL/min; Gradient: 13% B to 33% B in 13.5 min; Wave Length: 254/220 nm; RT1 (min): 12.53) to afford ((4-hydroxy-1-(8-methoxy-1,7-naphthyridin-4-yl) piperidin-4-yl)methyl) (imino)(methyl)-lambda6-sulfanone (50.7 mg, 32%) as a white solid. MS (ESI, pos. ion) m/z: 351.1 (M+1). ¹H NMR (400 MHZ, DMSO-d₆, ppm) δ 8.66 (d, J=5.2 Hz, 1H), 8.02 (d, J=6.0 Hz, 1H), 7.33 (d, J=5.6 Hz, 1H), 7.17 (d, J=5.2 Hz, 1H), 5.61 (s, 1H), 4.02 (d, J=4.4 Hz, 4H), 3.44 (d, J=14.4 Hz, 1H), 3.33 (s, 1H), 3.32 (d, J=14.6 Hz, 2H), 3.22-3.11 (m, 2H), 3.05 (s, 3H), 2.09-1.88 (m, 4H).

### Example 24

### Synthesis of imino ((1-(8-methoxy-1,7-naphthyridin-4-yl) piperidin-4-yl)methyl)(methyl)-lambda6-sulfanone

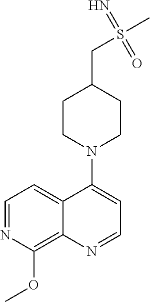 [SMILES: COc1nccc2c(N3CCC(CS(C)(=N)=O)CC3)ccnc12]

### Step 1: [1-(8-Methoxy-1,7-naphthyridin-4-yl) piperidin-4-yl]methanol

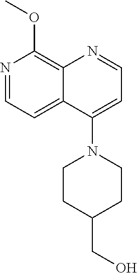 [SMILES: COc1nccc2c(N3CCC(CO)CC3)ccnc12]

The title compound was synthesized by proceeding analogously as described in Example 1, Step 4 except 4-chloro-8-methoxy-1,7-naphthyridine (300 mg, 1.54 mmol) and piperidin-4-ylmethanol (355 mg, 3.08) were used and stirred at 130° C. for 2 h. [1-(8-methoxy-1,7-naphthyridin-4-yl) piperidin-4-yl]methanol (272 mg, 45%) was obtained as a yellow solid.

### Step 2: [1-(8-Methoxy-1,7-naphthyridin-4-yl) piperidin-4-yl]methyl methanesulfonate

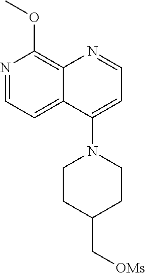 [SMILES: COc1nccc2c(N3CCC(COS(C)(=O)=O)CC3)ccnc12]

To a stirred solution of [1-(8-methoxy-1,7-naphthyridin-4-yl) piperidin-4-yl]methanol (400 mg, 1.46 mmol, 1 equiv) in DCM (8 mL) was added TEA (296 mg, 2.926 mmol, 2 equiv), DMAP (17 mg, 0.14 mmol, 0.1 equiv) and methanesulfonyl methanesulfonate (382 mg, 2.19 mmol, 1.5 equiv) at 0° C. After stirring at room temperature for 2 h, the reaction mixture was concentrated under reduced pressure and purified by silica gel column chromatography, eluted with MeOH/DCM (12:88) to afford [1-(8-methoxy-1,7-naphthyridin-4-yl) piperidin-4-yl]methyl methanesulfonate (1.10 g, crude) as a yellow solid.

### Step 3:8-Methoxy-4-[4-[(methylsulfanyl)methyl]piperidin-1-yl]-1,7-naphthyridine

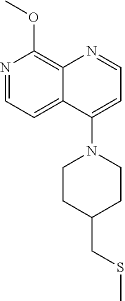 [SMILES: COc1nccc2c(N3CCC(CSC)CC3)ccnc12]

The title compound was synthesized by proceeding analogously as described in Example 1, Step 2 except [1-(8-methoxy-1,7-naphthyridin-4-yl) piperidin-4-yl]methyl methanesulfonate (1.00 g, 2.84 mmol) was used. 8-methoxy-4-[4-[(methylsulfanyl)-methyl]piperidin-1-yl]-1,7-naphthyridine (323 mg, 37%) was obtained as a yellow solid.

### Step 4: Imino ((1-(8-methoxy-1,7-naphthyridin-4-yl) piperidin-4-yl)methyl)(methyl)-lambda6-sulfanone

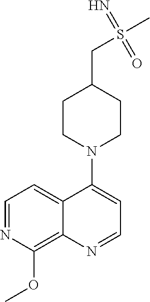 [SMILES: COc1nccc2c(N3CCC(CS(C)(=N)=O)CC3)ccnc12]

The title compound was synthesized by proceeding analogously as described in Example 1, Step 5 except 8-methoxy-4-[4-[(methylsulfanyl)methyl]piperidin-1-yl]-1,7-naphthyridine (150 mg, 0.42 mmol) was used. The crude product was purified by prep-HPLC with the following conditions: (Column: XBridge Prep OBD C18 Column, 30*150 mm, 5 μm; Mobile Phase A: Water (10 nmol/L NH₄HCO₃), Mobile Phase B: ACN; Flow rate: 60 mL/min; Gradient: 2% B to 27% B in 10 min; Wavelength: 254/220 nm; RT1 (min): 8.83) to afford imino ((1-(8-methoxy-1,7-naphthyridin-4-yl) piperidin-4-yl)methyl)(methyl)-lambda6-sulfanone (56.3 mg, 39%) as a yellow solid. MS (ESI, pos. ion) m/z: 335.1 (M+1). ¹H NMR (400 MHZ, DMSO-d₆, ppm) δ 8.67 (d, J=5.0 Hz, 1H), 8.02 (d, J=5.9 Hz, 1H), 7.32 (d, J=6.0 Hz, 1H), 7.15 (d, J=5.0 Hz, 1H), 4.03 (s, 3H), 3.73 (s, 1H), 3.52 (d, J=12.1 Hz, 2H), 3.14 (d, J=6.3 Hz, 2H), 2.97 (s, 3H), 2.88-2.85 (m, 2H), 2.21 (d, J=10.9 Hz, 1H), 2.08-2.05 (m, 2H), 1.64-1.61 (m, 2H).

### Example 25

### Synthesis of imino (2-(1-(8-methoxy-1,7-naphthyridin-4-yl) piperidin-4-yl)ethyl)(methyl)-lambda6-sulfanone)

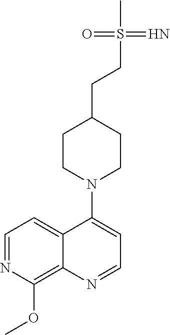 [SMILES: COc1nccc2c(N3CCC(CCS(C)(=N)=O)CC3)ccnc12]

### Step 1:2-[1-(8-Methoxy-1,7-naphthyridin-4-yl) piperidin-4-yl]ethanol

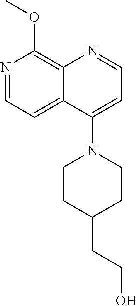 [SMILES: COc1nccc2c(N3CCC(CCO)CC3)ccnc12]

The title compound was synthesized by proceeding analogously as described in Example 1, Step 4 except 4-chloro-8-methoxy-1,7-naphthyridine (350 mg, 1.79 mmol) and 4-piperidineethanol (697 mg, 5.39 mmol) were used. 2-[1-(8-methoxy-1,7-naphthyridin-4-yl) piperidin-4-yl]ethanol (350 mg, 67%) was obtained as a brown solid.

### Step 2:2-[1-(8-Methoxy-1,7-naphthyridin-4-yl) piperidin-4-yl]ethyl methanesulfonate

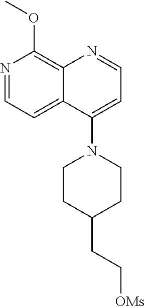 [SMILES: COc1nccc2c(N3CCC(CCOS(C)(=O)=O)CC3)ccnc12]

The title compound was synthesized by proceeding analogously as described in Example 24, Step 2 except 2-[1-(8-methoxy-1,7-naphthyridin-4-yl) piperidin-4-yl]ethanol (330 mg, 1.14 mmol) was used. 2-[1-(8-methoxy-1,7-naphthyridin-4-yl) piperidin-4-yl]ethyl methanesulfonate (300 mg, 71%) was obtained as a brown oil.

### Step 3:8-Methoxy-4-[4-[2-(methylsulfanyl)ethyl]piperidin-1-yl]-1,7-naphthyridine

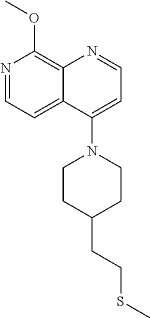 [SMILES: COc1nccc2c(N3CCC(CCSC)CC3)ccnc12]

The title compound was synthesized by proceeding analogously as described in Example 1, Step 2 except 2-[1-(8-methoxy-1,7-naphthyridin-4-yl) piperidin-4-yl]ethyl methanesulfonate (280 mg, 0.76 mmol) was used. 8-methoxy-4-[4-[2-(methylsulfanyl)-ethyl]piperidin-1-yl]-1,7-naphthyridine (200 mg, 82) was obtained as an off-white solid.

### Step 4: Imino (2-(1-(8-methoxy-1,7-naphthyridin-4-yl) piperidin-4-yl)ethyl)(methyl)-lambda6-sulfanone

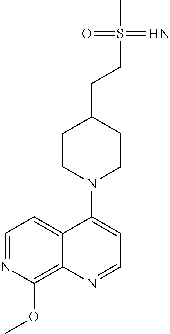 [SMILES: COc1nccc2c(N3CCC(CCS(C)(=N)=O)CC3)ccnc12]

The title compound was synthesized by proceeding analogously as described in Example 1 Step 5 except 8-methoxy-4-[4-[2-(methylsulfanyl)ethyl]piperidin-1-yl]-1,7-naphthyridine (180 mg, 0.56 mmol) was used. The crude product was purified by prep-HPLC with the following conditions: (Column: YMC-Actus Triart C18 ExRS Column, 30*150 mm, 5 μm; Mobile Phase A: Water (10 mmol/L NH₄HCO₃), Mobile Phase B: ACN; Flow rate: 60 mL/min; Gradient: 7% B to 28% B in 7 min; Wave Length: 254/220 nm; RT1 (min): 7.07) to afford imino (2-(1-(8-methoxy-1,7-naphthyridin-4-yl) piperidin-4-yl)ethyl)(methyl)-lambda6-sulfanone (90.5 mg, 45%) as a light yellow solid. MS (ESI, pos. ion) m/z: 349.1 (M+1). ¹H NMR (400 MHZ, DMSO-d₆, ppm) δ 8.66 (d, J=5.2 Hz, 1H), 8.01 (d, J=6.0 Hz, 1H), 7.31 (d, J=6.0 Hz, 1H), 7.14 (d, J=4.8 Hz, 1H), 4.03 (s, 3H), 3.63 (s, 1H), 3.54-3.51 (m, 2H), 3.13-3.09 (m, 2H), 2.92 (s, 3H), 2.84-2.76 (m, 2H), 1.87-1.84 (m, 2H), 1.78-1.72 (m, 2H), 1.65-1.56 (m, 1H), 1.51-1.49 (m, 2H).

### Example 26

### Synthesis of cyclopropyl(imino) ((1-(8-methoxy-1,7-naphthyridin-4-yl) piperidin-4-yl)methyl)-lambda6-sulfanone

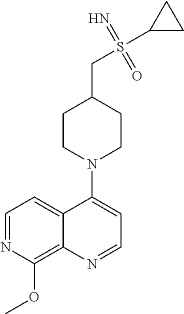 [SMILES: COc1nccc2c(N3CCC(CS(=N)(=O)C4CC4)CC3)ccnc12]

### Step 1: tert-Butyl 4-[(cyclopropylsulfanyl)methyl]piperidine-1-carboxylate

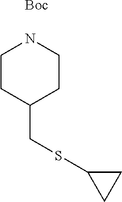 [SMILES: C1CC(CSC2CC2)CCN1 ; "Boc"]

The title compound was synthesized by proceeding analogously as described in Example 9, Step 1 except tert-butyl 4-(sulfanylmethyl) piperidine-1-carboxylate (200 mg, 0.86 mmol) was used and stirred at 120° C. for 3 days, tert-butyl 4-[(cyclopropylsulfanyl)methyl]piperidine-1-carboxylate (163 mg, 69%) was obtained as yellow oil.

### Step 2:4-[(Cyclopropylsulfanyl)methyl]piperidine hydrochloride

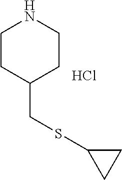 [SMILES: C1CC(CSC2CC2)CCN1 ; Cl]

A solution of tert-butyl 4-[(cyclopropylsulfanyl)methyl]piperidine-1-carboxylate (163 mg, 0.60 mmol) in 4 M HCl in 1,4-dioxane (2 mL) was stirred at room temperature for 1 h. The resulting mixture was concentrated under reduced pressure to afford 4-[(cyclopropylsulfanyl)-methyl]piperidine hydrochloride (140 mg, crude) as a white solid.

### Step 3:4-[4-[(Cyclopropylsulfanyl)methyl]piperidin-1-yl]-8-methoxy-1,7-naphthyridine

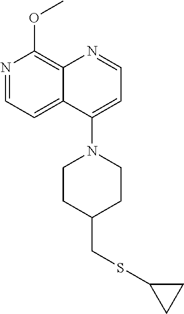 [SMILES: COc1nccc2c(N3CCC(CSC4CC4)CC3)ccnc12]

The title compound was synthesized by proceeding analogously as described in Example 1, Step 4 except 4-[(cyclopropylsulfanyl)methyl]piperidine hydrochloride (344 mg, 1.65 mmol) and 4-chloro-8-methoxy-1,7-naphthyridine (100 mg, 0.51 mmol) were used and stirred for 130° C. for 2 h. 4-[4-[(cyclopropylsulfanyl)methyl]piperidin-1-yl]-8-methoxy-1,7-naphthyridine (73 mg, 43%) was obtained as a yellow solid.

### Step 4: Cyclopropyl(imino) ((1-(8-methoxy-1,7-naphthyridin-4-yl) piperidin-4-yl)methyl)-lambda6-sulfanone

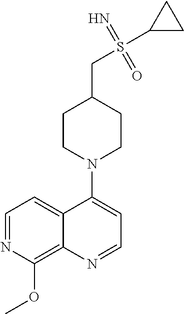 [SMILES: COc1nccc2c(N3CCC(CS(=N)(=O)C4CC4)CC3)ccnc12]

The title compound was synthesized by proceeding analogously as described in Example 1, Step 5 except 4-[4-[(cyclopropylsulfanyl)methyl]piperidin-1-yl]-1,7-naphthyridine (63 mg, 0.21 mmol) was used. The crude product was purified by prep-HPLC with the following conditions: (Column: XBridge Prep Shield RP C18 Column, 30*150 mm, 5 μm; Mobile Phase A: Water (10 mmol/L NH₄HCO₃), Mobile Phase B: ACN; Flow rate: 60 mL/min; Gradient: 8% B to 29% B in 8 min; Wavelength: 254/220 nm; RT1 (min): 8) to afford cyclopropyl(imino) ((1-(8-methoxy-1,7-naphthyridin-4-yl) piperidin-4-yl)methyl)-lambda6-sulfanone (28.1 mg, 36%) as a yellow solid. MS (ESI, pos. ion) m/z: 361.1 (M+1). ¹H NMR (400 MHZ, DMSO-d₆, ppm) δ 8.67 (d, J=5.0 Hz, 1H), 8.02 (d, J=5.9 Hz, 1H), 7.32 (d, J=6.0 Hz, 1H), 7.16 (d, J=5.1 Hz, 1H), 4.03 (s, 3H), 3.54-3.50 (m, 3H), 3.11 (d, J=6.3 Hz, 2H), 2.87 (t, J=12.0 Hz, 2H), 2.69-2.60 (m, 1H), 2.27 (s, 1H), 2.16-2.10 (m, 2H), 1.65 (q, J=12.3 Hz, 2H), 1.13-0.76 (m, 4H).

### Example 27

### Synthesis of imino (3-(((8-methoxy-1,7-naphthyridin-4-yl)oxy)methyl)phenyl)(methyl)-lambda6-sulfanone

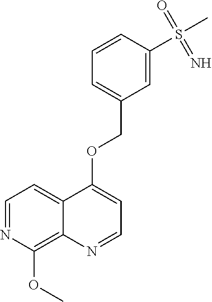 [SMILES: COc1nccc2c(OCc3cccc(S(C)(=N)=O)c3)ccnc12]

### Step 1:8-Methoxy-4-[[3-(methylsulfanyl)phenyl]methoxy]-1,7-naphthyridine

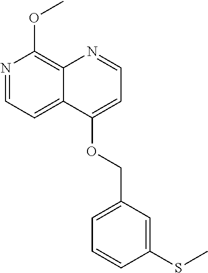 [SMILES: COc1nccc2c(OCc3cccc(SC)c3)ccnc12]

The title compound was synthesized by proceeding analogously as described in Example 11, Step 3 except 8-methoxy-1,7-naphthyridin-4-ol (300 mg, 1.70 mmol) and [3-(methylsulfanyl)phenyl]methanol (525 mg, 3.41 mmol) were used. A mixture of 8-methoxy-4-[3-(methylsulfanyl)phenyl]methoxy]-1,7-naphthyridine and 8-methoxy-1-(3-(methylthio)benzyl)-1,7-naphthyridin-4 (1H)-one (102 mg, 19%) was obtained as a yellow solid.

### Step 2: Imino (3-(((8-methoxy-1,7-naphthyridin-4-yl)oxy)methyl)phenyl)(methyl)-lambda6-sulfanone

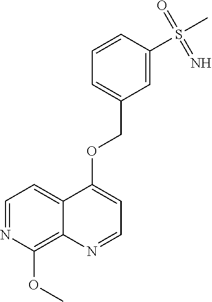 [SMILES: COc1nccc2c(OCc3cccc(S(C)(=N)=O)c3)ccnc12]

The title compound was synthesized by proceeding analogously as described in Example 1, Step 5 except mixture of 8-methoxy-4-[[3-(methylsulfanyl)-phenyl]methoxy]-1,7-naphthyridine and 8-methoxy-1-(3-(methylthio)benzyl)-1,7-naphthyridin-4 (1H)-one (90 mg, 0.29 mmol) was used. The crude product was purified by prep-HPLC with the following conditions: (Column: YMC-Actus Triart C18 ExRS Column, 30*150 mm, 5 μm; Mobile Phase A: Water (10 mmol/L NH₄HCO₃), Mobile Phase B: ACN; Flow rate: 60 mL/min; Gradient: 8% B to 38% B in 7 min; Wavelength: 254/220 nm; RT (min): 5.48, 6.63) The fractions containing the desired product were combined and lyophilized to afford: Imino (3-(((8-methoxy-1,7-naphthyridin-4-yl)oxy)methyl)phenyl)(methyl)-lambda6-sulfanone (29.8 mg, 30%) as a white solid. MS (ESI, pos. ion) m/z: 344.1 (M+1). ¹H NMR (400 MHZ, DMSO-d₆, ppm) δ 8.79 (d, J=5.2 Hz, 1H), 8.13 (d, J=1.8 Hz, 1H), 8.08 (d, J=5.6 Hz, 1H), 7.99-7.92 (m, 1H), 7.88-7.81 (m, 1H), 7.73-7.64 (m, 1H), 7.55 (d, J=5.8 Hz, 1H), 7.38 (d, J=5.2 Hz, 1H), 5.52 (s, 2H), 4.29 (s, 1H), 4.05 (s, 3H), 3.10 (d, J=1.1 Hz, 3H).

### Example 28

### Synthesis of cyclopropyl(3-(((8-ethoxy-1,7-naphthyridin-4-yl)oxy)methyl)phenyl) (imino)-lambda6-sulfanone

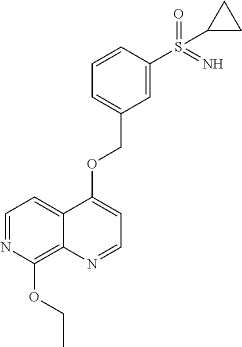 [SMILES: CCOc1nccc2c(OCc3cccc(S(=N)(=O)C4CC4)c3)ccnc12]

### Step 1:5-[[(2-Ethoxypyridin-3-yl)amino]methylidene]-2,2-dimethyl-1,3-dioxane-4,6-dione

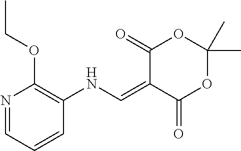 [SMILES: CCOc1ncccc1NC=C1C(=O)OC(C)(C)OC1=O]

The title compound was synthesized by proceeding analogously as described in Example 12, Step 1 except 2-ethoxypyridin-3-amine (2.00 g, 14.47 mmol) was used. 5-[[(2-ethoxypyridin-3-yl)amino]methylidene]-2,2-dimethyl-1,3-dioxane-4,6-dione (4.20 g, 99%) was obtained as a black solid.

### Step 2:8-Ethoxy-1,7-naphthyridin-4-ol

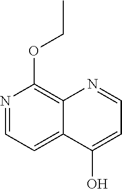

The title compound was synthesized by proceeding analogously as described in Example 12, Step 2 except 5-[[(2-ethoxypyridin-3-yl)amino]methylidene]-2,2-dimethyl-1,3-dioxane-4,6-dione (4.10 g, 14.03 mmol) was used. 8-ethoxy-1,7-naphthyridin-4-ol (552 mg, 20%) was obtained as a brown solid.

### Step 3:4-Chloro-8-ethoxy-1,7-naphthyridine

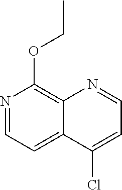

The title compound was synthesized by proceeding analogously as described in Example 3, Step 4 except 8-ethoxy-1,7-naphthyridin-4-ol (200 mg, 1.05 mmol) was used. 4-chloro-8-ethoxy-1,7-naphthyridine (100 mg, 45%) was obtained as a yellow solid.

### Step 4:4-[3-(Cyclopropylsulfanyl)phenyl]methoxy]-8-ethoxy-1,7-naphthyridine

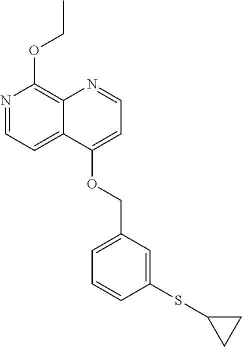 [SMILES: CCOc1nccc2c(OCc3cccc(SC4CC4)c3)ccnc12]

The title compound was synthesized by proceeding analogously as described in Example 5 Step 4 except [3-(cyclopropylsulfanyl)phenyl]methanol (98 mg, 0.55 mmol) and 4-chloro-8-ethoxy-1,7-naphthyridine (80 mg, 0.42 mmol) were used. 4-[[3-(cyclopropylsulfanyl)phenyl]methoxy]-8-ethoxy-1,7-naphthyridine (78 mg, 52%) was obtained as a brown oil.

### Step 5: Cyclopropyl(3-(((8-ethoxy-1,7-naphthyridin-4-yl)oxy)methyl)phenyl) (imino)-lambda6-sulfanone

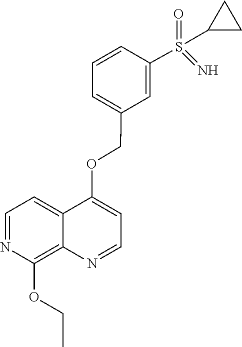 [SMILES: CCOc1nccc2c(OCc3cccc(S(=N)(=O)C4CC4)c3)ccnc12]

The title compound was synthesized by proceeding analogously as described in Example 1, Step 5 except 4-[3-(cyclopropylsulfanyl)phenyl]methoxy]-8-ethoxy-1,7-naphthyridine (68 mg, 0.19 mmol) was used. The crude product was purified by prep-HPLC with the following conditions: (Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 20% B to 41% B in 7 min; Wave Length: 254/220 nm; RT1 (min): 6.27) to afford cyclopropyl(3-(((8-ethoxy-1,7-naphthyridin-4-yl)oxy)methyl)phenyl) (imino)-lambda6-sulfanone (30.6 mg, 40%) as a white solid. MS (ESI, pos. ion) m/z: 384.1 (M+1). ¹H NMR (400 MHZ, DMSO-d₆, ppm) δ 8.79 (d, J=5.2 Hz, 1H), 8.10-8.03 (m, 2H), 7.94-7.87 (m, 1H), 7.84 (d, J=7.6 Hz, 1H), 7.72-7.64 (m, 1H), 7.53 (d, J=5.8 Hz, 1H), 7.37 (d, J=5.3 Hz, 1H), 5.53 (s, 2H), 4.56-4.46 (m, 2H), 4.28 (s, 1H), 2.72-2.61 (m, 1H), 1.47-1.39 (m, 3H), 1.16-1.06 (m, 1H), 1.03-0.82 (m, 3H).

### Example 29

### Synthesis of imino (3-(((8-methoxypyrido[3,4-d]pyrimidin-4-yl)oxy)methyl)phenyl)(methyl)-lambda6-sulfanone

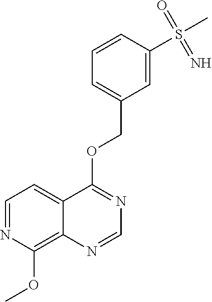 [SMILES: COc1nccc2c(OCc3cccc(S(C)(=N)=O)c3)ncnc12]

### Step 1:8-Methoxy-4-[3-(methylsulfanyl)phenyl]methoxy]pyrido[3,4-d]pyrimidine

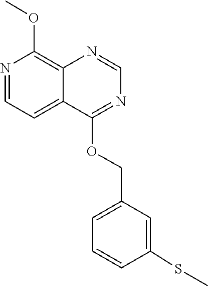 [SMILES: COc1nccc2c(OCc3cccc(SC)c3)ncnc12]

The title compound was synthesized by proceeding analogously as described in Example 14, Step 5 except [3-(methylsulfanyl)phenyl]methanol (300 mg, 1.94 mmol) and 4-chloro-8-methoxypyrido[3,4-d]pyrimidine (570 mg, 2.91 mmol) were used. 8-methoxy-4-[[3-(methylsulfanyl)phenyl]methoxy]pyrido[3,4-d]pyrimidine (250 mg, 41%) was obtained as an off-white solid.

### Step 2: Imino (3-(((8-methoxypyrido[3,4-d]pyrimidin-4-yl)oxy)methyl)phenyl)(methyl)-lambda6-sulfanone

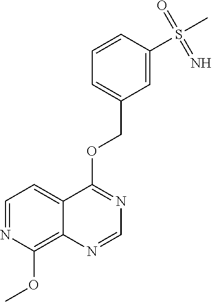 [SMILES: COc1nccc2c(OCc3cccc(S(C)(=N)=O)c3)ncnc12]

The title compound was synthesized by proceeding analogously as described in Example 1 Step 5 except 8-methoxy-4-[3-(methylsulfanyl)phenyl]-methoxy]pyrido[3,4-d]pyrimidine (230 mg, 0.73 mmol) was used. The crude product was purified by prep-HPLC with the following conditions: (Column: XBridge Prep Shield RP C18 Column, 30*150 mm, 5 μm; Mobile Phase A: Water (10 mmol/L NH₄HCO₃), Mobile Phase B: ACN; Flow rate: 60 mL/min; Gradient: 11% B to 32% B in 7 min; Wave Length: 254/220 nm; RT1 (min): 6.95) to afford imino (3-(((8-methoxypyrido[3,4-d]pyrimidin-4-yl)oxy)methyl)phenyl)(methyl)-lambda6-sulfanone (115.0 mg, 45%) as an off-white solid. MS (ESI, pos. ion) m/z: 345.1 (M+1). ¹H NMR (400 MHZ, DMSO-d₆, ppm) δ 8.94 (s, 1H), 8.25 (d, J=5.6 Hz, 1H), 8.12 (d, J=1.6 Hz, 1H), 7.94 (dt, J=8.0, 1.6 Hz, 1H), 7.85 (dt, J=7.6, 1.2 Hz, 1H), 7.67 (t, J=7.6 Hz, 1H), 7.55 (d, J=5.6 Hz, 1H), 5.76 (s, 2H), 4.28 (s, 1H), 4.08 (s, 3H), 3.09 (d, J=1.2 Hz, 3H).

### Example 30

### Synthesis of (4-((1,7-naphthyridin-4-yl)oxy)phenyl) (cyclopropyl) (imino)-lambda6-sulfanone

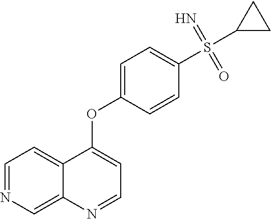 [SMILES: N=S(=O)(c1ccc(Oc2ccnc3cnccc23)cc1)C1CC1]

### Step 1:4-[4-(Cyclopropylsulfanyl) phenoxy]-1,7-naphthyridine

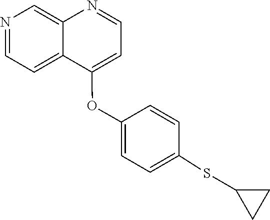 [SMILES: c1cc2c(Oc3ccc(SC4CC4)cc3)ccnc2cn1]

To a solution of 4-chloro-1,7-naphthyridine (100 mg, 0.61 mmol, 1 equiv) in DMSO (2 mL) was added 4-(cyclopropylsulfanyl) phenol (151 mg, 0.91 mmol, 1.5 equiv) and Cs₂CO₃ (297 mg, 0.91 mmol, 1.5 equiv). The resulting mixture was stirred at 100° C. for 2 h. After cooling to room temperature, the resulting mixture was diluted with H₂O (50 mL) and extracted with EtOAc. The combined organic layer was washed with brine, dried over Na₂SO₄ and concentrated under reduced pressure to afford 4-[4-(cyclopropylsulfanyl) phenoxy]-1,7-naphthyridine (208 mg, 90%) as a grey solid.

### Step 2: (4-((1,7-Naphthyridin-4-yl)oxy)phenyl) (cyclopropyl) (imino)-lambda6-sulfanone

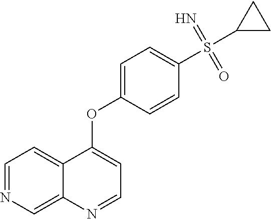 [SMILES: N=S(=O)(c1ccc(Oc2ccnc3cnccc23)cc1)C1CC1]

The title compound was synthesized by proceeding analogously as described in Example 1, Step 5 except 4-[4-(cyclopropylsulfanyl) phenoxy]-1,7-naphthyridine (188 mg, 0.64 mmol) was used. The crude product was purified by prep-HPLC with the following conditions: (Column: XselectCSH Prep OBD C18 Column, 30*150 mm, 5 μm; Mobile Phase A: Water (0.1% FA), Mobile Phase B: ACN; Flow rate: 60 mL/min; Gradient: 13% B to 43% B in 8 min; Wave Length: 254/220 nm; RT1 (min): 7.7) to afford (4-((1,7-naphthyridin-4-yl)oxy)phenyl) (cyclopropyl) (imino)-lambda6-sulfanone (59.2 mg, 28%) as a brown solid. MS (ESI, pos. ion) m/z: 326.2 (M+1). ¹H NMR (400 MHZ, DMSO-d₆, ppm) δ 9.46 (d, J=0.9 Hz, 1H), 8.93 (d, J=5.2 Hz, 1H), 8.70 (d, J=5.6 Hz, 1H), 8.14-8.11 (m, 1H), 8.07-7.99 (m, 2H), 7.57-7.49 (m, 2H), 7.06 (d, J=5.2 Hz, 1H), 4.31 (s, 1H), 2.78-2.67 (m, 1H), 1.20-1.10 (m, 1H), 1.06-0.87 (m, 3H).

### Example 31

### Synthesis of 4-[4-[imino(methyl)oxo-lambda6-sulfanyl]phenoxy]-8-methoxy-1,7-naphthyridine-3-carbonitrile

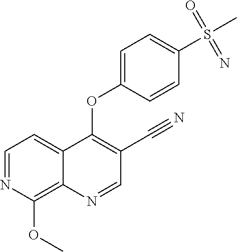 [SMILES: COc1nccc2c(Oc3ccc(S(C)(=N)=O)cc3)c(C#N)cnc12]

### Step 1:8-Methoxy-4-[4-(methylsulfanyl) phenoxy]-1,7-naphthyridine-3-carbonitrile

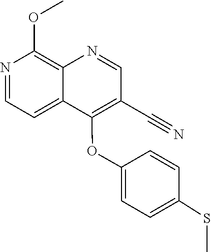 [SMILES: COc1nccc2c(Oc3ccc(SC)cc3)c(C#N)cnc12]

The title compound was synthesized by proceeding analogously as described in Example 30, Step 1 except 4-chloro-8-methoxy-1,7-naphthyridine-3-carbonitrile (300 mg, 1.36 mmol) and 4-(methylthio)-phenol (287 mg, 2.04 mmol) were used and stirred at 80° C. for 3 h. 8-methoxy-4-[4-(methylsulfanyl) phenoxy]-1,7-naphthyridine-3-carbonitrile (100 mg, 22%) was obtained as a light-yellow solid.

### Step 2:4-[4-[Imino(methyl)oxo-lambda6-sulfanyl]phenoxy]-8-methoxy-1,7-naphthyridine-3-carbonitrile

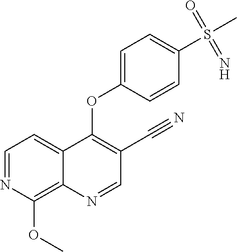 [SMILES: COc1nccc2c(Oc3ccc(S(C)(=N)=O)cc3)c(C#N)cnc12]

The title compound was synthesized by proceeding analogously as described in Example 1, Step 5 except 8-methoxy-4-[4-(methylsulfanyl) phenoxy]-1,7-naphthyridine-3-carbonitrile (80 mg, 0.24 mmol) was used. The crude product was purified by prep-HPLC with the following conditions: (Column: Xselect CSH Prep C18 C18 Column, 30*150 mm, 5 μm; Mobile Phase A: Water (0.1% FA), Mobile Phase B: ACN; Flow rate: 60 mL/min; Gradient: 10% B to 35% B in 10 min; Wavelength: 254/220 nm; RT1 (min): 8.68) to afford 4-[4-[imino(methyl)oxo-lambda6-sulfanyl]phenoxy]-8-methoxy-1,7-naphthyridine-3-carbonitrile (9.4 mg, 10.64%) as an off-white solid. MS (ESI, pos. ion) m/z: 355.0 (M+1). ¹H NMR (400 MHZ, DMSO-d₆, ppm) δ 9.28 (s, 1H), 8.23 (d, J=5.9 Hz, 1H), 7.97 (d, J=8.0 Hz, 2H), 7.39-9.35 (m, 3H), 4.29 (s, 1H), 4.13 (s, 3H), 3.08 (s, 3H).

### Example 32

### Synthesis of 2-(S-methylsulfonimidoyl)-8-(1,7-naphthyridin-4-yl)-2,8-diazaspiro[4.5]decane

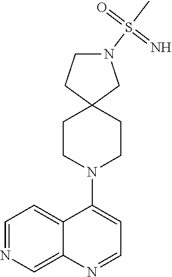 [SMILES: CS(=N)(=O)N1CCC2(CCN(c3ccnc4cnccc34)CC2)C1]

### Step 1: tert-Butyl 8-(1,7-naphthyridin-4-yl)-2,8-diazaspiro[4.5]decane-2-carboxylate

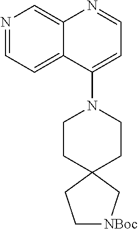 [SMILES: CC(C)(C)OC(=O)N1CCC2(CCN(c3ccnc4cnccc34)CC2)C1]

The title compound was synthesized by proceeding analogously as described in Example 8, Step 1 except 4-chloro-1,7-naphthyridine (450 mg, 2.73 mmol) and tert-butyl 2,8-diazaspiro[4.5]decane-2-carboxylate (1.31 g, 5.47 mmol) were used, tert-butyl 8-(1,7-naphthyridin-4-yl)-2,8-diazaspiro[4.5]decane-2-carboxylate (836 mg, 72%) was obtained as an orange solid.

### Step 2:8-(1,7-Naphthyridin-4-yl)-2,8-diazaspiro[4.5]decane hydrochloride

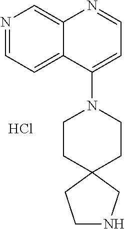 [SMILES: c1cc2c(N3CCC4(CCNC4)CC3)ccnc2cn1 ; Cl]

A solution of tert-butyl 8-(1,7-naphthyridin-4-yl)-2,8-diazaspiro[4.5]decane-2-carboxylate (800 mg, 2.17 mmol) in 14 mL HCl (g, 4 M in EtOAc) was stirred at room temperature for 1 h. Then the mixture was concentrated under reduced pressure to afford 8-(1,7-naphthyridin-4-yl)-2,8-diazaspiro[4.5]decane hydrochloride (1.00 g, crude) as a yellow solid.

### Step 3:2-(N-(tert-Butyldimethylsilyl)-S-methylsulfonimidoyl)-8-(1,7-naphthyridin-4-yl)-2,8-diazaspiro[4.5]decane

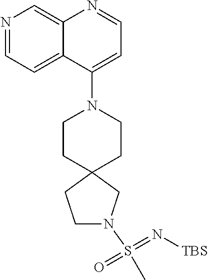

To a mixture of (diphenylphosphoryl)benzene (2.46 g, 8.86 mmol, 3 equiv) in DCM (24 mL) was added oxalic dichloride (1.20 g, 9.45 mmol, 3.2 equiv) under N₂ at 0° C. After stirring for 30 min at 0° C., DIEA (3.82 g, 29.53 mmol, 10 equiv) was added and stirred for 15 min at the same temperature. N-(tert-Butyldimethylsilyl) methanesulfonamide (927 mg, 4.43 mmol, 1.5 equiv) was added and the resulting mixture was stirred at 0° C. for 20 min before being added to a solution of 8-(1,7-naphthyridin-4-yl)-2,8-diazaspiro[4.5]decane hydrochloride (900 mg, 2.95 mmol, 1 equiv) and DIEA (1.14 g, 8.86 mmol, 3 equiv) in DCM (10 mL). After stirring at 0° C. for 30 min under N₂ atmosphere, the resulting mixture was concentrated under reduced pressure. The residue was purified by reversed phase chromatography, eluted with ACN/H₂O (84:16) to afford 2-(N-(tert-butyldimethylsilyl)-S-methylsulfonimidoyl)-8-(1,7-naphthyridin-4-yl)-2,8-diazaspiro[4.5]decane (454 mg, 25%) as a brown oil.

### Step 4:2-(S-Methylsulfonimidoyl)-8-(1,7-naphthyridin-4-yl)-2,8-diazaspiro[4.5]decane

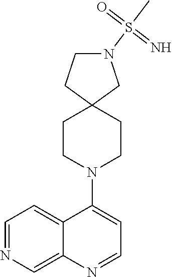 [SMILES: CS(=N)(=O)N1CCC2(CCN(c3ccnc4cnccc34)CC2)C1]

A solution of 2-(N-(tert-butyldimethylsilyl)-S-methylsulfonimidoyl)-8-(1,7-naphthyridin-4-yl)-2,8-diazaspiro[4.5]decane (165 mg, 0.36 mmol) in DMF (2 mL) was added 25% formic acid (0.4 mL, v/v). The resulting mixture was stirred for 10 min at room temperature. The resulting mixture was concentrated under reduced pressure. The crude product was purified by prep-HPLC with the following conditions (Column: Xselect CSH Prep Fluoro-Phenyl Column, 19*250 mm, 5 μm; Mobile Phase A: Water (10 mmol/L NH₄HCO₃), Mobile Phase B: ACN; Flow rate: 25 mL/min; Gradient: 11% B to 32% B in 8 min; Wavelength: 254/220 nm; RT1 (min): 12.57) to afford 2-(S-methylsulfonimidoyl)-8-(1,7-naphthyridin-4-yl)-2,8-diazaspiro[4.5]decane (54.4 mg, 43%) as a white solid. MS (ESI, pos. ion) m/z: 346.2 (M+1). ¹H NMR (400 MHZ, DMSO-d₆, ppm) δ 9.29 (d, J=0.7 Hz, 1H), 8.79 (d, J=4.8 Hz, 1H), 8.54 (d, J=5.6 Hz, 1H), 7.85-7.78 (m, 1H), 7.16 (d, J=5.2 Hz, 1H), 3.63 (s, 1H), 3.43-3.29 (m, 3H), 3.28-3.19 (m, 3H), 3.14 (d, J=4.3 Hz, 2H), 2.81 (s, 3H), 1.91-1.73 (m, 6H).

### Example 33

### Synthesis of 4-(2-(S-methylsulfonimidoyl)-1,2,3,4-tetrahydroisoquinolin-7-yl)-1,7-naphthyridine

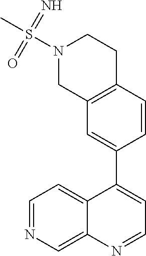 [SMILES: CS(=N)(=O)N1CCc2ccc(-c3ccnc4cnccc34)cc2C1]

### Step 1: tert-Butyl 7-(1,7-naphthyridin-4-yl)-3,4-dihydroisoquinoline-2 (1H)-carboxylate

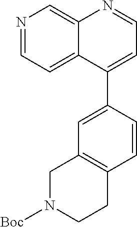 [SMILES: CC(C)(C)OC(=O)N1CCc2ccc(-c3ccnc4cnccc34)cc2C1]

To a mixture of 4-chloro-1,7-naphthyridine (570 mg, 3.5 mmol, 1 equiv) in 1,4-dioxane (15 mL) and water (5 mL) was added tert-butyl 7-(4,4,5,5-tetramethyl-1,3,2-dioxaborolan-2-yl)-3,4-dihydroisoquinoline-2 (1H)-carboxylate (1.85 g, 4.94 mmol, 1.5 equiv), Pd(dppf)Cl₂. CH₂Cl₂ (284 mg, 0.35 mmol, 0.1 equiv) and K₂CO₃ (1.45 g, 10.5 mmol, 3 equiv). The mixture was stirred at 80° C. for 2 h under nitrogen atmosphere. After cooling to room temperature, the resulting mixture was concentrated under reduced pressure. The residue was purified by silica gel column chromatography, eluted with EA/PE (50:50). The fractions containing the desired product were combined and concentrated under reduced pressure to give tert-butyl 7-(1,7-naphthyridin-4-yl)-3,4-dihydroisoquinoline-2 (1H)-carboxylate (910 mg, 73%) as a yellow solid.

### Step 2:4-(1,2,3,4-Tetrahydroisoquinolin-7-yl)-1,7-naphthyridine hydrochloride

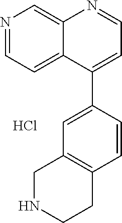 [SMILES: c1cc2c(-c3ccc4c(c3)CNCC4)ccnc2cn1 ; Cl]

A mixture tert-butyl 7-(1,7-naphthyridin-4-yl)-3,4-dihydroisoquinoline-2 (1H)-carboxylate (890 mg, 2.47 mmol, 1 equiv) in 14 mL HCl (g, 4 M in EtOAc) was stirred at room temperature for 2 h. Then the resulting mixture was concentrated under reduced pressure to afford 4-(1,2,3,4-tetrahydroisoquinolin-7-yl)-1,7-naphthyridine hydrochloride (1.02 g, crude) as a yellow solid.

### Step 3:4-(2-(N-(tert-Butyldimethylsilyl)-S-methylsulfonimidoyl)-1,2,3,4-tetrahydroiso-quinolin-7-yl)-1,7-naphthyridine

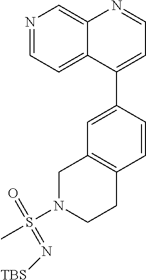

The title compound was synthesized by proceeding analogously as described in Example 32, Step 3 except 4-(1,2,3,4-tetrahydroisoquinolin-7-yl)-1,7-naphthyridine hydrochloride (902 mg, crude) was used. 4-(2-(N-(tert-butyldimethylsilyl)-S-methylsulfonimidoyl)-1,2,3,4-tetrahydroisoquinolin-7-yl)-1,7-naphthyridine (200 mg, 18% of two steps) was obtained as a yellow solid.

### Step 4:4-(2-(S-methylsulfonimidoyl)-1,2,3,4-tetrahydroisoquinolin-7-yl)-1,7-naphthyridine

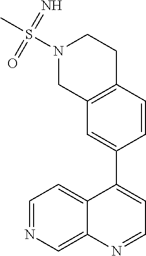 [SMILES: CS(=N)(=O)N1CCc2ccc(-c3ccnc4cnccc34)cc2C1]

The title compound was synthesized by proceeding analogously as described in Example 34 Step 4 except 4-(2-(N-(tert-butyldimethylsilyl)-S-methylsulfonimidoyl)-1,2,3,4-tetrahydroisoquinolin-7-yl)-1,7-naphthyridine (180 mg, 0.40 mmol) was used. The crude product was purified by prep-HPLC with the following conditions: (Column: XB ridge Prep OBD C18 Column, 30*150 mm, 5 μm; Mobile Phase A: Water (0.1% FA), Mobile Phase B: ACN; Flow rate: 60 mL/min; Gradient: 10% B to 40% B 10 min; Wave length: 254/220 nm. The fractions containing the desired product was concentrated under reduced pressure and lyophilized to afford 4-(2-(S-methylsulfonimidoyl)-1,2,3,4-tetrahydroisoquinolin-7-yl)-1,7-naphthyridine (67.1 mg, 50%) as an off-white solid. MS (ESI, pos. ion) m/z: 339.2 (M+1). ¹H NMR (400 MHZ, DMSO-d₆, ppm) δ 9.47 (s, 1H), 9.10 (d, J=4.4 Hz, 1H), 8.61 (d, J=5.6 Hz, 1H), 7.80 (d, J=6 Hz, 1H), 7.73 (d, J=4.4 Hz, 1H), 7.42 (d, J=6.4 Hz, 3H), 4.46 (s, 2H), 3.79 (s, 1H), 3.57-3.51 (m, 2H), 3.03-3.00 (m, 2H), 2.84 (s, 3H).

### Example 34

### Synthesis of 6-methoxy-4-(2-(S-methylsulfonimidoyl)-2,8-diazaspiro[4.5]decan-8-yl)pyrido[3,4-d]pyrimidine

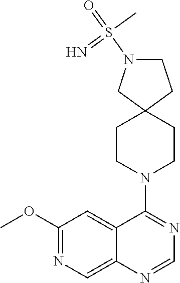 [SMILES: COc1cc2c(N3CCC4(CC3)CCN(S(C)(=N)=O)C4)ncnc2cn1]

### Step 1:6-Methoxypyrido[3,4-d]pyrimidin-4 (3H)-one

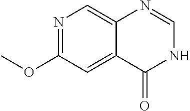

The title compound was synthesized by proceeding analogously as described in Example 14, Step 3 except 5-amino-2-methoxypyridine-4-carboxylic acid (5.00 g, 29.74 mmol) was used. 6-methoxypyrido[3,4-d]pyrimidin-4 (3H)-one (5.00 g, 95%) was obtained as a white solid.

### Step 2:4-Chloro-6-methoxypyrido[3,4-d]pyrimidine

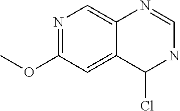

The title compound was synthesized by proceeding analogously as described in Example 14, Step 4 except 6-methoxypyrido[3,4-d]pyrimidin-4 (3H)-one (2.00 g, 11.28 mmol) in CHCl₃ (30 mL) was used and stirred overnight at 80° C. 4-chloro-6-methoxypyrido[3,4-d]pyrimidine (2.00 g, 85%) as a yellow solid.

### Step 3: tert-Butyl 8-[6-methoxypyrido[3,4-d]pyrimidin-4-yl]-2,8-diazaspiro[4.5]decane-2-carboxylate

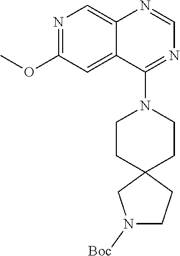 [SMILES: COc1cc2c(N3CCC4(CCN(C(=O)OC(C)(C)C)C4)CC3)ncnc2cn1]

The title compound was synthesized by proceeding analogously as described in Example 1, Step 4 except 4-chloro-6-methoxypyrido[3,4-d]pyrimidine (500 mg, 2.56 mmol) and tert-butyl 2,8-diazaspiro[4.5]decane-2-carboxylate (1.23 g, 5.11 mmol) were used and stirred at 100° C. for 2 h. tert-Butyl 8-[6-methoxypyrido[3,4-d]pyrimidin-4-yl]-2,8-diazaspiro[4.5]decane-2-carboxylate (1.00 g, 95%) was obtained as a yellow solid.

### Step 4:6-Methoxy-4-(2,8-diazaspiro[4.5]decan-8-yl)pyrido[3,4-d]pyrimidine

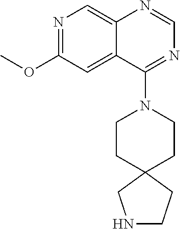 [SMILES: COc1cc2c(N3CCC4(CCNC4)CC3)ncnc2cn1]

A solution of tert-butyl 8-[6-methoxypyrido[3,4-d]pyrimidin-4-yl]-2,8-diaza -spiro[4.5]decane-2-carboxylate (1.00 g, 2.50 mmol) in 15 mL HCl (g, 4 M in EtOAc) was stirred at room temperature for 1 h. The mixture was concentrated by reduced pressure. The residue was purified by reverse phase flash chromatography with the following conditions: column, C18 silica gel; mobile phase, water of NH₄HCO₃ in ACN, 5% to 40% gradient in 20 min; detector, UV 254/220 nm. The fractions containing the desired product were concentrated under reduced pressure to afford 6-methoxy-4-(2,8-diazaspiro[4.5]decan-8-yl)pyrido[3,4-d]pyrimidine (271 mg, 32%) as a yellow solid.

### Step 5:4-(2-(N-(tert-Butyldimethylsilyl)-S-methylsulfonimidoyl)-2,8-diazaspiro[4.5]decan-8-yl)-6-methoxypyrido[3,4-d]pyrimidine

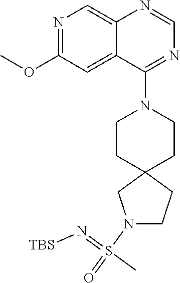

The title compound was synthesized by proceeding analogously as described in Example 32, Step 3 except 6-methoxy-4-(2,8-diazaspiro[4.5]decan-8-yl)pyrido[3,4-d]pyrimidine (217 mg, 0.72 mmol) was used. 4-(2-(N-(tert-butyldimethylsilyl)-S-methylsulfonimidoyl)-2,8-diazaspiro[4.5]decan-8-yl)-6-methoxypyrido[3,4-d]pyrimidine (122 mg, 34%) was obtained as a yellow oil.

### Step 6:6-Methoxy-4-(2-(S-methylsulfonimidoyl)-2,8-diazaspiro[4.5]decan-8-yl)pyrido[3,4-d]pyrimidine

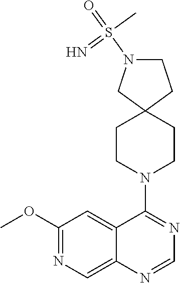 [SMILES: COc1cc2c(N3CCC4(CC3)CCN(S(C)(=N)=O)C4)ncnc2cn1]

The title compound was synthesized by proceeding analogously as described in Example 32, Step 4 except 4-(2-(N-(tert-butyldimethylsilyl)-S-methylsulfonimidoyl)-2,8-diazaspiro[4.5]decan-8-yl)-6-methoxypyrido[3,4-d]pyrimidine (112 mg, 0.23 mmol) was used. The crude product was purified by prep-HPLC with the following conditions: (Column: XBridge Prep OBD C18 Column, 30*150 mm, 5 μm; Mobile Phase A: Water (10 nmol/L NH₄HCO₃), Mobile Phase B: ACN; Flow rate: 60 mL/min; Gradient: 5% B to 35% B in 10 min; Wave Length: 254/220 nm; RT1 (min): 8.82) to afford 6-methoxy-4-(2-(S-methylsulfonimidoyl)-2,8-diazaspiro[4.5]decan-8-yl)pyrido[3,4-d]pyrimidine (29.3 mg, 33%) as a white solid. MS (ESI, pos. ion) m/z: 377.3 (M+1). ¹H NMR (400 MHZ, DMSO-d₆, ppm) δ 8.93 (s, 1H), 8.53 (s, 1H), 7.10 (s, 1H), 3.97 (s, 3H), 3.85-3.71 (m, 4H), 3.63 (s, 1H), 3.32-3.23 (m, 2H), 3.18-3.07 (m, 2H), 2.80 (d, J=1.2 Hz, 3H), 1.90-1.80 (m, 2H), 1.80-1.64 (m, 4H).

### Example 35

### Synthesis of 8-Methoxy-4-(2-(S-methylsulfonimidoyl)-2,8-diazaspiro[4.5]decan-8-yl)pyrido[3,4-d]pyrimidine

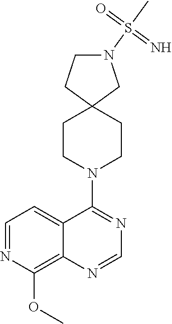 [SMILES: COc1nccc2c(N3CCC4(CC3)CCN(S(C)(=N)=O)C4)ncnc12]

### Step 1: tert-Butyl 8-(8-methoxypyrido[3,4-d]pyrimidin-4-yl)-2,8-diazaspiro[4.5]decane-2-carboxylate

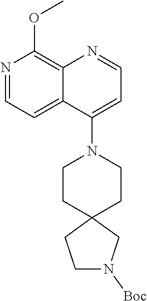 [SMILES: COc1nccc2c(N3CCC4(CCN(C(=O)OC(C)(C)C)C4)CC3)ccnc12]

The title compound was synthesized by proceeding analogously as described in Example 30, Step 1 except 4-chloro-8-methoxypyrido[3,4-d]pyrimidine (500 mg, 2.56 mmol) and tert-butyl 2,8-diazaspiro[4.5]decane-2-carboxylate (1.23 g, 5.11 mmol) in DMF (5 mL) were used and stirred at 80° C. for 2 h. tert-butyl 8-[8-methoxypyrido[3,4-d]pyrimidin-4-yl]-2,8-diazaspiro[4.5]decane-2-carboxylate (759 mg, 74%) as an off-white solid.

### Step 2:8-Methoxy-4-(2,8-diazaspiro[4.5]decan-8-yl)pyrido[3,4-d]pyrimidine

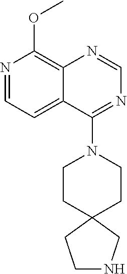 [SMILES: COc1nccc2c(N3CCC4(CCNC4)CC3)ncnc12]

To a solution of tert-butyl 8-[8-methoxypyrido[3,4-d]pyrimidin-4-yl]-2,8-diazaspiro[4.5]decane-2-carboxylate (200 mg, 0.50 mmol, 1 equiv) in dichloromethane (2 mL) was added 2,4-dimethylpyridine (53 mg, 0.50 mmol, 1 equiv) and trimethylsilyl trifluoromethanesulfonate (333 mg, 1.50 mmol, 3 equiv). The resulting mixture was stirred at room temperature for 1 h. The solution was concentrated under reduced pressure to afford 8-methoxy-4-(2,8-diazaspiro[4.5]decan-8-yl)pyrido[3,4-d]pyrimidine (495 mg, crude) was obtained as a white solid.

### Step 3:4-(2-(N-(tert-Butyldimethylsilyl)-S-methylsulfonimidoyl)-2,8-diazaspiro[4.5]decan-8-yl)-8-methoxypyrido[3,4-d]pyrimidine

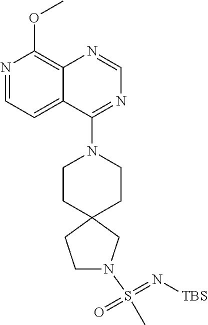

The title compound was synthesized by proceeding analogously as described in Example 32, Step 3 except 8-methoxy-4-(2,8-diazaspiro[4.5]decan-8-yl)pyrido[3,4-d]pyrimidine (459 mg, crude) was used. 4-(2-(N-(tert-butyldimethylsilyl)-S-methylsulfonimidoyl)-2,8-diazaspiro[4.5]decan-8-yl)-8-methoxypyrido[3,4-d]pyrimidine (174 mg, 23%) was obtained as a yellow oil.

### Step 4:8-Methoxy-4-(2-(S-methylsulfonimidoyl)-2,8-diazaspiro[4.5]decan-8-yl)pyrido[3,4-d]pyrimidine

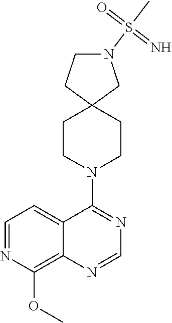 [SMILES: COc1nccc2c(N3CCC4(CC3)CCN(S(C)(=N)=O)C4)ncnc12]

The title compound was synthesized by proceeding analogously as described in Example 32, Step 4 except 4-(2-(N-(tert-butyldimethylsilyl)-S-methyl-sulfonimidoyl)-2,8-diazaspiro[4.5]decan-8-yl)-8-methoxypyrido[3,4-d]pyrimidine (140 mg, 0.28 mmol) was used. The crude product was purified by prep-HPLC with the following conditions: (Column: XBridge Prep Shield RP C18 Column, 30*150 mm, 5 μm; Mobile Phase A: Water (10 mmol/L NH₄HCO₃), Mobile Phase B: ACN; Flow rate: 60 mL/min; Gradient: 4% B to 34% B in 7 min; Wave Length: 254/220 nm; RT1 (min): 6.62) to afford 8-methoxy-4-(2-(S-methylsulfonimidoyl)-2,8-diazaspiro[4.5]decan-8-yl)pyrido[3,4-d]pyrimidine (30.9 mg, 28%) as a white solid. MS (ESI, pos. ion) m/z: 377.2 (M+1). ¹H NMR (400 MHZ, DMSO-d₆, ppm) δ 8.65 (s, 1H), 8.05 (d, J=6.0 Hz, 1H), 7.32 (d, J=6.0 Hz, 1H), 4.02 (s, 3H), 3.82-3.68 (m, 4H), 3.63 (s, 1H), 3.35-3.21 (m, 2H), 3.18-3.07 (m, 2H), 2.80 (s, 3H), 1.88-1.80 (m, 2H), 1.80-1.64 (m, 4H).

### Example 36

### Synthesis of 8-(8-methoxy-1,7-naphthyridin-4-yl)-2-(S-methylsulfonimidoyl)-2,8-diazaspiro[4.5]decane

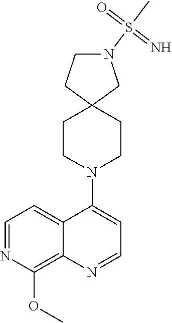 [SMILES: COc1nccc2c(N3CCC4(CC3)CCN(S(C)(=N)=O)C4)ccnc12]

### Step 1: tert-Butyl 8-(8-methoxy-1,7-naphthyridin-4-yl)-2,8-diazaspiro[4.5]decane-2-carboxylate

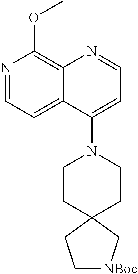 [SMILES: COc1nccc2c(N3CCC4(CCN(C(=O)OC(C)(C)C)C4)CC3)ccnc12]

The title compound was synthesized by proceeding analogously as described in Example 1, Step 4 except 4-chloro-8-methoxy-1,7-naphthyridine (400 mg, 2.055 mmol, 1 equiv) was used and stirred at 130° C. for 2 h. tert-butyl 8-(8-methoxy-1,7-naphthyridin-4-yl)-2,8-diazaspiro[4.5]decane-2-carboxylate (692 mg, 84%) was obtained as a yellow solid.

### Step 2:8-(8-Methoxy-1,7-naphthyridin-4-yl)-2,8-diazaspiro[4.5]decane

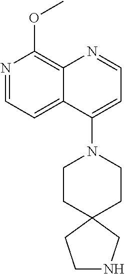 [SMILES: COc1nccc2c(N3CCC4(CCNC4)CC3)ccnc12]

The title compound was synthesized by proceeding analogously as described in Example 35, Step 2 except tert-butyl 8-(8-methoxy-1,7-naphthyridin-4-yl)-2,8-diazaspiro[4.5]decane-2-carboxylate (200 mg, 0.50 mmol) was used. 8-(8-methoxy-1,7-naphthyridin-4-yl)-2,8-diazaspiro[4.5]decane (285 mg, crude) was obtained as a colorless oil.

### Step 3: (tert-Butyldimethylsilyl) ([[8-(8-methoxy-1,7-naphthyridin-4-yl)-2,8-diazaspiro-[4.5]decan-2-yl](methyl)oxo-lambda6-sulfanylidene])amine

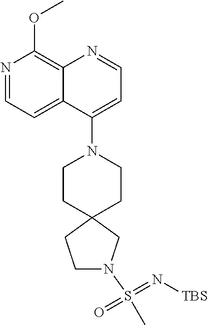

The title compound was synthesized by proceeding analogously as described in Example 32, Step 3 except 8-(8-methoxy-1,7-naphthyridin-4-yl)-2,8-diazaspiro[4.5]decane (149 mg, crude) was used. (tert-butyldimethylsilyl) ([[8-(8-methoxy-1,7-naphthyridin-4-yl)-2,8-diazaspiro[4.5]decan-2-yl](methyl)oxo-lambda6-sulfanylidene])amine (83 mg, 37%) as a yellow solid.

### Step 4:8-(8-Methoxy-1,7-naphthyridin-4-yl)-2-(S-methylsulfonimidoyl)-2,8-diaza -spiro[4.5]decane

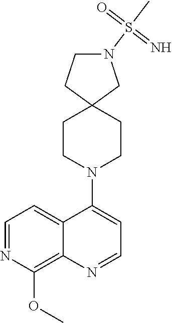 [SMILES: COc1nccc2c(N3CCC4(CC3)CCN(S(C)(=N)=O)C4)ccnc12]

The title compound was synthesized by proceeding analogously as described in Example 32, Step 4 except (tert-butyldimethylsilyl) ([[8-(8-methoxy-1,7-naphthyridin-4-yl)-2,8-diazaspiro[4.5]decan-2-yl](methyl)oxo-26-sulfanylidene])amine (73 mg, 0.149 mmol) was used. The crude product was purified by prep-HPLC with the following conditions: (Column: XBridge Prep Shield RP C18 Column, 30*150 mm, 5 μm; Mobile Phase A: Water (10 mmol/L NH₄HCO₃), Mobile Phase B: ACN; Flow rate: 60 mL/min; Gradient: 10% B to 31% B in 7 min; Wavelength: 254/220 nm; RT1 (min): 6.35). The fractions containing the desired product were combined and lyophilized to give 8-(8-methoxy-1,7-naphthyridin-4-yl)-2-(S-methylsulfonimidoyl)-2,8-diazaspiro[4.5]decane (20.2 mg, 36%) as a white solid. MS (ESI, pos. ion) m/z: 376.1 (M+1). ¹H NMR (400 MHZ, DMSO-d₆, ppm) δ 8.68 (d, J=5.0 Hz, 1H), 8.03 (d, J=5.9 Hz, 1H), 7.35 (d, J=5.9 Hz, 1H), 7.18 (d, J=5.1 Hz, 1H), 4.03 (s, 3H), 3.63 (s, 1H), 3.29 (t, J=7.0 Hz, 2H), 3.17-3.14 (m, 6H), 2.81 (d, J=1.6 Hz, 3H), 1.84-1.81 (m, 6H).

### Example 37

### Synthesis of 2-(8-methoxy-1,7-naphthyridin-4-yl)-8-(S-methylsulfonimidoyl)-2,8-diazaspiro[4.5]decane

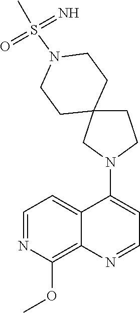 [SMILES: COc1nccc2c(N3CCC4(CCN(S(C)(=N)=O)CC4)C3)ccnc12]

### Step 1: tert-butyl 2-(8-methoxy-1,7-naphthyridin-4-yl)-2,8-diazaspiro[4.5]decane-8-carboxylate

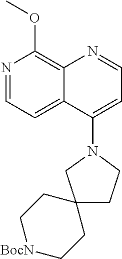 [SMILES: COc1nccc2c(N3CCC4(CCN(C(=O)OC(C)(C)C)CC4)C3)ccnc12]

The title compound was synthesized by proceeding analogously as described in Example 1, Step 4 except 4-chloro-8-methoxy-1,7-naphthyridine (230 mg, 1.18 mmol) and tert-butyl 2,8-diazaspiro[4.5]decane-8-carboxylate (568 mg, 2.36 mmol) were used and stirred at 130° C. for 2 h. tert-Butyl 2-(8-methoxy-1,7-naphthyridin-4-yl)-2,8-diazaspiro[4.5]decane-8-carboxylate (486 mg, 90%) was obtained as a yellow solid.

### Step 2:2-(8-Methoxy-1,7-naphthyridin-4-yl)-2,8-diazaspiro[4.5]decane

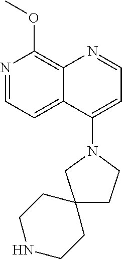 [SMILES: COc1nccc2c(N3CCC4(CCNCC4)C3)ccnc12]

The title compound was synthesized by proceeding analogously as described in Example 35, Step 2 except tert-butyl 2-(8-methoxy-1,7-naphthyridin-4-yl)-2,8-diazaspiro[4.5]decane-8-carboxylate (240 mg, 0.60 mmol) was used. 2-(8-Methoxy-1,7-naphthyridin-4-yl)-2,8-diazaspiro[4.5]decane (527 mg, crude) was obtained as a brown oil.

### Step 3:8-(N-(tert-Butyldimethylsilyl)-S-methylsulfonimidoyl)-2-(8-methoxy-1,7-naphthyridin-4-yl)-2,8-diazaspiro[4.5]decane

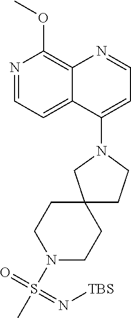

The title compound was synthesized by proceeding analogously as described in Example 32, Step 3 except 2-(8-methoxy-1,7-naphthyridin-4-yl)-2,8-diazaspiro[4.5]decane (527 mg, 1.76 mmol) was used. 8-(N-(tert-butyldimethylsilyl)-S-methylsulfonimidoyl)-2-(8-methoxy-1,7-naphthyridin-4-yl)-2,8-diazaspiro[4.5]decane (168 mg, 19%) was obtained as a brown solid.

### Step 4:2-(8-Methoxy-1,7-naphthyridin-4-yl)-8-(S-methylsulfonimidoyl)-2,8-diaza -spiro[4.5]decane

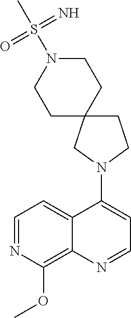 [SMILES: COc1nccc2c(N3CCC4(CCN(S(C)(=N)=O)CC4)C3)ccnc12]

The title compound was synthesized by proceeding analogously as described in Example 32, Step 4 except 8-(N-(tert-butyldimethylsilyl)-S-methylsulfonimidoyl)-2-(8-methoxy-1,7-naphthyridin-4-yl)-2,8-diazaspiro[4.5]decane (168 mg, 0.34 mmol) was used. The crude product was purified by prep-HPLC with the following conditions: (Column: XBridge Prep Shield RP C18 Column, 30*150 mm, 5 μm; Mobile Phase A: Water (10 mmol/L NH₄HCO₃), Mobile Phase B: ACN; Flow rate: 60 mL/min; Gradient: 61% B to 31% B in 7 min; Wave Length: 254/220 nm; RT1 (min): 7.17) to afford 2-(8-methoxy-1,7-naphthyridin-4-yl)-8-(S-methyl-sulfonimidoyl)-2,8-diazaspiro[4.5]decane (44.7 mg, 33%) as a white solid. MS (ESI, pos. ion) m/z: 376.2 (M+1). ¹H NMR (400 MHZ, DMSO-d₆, ppm) δ 8.42 (d, J=5.2 Hz, 1H), 7.87 (d, J=6.4 Hz, 1H), 7.69 (d, J=6.4 Hz, 1H), 6.71 (d, J=5.2 Hz, 1H), 4.00 (s, 3H), 3.79-3.71 (m, 2H), 3.51 (d, J=10.8 Hz, 3H), 3.27-3.07 (m, 4H), 2.75 (d, J=1.6 Hz, 3H), 1.95-1.87 (m, 2H), 1.73-1.60 (m, 4H).

### Example 38

### Synthesis of 8-methoxy-4-(7-(S-methylsulfonimidoyl)-2,7-diazaspiro[4.4]nonan-2-yl)-1,7-naphthyridine

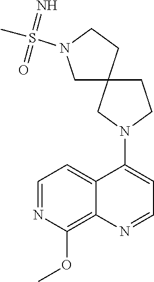 [SMILES: COc1nccc2c(N3CCC4(CCN(S(C)(=N)=O)C4)C3)ccnc12]

### Step 1: tert-butyl 8-(8-methoxy-1,7-naphthyridin-4-yl)-2,8-diazaspiro[4.5]decane-2-carboxylate

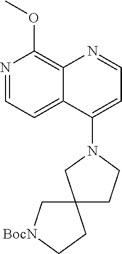 [SMILES: COc1nccc2c(N3CCC4(CCN(C(=O)OC(C)(C)C)C4)C3)ccnc12]

The title compound was synthesized by proceeding analogously as described in Example 1, Step 4 except 4-chloro-8-methoxy-1,7-naphthyridine (250 mg, 1.285 mmol) and tert-butyl 2,7-diazaspiro[4.4]nonane-2-carboxylate (436 mg, 1.92 mmol) were used and stirred overnight at 130° C. tert-Butyl 7-(8-methoxy-1,7-naphthyridin-4-yl)-2,7-diazaspiro[4.4]nonane-2-carboxylate (423 mg, 85%) was obtained as a yellow oil.

### Step 2:4-[2,7-Diazaspiro[4.4]nonan-2-yl]-8-methoxy-1,7-naphthyridine

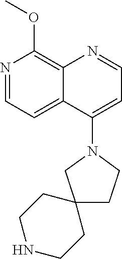 [SMILES: COc1nccc2c(N3CCC4(CCNCC4)C3)ccnc12]

The title compound was synthesized by proceeding analogously as described in Example 35, Step 2 except tert-butyl 7-(8-methoxy-1,7-naphthyridin-4-yl)-2,7-diazaspiro[4.4]nonane-2-carboxylate (220 mg, 0.57 mmol) was used. 4-[2,7-diazaspiro[4.4]nonan-2-yl]-8-methoxy-1,7-naphthyridine (380 mg, crude) was obtained as a yellow solid.

### Step 3: (tert-butyldimethylsilyl) (([8-(8-methoxy-1,7-naphthyridin-4-yl)-2,8-diazaspiro-[4.5]decan-2-yl](methyl)oxo-lambda6-sulfanylidene))amine

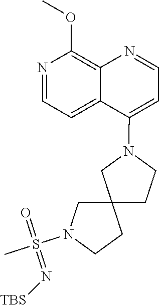

The title compound was synthesized by proceeding analogously as described in Example 32, Step 3 except 4-[2,7-diazaspiro[4.4]nonan-2-yl]-8-methoxy-1,7-naphthyridine (155 mg, 0.545 mmol) was used. (tert-butyldimethylsilyl) ([[7-(8-methoxy-1,7-naphthyridin-4-yl)-2,7-diazaspiro[4.4]nonan-2-yl](methyl)oxo-lambda6-sulfanylidene])amine (82 mg, 31%) was obtained as a yellow solid.

### Step 4:8-Methoxy-4-(7-(S-methylsulfonimidoyl)-2,7-diazaspiro[4.4]nonan-2-yl)-1,7-naphthyridine

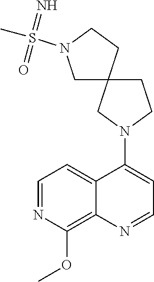 [SMILES: COc1nccc2c(N3CCC4(CCN(S(C)(=N)=O)C4)C3)ccnc12]

The title compound was synthesized by proceeding analogously as described in Example 32, Step 4 except (tert-butyldimethylsilyl) ([[7-(8-methoxy-1,7-naphthyridin-4-yl)-2,7-diazaspiro[4.4]nonan-2-yl](methyl)oxo-lambda6-sulfanylidene])amine (77 mg, 0.16 mmo) was used. The crude product was purified by prep-HPLC with the following conditions: (Column: YMC-Actus Triart C18 ExRS Column, 30*150 mm, 5 μm; Mobile Phase A: Water (10 mmol/L NH₄HCO₃), Mobile Phase B: ACN; Flow rate: 60 mL/min; Gradient: 3% B to 33% B in 7 min; Wavelength: 254/220 nm; RT1 (min): 6.97). The fractions containing the desired product were combined and lyophilized to give 8-methoxy-4-(7-(S-methylsulfonimidoyl)-2,7-diazaspiro[4.4]nonan-2-yl)-1,7-naphthyridine (22.1 mg, 37%) as a white solid.MS (ESI, pos. ion) m/z: 362.1 (M+1). ¹H NMR (400 MHZ, DMSO-d₆, ppm) δ 8.43 (d, J=5.4 Hz, 1H), 7.87 (d, J=6.1 Hz, 1H), 7.67 (dd, J=6.3, 2.5 Hz, 1H), 6.71 (d, J=5.5 Hz, 1H), 4.00 (s, 3H), 3.79-3.60 (m, 5H), 3.29-3.16 (m, 4H), 2.81 (d, J=1.6 Hz, 3H), 2.11-1.89 (m, 4H).

### Example 39

### Synthesis of 8-methoxy-4-(2-(S-methylsulfonimidoyl)-1,2,3,4-tetrahydroisoquinolin-7-yl)-1,7-naphthyridine

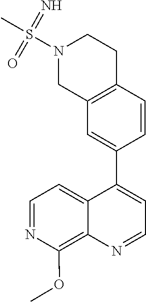 [SMILES: COc1nccc2c(-c3ccc4c(c3)CN(S(C)(=N)=O)CC4)ccnc12]

### Step 1: tert-Butyl 7-(8-methoxy-1,7-naphthyridin-4-yl)-3,4-dihydro-1H-isoquinoline-2-carboxylate

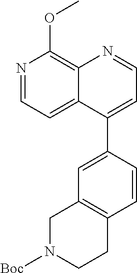 [SMILES: COc1nccc2c(-c3ccc4c(c3)CN(C(=O)OC(C)(C)C)CC4)ccnc12]

The title compound was synthesized by proceeding analogously as described in Example 33, Step 1 except 4-chloro-8-methoxy-1,7-naphthyridine (300 mg, 1.54 mmol) and tert-butyl 7-(4,4,5,5-tetramethyl-1,3,2-dioxaborolan-2-yl)-3,4-dihydro-1H-isoquinoline-2-carboxylate (664 mg, 1.85 mmol) were used, tert-Butyl 7-(8-methoxy-1,7-naphthyridin-4-yl)-3,4-dihydro-1H-isoquinoline-2-carboxylate (793 mg, crude) was obtained as a yellow oil.

### Step 2:8-Methoxy-4-(1,2,3,4-tetrahydroisoquinolin-7-yl)-1,7-naphthyridine

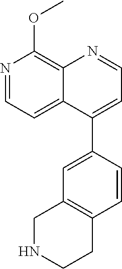 [SMILES: COc1nccc2c(-c3ccc4c(c3)CNCC4)ccnc12]

The title compound was synthesized by proceeding analogously as described in Example 35, Step 2 except tert-butyl 7-(8-methoxy-1,7-naphthyridin-4-yl)-3,4-dihydro-1H-isoquinoline-2-carboxylate (760 mg, crude) was used. 8-Methoxy-4-(1,2,3,4-tetrahydroisoquinolin-7-yl)-1,7-naphthyridine (1.69 g, crude) was obtained as a yellow oil.

### Step 3:4-(2-(N-(tert-Butyldimethylsilyl)-S-methylsulfonimidoyl)-1,2,3,4-tetrahydro-isoquinolin-7-yl)-8-methoxy-1,7-naphthyridine

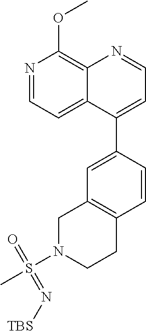

The title compound was synthesized by proceeding analogously as described in Example 32, Step 3 except 8-methoxy-4-(1,2,3,4-tetrahydroisoquinolin-7-yl)-1,7-naphthyridine (1.59 g, crude) was used. 4-(2-(N-(tert-butyldimethylsilyl)-S-methylsulfonimidoyl)-1,2,3,4-tetrahydroisoquinolin-7-yl)-8-methoxy-1,7-naphthyridine (422 mg, 16%) was obtained as a brown solid.

### Step 4:8-Methoxy-4-(2-(S-methylsulfonimidoyl)-1,2,3,4-tetrahydroisoquinolin-7-yl)-1,7-naphthyridine

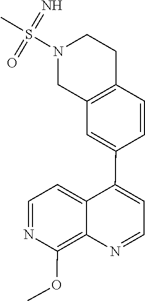 [SMILES: COc1nccc2c(-c3ccc4c(c3)CN(S(C)(=N)=O)CC4)ccnc12]

The title compound was synthesized by proceeding analogously as described in Example 32, Step 4 except 4-(2-(N-(tert-butyldimethylsilyl)-S-methyl-sulfonimidoyl)-1,2,3,4-tetrahydroisoquinolin-7-yl)-8-methoxy-1,7-naphthyridine (150 mg, 0.31 mmol) was used. The crude product was purified by prep-HPLC with the following conditions: (Column: YMC-Actus Triart C18 ExRS Column, 30*150 mm, 5 μm; Mobile Phase A: Water (10 mmol/L NH₄HCO₃), Mobile Phase B: ACN; Flow rate: 60 mL/min; Gradient: 18% B to 39% B in 7 min; Wavelength: 254/220 nm; RT1 (min): 6.6) to afford 8-methoxy-4-(2-(S-methylsulfonimidoyl)-1,2,3,4-tetrahydroisoquinolin-7-yl)-1,7-naphthyridine (38.7 mg, 33%) as a white solid. MS (ESI, pos. ion) m/z: 369.1 (M+1). ¹H NMR (400 MHZ, DMSO-d₆, ppm) δ 8.98 (d, J=4.4 Hz, 1H), 8.12-8.05 (m, 1H), 7.72-7.66 (m, 1H), 7.41-7.35 (m, 3H), 7.31 (d, J=6.1 Hz, 1H), 4.46 (d, J=2.4 Hz, 2H), 4.10 (s, 3H), 3.77 (d, J=1.9 Hz, 1H), 3.59-3.42 (m, 2H), 3.05-2.97 (m, 2H), 2.84 (d, J=1.6 Hz, 3H).

### Example 40

### Synthesis of 8-methoxy-4-(2-(S-methylsulfonimidoyl)-1,2,3,4-tetrahydroisoquinolin-7-yl)-pyrido[3,4-d]pyrimidine

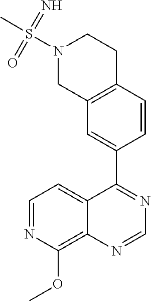 [SMILES: COc1nccc2c(-c3ccc4c(c3)CN(S(C)(=N)=O)CC4)ncnc12]

### Step 1: tert-Butyl 7-[8-methoxypyrido[3,4-d]pyrimidin-4-yl]-3,4-dihydro-1H-isoquinoline-2-carboxylate

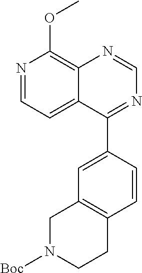 [SMILES: COc1nccc2c(-c3ccc4c(c3)CN(C(=O)OC(C)(C)C)CC4)ncnc12]

The title compound was synthesized by proceeding analogously as described in Example 33, Step 1 except 4-chloro-8-methoxypyrido[3,4-d]pyrimidine (300 mg, 1.53 mmol) and tert-butyl 7-(4,4,5,5-tetramethyl-1,3,2-dioxaborolan-2-yl)-3,4-dihydro-1H-isoquinoline-2-carboxylate (664 mg, 1.85 mmol) were used, tert-Butyl 7-[8-methoxypyrido[3,4-d]pyrimidin-4-yl]-3,4-dihydro-1H-isoquinoline-2-carboxylate (407 mg, 67%) was obtained as a yellow solid.

### Step 2:7-[8-Methoxypyrido[3,4-d]pyrimidin-4-yl]-1,2,3,4-tetrahydroisoquinoline

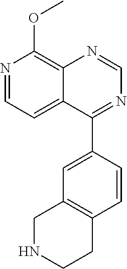 [SMILES: COc1nccc2c(-c3ccc4c(c3)CNCC4)ncnc12]

The title compound was synthesized by proceeding analogously as described in Example 35, Step 2 except tert-butyl 7-[8-methoxypyrido[3,4-d]pyrimidin-4-yl]-3,4-dihydro-1H-isoquinoline-2-carboxylate (387 mg, 0.98 mmol) was used. 7-[8-Methoxypyrido[3,4-d]pyrimidin-4-yl]-1,2,3,4-tetrahydroisoquinoline (1.00 g, crude) was obtained as a yellow solid.

### Step 3: (tert-Butyldimethylsilyl) [(7-[8-methoxypyrido[3,4-d]pyrimidin-4-yl]-3,4-dihydro-1H-isoquinolin-2-yl)(methyl)oxo-lambda6-sulfanylideneJamine

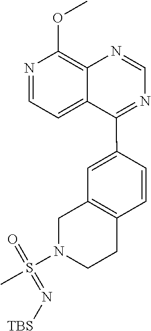

The title compound was synthesized by proceeding analogously as described in Example 32, Step 3 except 7-[8-methoxypyrido[3,4-d]pyrimidin-4-yl]-1,2,3,4-tetrahydroisoquinoline (500 mg, crude) was used. (tert-Butyldimethylsilyl) [(7-[8-methoxypyrido[3,4-d]pyrimidin-4-yl]-3,4-dihydro-1H-isoquinolin-2-yl)(methyl)oxo-lambda6-sulfanylideneJamine (79 mg, 9%) was obtained as a yellow solid.

### Step 4:8-Methoxy-4-(2-(S-methylsulfonimidoyl)-1,2,3,4-tetrahydroisoquinolin-7-yl)-pyrido[3,4-d]pyrimidine

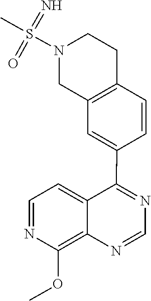 [SMILES: COc1nccc2c(-c3ccc4c(c3)CN(S(C)(=N)=O)CC4)ncnc12]

The title compound was synthesized by proceeding analogously as described in Example 32, Step 4 except (tert-butyldimethylsilyl) [(7-[8-methoxypyrido[3,4-d]-pyrimidin-4-yl]-3,4-dihydro-1H-isoquinolin-2-yl)(methyl)oxo-26-sulfanylideneJamine (69 mg, 0.143 mmol) was used. The crude product was purified by prep-HPLC with the following conditions: (Column: XBridge Prep Shield RP C18 Column, 30*150 mm, 5 μm; Mobile Phase A: Water (10 mmol/L NH₄HCO₃), Mobile Phase B: ACN; Flow rate: 60 mL/min; Gradient: 9% B to 39% B in 7 min; Wavelength: 254/220 nm; RT1 (min): 6.7). The fractions containing the desired product were combined and lyophilized to give 8-methoxy-4-(2-(S-methylsulfonimidoyl)-1,2,3,4-tetrahydroisoquinolin-7-yl)pyrido[3,4-d]pyrimidine (46.4 mg, 85%) as a white solid. MS (ESI, pos. ion) m/z: 370.1 (M+1). ¹H NMR (400 MHZ, DMSO-d₆, ppm) δ 9.45 (s, 1H), 8.24 (d, J=6.0 Hz, 1H), 7.68-7.61 (m, 2H), 7.52 (d, J=6.0 Hz, 1H), 7.45 (d, J=8.4 Hz, 1H), 4.49 (d, J=2.1 Hz, 2H), 4.13 (s, 3H), 3.78 (d, J=1.8 Hz, 1H), 3.58-3.42 (m, 2H), 3.04 (t, J=6.0 Hz, 2H), 2.85 (d, J=1.7 Hz, 3H).

### Example 41, 42 and 43

### Synthesis of cyclopropyl(3-(((8-(fluoromethoxy)-1,7-naphthyridin-4-yl)oxy)methyl)phenyl)-(imino)-lambda6-sulfanone and (R)-cyclopropyl(3-(((8-(fluoromethoxy)-1,7-naphthyridin-4-yl)oxy)methyl)phenyl) (imino)-lambda6-sulfanone and(S)-cyclopropyl(3-(((8-(fluoromethoxy)-1,7-naphthyridin-4-yl)oxy)methyl)phenyl) (imino)-lambda6-sulfanone

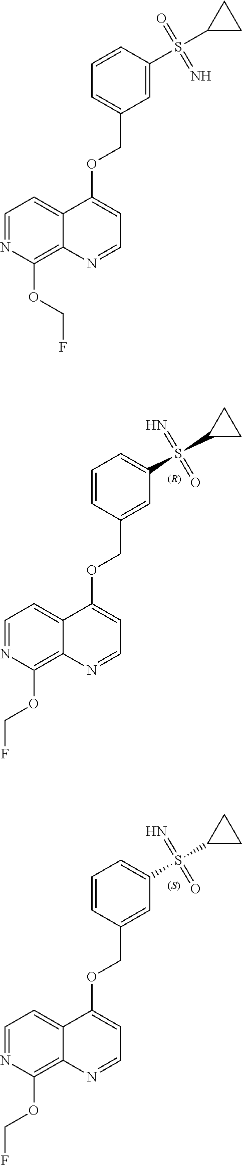

### Step 1:3-(Cyclopropylsulfanyl)benzoic acid

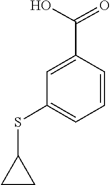 [SMILES: O=C(O)c1cccc(SC2CC2)c1]

To a mixture of 3-sulfanylbenzoic acid (20.00 g, 129.72 mmol, 1.0 equiv) in DMSO (200 mL) were added and bromocyclopropane (23.54 g, 194.57 mmol, 1.5 equiv) and potassium tert-butoxide (36.39 g, 324.29 mmol, 2.5 equiv). The reaction mixture was stirred at 80° C. for 24 h. After cooling down to room temperature, the reaction mixture was diluted with water, adjusted the pH to 5 with 1 N HCl (aq) and extracted with EtOAc. The combined organic layers were washed with brine, dried over anhydrous sodium sulfate and filtered. The filtrate was concentrated under reduced pressure. The residue was purified by silica gel column chromatography, eluted with EtOAc/PE (40:60) to afford 3-(cyclopropylsulfanyl)benzoic acid (20.00 g, 69%) as a colorless oil.

### Step 2: (3-(Cyclopropylsulfanyl)phenyl) methanol

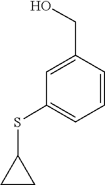 [SMILES: OCc1cccc(SC2CC2)c1]

To a solution of 3-(cyclopropylsulfanyl)benzoic acid (24.0 g, 117.37 mmol, 1 equiv) in THF (250 mL) was added lithium aluminum hydride (110 mL, 2 equiv, 2 M in THF) at 0° C. The resulting mixture was stirred overnight at room temperature under nitrogen atmosphere. The reaction mixture was quenched with saturated ammonium bicarbonate solution at 0° C. and extracted with EtOAc. The combined organic layers were washed with brine, dried over anhydrous sodium sulfate and filtered. The filtrate was concentrated under reduced pressure. The residue was purified by silica gel column chromatography, eluted with EtOAc/PE (10:90) to afford (3-(cyclopropylsulfanyl)-phenyl) methanol (20.0 g, 86%) as a yellow oil.

### Step 3:2-(Fluoromethoxy)-3-nitropyridine

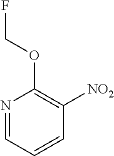 [SMILES: O=[N+]([O-])c1cccnc1OCF]

To a solution of 3-nitropyridin-2-ol (65.00 g, 463.96 mmol, 1.0 equiv) in DMF (650 mL) were added DIEA (242 mL, 1.39 mol, 3.0 equiv) and bromofluoromethane (104.80 g, 927.92 mmol, 2.0 equiv). The resulting mixture was stirred at 80° C. for 16 h. After cooling down to room temperature, the reaction mixture was diluted with EtOAc and filtered. The filtrate was washed with H₂O and brine. The organic layer was dried over anhydrous sodium sulfate and concentrated under reduced pressure. The residue was purified by silica gel column chromatography, eluted with EtOAc/PE (1:4) to afford 2-(fluoromethoxy)-3-nitropyridine (16.31 g, 16%) as a yellow oil.

### Step 4:2-(Fluoromethoxy)pyridin-3-amine

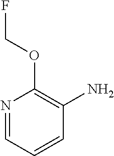

To a solution of 2-(fluoromethoxy)-3-nitropyridine (16.31 g, 92.96 mmol, 1.0 equiv) in MeOH (200 mL) was added Pd/C (4.20 g, 11.15 mmol, 0.2 equiv). After stirring overnight at room temperature under hydrogen atmosphere by using a hydrogen balloon, the reaction mixture was filtered through a Celite pad and concentrated under reduced pressure to afford 2-(fluoromethoxy)pyridin-3-amine (12.30 g, crude) as a yellow oil, which was used in the next step directly without further purification.

### Step 5:5-(((2-(Fluoromethoxy)pyridin-3-yl)amino)methylidene)-2,2-dimethyl-1,3-dioxane-4,6-dione

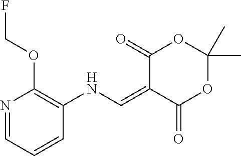 [SMILES: CC1(C)OC(=O)C(=CNc2cccnc2OCF)C(=O)O1]

To a solution of 2-(fluoromethoxy)pyridin-3-amine (12.00 g, 84.42 mmol, 1.0 equiv) in ACN (150 mL) were added 2,2-dimethyl-1,3-dioxane-4,6-dione (15.82 g, 109.75 mmol, 1.3 equiv) and trimethoxymethane (12.54 g, 118.20 mmol, 1.4 equiv). The reaction mixture was stirred at 80° C. for 2 h. After cooling down to room temperature, the resulting mixture was diluted with PE. The precipitate was collected by filtration and washed with PE to afford 5-(((2-(fluoromethoxy)pyridin-3-yl)amino)methylidene)-2,2-dimethyl-1,3-dioxane-4,6-dione (22.21 g, 87%) as a brown solid.

### Step 6:8-(Fluoromethoxy)-1,7-naphthyridin-4-ol

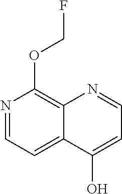 [SMILES: Oc1ccnc2c(OCF)nccc12]

A mixture of 5-(((2-(fluoromethoxy)pyridin-3-yl)amino)methylidene)-2,2-dimethyl-1,3-dioxane-4,6-dione (22.00 g, 54.01 mmol, 1.0 equiv) in diphenyl ether (200 mL) was stirred at 225° C. for 1.5 h. After cooling down to room temperature, the reaction mixture was diluted with PE. The precipitate was collected by filtration, washed with PE and purified by silica gel column chromatography, eluted with MeOH/DCM (7:93) to afford 8-(fluoromethoxy)-1,7-naphthyridin-4-ol (1.81 g, 17%) as a brown solid.

### Step 7:4-((3-(Cyclopropylsulfanyl)phenyl) methoxy)-8-(fluoromethoxy)-1,7-naphthyridine

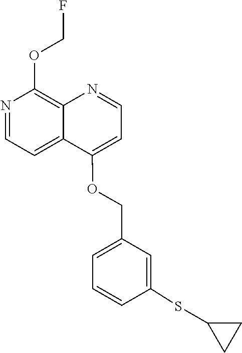 [SMILES: FCOc1nccc2c(OCc3cccc(SC4CC4)c3)ccnc12]

To a solution of 8-(fluoromethoxy)-1,7-naphthyridin-4-ol (1.00 g, 5.15 mmol, 1.0 equiv) in toluene (10 mL) were added 2-(tributyl-lambda5-phosphaneylidene) acetonitrile (2.49 g, 10.30 mmol, 2.0 equiv) and (3-(cyclopropylsulfanyl)phenyl) methanol (1.39 g, 7.72 mmol, 1.5 equiv). The resulting mixture was stirred at 130° C. for 2 h under N₂ atmosphere. After cooling down to room temperature, the reaction mixture was concentrated under reduced pressure and purified by silica gel column chromatography, eluted with EtOAc/PE (53:47) to afford a crude product, which was further purified by reversed-phase CombiFlash, eluted with ACN/H₂O (67:33) to afford 4-((3-(cyclopropylsulfanyl)phenyl) methoxy)-8-(fluoromethoxy)-1,7-naphthyridine (550 mg, 29%) as a yellow solid.

### Step 8: Cyclopropyl(3-(((8-(fluoromethoxy)-1,7-naphthyridin-4-yl)oxy)methyl)phenyl)-(imino)-lambda6-sulfanone

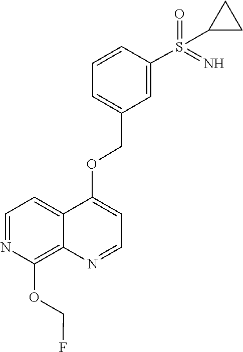 [SMILES: N=S(=O)(c1cccc(COc2ccnc3c(OCF)nccc23)c1)C1CC1]

To a solution of 4-((3-(cyclopropylsulfanyl)phenyl) methoxy)-8-(fluoromethoxy)-1,7-naphthyridine (550 mg, 1.53 mmol, 1.0 equiv) in MeOH (10 mL) were added (acetyloxy) (phenyl)-lambda3-iodanyl acetate (1.49 g, 4.62 mmol, 3.0 equiv) and ammonium carbamate (481 mg, 6.17 mmol, 4.0 equiv). After stirring at room temperature for 2 h, the resulting mixture was concentrated under reduced pressure. The residue was purified by reverse phase flash, eluted with ACN/H₂O (43:57) to afford cyclopropyl(3-(((8-(fluoromethoxy)-1,7-naphthyridin-4-yl)oxy)methyl)phenyl) (imino)-lambda6-sulfanone (450 mg, 75%) as a brown solid. MS (ESI, pos. ion) m/z: 388.1 (M+1); ¹H NMR (400 MHZ, DMSO-do, ppm) δ 8.88 (d, J=5.2 Hz, 1H), 8.16 (d, J=5.6 Hz, 1H), 8.09 (s, 1H), 7.92 (d, J=7.6 Hz, 1H), 7.86 (d, J=7.6 Hz, 1H), 7.76 (d, J=5.6 Hz, 1H), 7.69 (t, J=7.6 Hz, 1H), 7.45 (d, J=5.2 Hz, 1H), 6.38 (s, 1H), 6.25 (s, 1H), 5.57 (s, 2H), 4.29 (s, 1H), 2.73-2.63 (m, 1H), 1.15-1.03 (m, 1H), 1.04-0.83 (m, 3H).

¹⁹F NMR (376 MHz, DMSO-d₆, ppm) 8-155.95 (s, 1 F).

### Step 9: (R)-Cyclopropyl(3-(((8-(fluoromethoxy)-1,7-naphthyridin-4-yl)oxy)methyl)-phenyl) (imino)-lambda6-sulfanone and(S)-cyclopropyl(3-(((8-(fluoromethoxy)-1,7-naphthyridin-4-yl)oxy)methyl)phenyl) (imino)-lambda6-sulfanone

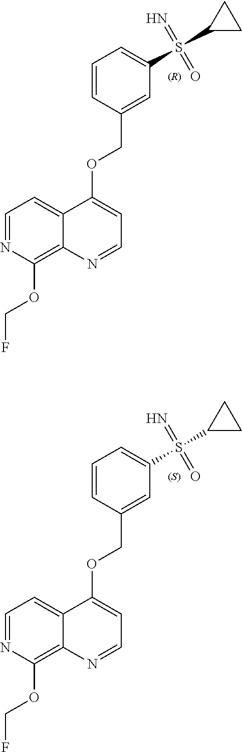

The racemic cyclopropyl(3-(((8-(fluoromethoxy)-1,7-naphthyridin-4-yl)oxy)methyl)-phenyl)imino-lambda6-sulfanylone (498 mg, 1.28 mmol) was purified by chiral SFC with the following conditions (Column: CHIRAL ART Amylose-C NEO 3*25 cm, 5 μm; Mobile Phase A: CO₂, Mobile Phase B: IPA (1%-2M-NH₃-MeOH); Flow rate: 100 mL/min; Gradient: isocratic 48% B; Column Temperature (C): 35; Back Pressure (bar): 100; Wave Length: 220 nm; Sample Solvent: MeOH; Injection Volume: 3 mL) to afford two fractions.

Fraction 1: (R)-cyclopropyl(3-(((8-(fluoromethoxy)-1,7-naphthyridin-4-yl)oxy)methyl)-phenyl) (imino)-lambda6-sulfanone (180 mg, 35%) as an off-white solid. RT1:7.12 min, MS (ESI, pos. ion) m/z: 388.1 (M+1). ¹H NMR (400 MHZ, DMSO-d₆, ppm) δ 8.88 (d, J=5.2 Hz, 1H), 8.16 (d, J=5.6 Hz, 1H), 8.09 (s, 1H), 7.92 (d, J=7.6 Hz, 1H), 7.86 (d, J=7.6 Hz, 1H), 7.76 (d, J=5.6 Hz, 1H), 7.69 (t, J=7.6 Hz, 1H), 7.45 (d, J=5.2 Hz, 1H), 6.38 (s, 1H), 6.25 (s, 1H), 5.57 (s, 2H), 4.29 (s, 1H), 2.73-2.63 (m, 1H), 1.15-1.03 (m, 1H), 1.04-0.83 (m, 3H).

¹⁹F NMR (377 MHz, DMSO-d₆, ppm) 8-155.94 (s, 1 F).

Fraction 2: (S)-cyclopropyl(3-(((8-(fluoromethoxy)-1,7-naphthyridin-4-yl)oxy)methyl)-phenyl) (imino)-lambda6-sulfanone (178 mg, 35%) as a yellow solid. RT2:10. 18 min, MS (ESI, pos. ion) m/z: 388.1 (M+1). ¹H NMR (400 MHZ, DMSO-d₆, ppm) δ 8.88 (d, J=5.2 Hz, 1H), 8.16 (d, J=5.6 Hz, 1H), 8.09 (s, 1H), 7.92 (d, J=7.6 Hz, 1H), 7.86 (d, J=7.6 Hz, 1H), 7.76 (d, J=5.6 Hz, 1H), 7.69 (t, J=7.6 Hz, 1H), 7.45 (d, J=5.2 Hz, 1H), 6.38 (s, 1H), 6.25 (s, 1H), 5.57 (s, 2H), 4.29 (s, 1H), 2.73-2.63 (m, 1H), 1.15-1.03 (m, 1H), 1.04-0.83 (m, 3H).

¹⁹F NMR (377 MHz, DMSO-d₆, ppm) 8-155.95 (s, 1 F).

### Example 44

### Synthesis of 8-methoxy-4-(2-(S-methylsulfonimidoyl)-2,6-diazaspiro(3.4) octan-6-yl)-1,7-naphthyridine

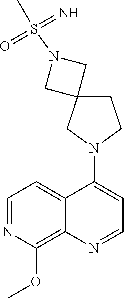 [SMILES: COc1nccc2c(N3CCC4(C3)CN(S(C)(=N)=O)C4)ccnc12]

### Step 1: tert-butyl 6-(8-Methoxy-1,7-naphthyridin-4-yl)-2,6-diazaspiro(3.4) octane-2-carboxylate

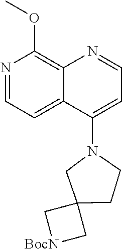 [SMILES: COc1nccc2c(N3CCC4(CN(C(=O)OC(C)(C)C)C4)C3)ccnc12]

To a solution of 4-chloro-8-methoxy-1,7-naphthyridine (250 mg, 1.28 mmol, 1.0 equiv.) in NMP (5 mL) were added DIEA (498 mg, 3.85 mmol, 3.0 equiv) and tert-butyl 2,6-diazaspiro-(3.4) octane-2-carboxylate (409 mg, 1.92 mmol, 1.5 equiv). The reaction mixture was stirred at 130° C. for 2 h. After cooling down to room temperature, the resulting mixture was purified by reverse phase column chromatography, eluted with ACN/H₂O (45:55) to afford tert-butyl 6-(8-methoxy-1,7-naphthyridin-4-yl)-2,6-diazaspiro(3.4) octane-2-carboxylate (420 mg, 88%) as a brown solid.

### Step 2:4-(2,6-Diazaspiro(3.4) octan-6-yl)-8-methoxy-1,7-naphthyridine

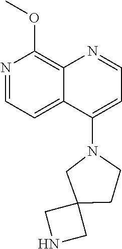 [SMILES: COc1nccc2c(N3CCC4(CNC4)C3)ccnc12]

To a solution of tert-butyl 6-(8-methoxy-1,7-naphthyridin-4-yl)-2,6-diazaspiro(3.4) octane-2-carboxylate (400 mg, 1.08 mmol, 1.0 equiv) in DCM (10 mL) were added lutidine (115 mg, 1.08 mmol, 1.0 equiv) and TMSOTf (719 mg, 3.24 mmol, 3.0 equiv). After stirring at room temperature for 1 h, the resulting mixture was concentrated under reduced pressure to afford 4-(2,6-diazaspiro(3.4) octan-6-yl)-8-methoxy-1,7-naphthyridine (400 mg, crude) as a brown solid, which was used in the next step directly without further purification.

### Step 3: (tert-butyl Dimethylsilyl) (((6-(8-methoxy-1,7-naphthyridin-4-yl)-2,6-diazaspiro(3.4)-octan-2-yl)(methyl)oxo-lambda6-sulfanylidene))amine

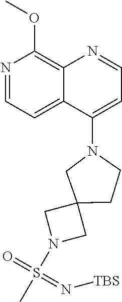

To a solution of Ph₃PO (926 mg, 3.33 mmol, 3.0 equiv) in DCM (10 mL) was added (COCl)₂ (450 mg, 3.55 mmol, 3.2 equiv). The reaction mixture was stirred at 0° C. for 0.5 h under nitrogen atmosphere. DIEA (1.43 g, 11.10 mmol, 10.0 equiv) was added, the resulting mixture was stirred at 0° C. for 10 min. N-(tert-butyl Dimethylsilyl) methanesulfonamide (348 mg, 1.66 mmol, 1.5 equiv) was added and the mixture was stirred at 0° C. for 20 min before being added to a mixture of 4-(2,6-diazaspiro(3.4) octan-6-yl)-8-methoxy-1,7-naphthyridine (300 mg, 1.11 mmol, 1.0 equiv) and DIEA (1.43 mg, 11.10 mmol, 10.0 equiv) in DCM (10 mL) at 0° C. under nitrogen atmosphere. After stirring for 0.5 h at room temperature under nitrogen atmosphere, the resulting mixture was concentrated under reduced pressure. The residue was purified by reversed phase flash, eluted with ACN/H₂O (72:28) to afford (tert-butyldimethylsilyl) (((6-(8-methoxy-1,7-naphthyridin-4-yl)-2,6-diazaspiro(3.4) octan-2-yl)(methyl)oxo-lambda6-sulfanylidene))amine (350 mg, 40%) as a brown oil.

### Step 4:8-Methoxy-4-(2-(S-methylsulfonimidoyl)-2,6-diazaspiro(3.4) octan-6-yl)-1,7-naphthyridine

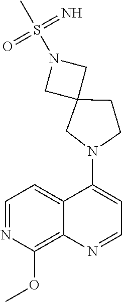 [SMILES: COc1nccc2c(N3CCC4(C3)CN(S(C)(=N)=O)C4)ccnc12]

To a solution of (tert-butyldimethylsilyl) (((6-(8-methoxy-1,7-naphthyridin-4-yl)-2,6-diazaspiro(3.4) octan-2-yl)(methyl)oxo-lambda6-sulfanylidene))amine (300 mg, 0.39 mmol, 1.0 equiv) in DMF (5 mL) was added formic acid (0.2 mL, 25% (v/v)). After stirring at room temperature for 10 min, the resulting mixture was concentrated under reduced pressure to afford crude product, which was further purified by prep-HPLC with the following conditions (Column: YMC-Actus Triart C18 ExRS Column, 30*150 mm, 5 μm; Mobile Phase A: Water (10 mmol/L NH₄HCO₃), Mobile Phase B: ACN; Flow rate: 60 mL/min; Gradient: 3% B to 26% B in 7 min; Wave Length: 254 nm/220 nm; RT: 6.95 min) to afford 8-methoxy-4-(2-(S-methylsulfonimidoyl)-2,6-diazaspiro(3.4) octan-6-yl)-1,7-naphthyridine (50 mg, 37%) as an off-white solid. MS (ESI, pos. ion) m/z: 348.1 (M+1). ¹H NMR (400 MHZ, DMSO-do, ppm) δ 8.43 (d, J=5.6 Hz, 1H), 7.89 (d, J=6.0 Hz, 1H), 7.65 (d, J=6.4, 1H), 6.73-6.67 (m, 1H), 4.00 (s, 3H), 3.91-3.80 (m, 4H), 3.75-3.71 (m, 2H), 3.66 (s, 2H), 3.57 (d, J=2.0 Hz, 1H), 2.89 (d, J=1.6 Hz, 3H), 2.21 (t, J=6.7 Hz, 2H).

### Example 45

### Synthesis of 8-methoxy-4-(6-(S-methylsulfonimidoyl)-2,6-diazaspiro(3.4) octan-2-yl)-1,7-naphthyridine

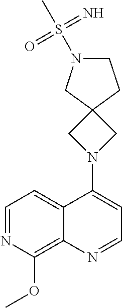 [SMILES: COc1nccc2c(N3CC4(CCN(S(C)(=N)=O)C4)C3)ccnc12]

### Step 1: tert-butyl 2-(8-Methoxy-1,7-naphthyridin-4-yl)-2,6-diazaspiro(3.4) octane-6-carboxylate

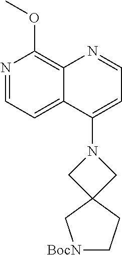 [SMILES: COc1nccc2c(N3CC4(CCN(C(=O)OC(C)(C)C)C4)C3)ccnc12]

To a solution of 4-chloro-8-methoxy-1,7-naphthyridine (250 mg, 1.28 mmol, 1.0 equiv) in NMP (5 mL) were added DIEA (498 mg, 3.85 mmol, 3.0 equiv) and tert-butyl 2,6-diazaspiro(3.4) octane-6-carboxylate (409 mg, 1.92 mmol, 1.5 equiv). The reaction mixture was stirred at 130° C. for 2 h. After cooling down to room temperature, the resulting mixture was concentrated under reduced pressure. The residue was purified by reverse phase flash, eluted with ACN/H₂O (60:40) to afford tert-butyl 2-(8-methoxy-1,7-naphthyridin-4-yl)-2,6-diazaspiro(3.4) octane-6-carboxylate (400 mg, 84%) as a brown solid.

### Step 2:8-Methoxy-4-(2,6-diazaspiro(3.4) octan-2-yl)-1,7-naphthyridine

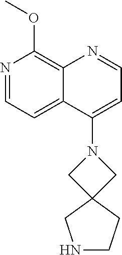 [SMILES: COc1nccc2c(N3CC4(CCNC4)C3)ccnc12]

To a solution of tert-butyl 2-(8-methoxy-1,7-naphthyridin-4-yl)-2,6-diazaspiro(3.4) octane-6-carboxylate (400 mg, 1.08 mmol, 1.0 equiv) in DCM (10 mL) were added Lutidine (115 mg, 1.08 mmol, 1.0 equiv) and TMSOTf (720 mg, 3.24 mmol, 3.0 equiv). After stirring at room temperature for 1 h, the resulting mixture was concentrated under reduced pressure to afford 8-methoxy-4-(2,6-diazaspiro(3.4) octan-2-yl)-1,7-naphthyridine (350 mg, crude), which was used in the next step directly without further purification.

### Step 3: (tert-butyl Dimethylsilyl) (((2-(8-methoxy-1,7-naphthyridin-4-yl)-2,6-diazaspiro(3.4)-octan-6-yl)(methyl)oxo-lambda6-sulfanylidene))amine

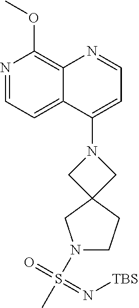

To a solution of Ph₃PO (926 mg, 3.33 mmol, 3.0 equiv) in DCM (5 mL) was added (COCl)₂ (450 mg, 3.55 mmol, 3.2 equiv) at 0° C. under nitrogen atmosphere. The reaction mixture was stirred at 0° C. for 30 min. DIEA (1.43 mg, 11.10 mmol, 10.0 equiv) was added and the resulting mixture was stirred at 0° C. for 15 min. N-(tert-butyldimethylsilyl) methanesulfonamide (348 mg, 1.66 mmol, 1.5 equiv) was added and the mixture was stirred at 0° C. for 20 min before being added to a mixture of 8-methoxy-4-(2,6-diazaspiro(3.4) octan-2-yl)-1,7-naphthyridine (300 mg, 1.11 mmol, 1.0 equiv) and DIEA (1.43 g, 11.10 mmol, 10.0 equiv) in DCM (5 mL) at 0° C. under nitrogen atmosphere. After stirring for 30 min at room temperature under nitrogen atmosphere, the resulting mixture was concentrated under reduced pressure and the residue was purified by reversed phase flash, eluted with ACN/H₂O (65:35) to afford (tert-butyldimethylsilyl) (((2-(8-methoxy-1,7-naphthyridin-4-yl)-2,6-diazaspiro(3.4) octan-6-yl)(methyl)oxo-lambda6-sulfanylidene))amine (350 mg, 40%) as a brown oil.

### Step 4:8-Methoxy-4-(6-(S-methylsulfonimidoyl)-2,6-diazaspiro(3.4) octan-2-yl)-1,7-naphthyridine

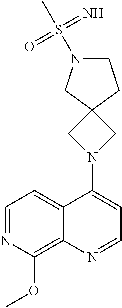 [SMILES: COc1nccc2c(N3CC4(CCN(S(C)(=N)=O)C4)C3)ccnc12]

To a solution of (tert-butyldimethylsilyl) (((2-(8-methoxy-1,7-naphthyridin-4-yl)-2,6-diazaspiro(3.4) octan-6-yl)(methyl)oxo-lambda6-sulfanylidene))amine (300 mg, 0.65 mmol, 1.0 equiv) in DMF (5 mL) was added formic acid (0.2 mL, 25% (v/v)). After stirring at room temperature for 10 min, the resulting mixture was concentrated under reduced pressure to afford crude product, which was further purified by prep-HPLC with the following conditions: (Column: XBridge Prep OBD C18 Column, 50*250 mm, 10 μm; Mobile Phase A: Water (10 mmol/L NH₄HCO₃), Mobile Phase B: ACN; Flow rate: 60 mL/min; Gradient: 8% B to 32% B in 20 min; Wave Length: 254 nm/220 nm; RT: 20.33 min) to afford 8-methoxy-4-(6-(S-methylsulfon-imidoyl)-2,6-diazaspiro(3.4) octan-2-yl)-1,7-naphthyridine (96 mg, 42%) as an off-white solid. MS (ESI, pos. ion) m/z: 348.1 (M+1). ¹H NMR (400 MHZ, DMSO-d₆, ppm) δ 8.44 (d, J=5.2 Hz, 1H), 7.88 (d, J=6.0 Hz, 1H), 7.38 (d, J=6.0 Hz, 1H), 6.47 (d, J=5.2 Hz, 1H), 4.35-4.32 (m, 2H), 4.27 (d, J=8.0 Hz, 2H), 4.00 (s, 3H), 3.66 (s, 1H), 3.50-3.42 (m, 2H), 3.32-3.20 (m, 2H), 2.82 (d, J=1.6 Hz, 3H), 2.20 (t, J=6.8 Hz, 2H).

### Example 46

### Synthesis of ((1-(8-ethoxy-1,7-naphthyridin-4-yl)-4-hydroxypiperidin-4-yl)methyl) (imino)-(methyl)-lambda6-sulfanone

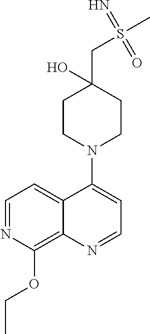 [SMILES: CCOc1nccc2c(N3CCC(O)(CS(C)(=N)=O)CC3)ccnc12]

### Step 1: tert-butyl 4-Hydroxy-4-((methylsulfanyl)methyl) piperidine-1-carboxylate

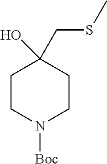 [SMILES: CSCC1(O)CCN(C(=O)OC(C)(C)C)CC1]

To a solution of tert-butyl 1-oxa -6-azaspiro(2.5) octane-6-carboxylate (5.50 g, 24.49 mmol, 1.0 equiv) in EtOH (50 mL) was added sodiummethanethiolate (85.84 g, 244.99 mmol, 10.0 equiv, 20%) at 0° C. After stirring at room temperature for 2 h, the resulting solution was concentrated under reduced pressure. The residue was purified by silica gel column chromatography, eluted with EtOAc/PE (25:75) to afford tert-butyl 4-hydroxy-4-((methylsulfanyl)methyl) piperidine-1-carboxylate (6.47 g, 95%) as colorless oil.

### Step 2:4-((Methylsulfanyl)methyl) piperidin-4-ol hydrochloride

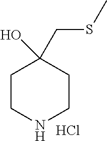

A solution of tert-butyl 4-hydroxy-4-((methylsulfanyl)methyl) piperidine-1-carboxylate (24.00 g, 91.82 mmol, 1.0 equiv) in 4 M HCl in 1,4-dioxane (200 mL) was stirred at room temperature for 2 h and was concentrated under reduced pressure to give 4-((methylsulfanyl)-methyl) piperidin-4-ol hydrochloride (19.00 g, crude) as a white solid. ¹H NMR (400 MHZ, DMSO-d₆, ppm) δ 3.12-2.94 (m, 4H), 2.60 (s, 2H), 2.12 (s, 3H), 1.83-1.65 (m, 4H).

### Step 3:5-(((2-Ethoxypyridin-3-yl)amino)methylidene)-2,2-dimethyl-1,3-dioxane-4,6-dione

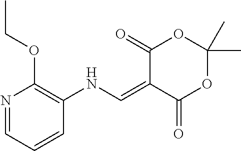 [SMILES: CCOc1ncccc1NC=C1C(=O)OC(C)(C)OC1=O]

A solution of 2-ethoxypyridin-3-amine (2.00 g, 14.47 mmol, 1 equiv), 2,2-dimethyl-1,3-dioxane-4,6-dione (2.71 g, 18.82 mmol, 1.3 equiv) and trimethoxymethane (2.15 g, 20.26 mmol, 1.4 equiv) in ACN (30 mL) was stirred at 80° C. for 2 h under N₂ atmosphere. After cooling down to room temperature, the mixture was diluted with PE. The resulting precipitate was filtered and washed with PE to afford 5-(((2-ethoxypyridin-3-yl)amino)methylidene)-2,2-dimethyl-1,3-dioxane-4,6-dione (4.20 g, 99%) as a black solid.

### Step 4:8-Ethoxy-1,7-naphthyridin-4-ol

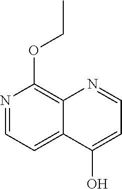

A solution of 5-(((2-ethoxypyridin-3-yl)amino)methylidene)-2,2-dimethyl-1,3-dioxane-4,6-dione (4.10 g, 14.03 mmol) in diphenyl ether (40 mL) was stirred at 225° C. for 1.5 h. After cooling down to room temperature, the mixture was diluted with PE. The precipitate was collected by filtration, washed with PE (200 mL) and purified by reversed phase flash, eluted with ACN/H₂O (32:68) to afford 8-ethoxy-1,7-naphthyridin-4-ol (552 mg, 20%) as a brown solid.

### Step 5:4-Chloro-8-ethoxy-1,7-naphthyridine

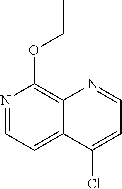

To a solution of 8-ethoxy-1,7-naphthyridin-4-ol (200 mg, 1.05 mmol, 1.0 equiv) in toluene (5 mL) were added DIEA (271 mg, 2.10 mmol, 2.0 equiv) and POCl₃ (241.83 mg, 1.57 mmol, 1.5 equiv) dropwise. The resulting mixture was stirred at 70° C. for 2 h. After cooling down to room temperature, the mixture was concentrated under reduced pressure. The residue was purified by reversed phase flash, eluted with ACN/H₂O (41:59) to afford 4-chloro-8-ethoxy-1,7-naphthyridine (100 mg, 45%) as a yellow solid.

### Step 6:1-(8-Ethoxy-1,7-naphthyridin-4-yl)-4-((methylsulfanyl)methyl) piperidin-4-ol

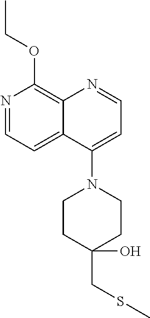 [SMILES: CCOc1nccc2c(N3CCC(O)(CSC)CC3)ccnc12]

To a stirred solution of 4-chloro-8-ethoxy-1,7-naphthyridine (200 mg, 0.95 mmol, 1.0 equiv) in NMP (4 mL) were added DIEA (247 mg, 1.91 mmol, 2.0 equiv) and 4-((methyl-sulfanyl)methyl) piperidin-4-ol hydrochloride (284 mg, 1.43 mmol, 1.5 equiv, from step 2) at room temperature. The reaction mixture was stirred overnight at 130° C. After cooling down to room temperature, the resulting mixture was purified by reversed phase flash, eluted with ACN/H₂O (3:2) to give 1-(8-ethoxy-1,7-naphthyridin-4-yl)-4-((methylsulfanyl)methyl) piperidin-4-ol (180 mg, 56%) as a white solid.

### Step 7: ((1-(8-Ethoxy-1,7-naphthyridin-4-yl)-4-hydroxypiperidin-4-yl)methyl) (imino)-(methyl)-lambda6-sulfanone

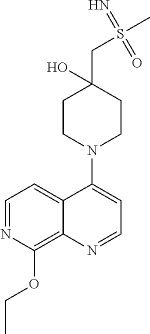 [SMILES: CCOc1nccc2c(N3CCC(O)(CS(C)(=N)=O)CC3)ccnc12]

To a stirred solution of 1-(8-ethoxy-1,7-naphthyridin-4-yl)-4-((methylsulfanyl)-methyl) piperidin-4-ol (160 mg, 0.48 mmol, 1.0 equiv) in MeOH (3 mL) were added (acetyloxy) (phenyl)-lambda3-iodanyl acetate (463 mg, 1.44 mmol, 3.0 equiv) and ammonium carbamate (149 mg, 1.92 mmol, 4.0 equiv) at room temperature. After stirring at the same temperature for 2 h, the resulting mixture was concentrated under reduced pressure and purified by prep-HPLC (UV 254 nm/220 nm Xbridge Prep OBD C18 Column, 19*250 mm, 5 μm Water (10 mmol/L NH₄HCO₃) MeOH 25 mL/min 20% B to 40% B in 13 min; Wave Length: 254 nm/220 nm; RT: 12.3 min). The fractions containing the desired product were combined and lyophilized to give (1-(8-ethoxy-1,7-naphthyridin-4-yl)-4-hydroxypiperidin-4-yl)methyl) (imino)(methyl)-lambda6-sulfanone (54.1 mg, 30%) as a yellow solid. MS (ESI, pos. ion) m/z: 365.1 (M+1). ¹H NMR (400 MHZ, DMSO-d₆, ppm) δ 8.67 (d, J=5.2 Hz, 1H), 8.00 (d, J=6.0 Hz, 1H), 7.31 (d, J=6.0 Hz, 1H), 7.17 (d, J=5.2 Hz, 1H), 5.62 (s, 1H), 4.53-4.44 (m, 2H), 4.03 (s, 1H), 3.46-3.43 (m, 1H), 3.35-3.25 (m, 3H), 3.22-3.11 (m, 2H), 3.05 (s, 3H), 2.08-1.96 (m, 4H), 1.42 (t, J=7.2 Hz, 3H).

### Examples 47, 48 and 49

### Synthesis of ((1-(8-(fluoromethoxy)-1,7-naphthyridin-4-yl)-4-hydroxypiperidin-4-yl)methyl) (imino)(methyl)-lambda6-sulfanone and (R)-((1-(8-(fluoromethoxy)-1,7-naphthyridin-4-yl)-4-hydroxypiperidin-4-yl)methyl) (imino)(methyl)-lambda6-sulfanone and(S) ((1-(8-(fluoromethoxy)-1,7-naphthyridin-4-yl)-4-hydroxypiperidin-4-yl)methyl) (imino)(methyl)-lambda6-sulfanone

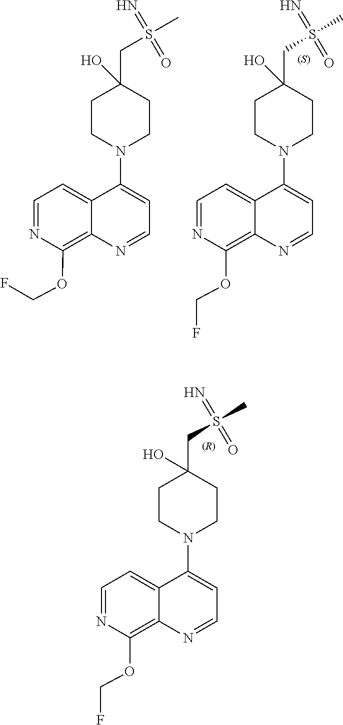

### Step 1:4-Chloro-8-(fluoromethoxy)-1,7-naphthyridine

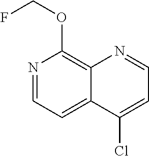 [SMILES: FCOc1nccc2c(Cl)ccnc12]

To a solution of 8-(fluoromethoxy)-1,7-naphthyridin-4-ol (2.20 g, 11.33 mmol, 1.0 equiv) in toluene (10 mL) were added DIEA (2.93 g, 22.66 mmol, 2.0 equiv) and POCl₃ (2.08 g, 13.59 mmol, 1.2 equiv) dropwise. The resulting mixture was stirred at 70° C. for 2 h. After cooling down to room temperature, the reaction mixture was concentrated under reduced pressure and purified by silica gel column chromatography, eluted with EtOAc/PE (18:82) to afford 4-chloro-8-(fluoromethoxy)-1,7-naphthyridine (900 mg, 35%) as a brown solid.

### Step 2:1-(8-(fluoromethoxy)-1,7-naphthyridin-4-yl)-4-((methylsulfanyl)methyl)-piperidin-4-ol

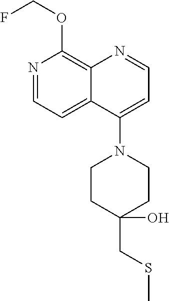 [SMILES: CSCC1(O)CCN(c2ccnc3c(OCF)nccc23)CC1]

To a solution of 4-chloro-8-(fluoromethoxy)-1,7-naphthyridine (900 mg, 4.23 mmol, 1.0 equiv) in NMP (10 mL) were added DIEA (1.09 g, 8.46 mmol, 2.0 equiv) and 4-((methylthio)-methyl) piperidin-4-ol hydrochloride (1.02 g, 6.34 mmol, 1.5 equiv). The resulting mixture was stirred at 130° C. for 2 h. After cooling down to room temperature, the reaction mixture was purified by reversed phase flash, eluted with ACN/H₂O (65:35) to afford 1-(8-(fluoromethoxy)-1,7-naphthyridin-4-yl)-4-((methylsulfanyl)methyl) piperidin-4-ol (1.02 g, 63%) as brown solid.

### Step 3: ((1-(8-(fluoromethoxy)-1,7-naphthyridin-4-yl)-4-hydroxypiperidin-4-yl)methyl)-(imino)(methyl)-lambda6-sulfanone

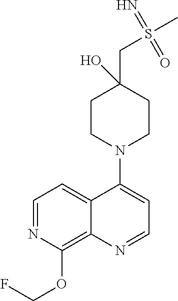 [SMILES: CS(=N)(=O)CC1(O)CCN(c2ccnc3c(OCF)nccc23)CC1]

To a solution of 1-(8-(fluoromethoxy)-1,7-naphthyridin-4-yl)-4-((methylsulfanyl)-methyl) piperidin-4-ol (1.00 g, 2.96 mmol, 1.0 equiv) in MeOH (10 mL) were added (acetyloxy) (phenyl)-lambda3-iodanyl acetate (2.86 g, 8.89 mmol, 3.0 equiv) and ammonium carbamate (0.93 g, 11.85 mmol, 4.0 equiv). After stirring at room temperature for 2 h, the resulting mixture was concentrated under reduced pressure. The crude product was purified by reversed phase flash, eluted with ACN/H₂O (35:65) to afford (1-(8-(fluoromethoxy)-1,7-naphthyridin-4-yl)-4-hydroxypiperidin-4-yl)methyl) (imino)(methyl)-lambda6-sulfanone (800 mg, 73%) as a brown solid. MS (ESI, pos. ion) m/z: 369.1 (M+1). ¹H NMR (400 MHZ, DMSO-d₆, ppm) δ 8.73 (d, J=5.2 Hz, 1H), 8.09 (d, J=5.6 Hz, 1H), 7.54 (d, J=5.6 Hz, 1H), 7.22 (d, J=5.2 Hz, 1H), 6.36 (s, 1H), 6.23 (s, 1H), 5.63 (s, 1H), 4.03 (s, 1H), 3.48-3.46 (m, 1H), 3.45-3.41 (m, 2H), 3.34-3.31 (m, 1H), 3.26-3.14 (m, 2H), 3.06 (s, 3H), 2.10-2.02 (m, 2H), 2.02-1.92 (m, 2H). ¹⁹F NMR (376 MHz, DMSO-d₆, ppm) 8-155.74 (s, 1 F).

### Step 4: (R)-((1-(8-(fluoromethoxy)-1,7-naphthyridin-4-yl)-4-hydroxypiperidin-4-yl)methyl)-(imino)(methyl)-lambda6-sulfanone and(S)-((1-(8-(fluoromethoxy)-1,7-naphthyridin-4-yl)-4-hydroxypiperidin-4-yl)methyl) (imino)(methyl)-lambda6-sulfanone

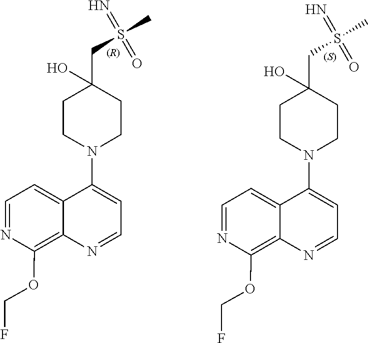

The racemic ((1-(8-(fluoromethoxy)-1,7-naphthyridin-4-yl)-4-hydroxypiperidin-4-yl)methyl) (imino)(methyl)-lambda6-sulfanone (800 mg, 2.17 mmol) was purified by Chrial-SFC with the following conditions (Column: Lux 5 μm Cellulose-4 30*250 mm, 5.0 μm; Mobile Phase A: CO₂, Mobile Phase B: MeOH (1%-2M-NH₃-MeOH); Flow rate: 90 mL/min; Gradient: isocratic 44% B; Column Temperature (° C.): 35; Back Pressure (bar): 100; Wave Length: 220 nm; Sample Solvent: MEOH; Injection Volume: 3 mL; Number Of Runs: 10) to afford two fractions.

Fraction 1: (R)-((1-(8-(fluoromethoxy)-1,7-naphthyridin-4-yl)-4-hydroxypiperidin-4-yl)methyl) (imino)(methyl)-lambda6-sulfanone (335.1 mg, 40%) as an off-white solid. RT1:9. 47 min, MS (ESI, pos. ion) m/z: 369.1 (M+1). ¹H NMR (400 MHZ, DMSO-d₆, ppm) δ 8.73 (d, J=5.2 Hz, 1H), 8.09 (d, J=5.6 Hz, 1H), 7.54 (d, J=5.6 Hz, 1H), 7.22 (d, J=5.2 Hz, 1H), 6.36 (s, 1H), 6.23 (s, 1H), 5.63 (s, 1H), 4.03 (s, 1H), 3.48-3.46 (m, 1H), 3.45-3.41 (m, 2H), 3.34-3.31 (m, 1H), 3.26-3.14 (m, 2H), 3.06 (s, 3H), 2.10-2.02 (m, 2H), 2.02-1.92 (m, 2H).

¹⁹F NMR (376 MHZ, DMSO-d₆, ppm) 8-155.74 (s, 1 F).

Fraction 2: (S)-((1-(8-(fluoromethoxy)-1,7-naphthyridin-4-yl)-4-hydroxypiperidin-4-yl)methyl) (imino)(methyl)-lambda6-sulfanone (360.5 mg, 43%) as an off-white solid. RT2:12. 08 min, MS (ESI, pos. ion) m/z: 369.1 (M+1). ¹H NMR (400 MHZ, DMSO-d₆, ppm) δ 8.73 (d, J=5.2 Hz, 1H), 8.09 (d, J=5.6 Hz, 1H), 7.54 (d, J=5.6 Hz, 1H), 7.22 (d, J=5.2 Hz, 1H), 6.36 (s, 1H), 6.23 (s, 1H), 5.63 (s, 1H), 4.03 (s, 1H), 3.48-3.46 (m, 1H), 3.45-3.41 (m, 2H), 3.34-3.31 (m, 1H), 3.26-3.14 (m, 2H), 3.06 (s, 3H), 2.10-2.02 (m, 2H), 2.02-1.92 (m, 2H).

¹⁹F NMR (377 MHz, DMSO-d₆, ppm) 8-155.74 (s, 1 F).

### Example 50

### Synthesis of imino (4-((8-methoxy-1,7-naphthyridin-4-yl)oxy)phenyl)(methyl)-lambda6-sulfanone

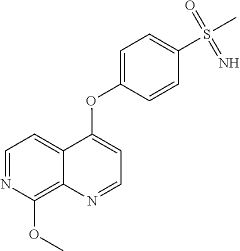 [SMILES: COc1nccc2c(Oc3ccc(S(C)(=N)=O)cc3)ccnc12]

### Step 1:8-Methoxy-4-(4-(methylthio) phenoxy)-1,7-naphthyridine

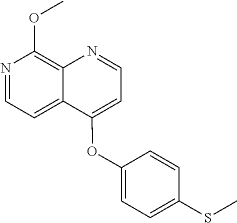 [SMILES: COc1nccc2c(Oc3ccc(SC)cc3)ccnc12]

To a solution of 4-chloro-8-methoxy-1,7-naphthyridine (150 mg, 0.77 mmol, 1.0 equiv) in DMSO (3 mL) were added Cs₂CO₃ (502 mg, 1.54 mmol, 2.0 equiv) and 4-(methylthio)-phenol (216 mg, 1.54 mmol, 2.0 equiv). The resulting mixture was stirred at 100° C. for 3 h. After cooling to room temperature, the reaction mixture was diluted with H₂O (100 mL) and extracted with EtOAc. The combined organic layers were washed with brine, dried over anhydrous sodium sulfate and filtered. The filtrate was concentrated under reduced pressure. The residue was purified by silica gel column chromatography, eluted with EtOAc/PE (3:7) to afford 8-methoxy-4-(4-(methylthio) phenoxy)-1,7-naphthyridine (200 mg, 86%) as a light-yellow solid.

### Step 2: Imino (4-((8-methoxy-1,7-naphthyridin-4-yl)oxy)phenyl)(methyl)-lambda6-sulfanone

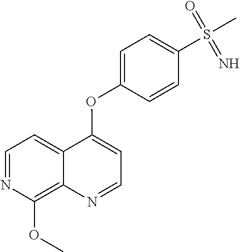 [SMILES: COc1nccc2c(Oc3ccc(S(C)(=N)=O)cc3)ccnc12]

To a solution of 8-methoxy-4-(4-(methylthio) phenoxy)-1,7-naphthyridine (180 mg, 0.60 mmol, 1.0 equiv) in MeOH (3 mL) were added (acetyloxy) (phenyl)-lambda3-iodanyl acetate (582 mg, 1.80 mmol, 3.0 equiv) and ammonium carbamate (188 mg, 2.41 mmol, 4.0 equiv). The reaction mixture was stirred at room temperature for 2 h. The resulting mixture was concentrated under reduced pressure. The crude product was purified by prep-HPLC with the following conditions (Column: XBridge Prep OBD C18 Column, 50*250 mm, 10 μm; Mobile Phase A: Water (10 mmol/LNH₄HCO₃), Mobile Phase B: ACN; Flow rate: 60 mL/min; Gradient: 12% B to 42% B in 20 min; Wave Length: 254 nm/220 nm; RT: 21.07 min) to afford imino (4-((8-methoxy-1,7-naphthyridin-4-yl)oxy)phenyl)(methyl)-lambda6-sulfanone (85.8 mg, 43%) as an off-white solid. MS (ESI, pos. ion) m/z: 330.1 (M+1). ¹H NMR (400 MHZ, DMSO-d₆, ppm) δ 8.81 (d, J=5.2 Hz, 1H), 8.16 (d, J=5.6 Hz, 1H), 8.09-8.01 (m, 2H), 7.61 (d, J=6.0 Hz, 1H), 7.53-7.45 (m, 2H), 7.08 (d, J=5.2 Hz, 1H), 4.30 (s, 1H), 4.09 (s, 3H), 3.12 (d, J=1.2 Hz, 3H).

### Example 51

### Synthesis of imino (3-((8-methoxy-1,7-naphthyridin-4-yl)oxy)phenyl)(methyl)-lambda6-sulfanone

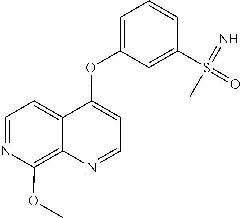 [SMILES: COc1nccc2c(Oc3cccc(S(C)(=N)=O)c3)ccnc12]

### Step 1:3-(Methylthio) phenol

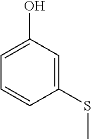

To a solution of 1-methoxy-3-(methylsulfanyl)benzene (500 mg, 3.24 mmol, 1.0 equiv) in DCM (10 mL) was added BBr₃ (9.73 mL, 9.72 mmol, 3.0 equiv, 1M in DCM) at 0° C. After stirring at room temperature for 1 h, the resulting mixture was concentrated under reduced pressure. The residue was quenched with H₂O (100 mL) and extracted with EtOAc. The combined organic layers were washed with brine, dried over anhydrous sodium sulfate and filtered. The filtrate was concentrated under reduced pressure to afford 3-(methylthio) phenol (400 mg, 88%) as a green oil.

### Step 2:8-Methoxy-4-(3-(methylthio) phenoxy)-1,7-naphthyridine

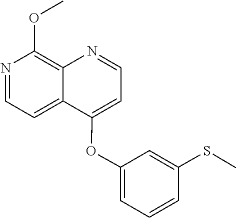 [SMILES: COc1nccc2c(Oc3cccc(SC)c3)ccnc12]

To a solution of 3-(methylthio) phenol (108 mg, 0.77 mmol, 1.0 equiv) in DMSO (3 mL) were added Cs₂CO₃ (502 mg, 1.54 mmol, 2.0 equiv) and 4-chloro-8-methoxy-1,7-naphthyridine (150 mg, 0.77 mmol, 1.0 equiv). The reaction mixture was stirred at 100° C. for 3 h. After cooling down to room temperature, the reaction mixture was diluted with H₂O and extracted with EtOAc. The combined organic layers were washed with brine, dried over anhydrous sodium sulfate and filtered. The filtrate was concentrated under reduced pressure. The residue was purified by silica gel column chromatography, eluted with EtOAc/PE (44:56) to afford 8-methoxy-4-(3-(methylthio) phenoxy)-1,7-naphthyridine (200 mg, 86%) as a brown solid.

### Step 3: Imino (3-((8-methoxy-1,7-naphthyridin-4-yl)oxy)phenyl)(methyl)-lambda6-sulfanone

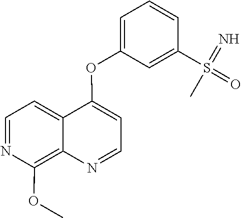 [SMILES: COc1nccc2c(Oc3cccc(S(C)(=N)=O)c3)ccnc12]

To a solution of 8-methoxy-4-(3-(methylthio) phenoxy)-1,7-naphthyridine (200 mg, 0.67 mmol, 1.0 equiv) in MeOH (5 mL) were added (acetyloxy) (phenyl)-lambda3-iodanyl acetate (647 mg, 2.01 mmol, 3.0 equiv) and ammonium carbamate (209 mg, 2.68 mmol, 4.0 equiv). After stirring at room temperature for 2 h, the resulting mixture was concentrated under reduced pressure to afford crude product, which was purified by prep-HPLC with the following conditions (Column: XBridge Prep Shield RP C18 Column, 30*150 mm, 5 μm; Mobile Phase A: Water (10 mmol/L NH₄HCO₃), Mobile Phase B: ACN; Flow rate: 60 mL/min; Gradient: 7% B to 32% B in 7 min; Wave Length: 254 nm/220 nm; RT: 7.0 min) to afford imino (3-((8-methoxy-1,7-naphthyridin-4-yl)oxy)phenyl)(methyl)-lambda6-sulfanone (82.0 mg, 37%) as an off-white solid. MS (ESI, pos. ion) m/z: 330.1 (M+1). ¹H NMR (400 MHZ, DMSO-d₆, ppm) δ 8.78 (d, J=5.2 Hz, 1H), 8.18 (d, J=5.6 Hz, 1H), 7.95-7.88 (m, 1H), 7.83-7.75 (m, 2H), 7.68 (d, J=5.6 Hz, 1H), 7.65-7.59 (m, 1H), 6.99 (d, J=5.2 Hz, 1H), 4.37 (s, 1H), 4.10 (s, 3H), 3.17-3.12 (m, 3H).

### Example 52

### Synthesis of ((1-(8-cyclopropoxy-1,7-naphthyridin-4-yl)-4-hydroxypiperidin-4-yl)methyl) (imino)(methyl)-lambda6-sulfanone

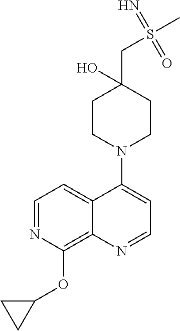 [SMILES: CS(=N)(=O)CC1(O)CCN(c2ccnc3c(OC4CC4)nccc23)CC1]

### Step 1:2-Cyclopropoxy-3-nitropyridine

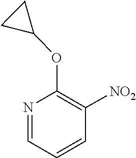 [SMILES: O=[N+]([O-])c1cccnc1OC1CC1]

To a cooled solution of cyclopropanol (3.07 g, 52.78 mmol, 1.5 equiv) in THF (50 mL) was added NaH (2.11 g, 52.78 mmol, 1.5 equiv, 60%), followed by addition of 2-fluoro-3-nitropyridine (5.00 g, 35.18 mmol, 1.0 equiv) at 0° C. The resulting mixture was stirred at room temperature for 2 h. After completion of reaction, the reaction mixture was quenched with ice water and extracted with EtOAc. The combined organic layers were washed with brine, dried over anhydrous sodium sulfate and filtered. The filtrate was concentrated under reduced pressure. The residue was purified by silica gel column chromatography, eluted with EtOAc/PE (8:92) to give 2-cyclopropoxy-3-nitropyridine (5.50 g, 86%) as a colorless oil.

### Step 2:2-Cyclopropoxypyridin-3-amine

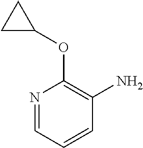 [SMILES: Nc1cccnc1OC1CC1]

To a solution of 2-cyclopropoxy-3-nitropyridine (5.20 g, 28.86 mmol, 1.0 equiv) in MeOH (80 mL) was added Pd/C (3.07 g, 2.88 mmol, 0.1 equiv, 10%) under nitrogen atmosphere. The mixture was stirred overnight at room temperature under hydrogen atmosphere by using a hydrogen balloon, then filtered through a celite pad and concentrated under reduced pressure to afford 2-cyclopropoxypyridin-3-amine (3.80 g, 87%) as a grey oil.

### Step 3:5-(((2-Cyclopropoxypyridin-3-yl)amino)methylidene)-2,2-dimethyl-1,3-dioxane-4,6-dione

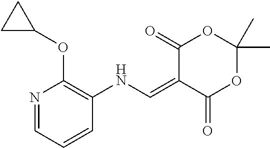 [SMILES: CC1(C)OC(=O)C(=CNc2cccnc2OC2CC2)C(=O)O1]

To a stirred mixture of 2-cyclopropoxypyridin-3-amine (3.80 g, 25.30 mmol, 1.0 equiv) in ACN (50 mL) were added 2,2-dimethyl-1,3-dioxane-4,6-dione (5.11 g, 35.42 mmol, 1.4 equiv) and trimethoxymethane (3.76 g, 35.42 mmol, 1.4 equiv) at room temperature. After stirring at 80° C. for 2 h, the resulting mixture was cooled to 0° C. The precipitate was collected by filtration and washed with PE (50 mL) to afford 5-(((2-cyclopropoxypyridin-3-yl)amino)methylidene)-2,2-dimethyl-1,3-dioxane-4,6-dione (6.10 g, 79%) as an off-white solid.

### Step 4:8-Cyclopropoxy-1,7-naphthyridin-4-ol

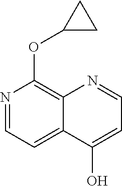 [SMILES: Oc1ccnc2c(OC3CC3)nccc12]

A solution of 5-(((2-cyclopropoxypyridin-3-yl)amino)methylidene)-2,2-dimethyl-1,3-dioxane-4,6-dione (3.50 g, 11.50 mmol, 1.0 equiv) in diphenyl ether (35 mL) was stirred at 225° C. for 1.5 h under nitrogen atmosphere. After cooling down to room temperature, PE was poured into the reaction mixture. The precipitate was collected by filtration, washed with PE and purified by reversed phase flash, eluted with ACN/H₂O (23:77) to afford 8-cyclopropoxy-1,7-naphthyridin-4-ol (400 mg, 17%) as a white solid.

### Step 5:4-Chloro-8-cyclopropoxy-1,7-naphthyridine

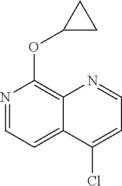 [SMILES: Clc1ccnc2c(OC3CC3)nccc12]

To a stirred solution of 8-cyclopropoxy-1,7-naphthyridin-4-ol (380 mg, 1.87 mmol, 1.0 equiv) in toluene (8 mL) were added DIEA (485 mg, 3.75 mmol, 2.0 equiv) and POCl₃ (432 mg, 2.81 mmol, 1.5 equiv) at room temperature. After stirring at 70° C. for 2 h, the resulting mixture was cooled to room temperature and concentrated under reduced pressure. The residue was purified by reversed phase flash, eluted with ACN/H₂O (42:58) to give 4-chloro-8-cyclopropoxy-1,7-naphthyridine (280 mg, 67%) as a yellow solid.

### Step 6:1-(8-Cyclopropoxy-1,7-naphthyridin-4-yl)-4-((methylsulfanyl)methyl) piperidin-4-ol

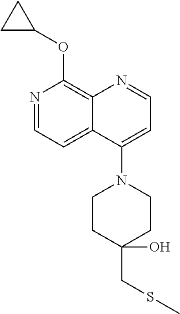 [SMILES: CSCC1(O)CCN(c2ccnc3c(OC4CC4)nccc23)CC1]

To a stirred solution of 4-chloro-8-cyclopropoxy-1,7-naphthyridine (260 mg, 1.17 mmol, 1.0 equiv) in NMP (5 mL) were added DIEA (304 mg, 2.35 mmol, 2.0 equiv) and 4-((methylsulfanyl)methyl) piperidin-4-ol hydrochloride (349 mg, 1.76 mmol, 1.5 equiv, prepared as described in Example 46 Steps 1 and 2) at room temperature. After stirring overnight at 130° C., the resulting solution was purified by reversed phase flash, eluted with ACN/H₂O (51:49) to give 1-(8-cyclopropoxy-1,7-naphthyridin-4-yl)-4-((methylsulfanyl)methyl) piperidin-4-ol (301 mg, 73%) as a yellow solid.

### Step 7: ((1-(8-Cyclopropoxy-1,7-naphthyridin-4-yl)-4-hydroxypiperidin-4-yl)methyl) (imino)-(methyl)-lambda6-sulfanone

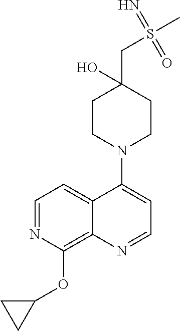 [SMILES: CS(=N)(=O)CC1(O)CCN(c2ccnc3c(OC4CC4)nccc23)CC1]

To a stirred solution of 1-(8-cyclopropoxy-1,7-naphthyridin-4-yl)-4-((methylsulfanyl)-methyl) piperidin-4-ol (150 mg, 0.43 mmol, 1.0 equiv) in MeOH (3 mL) were added (acetyloxy) (phenyl)-lambda3-iodanyl acetate (419 mg, 1.30 mmol, 3.0 equiv) and ammonium carbamate (135 mg, 1.73 mmol, 4.0 equiv) at room temperature. After stirring at the same temperature for 2 h, the resulting mixture was concentrated under reduced pressure. The residue was purified by prep-HPLC (Column: XBridge Prep OBD C18 Column, 50*250 mm, 10 μm; Mobile Phase A: Water (10 mmol/L NH₄HCO₃), Mobile Phase B: ACN; Flow rate: 60 mL/min; Gradient: 9% B to 34% B in 20 min; Wave Length: 254 nm/220 nm; RT: 21.17 min). The fractions containing the desired product were combined and lyophilized to give (1-(8-cyclopropoxy-1,7-naphthyridin-4-yl)-4-hydroxypiperidin-4-yl)methyl) (imino)(methyl)-lambda6-sulfanone (72.7 mg, 42%) as a white solid. MS (ESI, pos. ion) m/z: 377.2 (M+1). ¹H NMR (400 MHZ, DMSO-d₆, ppm) δ 8.64 (d, J=5.2 Hz, 1H), 8.05 (d, J=6.0 Hz, 1H), 7.36 (d, J=6.0 Hz, 1H), 7.17 (d, J=5.2 Hz, 1H), 5.61 (s, 1H), 4.51-4.42 (m, 1H), 4.02 (s, 1H), 3.44 (d, J=12.0 Hz, 1H), 3.32-3.27 (m, 3H), 3.24-3.13 (m, 2H), 3.05 (s, 3H), 2.10-1.92 (m, 4H), 0.88-0.81 (m, 2H), 0.79-0.73 (m, 2H).

### Example 53

### Synthesis of ((2-(8-methoxy-1,7-naphthyridin-4-yl)-2,8-diazaspiro(4.5) decan-8-yl)(methyl)oxo-lambda6-sulfanylidene)(methyl)amine

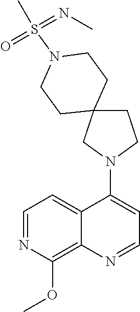 [SMILES: CN=S(C)(=O)N1CCC2(CCN(c3ccnc4c(OC)nccc34)C2)CC1]

To a solution of 2-(8-methoxy-1,7-naphthyridin-4-yl)-8-(S-methylsulfonimidoyl)-2,8-diazaspiro(4.5) decane (80 mg, 0.21 mmol, 1.0 equiv.) in DMF (2 mL) was added NaH (42 mg, 1.06 mmol, 5.0 equiv, 60%) at 0° C. After stirring at 0° C. for 0.5 h, Mel (60 mg, 0.42 mmol, 2.0 equiv) was added. After stirring at room temperature for 1 h, the resulting mixture was quenched with H₂O and extracted with EtOAc. The combined organic layers were washed with brine, dried over anhydrous sodium sulfate and filtered. The filtrate was concentrated under reduced pressure. The residue was purified by Prep-HPLC with the following conditions (Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 7% B to 37% B in 10 min; Wave Length: 254 nm/220 nm; RT: 9.1 min) to afford ((2-(8-methoxy-1,7-naphthyridin-4-yl)-2,8-diazaspiro(4.5) decan-8-yl)(methyl)oxo-lambda6-sulfanylidene)(methyl)amine (22.0 mg, 26%) as an off-white solid. MS (ESI, pos. ion) m/z: 390.2 (M+1). ¹H NMR (400 MHZ, DMSO-d₆, ppm) δ 8.42 (d, J=5.2 Hz, 1H), 7.87 (d, J=6.4 Hz, 1H), 7.69 (d, J=6.4 Hz, 1H), 6.71 (d, J=5.6 Hz, 1H), 3.99 (s, 3H), 3.79-3.71 (m, 2H), 3.53 (s, 2H), 3.17-3.03 (m, 4H), 2.77 (s, 3H), 2.46 (s, 3H), 1.96-1.88 (m, 2H), 1.71-1.67 (m, 4H).

### Example 54

### Synthesis of ((7-(8-methoxy-1,7-naphthyridin-4-yl)-2,7-diazaspiro(4.4) nonan-2-yl)(methyl)oxo-lambda6-sulfanylidene)(methyl)amine

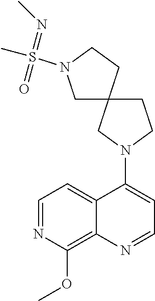 [SMILES: CN=S(C)(=O)N1CCC2(CCN(c3ccnc4c(OC)nccc34)C2)C1]

To a stirred solution of 8-methoxy-4-(7-(S-methylsulfonimidoyl)-2,7-diazaspiro(4.4)-nonan-2-yl)-1,7-naphthyridine (42 mg, 0.11 mmol, 1.0 equiv.) in DMF (2 mL) was added NaH (8 mg, 0.34 mmol, 3.0 equiv) at 0° C. After stirring for 30 min, Mel (32 mg, 0.23 mmol, 2.0 equiv) was added and the mixture was stirred for 1 h at room temperature. The resulting mixture was quenched with water and extracted with EtOAc. The combined organic layers were washed with brine, dried over anhydrous sodium sulfate and filtered. The filtrate was concentrated under reduced pressure. The residue was purified by prep-HPLC with the following conditions: (Column: XBridge Prep RP OBD C18 Column, 30*150 mm, 5 μm; Mobile Phase A: Water (10 mmol/L NH₄HCO₃), Mobile Phase B: ACN; Flow rate: 60 mL/min; Gradient: 2% B to 32% B in 10 min; Wave Length: 254 nm/220 nm; RT: 8.9 min). The fractions containing the desired product were combined and lyophilized to afford ((7-(8-methoxy-1,7-naphthyridin-4-yl)-2,7-diazaspiro(4.4) nonan-2-yl)(methyl)oxo-lambda6-sulfanylidene)(methyl)amine (3.9 mg, 8%) as a white solid. MS (ESI, pos. ion) m/z: 376.2 (M+1). ¹H NMR (400 MHZ, DMSO-d₆, ppm) δ 8.42 (d, J=5.2 Hz, 1H), 7.86 (d, J=6.0 Hz, 1H), 7.66 (d, J=6.0 Hz, 1H), 6.71 (d, J=5.6 Hz, 1H), 3.99 (s, 3H), 3.75 (s, 2H), 3.72-3.59 (m, 2H), 3.31-3.25 (m, 3H), 3.19 (s, 2H), 2.83 (d, J=2.0 Hz, 3H), 2.50 (s, 1H), 2.48 (s, 1H), 2.11-1.90 (m, 4H).

### Example 55

### Synthesis of cyclopropyl(3-(((8-methoxy-1,7-naphthyridin-4-yl)oxy)methyl)phenyl)-(methylimino)-lambda6-sulfanone

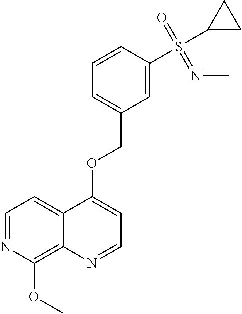 [SMILES: CN=S(=O)(c1cccc(COc2ccnc3c(OC)nccc23)c1)C1CC1]

To a solution of cyclopropyl(imino) (3-(((8-methoxy-1,7-naphthyridin-4-yl)oxy)-methyl)phenyl)-lambda6-sulfanone (120 mg, 0.32 mmol, 1.0 equiv.) in DMF (5 mL) was added NaH (19 mg, 0.48 mmol, 1.5 equiv, 60%) at 0° C. and then the mixture was stirred at 0° C. for 30 min. MeI (92 mg, 0.65 mmol, 2.0 equiv) was added. After stirring at room temperature for 1 h, the reaction mixture was quenched with water and extracted with DCM. The combined organic layers were washed with brine, dried over anhydrous sodium sulfate and filtered. The filtrate was concentrated under reduced pressure to afford crude product, which was purified by Prep-HPLC with the following conditions (Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 13% B to 43% B in 10 min; Wave Length: 254 nm/220 nm; RT: 8.5 min) to afford cyclopropyl(3-(((8-methoxy-1,7-naphthyridin-4-yl)oxy)methyl)phenyl)(methylimino)-lambda6-sulfanone (63.2 mg, 50%) as an off-white solid. MS (ESI, pos. ion) m/z: 384.1 (M+1). ¹H NMR (400 MHZ, DMSO-d₆, ppm) δ 8.80 (s, 1H), 8.10 (d, J=5.6 Hz, 1H), 7.97 (d, J=1.6 Hz, 1H), 7.90-7.84 (m, 1H), 7.84-7.77 (m, 1H), 7.76-7.67 (m, 1H), 7.56 (d, J=5.6 Hz, 1H), 7.38 (d, J=5.2 Hz, 1H), 5.55 (s, 2H), 4.06 (s, 3H), 2.79-2.68 (m, 1H), 2.53 (s, 3H), 1.26-1.13 (m, 1H), 1.12-1.00 (m, 1H), 0.97-0.78 (m, 2H).

### Example 56

### Synthesis of cyclopropyl(3-(((8-methoxypyrido[3,4-d]pyrimidin-4-yl)oxy)methyl)phenyl)-(methylimino)-lambda6-sulfanone

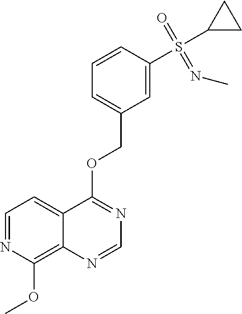 [SMILES: CN=S(=O)(c1cccc(COc2ncnc3c(OC)nccc23)c1)C1CC1]

To a stirred solution of cyclopropyl(imino) (3-(((8-methoxypyrido[3,4-d]pyrimidin-4-yl)oxy)methyl)phenyl)-lambda6-sulfanone (100 mg, 0.27 mmol, 1.0 equiv.) in DMF (2 mL) were added Cs₂CO₃ (175 mg, 0.54 mmol, 2.0 equiv) and MeI (76 mg, 0.54 mmol, 2.0 equiv) at room temperature. The reaction mixture was stirred at 80° C. for 2 h. After cooling down to room temperature, the resulting mixture was quenched with water at 0° C. and extracted with EtOAc. The combined organic layers were washed with brine, dried over anhydrous sodium sulfate and filtered. The filtrate was concentrated under reduced pressure. The residue was purified by prep-HPLC (Column: XBridge Prep RP OBD C18 Column, 30*150 mm, 5 μm; Mobile Phase A: Water (10 mmol/L NH₄HCO₃), Mobile Phase B: ACN; Flow rate: 60 mL/min; Gradient: 20% B to 50% B in 7 min; Wave Length: 254 nm/220 nm; RT1 (min): 6.4). The fractions containing the desired product were combined and lyophilized to give cyclopropyl(3-(((8-methoxypyrido[3,4-d]pyrimidin-4-yl)oxy)methyl)phenyl)(methylimino)-lambda6-sulfanone (1.6 mg, 1%) as a white solid. MS (ESI, pos. ion) m/z: 385.2 (M+1). ¹H NMR (400 MHZ, DMSO-d₆, ppm) δ 8.92 (s, 1H), 8.24 (d, J=5.6 Hz, 1H), 7.96 (d, J=1.6 Hz, 1H), 7.86 (d, J=7.6 Hz, 1H), 7.81-7.77 (m, 1H), 7.69 (t, J=7.6 Hz, 1H), 7.55-7.51 (m, 1H), 5.76 (s, 2H), 4.07 (s, 3H), 2.75-2.68 (m, 1H), 2.51 (s, 3H), 1.25-1.13 (m, 1H), 1.10-1.00 (m, 1H), 0.96-0.77 (m, 2H).

### Example 57

### Synthesis of ((4-hydroxy-1-(8-methoxy-1,7-naphthyridin-4-yl) piperidin-4-yl)methyl)(methyl)-(methylimino)-lambda6-sulfanone

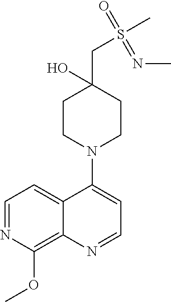 [SMILES: CN=S(C)(=O)CC1(O)CCN(c2ccnc3c(OC)nccc23)CC1]

To a stirred solution of ((4-hydroxy-1-(8-methoxy-1,7-naphthyridin-4-yl) piperidin-4-yl)methyl) (imino)(methyl)-lambda6-sulfanone (175 mg, 0.49 mmol, 1 equiv.) in DMF (5 mL) was added NaH (30 mg, 0.74 mmol, 1.5 equiv, 60%) at 0° C. The resulting mixture was stirred at 0° C. for 30 min. Then Mel (77 mg, 0.54 mmol, 1.5 equiv) was added. After stirred at room temperature for 1 h, the reaction mixture was quenched with water at 0° C. and extracted with EtOAc. The combined organic layers were washed with brine (2×50 mL), dried over anhydrous sodium sulfate and filtered. The filtrate was concentrated under reduced pressure. The residue was purified by prep-HPLC with the following conditions: (Column: YMC-Actus Triart C18ExRs, 30*150 mm, 5 μm; Mobile Phase A: Water (10 mmol/L NH₄HCO₃), Mobile Phase B: ACN; Flow rate: 60 mL/min mL/min; Gradient: 13% B to 23% B in 10 min; Wave Length: 254 nm/220 nm; RT: 8.6 min). The fractions containing the desired product were combined and lyophilized to afford ((4-hydroxy-1-(8-methoxy-1,7-naphthyridin-4-yl) piperidin-4-yl)methyl)-(methyl)(methylimino)-lambda6-sulfanone (32 mg, 17%) as an off-white solid. MS (ESI, pos. ion) m/z: 365.1 (M+1). ¹H NMR (400 MHZ, DMSO-d₆, ppm) δ 8.66 (d, J=5.2 Hz, 1H), 8.02 (d, J=6.0 Hz, 1H), 7.33 (d, J=6.0 Hz, 1H), 7.17 (d, J=5.2 Hz, 1H), 5.76 (s, 1H), 4.03 (s, 3H), 3.55-3.51 (m, 1H), 3.32-3.28 (m, 1H), 3.20-3.18 (m, 2H), 3.17-3.12 (m, 2H), 3.07 (s, 3H), 2.66 (s, 3H), 2.03-1.95 (m, 4H).

### Example 58

### Synthesis of ((2-(8-methoxy-1,7-naphthyridin-4-yl)-2-azaspiro(3.3) heptan-6-yl)methyl-methyl)(methylimino)-lambda6-sulfanone

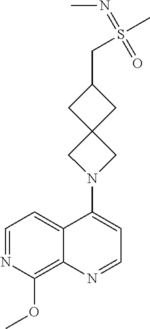 [SMILES: CN=S(C)(=O)CC1CC2(C1)CN(c1ccnc3c(OC)nccc13)C2]

To a solution of imino ((2-(8-methoxy-1,7-naphthyridin-4-yl)-2-azaspiro(3.3) heptan-6-yl)methyl)(methyl)-lambda6-sulfanone (120 mg, 0.34 mmol, 1.0 equiv.) in DMF (2 mL) was added NaH (41 mg, 1.03 mmol, 3.0 equiv, 60%) at 0° C. After stirring at 0° C. for 30 min, MeI (98 mg, 0.69 mmol, 2.0 equiv) was added. The resulting mixture was stirred at room temperature for 1 h, then quenched with water (50 mL) at 0° C. and extracted with EtOAc. The combined organic layers were washed with brine, dried over anhydrous sodium sulfate and filtered. The filtrate was concentrated under reduced pressure. The residue was purified by prep-HPLC with the following conditions (Column: YMC-Actus Triart C18ExRs, 30*150 mm, 5 μm; Mobile Phase A: Water (10 mmol/L NH₄HCO₃), Mobile Phase B: ACN; Flow rate: 60 mL/min Gradient: 14% B to 24% B in 10 min; Wave Length: 254 nm/220 nm; RT: 8.9 min) to afford ((2-(8-methoxy-1,7-naphthyridin-4-yl)-2-azaspiro(3.3) heptan-6-yl)methyl)(methyl)(methylimino)-lambda6-sulfanone (26.9 mg, 21.41%) as an off-white solid. MS (ESI, pos. ion) m/z: 361.1 (M+1). ¹H NMR (400 MHZ, DMSO-d₆, ppm) δ 8.41 (d, J=5.6 Hz, 1H), 7.87 (d, J=6.0 Hz, 1H), 7.35 (d, J=6.0 Hz, 1H), 6.41 (d, J=5.2 Hz, 1H), 4.39 (s, 2H), 4.23 (s, 2H), 3.98 (s, 3H), 3.32-3.17 (m, 2H), 2.84 (s, 3H), 2.69-2.62 (m, 1H), 2.60 (s, 3H), 2.50-2.40 (m, 2H), 2.18-2.07 (m, 2H).

### Example 59

### Synthesis of cyclopropyl(4-fluoro-3-(((8-methoxy-1,7-naphthyridin-4-yl)oxy)methyl)-phenyl) (imino)-lambda6-sulfanone

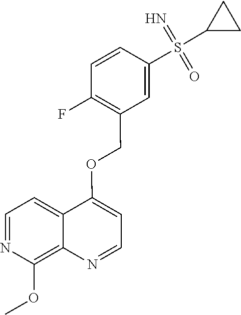 [SMILES: COc1nccc2c(OCc3cc(S(=N)(=O)C4CC4)ccc3F)ccnc12]

### Step 1:5-(Chlorosulfonyl)-2-fluorobenzoic acid

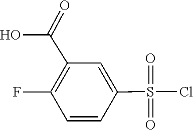 [SMILES: O=C(O)c1cc(S(=O)(=O)Cl)ccc1F]

To 2-fluorobenzoic acid (1.00 g, 1.43 mmol, 1.0 equiv) was added chlorosulfonic (4.29 g, 7.37 mmol, 5.0 equiv) dropwise over 5 min. The reaction mixture was stirred at room temperature for 30 min and then heated to 90° C. for 4.5 h. After cooling down to room temperature, the resulting mixture was quenched with water. The precipitate was collected by filtration, washed with water and dried under vacuum to afford 5-(chlorosulfonyl)-2-fluorobenzoic acid (1.10 g, 65%) as an off-white solid.

### Step 2:2-Fluoro-5-sulfanylbenzoic acid

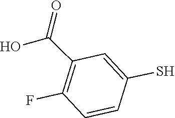 [SMILES: O=C(O)c1cc(S)ccc1F]

To a solution of 5-(chlorosulfonyl)-2-fluorobenzoic acid (1.10 g, 4.61 mmol, 1.0 equiv) in con. HCl (10 mL) was added SnCl₂·2H₂O (3.15 g, 13.83 mmol, 3.0 equiv). The reaction mixture was stirred at 100° C. for 3 h. After cooling down to room temperature, the reaction mixture was diluted water and basified with saturated sodium bicarbonate. The inorganic material was filtered off and filtrate was acidified with 1N HCl and extracted with dichloromethane. The combined organic layers were washed with brine, dried over anhydrous sodium sulfate and filtered. The filtrate was concentrated under reduced pressure to afford 2-fluoro-5-sulfanylbenzoic acid (550 mg, 69%) as a light yellow oil.

### Step 3:5-(Cyclopropylsulfanyl)-2-fluorobenzoic acid

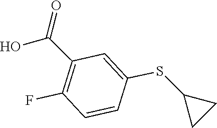 [SMILES: O=C(O)c1cc(SC2CC2)ccc1F]

To a solution of 2-fluoro-5-sulfanylbenzoic acid (550 mg, 3.19 mmol, 1.0 equiv) in DMSO (10 mL) were added bromocyclopropane (579 mg, 4.79 mmol, 1.5 equiv) and potassium tert-butoxide (716 mg, 6.39 mmol, 2.0 equiv). The reaction mixture was stirred overnight at 80° C. under nitrogen atmosphere. After cooling to room temperature, the resulting mixture was diluted water (100 mL) and acidified to pH 4-5 with 1 N HCl. The resulting mixture was extracted with EtOAc. The combined organic layers were washed with brine, dried over anhydrous sodium sulfate and filtered. The filtrate was concentrated under reduced pressure. The residue was purified by reversed phase flash, eluted with ACN/H₂O (45:55) to afford 5-(cyclopropylsulfanyl)-2-fluorobenzoic acid (460 mg, 54%) as a light yellow solid.

### Step 4: (5-(Cyclopropylsulfanyl)-2-fluorophenyl) methanol

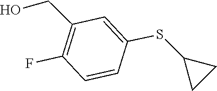 [SMILES: OCc1cc(SC2CC2)ccc1F]

To a solution of 5-(cyclopropylsulfanyl)-2-fluorobenzoic acid (440 mg, 2.07 mmol, 1.0 equiv) in THF (10 mL) was added LiAlH₄ (2.1 mL, 4.14 mmol, 2.0 equiv, 2 M in THF) dropwise at 0° C. After stirring overnight at room temperature under nitrogen atmosphere, the reaction mixture was quenched with water and extracted with DCM. The combined organic layers were washed with brine, dried over anhydrous sodium sulfate and filtered. The filtrate was concentrated under reduced pressure. The residue was purified by silica gel column chromatography, eluted with MeOH/DCM (14:86) to afford (5-(cyclopropylsulfanyl)-2-fluorophenyl) methanol (210 mg, 51%) as a brown oil.

### Step 5:4-((5-(Cyclopropylsulfanyl)-2-fluorophenyl) methoxy)-8-methoxy-1,7-naphthyridine

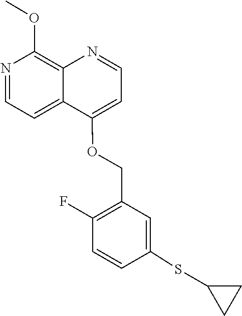 [SMILES: COc1nccc2c(OCc3cc(SC4CC4)ccc3F)ccnc12]

To a solution of (5-(cyclopropylsulfanyl)-2-fluorophenyl) methanol (190 mg, 0.95 mmol, 1.0 equiv) in DMF (5 mL) were added Cs₂CO₃ (624 mg, 1.91 mmol, 2.0 equiv) and 4-chloro-8-methoxy-1,7-naphthyridine (279 mg, 1.43 mmol, 1.5 equiv.). The reaction mixture was stirred at 100° C. for 2 h. After cooling down to room temperature, the reaction mixture was quenched with water and extracted with DCM. The combined organic layers were washed with brine, dried over anhydrous sodium sulfate and filtered. The filtrate was concentrated under reduced pressure. The residue was purified by silica gel column chromatography, eluted with EtOAc/PE (80:20) to afford 4-((5-(cyclopropylsulfanyl)-2-fluorophenyl) methoxy)-8-methoxy-1,7-naphthyridine (150 mg, 43%) as a light-yellow solid.

### Step 6: Cyclopropyl(4-fluoro-3-(((8-methoxy-1,7-naphthyridin-4-yl)oxy)methyl)phenyl)-(imino)-lambda6-sulfanone

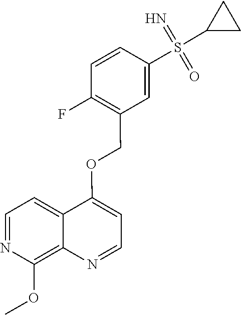 [SMILES: COc1nccc2c(OCc3cc(S(=N)(=O)C4CC4)ccc3F)ccnc12]

To a solution of 4-((5-(cyclopropylsulfanyl)-2-fluorophenyl) methoxy)-8-methoxy-1,7-naphthyridine (130 mg, 0.36 mmol, 1.0 equiv) in MeOH (3 mL) were added (acetyloxy) (phenyl)-lambda3-iodanyl acetate (352 mg, 1.09 mmol, 3.0 equiv) and ammonium carbamate (113 mg, 1.46 mmol, 4.0 equiv). The reaction mixture was stirred at room temperature for 2 h and then concentrated under reduced pressure. The residue was purified by prep-HPLC with the following conditions (Column: XBridge Prep RP OBD C18 Column, 30*150 mm, 5 μm; Mobile Phase A: Water (10 mmol/L NH₄HCO₃), Mobile Phase B: ACN; Flow rate: 60 mL/min; Gradient: 10% B to 40% B in 10 min; Wave Length: 254 nm/220 nm; RT: 9.3 min) to afford cyclopropyl(4-fluoro-3-(((8-methoxy-1,7-naphthyridin-4-yl)oxy)methyl)phenyl) (imino)-lambda6-sulfanone (57.1 mg, 40%) as an off-white solid. MS (ESI, pos. ion) m/z: 388.0 (M+1). ¹H NMR (400 MHZ, DMSO-d₆, ppm) δ 8.82 (d, J=5.2 Hz, 1H), 8.22 (d, J=6.8 Hz, 1H), 8.08 (d, J=5.6 Hz, 1H), 8.08-8.00 (m, 1H), 7.60-7.51 (m, 1H), 7.47 (d, J=6.8, 2H), 5.56 (s, 2H), 4.38 (s, 1H), 4.06 (s, 3H), 2.71 (d, J=8.6 Hz, 1H), 1.17-1.07 (m, 1H), 1.04-0.76 (m, 3H).

¹⁹F NMR (376 MHz, DMSO-d₆, ppm) 8-111.88 (s, 1 F).

### Example 60

### Synthesis of (3-(((8-cyclopropoxy-1,7-naphthyridin-4-yl)oxy)methyl)phenyl)-(cyclopropyl) (imino)-lambda6-sulfanone

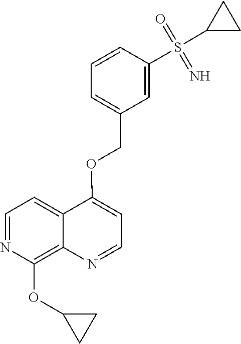 [SMILES: N=S(=O)(c1cccc(COc2ccnc3c(OC4CC4)nccc23)c1)C1CC1]

### Step 1:8-Cyclopropoxy-4-((3-(cyclopropylsulfanyl)phenyl) methoxy)-1,7-naphthyridine

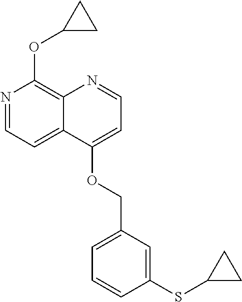 [SMILES: c1cc(COc2ccnc3c(OC4CC4)nccc23)cc(SC2CC2)c1]

To a stirred solution of (3-(cyclopropylsulfanyl)phenyl) methanol (114 mg, 0.63 mmol, 1.0 equiv) in DMF (3 mL) was added NaH (45 mg, 1.90 mmol, 3.0 equiv) at 0° C. After stirred for 10 min, 4-chloro-8-cyclopropoxy-1,7-naphthyridine (140 mg, 0.63 mmol, 1.0 equiv) was added. The reaction mixture was stirred at 80° C. for 2 h. After cooling down to room temperature, the resulting mixture was quenched with water at 0° C. and extracted with DCM. The combined organic layers were washed with brine, dried over anhydrous sodium sulfate and filtered. The filtrate was concentrated. The residue was purified by reversed phase flash, eluted with ACN/H₂O (45:55) to afford 8-cyclopropoxy-4-((3-(cyclopropyl-sulfanyl)phenyl) methoxy)-1,7-naphthyridine (58 mg, 25%) as a yellow solid.

### Step 2: (3-(((8-Cyclopropoxy-1,7-naphthyridin-4-yl)oxy)methyl)phenyl) (cyclopropyl)-(imino)-lambda6-sulfanone

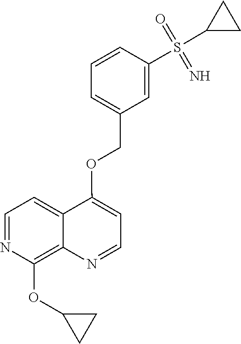 [SMILES: N=S(=O)(c1cccc(COc2ccnc3c(OC4CC4)nccc23)c1)C1CC1]

To a stirred solution of 8-cyclopropoxy-4-((3-(cyclopropylsulfanyl)phenyl) methoxy)-1,7-naphthyridine (48 mg, 0.13 mmol, 1.0 equiv) in MeOH (2 mL) were added (acetyloxy) (phenyl)-lambda3-iodanyl acetate (127 mg, 0.39 mmol, 3.0 equiv) and ammonium carbamate (41 mg, 0.52 mmol, 4.0 equiv) at room temperature. The resulting mixture was stirred at room temperature for 2 h, then concentrated under reduced pressure. The residue was purified by prep-HPLC (Column: Xbridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 13% B to 43% B in 10 min; Wave Length: 254 nm/220 nm; RT: 8.68 min). The fractions containing the desired product were combined and lyophilized to give (3-(((8-cyclopropoxy-1,7-naphthyridin-4-yl)oxy)methyl)phenyl) (cyclopropyl) (imino)-lambda6-sulfanone (26.8 mg, 50%) as a white solid. MS (ESI, pos. ion) m/z: 396.2 (M+1). ¹H NMR (400 MHZ, DMSO-d₆, ppm) δ 8.77 (d, J=5.2 Hz, 1H), 8.12 (d, J=5.6 Hz, 1H), 8.08 (t, J=1.6 Hz, 1H), 7.94-7.88 (m, 1H), 7.87-7.82 (m, 1H), 7.68 (t, J=7.2 Hz, 1H), 7.59 (d, J=5.6 Hz, 1H), 7.37 (d, J=5.2 Hz, 1H), 5.54 (s, 2H), 4.54-4.45 (m, 1H), 4.29 (s, 1H), 2.72-2.64 (m, 1H), 1.16-1.07 (m, 1H), 1.02-0.83 (m, 5H), 0.82-0.75 (m, 2H).

### Example 61

### Synthesis of cyclopropyl(imino) (3-(((8-isopropoxy-1,7-naphthyridin-4-yl)oxy)methyl)phenyl)-lambda6-sulfanone

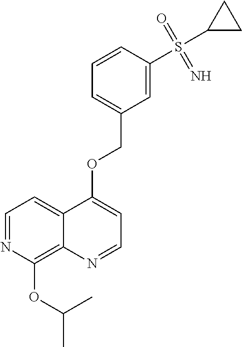 [SMILES: CC(C)Oc1nccc2c(OCc3cccc(S(=N)(=O)C4CC4)c3)ccnc12]

### Step 1:2-Isopropoxy-3-nitropyridine

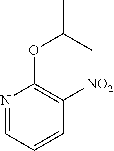

To a solution of isopropyl alcohol (17 mL) in THF (50 mL) was added NaH (2.52 g, 63.07 mmol, 2.0 equiv, 60%) at 0° C. After stirring at 0° C. for 30 min, 2-chloro-3-nitropyridine (5.00 g, 31.54 mmol, 1.0 equiv) was added. After stirring at room temperature for 2 h, the reaction mixture was quenched by the addition of water at 0° C. and extracted with DCM. The combined organic layers were washed with brine, dried over anhydrous sodium sulfate and filtered. The filtrate was concentrated. The residue was purified by silica gel column chromatography, eluted with EtOAc/PE (10:90) to afford 2-isopropoxy-3-nitropyridine (3.83 g, 66%) as an orange oil.

### Step 2:2-Isopropoxypyridin-3-amine

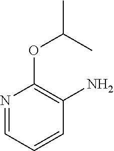

To a solution of 2-isopropoxy-3-nitropyridine (3.83 g, 21.02 mmol, 1.0 equiv) in MeOH (100 mL) was added Pd/C (220 mg, 2.10 mmol, 0.1 equiv). The resulting mixture was stirred at room temperature for 2 h under H₂ atmosphere. The reaction mixture was filtered through a celite pad and the filter cake was washed with MeOH. The filtrate was concentrated under reduced pressure to afford 2-isopropoxypyridin-3-amine (3.12 g, 92%) as a yellow oil.

### Step 3:5-(((2-Isopropoxypyridin-3-yl)amino)methylidene)-2,2-dimethyl-1,3-dioxane-4,6-dione

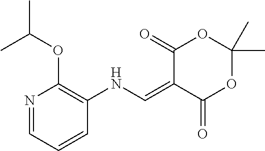 [SMILES: CC(C)Oc1ncccc1NC=C1C(=O)OC(C)(C)OC1=O]

To a solution of 2-isopropoxypyridin-3-amine (3.00 g, 19.71 mmol, 1.0 equiv) in ACN (30 mL) were added 2,2-dimethyl-1,3-dioxane-4,6-dione (3.69 g, 25.62 mmol, 1.3 equiv) and trimethoxymethane (2.93 g, 27.59 mmol, 1.4 equiv). The resulting mixture was stirred at 80° C. for 2 h under N₂ atmosphere. After cooling down to room temperature, the mixture was concentrated under reduced pressure and filtered, the filter cake was washed with water and dried under vacuum to afford 5-(((2-isopropoxypyridin-3-yl)amino)methylidene)-2,2-dimethyl-1,3-dioxane-4,6-dione (6.02 g, 99%) as a green solid.

### Step 4:8-Isopropoxy-1,7-naphthyridin-4-ol

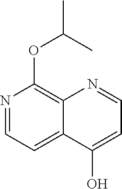

To a solution of 5-(((2-isopropoxypyridin-3-yl)amino)methylidene)-2,2-dimethyl-1,3-dioxane-4,6-dione (3.00 g, 9.79 mmol, 1.0 equiv)diphenyl ether (30 mL). The resulting mixture was stirred at 225° C. for 1.5 h. After cooling down to room temperature, the resulting mixture was filtered and the filter cake was washed with PE. The crude product was purified by reverse phase flash, eluted with ACN/H₂O (63:37) to afford 8-isopropoxy-1,7-naphthyridin-4-ol (518 mg, 25%) as a white solid. MS (ESI, pos. ion) m/z: 205.1 (M+1).

### Step 5:4-Chloro-8-isopropoxy-1,7-naphthyridine

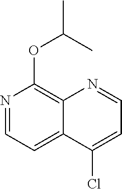

To a solution of 8-isopropoxy-1,7-naphthyridin-4-ol (478 mg, 2.34 mmol, 1.0 equiv) in toluene (6 mL) were added DIEA (605 mg, 4.68 mmol, 2.0 equiv) and POCl₃ (430 mg, 2.81 mmol, 1.2 equiv). The resulting mixture was stirred overnight at 70° C. After cooling down to room temperature, the reaction mixture was concentrated under reduced pressure and purified by reversed phase flash, eluted with ACN/H₂O (31:69) to afford 4-chloro-8-isopropoxy-1,7-naphthyridine (352 mg, 67%) as an off-white solid.

### Step 6:4-((3-(Cyclopropylthio)benzyl)oxy)-8-isopropoxy-1,7-naphthyridine

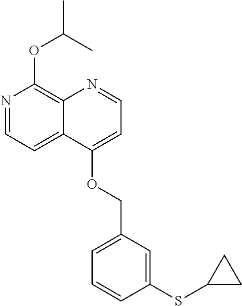 [SMILES: CC(C)Oc1nccc2c(OCc3cccc(SC4CC4)c3)ccnc12]

To a solution of (3-(cyclopropylsulfanyl)phenyl) methanol (267 mg, 1.48 mmol, 1.0 equiv) in DMF (5 mL) was added NaH (118 mg, 2.96 mmol, 2.0 equiv, 60%) at 0° C. After stirring at 0° C. for 10 min, 4-chloro-8-isopropoxy-1,7-naphthyridine (330 mg, 1.48 mmol, 1.0 equiv) was added. The resulting mixture was stirred at 80° C. for 2 h. After cooling down to room temperature, the reaction mixture was quenched by addition of water at 0° C. and extracted with DCM. The combined organic layers were washed with brine, dried over anhydrous sodium sulfate and filtered. The filtrate was concentrated. The residue was purified by reversed phase flash, eluted with ACN/H₂O (100:0) to afford 4-((3-(cyclopropylthio)benzyl)oxy)-8-isopropoxy-1,7-naphthyridine (472 mg, 84%) as colorless oil.

### Step 7: Cyclopropyl(imino) (3-(((8-isopropoxy-1,7-naphthyridin-4-yl)oxy)methyl)phenyl)-lambda6-sulfanone

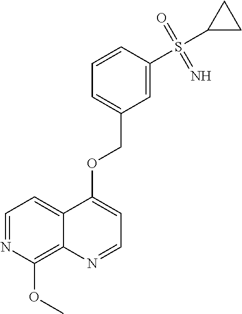 [SMILES: COc1nccc2c(OCc3cccc(S(=N)(=O)C4CC4)c3)ccnc12]

To a solution of 4-((3-(cyclopropylthio)benzyl)oxy)-8-isopropoxy-1,7-naphthyridine (200 mg, 0.54 mmol, 1.0 equiv) in MeOH (3 mL) were added (acetyloxy) (phenyl)-lambda3-iodanyl acetate (527 mg, 1.64 mmol, 3.0 equiv) and ammonium carbamate (170 mg, 2.18 mmol, 4.0 equiv). After stirring at room temperature for 1 h, the resulting mixture was concentrated under reduced pressure and the residue was purified by prep-HPLC with the following conditions (Column: XBridge Prep OBD C18 Column, 50*250 mm, 10 μm; Mobile Phase A: Water (10 mmol/LNH₄HCO₃), Mobile Phase B: ACN; Flow rate: 60 mL/min Gradient: 29% B to 59% B in 20 min; Wave Length: 254 nm/220 nm; RT: 18.17 min) to afford cyclopropyl(imino) (3-(((8-isopropoxy-1,7-naphthyridin-4-yl)oxy)methyl)phenyl)-lambda6-sulfanone (78.2 mg, 35%) as a white solid. MS (ESI, pos. ion) m/z: 398.2 (M+1). ¹H NMR (400 MHZ, DMSO-d₆, ppm) δ 8.77 (d, J=5.2 Hz, 1H), 8.10-8.02 (m, 2H), 7.94-7.87 (m, 1H), 7.86-7.79 (m, 1H), 7.71-7.63 (m, 1H), 7.50 (d, J=6.0 Hz, 1H), 7.36 (d, J=5.2 Hz, 1H), 5.59-5.46 (m, 3H), 4.28 (s, 1H), 2.72-2.61 (m, 1H), 1.40 (d, J=6.4 Hz, 6H), 1.16-1.06 (m, 1H), 1.03-0.83 (m, 3H).

### Example 62

### Synthesis of cyclopropyl(imino) (3-(((8-propoxy-1,7-naphthyridin-4-yl)oxy)methyl)phenyl)-lambda6-sulfanone

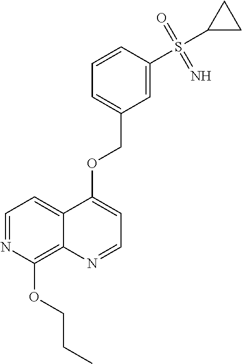 [SMILES: CCCOc1nccc2c(OCc3cccc(S(=N)(=O)C4CC4)c3)ccnc12]

### Step 1:3-Nitro-2-propoxypyridine

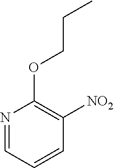

To a solution of propanol (16.6 mL, 222.97 mmol, 7.0 equiv) in THF (100 mL) was added NaH (2.27 g, 94.61 mmol, 3.0 equiv) at 0° C. After stirring at 0° C. for 0.5 h, 2-chloro-3-nitropyridine (5.00 g, 31.53 mmol, 1.0 equiv) was added. After stirring at room temperature for 2 h, the reaction mixture was quenched by the addition of water at 0° C. and extracted with EtOAc. The combined organic layers were washed with brine, dried over anhydrous sodium sulfate and filtered. The filtrate was concentrated. The residue was purified by silica gel column chromatography, eluted with EtOAc/PE (12:88) to afford 3-nitro-2-propoxypyridine (5.22 g, 90%) as a brown oil.

### Step 2:2-Propoxypyridin-3-amine

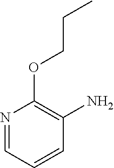

To a solution of 3-nitro-2-propoxypyridine (5.20 g, 28.54 mmol, 1.0 equiv) in MeOH (80 mL) was added Pd/C (2.61 g, 24.54 mmol). The mixture was stirred at room temperature for 2 h under hydrogen atmosphere by using a hydrogen balloon and then filtered through a Celite pad. The filtrate was concentrated under reduced pressure to afford 2-propoxypyridin-3-amine (5.10 g, crude) as a brown oil.

### Step 3:2,2-Dimethyl-5-(((2-propoxypyridin-3-yl)amino)methylidene)-1,3-dioxane-4,6-dione

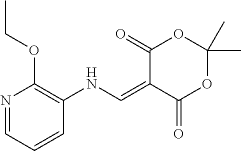 [SMILES: CCOc1ncccc1NC=C1C(=O)OC(C)(C)OC1=O]

To a solution of 2-propoxypyridin-3-amine (5.00 g, 32.85 mmol, 1.0 equiv) in ACN (50 mL) were added 2,2-dimethyl-1,3-dioxane-4,6-dione (6.16 g, 42.70 mmol, 1.3 equiv) and trimethoxymethane (4.88 g, 45.99 mmol, 1.4 equiv) under nitrogen atmosphere. The reaction mixture was stirred at 80° C. for 16 h. After cooling down to room temperature, the reaction mixture was diluted with PE. The precipitate was collected by filtration and washed with PE to afford 2,2-dimethyl-5-(((2-propoxypyridin-3-yl)amino)methylidene)-1,3-dioxane-4,6-dione (7.00 g, 69%) as a light yellow solid.

### Step 4:8-Propoxy-1,7-naphthyridin-4-ol

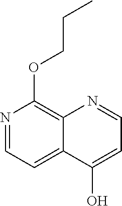

A mixture of 2,2-dimethyl-5-(((2-propoxypyridin-3-yl)amino)methylidene)-1,3-dioxane-4,6-dione (3.50 g, 11.42 mmol, 1.0 equiv) in diphenyl ether (50 mL) was stirred at 225° C. for 1.5 h. After cooling to room temperature, the resulting mixture was diluted with PE. The precipitate was collected by filtration and washed with PE. The crude product was purified by reversed phase flash, eluted with ACN/H₂O (60:40) to afford 8-propoxy-1,7-naphthyridin-4-ol (570 mg, 24%) as a yellow solid.

### Step 5:4-((3-(Cyclopropylsulfanyl)phenyl) methoxy)-8-propoxy-1,7-naphthyridine

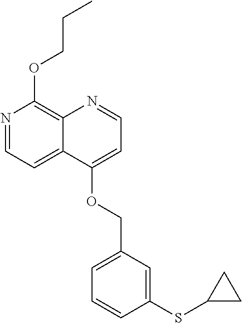 [SMILES: CCCOc1nccc2c(OCc3cccc(SC4CC4)c3)ccnc12]

To a solution of 8-propoxy-1,7-naphthyridin-4-ol (300 mg, 1.46 mmol, 1.0 equiv) in toluene (5 mL) were added (3-(cyclopropylsulfanyl)phenyl) methanol (397 mg, 2.20 mmol, 1.5 equiv) and 2-(tributyl-lambda5-phosphanylidene) acetonitrile (709 mg, 2.93 mmol, 2.0 equiv). The reaction mixture was stirred at 130° C. for 2 h under nitrogen atmosphere. After cooling down to room temperature, the resulting mixture was concentrated under reduced pressure. The residue was purified by reversed phase flash, eluted with ACN/H₂O (70:30) to afford 4-((3-(cyclopropylsulfanyl)phenyl) methoxy)-8-propoxy-1,7-naphthyridine (300 mg, 55%) as a brown solid.

### Step 6: Cyclopropyl(imino) (3-(((8-propoxy-1,7-naphthyridin-4-yl)oxy)methyl)phenyl)-lambda6-sulfanone

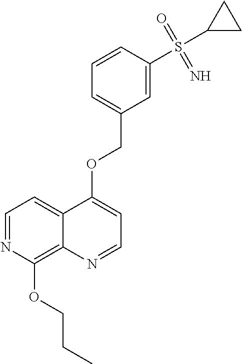 [SMILES: CCCOc1nccc2c(OCc3cccc(S(=N)(=O)C4CC4)c3)ccnc12]

To a solution of 4-((3-(cyclopropylsulfanyl)phenyl) methoxy)-8-propoxy-1,7-naphthyridine (180 mg, 0.49 mmol, 1.0 equiv) in MeOH (4 mL) were added (acetyloxy) (phenyl)-lambda3-iodanyl acetate (474 mg, 1.47 mmol, 3.0 equiv) and ammonium carbamate (153 mg, 1.96 mmol, 4.0 equiv). After stirring at room temperature for 2 h, the resulting mixture was concentrated under reduced pressure. The residue was purified by prep-HPLC with the following conditions (Column: XBridge Prep OBD C18 Column, 50*250 mm, 10 μm; Mobile Phase A: Water (10 mmol/L NH₄HCO₃), Mobile Phase B: ACN; Flow rate: 60 mL/min; Gradient: 31% B to 61% B in 20 min; Wave Length: 254 nm/220 nm; RT: 17.65 min) to afford cyclopropyl(imino) (3-(((8-propoxy-1,7-naphthyridin-4-yl)oxy)methyl)phenyl)-lambda6-sulfanone (70.3 mg, 35%) as an off-white solid. MS (ESI, pos. ion) m/z: 398.1 (M+1). ¹H NMR (400 MHZ, DMSO-d₆, ppm) δ 8.80 (d, J=5.2 Hz, 1H), 8.10-8.03 (m, 2H), 7.94-7.87 (m, 1H), 7.84 (d, J=7.6 Hz, 1H), 7.68 (d, J=7.6 Hz, 1H), 7.53 (d, J=5.6 Hz, 1H), 7.38 (d, J=5.2 Hz, 1H), 5.53 (s, 2H), 4.43-4.39 (m, 2H), 4.28 (s, 1H), 2.69-2.66 (m, 1H), 1.91-1.78 (m, 2H), 1.19-1.07 (m, 1H), 1.06-1.00 (m, 3H), 1.00-0.95 (m, 1H), 0.95-0.82 (m, 2H).

### Example 63

### Synthesis of (2-(1-(8-(fluoromethoxy)-1,7-naphthyridin-4-yl)-4-hydroxypiperidin-4-yl)ethyl)-(imino)(methyl)-lambda6-sulfanone

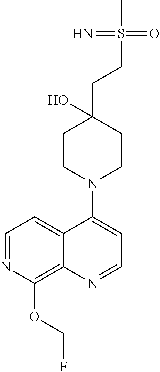 [SMILES: CS(=N)(=O)CCC1(O)CCN(c2ccnc3c(OCF)nccc23)CC1]

### Step 1: tert-butyl 4-Hydroxy-4-(2-hydroxyethyl) piperidine-1-carboxylate

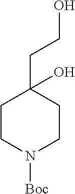 [SMILES: CC(C)(C)OC(=O)N1CCC(O)(CCO)CC1]

To a solution of 2-(1-(tert-butoxycarbonyl)-4-hydroxypiperidin-4-yl) acetic acid (5.00 g, 19.28 mmol, 1.0 equiv) in THF (50 mL) was added borane-tetrahydrofuran complex (77.1 mL, 77.13 mmol, 4.0 equiv, 1.0 M in THF) dropwise under nitrogen atmosphere at 0° C. After stirring at room temperature for 16 h, the reaction mixture was quenched with water, then concentrated under reduced pressure. The residue was purified by silica gel column chromatography, eluted with EtOAc/PE (60:40) to afford tert-butyl 4-hydroxy-4-(2-hydroxyethyl) piperidine-1-carboxylate (3.12 g, crude) as a colorless oil.

### Step 2: tert-butyl 4-Hydroxy-4-(2-(methanesulfonyloxy)ethyl) piperidine-1-carboxylate

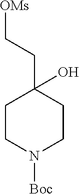 [SMILES: CC(C)(C)OC(=O)N1CCC(O)(CCOS(C)(=O)=O)CC1]

To a solution of tert-butyl 4-hydroxy-4-(2-hydroxyethyl) piperidine-1-carboxylate (3.10 g, 12.63 mmol, 1.0 equiv) and Et₃N (2.56 g, 25.27 mmol, 2.0 equiv) in DCM (30 mL) was added MsCl (1.60 g, 13.90 mmol, 1.1 equiv) dropwise at 0° C. After stirring at 0° C. for 10 min, the reaction mixture was allowed to warm to room temperature and stirred for 2 h. The resulting mixture was concentrated under reduced pressure. The residue was purified by silica gel column chromatography, eluted with EtOAc/PE (56:44) to afford tert-butyl 4-hydroxy-4-(2-(methanesulfonyloxy)ethyl) piperidine-1-carboxylate (3.30 g, crude) as a light yellow oil.

### Step 3: tert-butyl 4-Hydroxy-4-(2-(methylsulfanyl)ethyl) piperidine-1-carboxylate

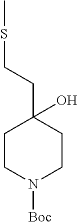 [SMILES: CSCCC1(O)CCN(C(=O)OC(C)(C)C)CC1]

To a solution of tert-butyl 4-hydroxy-4-(2-(methanesulfonyloxy)ethyl) piperidine-1-carboxylate (3.30 g, 10.20 mmol, 1.0 equiv) in EtOH (30 mL) was added sodiummethanethiolate (17.81 g, 51.02 mmol, 5.0 equiv, 20%). After stirring at room temperature for 2 h, the reaction mixture was concentrated under reduced pressure. The residue was purified by silica gel column chromatography, eluted with EtOAc/PE (28:72) to afford tert-butyl 4-hydroxy-4-(2-(methylsulfanyl)ethyl) piperidine-1-carboxylate (900 mg, 25%) as a brown oil.

### Step 4:4-(2-(Methylthio)ethyl) piperidin-4-ol hydrochloride

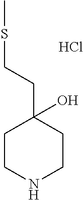

A solution of tert-butyl 4-hydroxy-4-(2-(methylsulfanyl)ethyl) piperidine-1-carboxylate (900 mg, 3.26 mmol, 1.0 equiv) in 4 M HCl (g) in 1,4-dioxane (10 mL) was stirred at room temperature for 1 h. The resulting mixture was concentrated under reduced pressure to afford 4-(2-(methylthio)ethyl) piperidin-4-ol hydrochloride (1.03 g, crude) as an off-white solid.

### Step 5:1-(8-(Fluoromethoxy)-1,7-naphthyridin-4-yl)-4-(2-(methylsulfanyl)ethyl) piperidin-4-ol

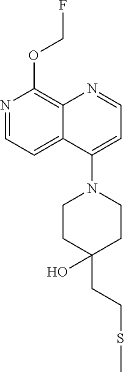 [SMILES: CSCCC1(O)CCN(c2ccnc3c(OCF)nccc23)CC1]

To a solution of 4-chloro-8-(fluoromethoxy)-1,7-naphthyridine (250 mg, 1.17 mmol, 1.0 equiv) in NMP (5 mL) were added 4-(2-(methylthio)ethyl) piperidin-4-ol hydrochloride (298 mg, 1.41 mmol, 1.2 equiv) and DIEA (759 mg, 5.88 mmol, 5.0 equiv). The reaction mixture was stirred at 130° C. for 3 h. After cooling down to room temperature, the reaction solution was purified by reversed phase flash, eluted with ACN/H₂O (47:53) to afford 1-(8-(fluoromethoxy)-1,7-naphthyridin-4-yl)-4-(2-(methylsulfanyl)ethyl) piperidin-4-ol (300 mg, 72%) as a light yellow solid.

### Step 6:4-(4-((tert-butyl Dimethylsilyl)oxy)-4-(2-(methylsulfanyl)ethyl) piperidin-1-yl)-8-(fluoromethoxy)-1,7-naphthyridine

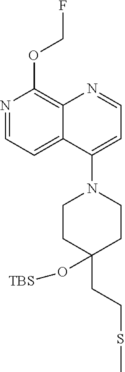

To a solution of 1-(8-(fluoromethoxy)-1,7-naphthyridin-4-yl)-4-(2-(methylsulfanyl)ethyl)-piperidin-4-ol (280 mg, 0.79 mmol, 1.0 equiv) in THF (5 mL) were added 2,6-lutidine (128 mg, 1.19 mmol, 1.5 equiv) and tert-butyldimethylsilyl trifluoromethanesulfonate (421 mg, 1.59 mmol, 2.0 equiv). After stirring overnight at room temperature, the resulting mixture was concentrated under reduced pressure. The residue was purified by silica gel column chromatography, eluted with MeOH/DCM (6:94) to afford 4-(4-((tert-butyldimethylsilyl)oxy)-4-(2-(methylsulfanyl)-ethyl) piperidin-1-yl)-8-(fluoromethoxy)-1,7-naphthyridine (200 mg, 53%) as a brown semi-solid.

### Step 7: (2-(4-((tert-butyl Dimethylsilyl)oxy)-1-(8-(fluoromethoxy)-1,7-naphthyridin-4-yl) piperidin-4-yl)ethyl) (imino)(methyl)-lambda6-sulfanone

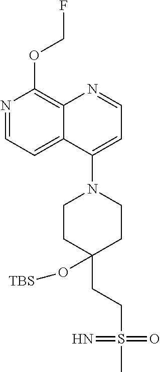

To a solution of 4-(4-((tert-butyldimethylsilyl)oxy)-4-(2-(methylsulfanyl)ethyl) piperidin-1-yl)-8-(fluoromethoxy)-1,7-naphthyridine (150 mg, 0.32 mmol, 1.0 equiv) in ACN (5 mL) was added ammonium hydroxide (225 mg, 25% (v/v)) at −15° C. The reaction mixture was stirred at −15° C. for 1 h. Tert-butyl hypochlorite (174 mg, 1.61 mmol, 5.0 equiv) was added and then the reaction mixture was stirred at −15° C. for another 1 h. Additional ammonium hydroxide (225 mg, 25% (v/v)) was added. The reaction mixture was stirred overnight at room temperature. The resulting mixture was concentrated under reduced pressure and purified by silica gel column chromatography, eluted with MeOH/DCM (18:82) to afford (2-(4-((tert-butyldimethylsilyl)oxy)-1-(8-(fluoromethoxy)-1,7-naphthyridin-4-yl) piperidin-4-yl)ethyl)(methyl)-lambda6-sulfanediimine (50 mg, 31%) as an off-white solid MS (ESI, pos. ion) m/z: 496.1 (M+1) and (2-(4-((tert-butyldimethylsilyl)oxy)-1-(8-(fluoromethoxy)-1,7-naphthyridin-4-yl) piperidin-4-yl)ethyl) (imino)(methyl)-lambda6-sulfanone (20 mg, 20%) as an off-white solid. MS (ESI, pos. ion) m/z: 497.1 (M+1).

### Step 8: (2-(1-(8-(Fluoromethoxy)-1,7-naphthyridin-4-yl)-4-hydroxypiperidin-4-yl)ethyl)-(imino)(methyl)-lambda6-sulfanone

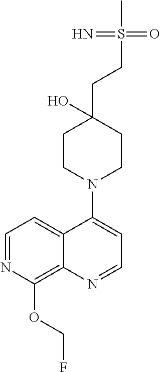 [SMILES: CS(=N)(=O)CCC1(O)CCN(c2ccnc3c(OCF)nccc23)CC1]

To a solution of (2-(4-((tert-butyldimethylsilyl)oxy)-1-(8-(fluoromethoxy)-1,7-naphthyridin-4-yl) piperidin-4-yl)ethyl) (imino)(methyl)-lambda6-sulfanone (20 mg, 0.04 mmol, 1.0 equiv) in THF (1 mL) was added TBAF (0.1 mL, 0.10 mmol, 2.5 equiv, 1.0 M in THF). After stirring at room temperature for 1 h, the resulting mixture was concentrated under reduced pressure. The residue was purified by prep-TLC, eluted with MeOH/DCM (5:95) to afford a crude product, which was re-purified by prep-HPLC with the following conditions (Column: Xbridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 2% B to 29% B in 10 min; Wave Length: 254 nm/220 nm; RT: 8.68 min) to afford (2-(1-(8-(fluoromethoxy)-1,7-naphthyridin-4-yl)-4-hydroxypiperidin-4-yl)ethyl) (imino)(methyl)-lambda6-sulfanone (3.8 mg, 24%) as an off-white solid. MS (ESI, pos. ion) m/z: 383.2 (M+1). ¹H NMR (400 MHZ, DMSO-d₆, ppm) δ 8.73 (d, J=5.2 Hz, 1H), 8.08 (d, J=5.6 Hz, 1H), 7.54 (d, J=6.0 Hz, 1H), 7.23 (d, J=5.2 Hz, 1H), 6.36 (s, 1H), 6.23 (s, 1H), 4.64 (s, 1H), 3.66 (s, 1H), 3.35 (s, 2H), 3.24-3.11 (m, 4H), 2.93 (s, 3H), 1.99-1.89 (m, 2H), 1.89-1.78 (m, 2H), 1.71 (d, J=11.6 Hz, 2H).

¹⁹F NMR (376 MHz, DMSO-d₆, ppm) 8-155.73 (s, 1 F).

### Example 64

### Synthesis of imino (2-(1-(8-methoxypyrido[3,4-d]pyrimidin-4-yl) piperidin-4-yl)ethyl)(methyl)-lambda6-sulfanone

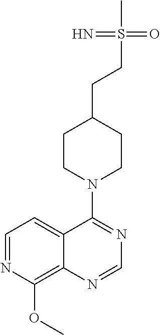 [SMILES: COc1nccc2c(N3CCC(CCS(C)(=N)=O)CC3)ncnc12]

### Step 1:1-(8-Methoxypyrido[3,4-d]pyrimidin-4-yl)-4-[2-(methylsulfanyl)ethyl]piperidine

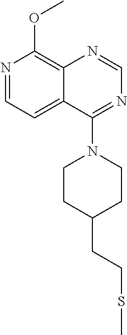 [SMILES: COc1nccc2c(N3CCC(CCSC)CC3)ncnc12]

To a solution of 4-chloro-8-methoxypyrido[3,4-d]pyrimidine (300 mg, 1.53 mmol, 1 equiv) in NMP (6 mL) were added DIEA (991 mg, 7.67 mmol, 5 equiv) and 4-[2-(methylsulfanyl)ethyl]piperidine hydrochloride (450 mg, 2.30 mmol, 1.5 equiv). The resulting mixture was stirred at 100° C. for 2 h. After cooled to room temperature, the crude product was purified by reverse phase flash, eluted with ACN/H₂O (60:40) to afford 1-(8-methoxypyrido[3,4-d]pyrimidin-4-yl)-4-[2-(methylsulfanyl)ethyl]piperidine (330 mg, 67%) as a yellow solid. MS (ESI, pos. ion) m/z: 319.1 (M+1).

### Step 2: Imino (2-(1-(8-methoxypyrido[3,4-d]pyrimidin-4-yl) piperidin-4-yl)ethyl)(methyl)-lambda6-sulfanone

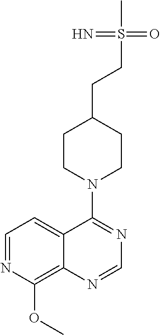 [SMILES: COc1nccc2c(N3CCC(CCS(C)(=N)=O)CC3)ncnc12]

To a mixture of 1-(8-methoxypyrido[3,4-d]pyrimidin-4-yl)-4-(2-(methylsulfanyl)-ethyl) piperidine (280 mg, 0.87 mmol, 1.0 equiv) in ACN (8 mL) was added ammonium hydroxide (1.19 g, 25% (v/v)) under N₂ atmosphere at −15° C. and the mixture was stirred at −15° C. for 1 h. Tert-butyl hypochlorite (477 mg, 4.39 mmol, 5.0 equiv) was added and the mixture was stirred at −15° C. for 1 h. Additional ammonium hydroxide (1.19 g, 25% (v/v)) was added at −15° C. and the mixture was stirred overnight at room temperature. The resulting mixture was concentrated under reduced pressure. The residue was purified by reversed phase flash, eluted with ACN/H₂O (20:80) and was concentrated under reduced pressure. The crude product was re-purified by prep-HPLC (Column: Xbridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 2% B to 29% B in 10 min; Wave Length: 254 nm/220 nm; RT: 8.68 min). The fractions containing the desired product were combined and lyophilized to give imino (2-(1-(8-methoxypyrido[3,4-d]pyrimidin-4-yl) piperidin-4-yl)ethyl)(methyl)-lambda6-sulfanone (17.0 mg, 5%) as yellow solid. MS (ESI, pos. ion) m/z: 350.1 (M+1). ¹H NMR (400 MHZ, DMSO-d₆, ppm) δ 8.64 (s, 1H), 8.05 (d, J=6.0 Hz, 1H), 7.31 (d, J=6.0 Hz, 1H), 4.34-4.31 (m, 2H), 4.02 (s, 3H), 3.61 (s, 1H), 3.21-3.05 (m, 4H), 2.89 (s, 3H), 1.84 (d, J=12.0 Hz, 2H), 1.75-1.64 (m, 3H), 1.44-1.26 (m, 2H).

### Example 65

### Synthesis of (2-(1-(8-(fluoromethoxy)-1,7-naphthyridin-4-yl) piperidin-4-yl)ethyl)-(imino)(methyl)-lambda6-sulfanone

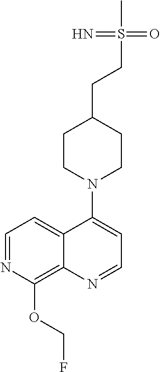 [SMILES: CS(=N)(=O)CCC1CCN(c2ccnc3c(OCF)nccc23)CC1]

### Step 1: tert-butyl 4-(2-((Methylsulfonyl)oxy)ethyl) piperidine-1-carboxylate

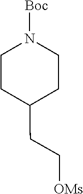 [SMILES: CC(C)(C)OC(=O)N1CCC(CCOS(C)(=O)=O)CC1]

To a solution of tert-butyl 4-(2-hydroxyethyl) piperidine-1-carboxylate (9.00 g, 39.24 mmol, 1 equiv) in DCM (150 mL) were added TEA (11.91 g, 117.73 mmol, 3 equiv), DMAP (0.48 g, 3.925 mmol, 0.1 equiv) and methanesulfonic anhydride (20.51 g, 117.74 mmol, 3 equiv) at 0° C. After stirring at room temperature for 2 h, the reaction mixture was diluted with water (500 mL) and extracted with DCM. The combined organic layers were washed with brine, dried over anhydrous sodium sulfate and filtered. The filtrate was concentrated under reduced pressure to afford tert-butyl 4-[2-(methanesulfonyloxy)ethyl]piperidine-1-carboxylate (10.00 g, crude) as a light yellow oil, which was used for next step directly without further purification.

### Step 2: tert-butyl 4-[2-(Methylsulfanyl)ethyl]piperidine-1-carboxylate

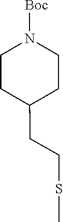 [SMILES: CSCCC1CCN(C(=O)OC(C)(C)C)CC1]

To a solution of tert-butyl 4-[2-(methanesulfonyloxy)ethyl]piperidine-1-carboxylate (10.00 g, 32.53 mmol, 1 equiv) in EtOH (150 mL) was added sodium methanethiolate (113.98 g, 325.30 mmol, 10 equiv, 20% aq). After stirring at room temperature for 2 h, the resulting mixture was concentrated under reduced pressure. The residue was diluted with water and extracted with EtOAc. The combined organic layers were washed with brine, dried over anhydrous sodium sulfate and filtered. The filtrate was concentrated under reduced pressure and purified by silica gel column chromatography, eluted with EA/PE (20:80) to afford tert-butyl 4-[2-(methylsulfanyl)ethyl]piperidine-1-carboxylate (7.00 g, 69% for two steps) as a colorless oil.

### Step 3:4-(2-(Methylthio)ethyl) piperidine hydrochloride

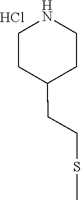

A solution of tert-butyl 4-(2-(methylsulfanyl)ethyl) piperidine-1-carboxylate (851 mg, 3.28 mmol) in HCl (10 mL, gas, 4 M in 4-dioxane) was stirred at room temperature for 1 h. The resulting mixture was concentrated under reduced pressure to afford 4-(2-(methylthio)ethyl)-piperidine hydrochloride (716 mg, crude) as a white solid.

### Step 4:8-(Fluoromethoxy)-4-(4-(2-(methylsulfanyl)ethyl) piperidin-1-yl)-1,7-naphthyridine

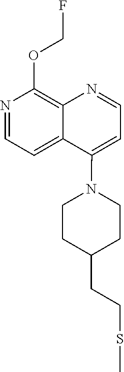 [SMILES: CSCCC1CCN(c2ccnc3c(OCF)nccc23)CC1]

To a stirred mixture of 4-chloro-8-(fluoromethoxy)-1,7-naphthyridine (200 mg, 0.94 mmol, 1.0 equiv) and 4-(2-(methylthio)ethyl) piperidine hydrochloride (320 mg, 1.63 mmol, 1.7 equiv.) in NMP (4 mL) was added DIEA (600 mg, 4.64 mmol, 4.9 equiv.). The resulting mixture was stirred at 130° C. for 4 h. After cooling down to room temperature, the reaction solution was purified by reverse phase flash, eluted with ACN/H₂O (57:33) to afford 8-(fluoromethoxy)-4-(4-(2-(methylsulfanyl)ethyl) piperidin-1-yl)-1,7-naphthyridine (220 mg, 69%) as a brown solid. MS

### Step 5: (2-(1-(8-(Fluoromethoxy)-1,7-naphthyridin-4-yl) piperidin-4-yl)ethyl) (imino)(methyl)-lambda6-sulfanone

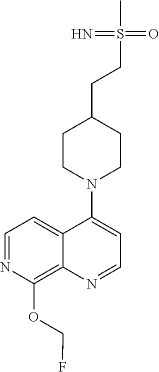 [SMILES: CS(=N)(=O)CCC1CCN(c2ccnc3c(OCF)nccc23)CC1]

A solution of 8-(fluoromethoxy)-4-(4-(2-(methylsulfanyl)ethyl) piperidin-1-yl)-1,7-naphthyridine (220 mg, 0.66 mmol, 1.0 equiv) in ACN (8 mL) was treated with ammonium hydroxide (880 mg, 25% (v/v)) at −15° C. for 1 h under nitrogen atmosphere followed by the addition of tert-butyl hypochlorite (356 mg, 3.28 mmol, 5.0 equiv) dropwise at −15° C. for 1 h. To the above mixture was added ammonium hydroxide (880 mg, 25% (v/v)) dropwise at −15° C. for 1 h. The resulting mixture was stirred overnight at room temperature. The resulting mixture was concentrated under reduced pressure. The residue was purified by reversed phase flash, eluted with ACN/H₂O (24:76) to afford crude product, which was purified by Prep-HPLC with the following conditions (Column: XBridge Prep phenyl OBD C18 Column, 30*150 mm, 5 μm; Mobile Phase A: Water (10 mmol/L NH₄HCO₃), Mobile Phase B: ACN; Flow rate: 60 mL/min; Gradient: 10% B to 25% B in 12 min; Wave Length: 254 nm/220 nm;). The fractions containing the desired product were combined and lyophilized to give (2-(1-(8-(fluoromethoxy)-1,7-naphthyridin-4-yl) piperidin-4-yl)ethyl) (imino)(methyl)-lambda6-sulfanone (29.0 mg, 12%) as a white solid. RT2:9.87 min, MS (ESI, pos. ion) m/z: 367.2 (M+1). ¹H NMR (400 MHZ, DMSO-d₆, ppm) δ 8.71 (d, J=4.0 Hz, 1H), 8.08 (d, J=4.0 Hz, 1H), 7.52 (d, J=8.0 Hz, 1H), 7.19 (d, J=4.0 Hz, 1H), 6.33 (s, 1H), 6.20 (s, 1H), 3.62-3.56 (m, 2H), 3.15-3.09 (m, 2H), 2.91 (s, 3H), 2.87-2.81 (m, 2H), 1.89-1.86 (m, 2H), 1.79-1.69 (m, 2H), 1.68-1.56 (m, 1H), 1.55-1.41 (m, 2H).

¹⁹F NMR (376 MHz, DMSO-d₆, ppm) 8-155.77 (s, 1 F).

### Examples 66, 67 and 68

### Synthesis of cyclopropyl(3-fluoro-5-(((8-methoxy-1,7-naphthyridin-4-yl)oxy)methyl)-phenyl) (imino)-lambda6-sulfanone and(S)-cyclopropyl(3-fluoro-5-(((8-methoxy-1,7-naphthyridin-4-yl)oxy)methyl)phenyl) (imino)-lambda6-sulfanone and (R)-cyclopropyl(3-fluoro-5-(((8-methoxy-1,7-naphthyridin-4-yl)oxy)methyl)phenyl) (imino)-lambda6-sulfanone

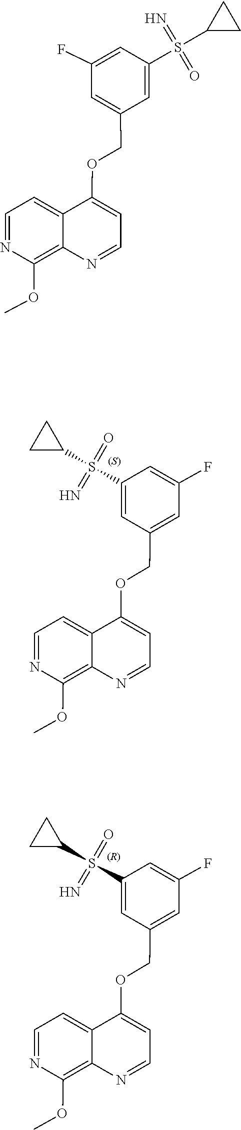

### Step 1:3-(Chlorosulfonyl)-5-fluorobenzoic acid

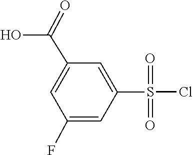 [SMILES: O=C(O)c1cc(F)cc(S(=O)(=O)Cl)c1]

Thionyl chloride (80.4 mL, 27.71 mmol, 4.3 equiv) was added slowly to H₂O (500 mL) and cooled to 5° C., maintaining the temperature between 4 and 7° C. (the addition took about 1.5 h). The solution was then kept stirring overnight while the temperature was allowed to slowly reach room temperature. CuCl (255 mg, 2.57 mmol, 0.01 equiv) was then added to the solution and it was cooled to −15° C. (dry ice/acetone bath), (resulting in solution A). In another flask cooled to 0° C., HCl (260 mL) was added dropwise to 3-amino-5-fluorobenzoic acid (40.00 g, 257.85 mmol, 1.00 equiv), keeping the temperature below 20° C. This slurry was cooled to −10° C. (dry ice/acetone bath) and a solution of sodium nitrite (19.21 g, 278.48 mmol, 1.08 equiv) in H₂O (80 mL) was added very slowly (1 drop/5 sec) to the slurry, keeping the temperature below −5° C. After addition, the orange mixture was allowed to warm to −2° C. for 5 min before cooling back to −15° C. (solution B). Solution B was then added portionwise (plastic pipette) to solution A, cooled to −10° C. After addition (˜30 min), the reaction mixture was stirred at 0° C. for 2 h. The resulting orange solid was filtered and rinsed with water to afford 3-(chlorosulfonyl)-5-fluorobenzoic acid (45.00 g, crude) as an orange solid.

### Step 2:3-Fluoro-5-sulfanylbenzoic acid

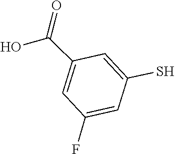 [SMILES: O=C(O)c1cc(F)cc(S)c1]

To a stirred solution of 3-(chlorosulfonyl)-5-fluorobenzoic acid (44.00 g, 184.40 mmol, 1.0 equiv) in con.HCl (400 mL) was added SnCl₂ (104.89 g, 553.20 mmol, 3.0 equiv) at room temperature. After stirred at 100° C. for 3 h, the resulting mixture was diluted with water and basified to 6.0-7.0 with Na₂CO₃. The resulting mixture was extracted with DCM. The combined organic layers were washed with brine, dried over anhydrous sodium sulfate and filtered. The filtrate was concentrated under reduced pressure to give 3-fluoro-5-sulfanylbenzoic acid (28.00 g, crude) as a yellow solid.

### Step 3:3-(Cyclopropylthio)-5-fluorobenzoic acid

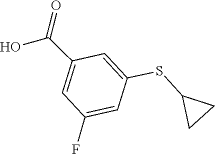 [SMILES: O=C(O)c1cc(F)cc(SC2CC2)c1]

To a stirred solution of 3-fluoro-5-sulfanylbenzoic acid (28.00 g, 162.63 mmol, 1.0 equiv) in DMSO (560 mL) were added t-BuOK (54.75 g, 487.89 mmol, 3.0 equiv) and bromo-cyclopropane (59.02 g, 487.89 mmol, 3.0 equiv) at room temperature. The reaction mixture was stirred overnight at 80° C. After cooling down to room temperature, the reaction mixture was quenched with H₂O and extracted with EtOAc. The combined organic layers were washed with brine, dried over anhydrous sodium sulfate and filtered. The filtrate was concentrated under reduced pressure. The residue was purified by silica gel column chromatography, eluted with MeOH/DCM (9:91) to give 3-(cyclopropylthio)-5-fluorobenzoic acid (16.00 g, 29% for three steps) as a yellow solid.

### Step 4: (3-(Cyclopropylsulfanyl)-5-fluorophenyl) methanol

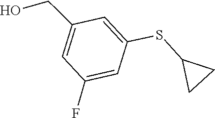 [SMILES: OCc1cc(F)cc(SC2CC2)c1]

To a stirred solution of 3-(cyclopropylthio)-5-fluorobenzoic acid (16.00 g, 75.38 mmol, 1.0 equiv) in THF (160 mL) was added Lithium aluminum hydride (75.4 mL, 150.77 mmol, 2.0 equiv. 2 M in THF) at 0° C. After stirred at room temperature for 2 h, the resulting mixture was quenched with water (50 mL) at 0° C., then concentrated under reduced pressure. The residue was purified by silica gel column chromatography, eluted with EtOAc/PE (44:56) to afford (3-(cyclopropylsulfanyl)-5-fluorophenyl) methanol (12.00 g, 80%) as a yellow oil.

### Step 5:4-((3-(Cyclopropylsulfanyl)-5-fluorophenyl) methoxy)-8-methoxy-1,7-naphthyridine

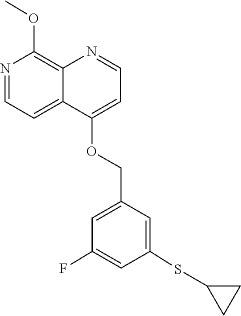 [SMILES: COc1nccc2c(OCc3cc(F)cc(SC4CC4)c3)ccnc12]

To a stirred solution of 4-chloro-8-methoxy-1,7-naphthyridine (400 mg, 2.05 mmol, 1.0 equiv) in DMF (8 mL) were added Cs₂CO₃ (2.00 g, 6.16 mmol, 3.0 equiv), (3-(cyclopropyl-sulfanyl)-5-fluorophenyl) methanol (488 mg, 2.46 mmol, 1.2 equiv) and KF (11 mg, 0.20 mmol, 0.1 equiv) at room temperature. The reaction mixture was stirred at 100° C. for 3 h. After cooling down to room temperature, the resulting mixture was diluted with H₂O and extracted with EtOAc. The combined organic layers were washed with brine, dried over anhydrous sodium sulfate and filtered. The filtrate was concentrated under reduced pressure. The residue was purified by silica gel column chromatography, eluted with EtOAc/PE (56:44) to give 4-((3-(cyclopropylsulfanyl)-5-fluorophenyl) methoxy)-8-methoxy-1,7-naphthyridine (490 mg, 66%) as a white solid.

### Step 6: Cyclopropyl(3-fluoro-5-(((8-methoxy-1,7-naphthyridin-4-yl)oxy)methyl)-phenyl) (imino)-lambda6-sulfanone

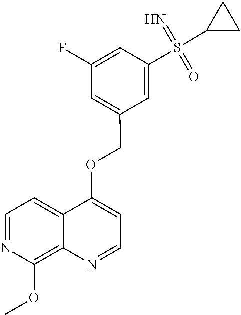 [SMILES: COc1nccc2c(OCc3cc(F)cc(S(=N)(=O)C4CC4)c3)ccnc12]

To a solution of 4-((3-(cyclopropylsulfanyl)-5-fluorophenyl) methoxy)-8-methoxy-1,7-naphthyridine (3.00 g, 8.41 mmol, 1.0 equiv) in MeOH (60 mL) were added (acetyloxy) (phenyl)-lambda3-iodanyl acetate (8.13 g, 25.25 mmol, 3.0 equiv) and ammonium carbamate (2.63 g, 33.66 mmol, 4.0 equiv) at room temperature. After stirred for 2 h, the resulting mixture was concentrated under reduced pressure and the residue was triturated with ACN. The precipitate was collected by filtration, washed with ACN and dried under vacuum to give cyclopropyl(3-fluoro-5-(((8-methoxy-1,7-naphthyridin-4-yl)oxy)methyl)phenyl) (imino)-lambda6-sulfanone (1.80 g, 55%) as a white solid. MS (ESI, pos. ion) m/z: 388.3 (M+1). ¹H NMR (400 MHZ, DMSO-d₆, ppm) δ 8.80 (d, J=5.2 Hz, 1H), 8.10 (d, J=5.6 Hz, 1H), 7.93 (d, J=1.6 Hz, 1H), 7.76-7.68 (m, 2H), 7.59 (d, J=5.6 Hz, 1H), 7.37 (d, J=5.2 Hz, 1H), 5.54 (s, 2H), 4.49 (s, 1H), 4.05 (s, 3H), 2.78-2.71 (m, 1H), 1.18-1.08 (m, 1H), 1.05-0.85 (m, 3H).

¹⁹F NMR (376 MHz, DMSO-do, ppm) 8-110.61 (s, 1 F).

### Step 7: (S)-Cyclopropyl(3-fluoro-5-(((8-methoxy-1,7-naphthyridin-4-yl)oxy)methyl)-phenyl) (imino)-lambda6-sulfanone and (R)-Cyclopropyl(3-fluoro-5-(((8-methoxy-1,7-naphthyridin-4-yl)oxy)methyl)phenyl) (imino)-lambda6-sulfanone

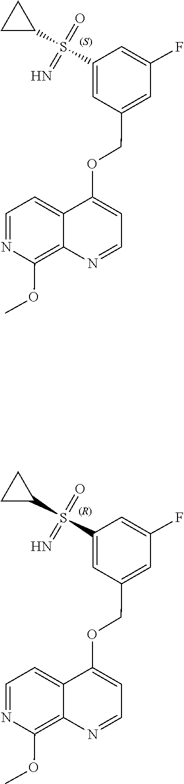

The racemic cyclopropyl(3-fluoro-5-(((8-methoxy-1,7-naphthyridin-4-yl)oxy)methyl)phenyl) (imino)-lambda6-sulfanone (1.80 g, 4.65 mmol) was separated by chiral SFC with the following conditions (Column: CHIRAL ART Amylose-C NEO 3*25 cm, 5 μm; Mobile Phase A: CO₂, Mobile Phase B: IPA (1%-2M-NH₃-IPA); Flow rate: 90 mL/min; Gradient: isocratic 44% B; Column Temperature (C): 35; Back Pressure (bar): 100; Wave Length: 220 nm; Sample Solvent: MeOH; Injection Volume: 10 mL; Number Of Runs: 10) to afford two fractions.

Fraction 1: (S)-cyclopropyl(3-fluoro-5-(((8-methoxy-1,7-naphthyridin-4-yl)oxy)methyl)-phenyl) (imino)-lambda6-sulfanone (751.6 mg, 23%) as a white solid. RT1:6. 57 min, MS (ESI, pos. ion) m/z: 388.2 (M+1). ¹H NMR (400 MHZ, DMSO-d₆, ppm) δ 8.80 (d, J=5.2 Hz, 1H), 8.10 (d, J=5.6 Hz, 1H), 7.93 (d, J=1.6 Hz, 1H), 7.76-7.68 (m, 2H), 7.59 (d, J=5.6 Hz, 1H), 7.37 (d, J=5.2 Hz, 1H), 5.54 (s, 2H), 4.49 (s, 1H), 4.05 (s, 3H), 2.78-2.71 (m, 1H), 1.18-1.08 (m, 1H), 1.05-0.85 (m, 3H).

¹⁹F NMR (377 MHZ, DMSO-do, ppm) 8-110.60 (s, 1 F).

Fraction 2: (R)-cyclopropyl(3-fluoro-5-(((8-methoxy-1,7-naphthyridin-4-yl)oxy)methyl)-phenyl) (imino)-lambda6-sulfanone (770.0 mg, 23%) as a white solid. RT2:10. 97 min, MS (ESI, pos. ion) m/z: 388.1 (M+1). ¹H NMR (400 MHZ, DMSO-d₆, ppm) δ 8.80 (d, J=5.2 Hz, 1H), 8.10 (d, J=5.6 Hz, 1H), 7.93 (d, J=1.6 Hz, 1H), 7.76-7.68 (m, 2H), 7.59 (d, J=5.6 Hz, 1H), 7.37 (d, J=5.2 Hz, 1H), 5.54 (s, 2H), 4.49 (s, 1H), 4.05 (s, 3H), 2.78-2.71 (m, 1H), 1.18-1.08 (m, 1H), 1.05-0.85 (m, 3H).

¹⁹F NMR (377 MHZ, DMSO-d₆, ppm) 8-110.58 (s, 1 F).

### Example 69

### Synthesis of cyclopropyl(3-fluoro-5-(((8-(fluoromethoxy)-1,7-naphthyridin-4-yl)oxy)-methyl)phenyl) (imino)-lambda6-sulfanone

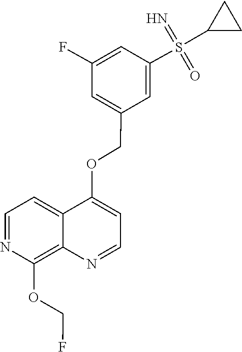 [SMILES: N=S(=O)(c1cc(F)cc(COc2ccnc3c(OCF)nccc23)c1)C1CC1]

### Step 1:4-((3-(Cyclopropylsulfanyl)-5-fluorophenyl) methoxy)-8-(fluoromethoxy)-1,7-naphthyridine

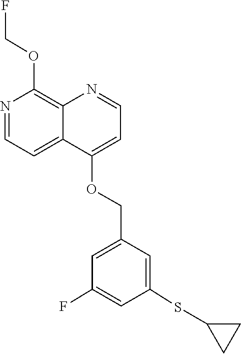 [SMILES: FCOc1nccc2c(OCc3cc(F)cc(SC4CC4)c3)ccnc12]

To a stirred solution of 8-(fluoromethoxy)-1,7-naphthyridin-4-ol (300 mg, 1.54 mmol, 1.0 equiv) in toluene (6 mL) were added (3-(cyclopropylsulfanyl)-5-fluorophenyl) methanol (367 mg, 1.85 mmol, 1.2 equiv) and 2-(tributyl-lambda5-phosphaneylidene) acetonitrile (745 mg, 3.09 mmol, 2.0 equiv) at room temperature. After stirred at 130° C. for 2 h, the reaction mixture was cooled to room temperature and concentrated under reduced pressure. The residue was purified by silica gel column chromatography, eluted with EtOAc/PE (89:11) to afford 4-((3-(cyclopropylsulfanyl)-5-fluorophenyl) methoxy)-8-(fluoromethoxy)-1,7-naphthyridine (400 mg, 69%) as a yellow solid.

### Step 2: Ccyclopropyl(3-fluoro-5-(((8-(fluoromethoxy)-1,7-naphthyridin-4-yl)oxy)methyl)-phenyl) (imino)-lambda6-sulfanone

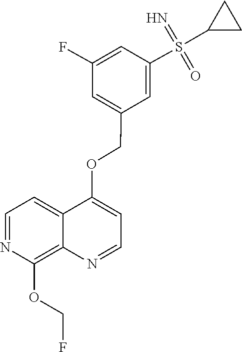 [SMILES: N=S(=O)(c1cc(F)cc(COc2ccnc3c(OCF)nccc23)c1)C1CC1]

To a solution of 4-((3-(cyclopropylsulfanyl)-5-fluorophenyl) methoxy)-8-(fluoromethoxy)-1,7-naphthyridine (50 mg, 0.13 mmol, 1.0 equiv) in MeOH (2 mL) were added (acetyloxy) (phenyl)-lambda3-iodanyl acetate (129 mg, 0.402 mmol, 3.0 equiv) and ammonium carbamate (41 mg, 0.53 mmol, 4.0 equiv). After stirring at room temperature for 2 h, reaction mixture was concentrated under reduced pressure and the residue was purified by prep-HPLC with the following conditions (Column: Xselect CSH Prep C18 OBD Column, 30*150 mm, 5 μm; Mobile Phase A: Water (0.1% FA), Mobile Phase B: ACN; Flow rate: 60 mL/min; Gradient: 11% B to 40% B in 10 min; Wave Length: 254 nm/220 nm; RT: 9.67 min) to afford cyclopropyl(3-fluoro-5-(((8-(fluoromethoxy)-1,7-naphthyridin-4-yl)oxy)methyl)phenyl) (imino)-lambda6-sulfanone (20.8 mg, 37%) as a white solid. MS (ESI, pos. ion) m/z: 406.0 (M+1). ¹H NMR (400 MHZ, DMSO-d₆, ppm) δ 8.88 (d, J=5.2 Hz, 1H), 8.17 (d, J=5.6 Hz, 1H), 7.94 (s, 1H), 7.82-7.67 (m, 3H), 7.42 (d, J=5.2 Hz, 1H), 6.37 (s, 1H), 6.24 (s, 1H), 5.57 (s, 2H), 4.49 (s, 1H), 2.81-2.68 (m, 1H), 1.19-1.08 (m, 1H), 1.05-0.87 (m, 3H).

¹⁹F NMR (376 MHz, DMSO-d₆, ppm) 8-110.58 (s, 1 F), 155.96 (s, 1 F).

### Examples 70 and 71

### Synthesis of (R)-(3-fluoro-5-(((8-methoxy-1,7-naphthyridin-4-yl)oxy)methyl)phenyl)-(imino)(methyl)-lambda6-sulfanone and(S)-(3-fluoro-5-(((8-methoxy-1,7-naphthyridin-4-yl)oxy)methyl)phenyl) (imino)(methyl)-lambda6-sulfanone

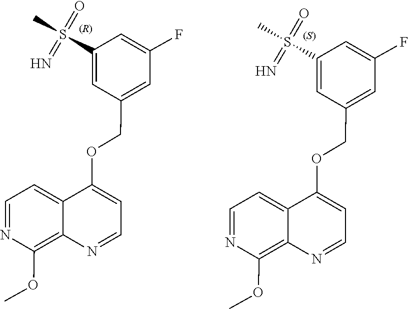

### Step 1: [3-Fluoro-5-(methylsulfanyl)phenyl]methanol

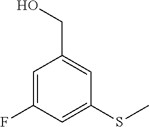

To a solution of (3,5-difluorophenyl) methanol (5.00 g, 34.69 mmol, 1.0 equiv) in DMF (50 mL) was added sodiummethanethiolate (3.16 g, 45.10 mmol, 1.3 equiv). The reaction mixture was stirred at 110° C. for 2 h. After cooling down to room temperature, the resulting mixture was diluted with DCM and washed with H₂O. The organic layer was washed with brine and dried over anhydrous sodium sulfate, filtered and concentrated under reduced pressure. The residue was purified by silica gel column chromatography, eluted with EtOAc/PE (18:82) to afford [3-fluoro-5-(methylsulfanyl)phenyl]methanol (4.50 g, 75%) as a colorless oil.

### Step 2:4-((3-Fluoro-5-(methylsulfanyl)phenyl) methoxy)-8-methoxy-1,7-naphthyridine

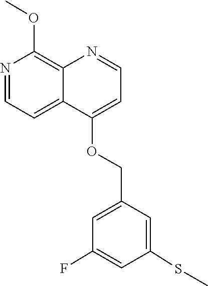 [SMILES: COc1nccc2c(OCc3cc(F)cc(SC)c3)ccnc12]

To a stirred solution of (3-fluoro-5-(methylsulfanyl)phenyl) methanol (600 mg, 3.48 mmol, 1.0 equiv.) in DMF (12 mL) were added Cs₂CO₃ (3.41 g, 10.45 mmol, 3.0 equiv), 4-chloro-8-methoxy-1,7-naphthyridine (1.02 g, 5.22 mmol, 1.5 equiv) and KF (20 mg, 0.34 mmol, 0.1 equiv) at room temperature. The reaction mixture was stirred at 100° C. for 3 h. After cooling down to room temperature, the resulting mixture was diluted with H₂O and extracted with EtOAc. The combined organic layers were washed with brine, dried over anhydrous sodium sulfate and filtered. The filtrate was concentrated under reduced pressure. The residue was purified by silica gel column chromatography, eluted with MeOH/DCM (9:91) to afford 4-((3-fluoro-5-(methylsulfanyl)phenyl) methoxy)-8-methoxy-1,7-naphthyridine (620 mg, 53%) as a yellow solid.

### Step 3: (3-Fluoro-5-(((8-methoxy-1,7-naphthyridin-4-yl)oxy)methyl)phenyl)-(imino)(methyl)-lambda6-sulfanone

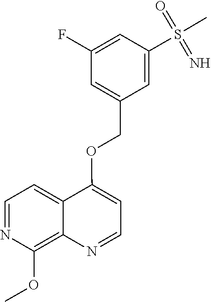

To a solution of 4-((3-fluoro-5-(methylsulfanyl)phenyl) methoxy)-8-methoxy-1,7-naphthyridine (1.00 g, 3.02 mmol, 1.0 equiv) in MeOH (20 mL) were added (acetyloxy) (phenyl)-lambda3-iodanyl acetate (2.92 g, 9.08 mmol, 3.0 equiv) and ammonium carbamate (945 mg, 12.10 mmol, 4.0 equiv). The resulting mixture was concentrated under reduced pressure. The residue was triturated with ACN. The precipitate was collected by filtration, washed with ACN and dried under vacuum to give (3-fluoro-5-(((8-methoxy-1,7-naphthyridin-4-yl)oxy)methyl)phenyl)-(imino)(methyl)-lambda6-sulfanone (0.60 g, 54%) as a white solid.

### Step 4: (R)-(3-Fluoro-5-(((8-methoxy-1,7-naphthyridin-4-yl)oxy)methyl)phenyl)-(imino)(methyl)-lambda6-sulfanone and(S)-(3-fluoro-5-(((8-methoxy-1,7-naphthyridin-4-yl)oxy)methyl)phenyl) (imino)(methyl)-lambda6-sulfanone

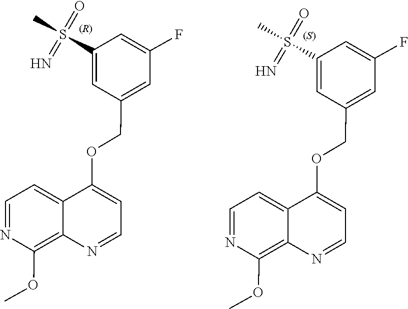

The racemic (3-fluoro-5-(((8-methoxy-1,7-naphthyridin-4-yl)oxy)methyl)phenyl)-(imino)(methyl)-lambda6-sulfanone (0.60 g, 1.66 mmol) was separated by chiral SFC with the following conditions (Column: CHIRAL ART Amylose-C NEO 3*25 cm, 5 μm; Mobile Phase A: CO₂, Mobile Phase B: IPA (1%-2M-NH₃-IPA); Flow rate: 90 mL/min; Gradient: isocratic 44% B; Column Temperature (° C.): 35; Back Pressure (bar): 100; Wave Length: 220 nm; RT1 (min): 6.57; RT2 (min): 10.97; Sample Solvent: MeOH; Injection Volume: 10 mL; Number Of Runs: 10) to afford two fractions. (S)-(3-fluoro-5-(((8-methoxy-1,7-naphthyridin-4-yl)oxy)methyl)phenyl)-(imino)(methyl)-lambda6-sulfanone (195.5 mg, 32%) as a white solid.

Fraction 1: (R)-(3-fluoro-5-(((8-methoxy-1,7-naphthyridin-4-yl)oxy)methyl)phenyl) (imino)(methyl)-lambda6-sulfanone (189.2 mg, 31%) as a white solid. RT1:6.57 min, MS (ESI, pos. ion) m/z: 362.0 (M+1). ¹H NMR (400 MHZ, DMSO-d₆, ppm) δ 8.80 (d, J=5.2 Hz, 1H), 8.09 (d, J=6.0 Hz, 1H), 7.98 (t, J=1.6 Hz, 1H), 7.81-7.70 (m, 2H), 7.58 (d, J=5.6 Hz, 1H), 7.37 (d, J=5.2 Hz, 1H), 5.52 (s, 2H), 4.49-4.44 (m, 1H), 4.05 (s, 3H), 3.15 (s, 3H).

¹⁹F NMR (376 MHZ, DMSO-d₆, ppm) 8-110.62 (s, 1 F).

Fraction 2: (S)-(3-fluoro-5-(((8-methoxy-1,7-naphthyridin-4-yl)oxy)methyl)phenyl) (imino)-(methyl)-lambda6-sulfanone (195.5 mg, 32%) as a white solid. RT2:10.97 min, MS (ESI, pos. ion) m/z: 362.1 (M+1). ¹H NMR (400 MHZ, DMSO-d₆, ppm) δ 8.80 (d, J=5.2 Hz, 1H), 8.09 (d, J=6.0 Hz, 1H), 7.98 (t, J=1.6 Hz, 1H), 7.81-7.70 (m, 2H), 7.58 (d, J=5.6 Hz, 1H), 7.37 (d, J=5.2 Hz, 1H), 5.52 (s, 2H), 4.49-4.44 (m, 1H), 4.05 (s, 3H), 3.15 (s, 3H).

¹⁹F NMR (376 MHz, DMSO-d₆, ppm) 8-110.62 (s, 1 F).

### Example 72

### Synthesis of (3-fluoro-5-(((8-(fluoromethoxy)-1,7-naphthyridin-4-yl)oxy)methyl)phenyl)-(imino)(methyl)-lambda6-sulfanone

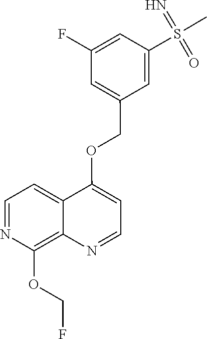 [SMILES: CS(=N)(=O)c1cc(F)cc(COc2ccnc3c(OCF)nccc23)c1]

### Step 1:4-((3-Fluoro-5-(methylthio)benzyl)oxy)-8-(fluoromethoxy)-1,7-naphthyridine

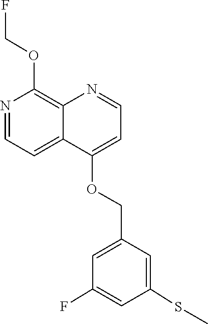 [SMILES: CSc1cc(F)cc(COc2ccnc3c(OCF)nccc23)c1]

To a solution of 8-(fluoromethoxy)-1,7-naphthyridin-4-ol (500 mg, 2.57 mmol, 1.0 equiv) in toluene (10 mL) were added (3-fluoro-5-(methylsulfanyl)phenyl) methanol (665 mg, 3.86 mmol, 1.5 equiv) and 2-(tributyl-lambda5-phosphaneylidene) acetonitrile (1.24 g, 5.15 mmol, 2.0 equiv). The reaction mixture was stirred at 130° C. for 2 h. After cooling down to room temperature, the resulting mixture was diluted EtOAc (150 mL) and washed with H₂O and brine. The organic layer was dried over anhydrous sodium sulfate and filtered. The filtrate was concentrated under reduced pressure. The residue was purified by silica gel column chromatography, eluted with EtOAc/PE (88:12) to afford 4-((3-fluoro-5-(methylthio)benzyl)oxy)-8-(fluoromethoxy)-1,7-naphthyridine (550 mg, 55%) as a yellow solid.

### Step 2: (3-fluoro-5-(((8-(fluoromethoxy)-1,7-naphthyridin-4-yl)oxy)methyl)phenyl)-(imino)(methyl)-lambda6-sulfanone

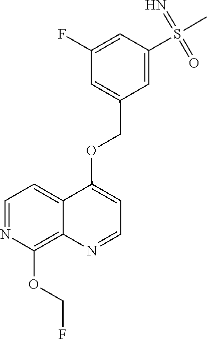 [SMILES: CS(=N)(=O)c1cc(F)cc(COc2ccnc3c(OCF)nccc23)c1]

To a solution of 4-((3-fluoro-5-(methylthio)benzyl)oxy)-8-(fluoromethoxy)-1,7-naphthyridine (80 mg, 0.23 mmol, 1.0 equiv) in MeOH (2 mL) were added (acetyloxy) (phenyl)-lambda3-iodanyl acetate (221 mg, 0.69 mmol, 3.0 equiv) and ammonium carbamate (71 mg, 0.92 mmol, 4.0 equiv). The reaction mixture was stirred at room temperature for 2 h. The precipitate was collected by filtration and washed with ACN (2×5 mL) to afford (3-fluoro-5-(((8-(fluoromethoxy)-1,7-naphthyridin-4-yl)oxy)methyl)phenyl) (imino)(methyl)-lambda6-sulfanone (40 mg, 43%) as an off-white solid. MS (ESI, pos. ion) m/z: 380.0 (M+1). ¹H NMR (400 MHZ, DMSO-d₆, ppm) δ 8.88 (d, J=5.2 Hz, 1H), 8.17 (d, J=5.6 Hz, 1H), 8.00 (d, J=1.6 Hz, 1H), 7.80 (d, J=6.0 Hz, 1H), 7.78-7.74 (m, 2H), 7.43 (d, J=5.2 Hz, 1H), 6.38 (s, 1H), 6.25 (s, 1H), 5.56 (s, 2H), 4.47 (s, 1H), 3.15 (s, 3H).

¹⁹F NMR (376 MHZ, DMSO-d₆, ppm) 8-110.60 (s, 1 F), 8-155.96 (s, 1 F).

### Example 73

### Synthesis of cyclopropyl(imino) (4-(((8-methoxy-1,7-naphthyridin-4-yl)oxy)methyl)phenyl)-lambda6-sulfanone

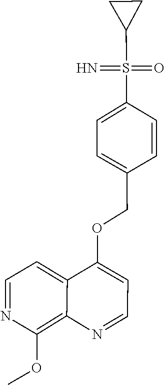 [SMILES: COc1nccc2c(OCc3ccc(S(=N)(=O)C4CC4)cc3)ccnc12]

### Step 1:4-(Cyclopropylsulfanyl)benzoic acid

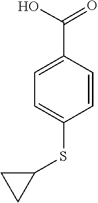 [SMILES: O=C(O)c1ccc(SC2CC2)cc1]

To a stirred solution of 4-mercaptobenzoic acid (2.00 g, 12.97 mmol, 1 equiv) in DMSO (20 mL) were added potassium tert-butoxide (3.64 g, 32.43 mmol, 2.5 equiv) and bromocyclopropane (1.6 mL) at room temperature. The resulting mixture was stirred overnight at 120° C. After cooling down to room temperature, the reaction mixture was dissolved in water and acidified to pH=5 with 1 M HCl (aq). The precipitate was collected by filtration, washed with water and dried under vacuum to give 4-(cyclopropylsulfanyl)benzoic acid (1.69 g, 67%) as a white solid.

### Step 2: (4-(Cyclopropylsulfanyl)phenyl) methanol

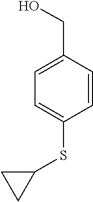 [SMILES: OCc1ccc(SC2CC2)cc1]

To a stirred solution of 4-(cyclopropylsulfanyl)benzoic acid (1.10 g, 5.66 mmol, 1.0 equiv) in THF (22 mL) was added LiAlH₄ (5.5 mL, 11.00 mmol, 2.0 equiv, 2 M in THF) dropwise at 0° C. After stirring for 2 h at room temperature, the resulting mixture was quenched by addition of saturated ammonium chloride solution and concentrated under reduced pressure. The residue was purified by silica gel column chromatography, eluted with MeOH/DCM (9:91) to afford (4-(cyclopropylsulfanyl)phenyl) methanol (593 mg, 58%) as a colorless oil.

### Step 3:4-((4-(Cyclopropylsulfanyl)phenyl) methoxy)-8-methoxy-1,7-naphthyridine

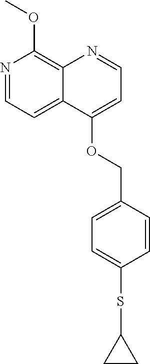 [SMILES: COc1nccc2c(OCc3ccc(SC4CC4)cc3)ccnc12]

To a stirred solution of (4-(cyclopropylsulfanyl)phenyl) methanol (370 mg, 2.05 mmol, 2.0 equiv) in DMF (4 mL) was added NaH (74 mg, 3.08 mmol, 3.0 equiv) at 0° C. After stirring for 10 min, 4-chloro-8-methoxy-1,7-naphthyridine (200 mg, 1.03 mmol, 1.0 equiv) was added and the mixture was stirred at 80° C. for 2 h, the resulting mixture was quenched with water and extracted with EtOAc. The combined organic layers were washed with brine, dried over anhydrous sodium sulfate and filtered. The filtrate was concentrated under reduced pressure. The residue was purified by silica gel column chromatography, eluted with EtOAc/PE (36:64) to afford 4-((4-(cyclopropylsulfanyl)phenyl) methoxy)-8-methoxy-1,7-naphthyridine (121 mg, 34%) as a yellow solid.

### Step 4: Cyclopropyl(imino) (4-(((8-methoxy-1,7-naphthyridin-4-yl)oxy)methyl)phenyl)-lambda6-sulfanone

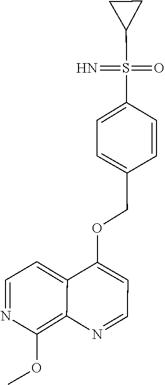 [SMILES: COc1nccc2c(OCc3ccc(S(=N)(=O)C4CC4)cc3)ccnc12]

To a stirred solution of 4-((4-(cyclopropylsulfanyl)phenyl) methoxy)-8-methoxy-1,7-naphthyridine (100 mg, 0.29 mmol, 1.0 equiv) in MeOH (3 mL) was added (acetyloxy) (phenyl)-lambda3-iodanyl acetate (285 mg, 0.88 mmol, 3.0 equiv) and ammonium carbamate (92 mg, 1.18 mmol, 4.0 equiv) at room temperature. After stirring for 1 h, the resulting mixture was concentrated under reduced pressure. The residue was purified by prep-HPLC (Column: XBridge Prep Shield RP C18 Column, 30*150 mm, 5 μm; Mobile Phase A: Water (10 mmol/L NH₄HCO₃), Mobile Phase B: ACN; Flow rate: 60 mL/min, Gradient: 14% B to 35% B in 7 min; Wave Length: 254 nm/220 nm; RT: 7.0 min). The fractions containing the desired product were combined and lyophilized to afford cyclopropyl(imino) (4-(((8-methoxy-1,7-naphthyridin-4-yl)oxy)-methyl)phenyl)-lambda6-sulfanone (42.0 mg, 38%) as a white solid. MS (ESI, pos. ion) m/z: 370.1 (M+1). ¹H NMR (400 MHZ, DMSO-d₆, ppm) δ 8.79 (d, J=5.2 Hz, 1H), 8.09 (d, J=5.6 Hz, 1H), 7.96 (d, J=8.0 Hz, 2H), 7.77 (d, J=8.0 Hz, 2H), 7.59 (d, J=5.6 Hz, 1H), 7.37 (d, J=5.2 Hz, 1H), 5.53 (s, 2H), 4.26 (s, 1H), 4.05 (s, 3H), 2.69-2.66 (m, 1H) 1.14-1.11 (m, 1H), 1.03-0.84 (m, 3H).

### Example 74

### Synthesis of(S)-tert-butyl(imino) (3-(((8-methoxy-1,7-naphthyridin-4-yl)oxy)methyl)phenyl)-lambda6-sulfanone

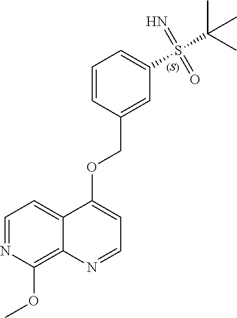

### Step 1:5-(((2-Methoxypyridin-3-yl)amino)methylene)-2,2-dimethyl-1,3-dioxane-4,6-dione

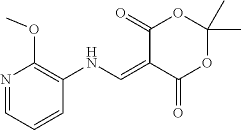 [SMILES: COc1ncccc1NC=C1C(=O)OC(C)(C)OC1=O]

A solution of 2-methoxypyridin-3-amine (3.00 g, 24.2 mmol, 1 equiv), 2,2-dimethyl-1,3-dioxane-4,6-dione (4.50 g, 31.46 mmol, 1.3 equiv) and trimethoxymethane (3.60 g, 33.8 mmol, 1.4 equiv) in acetonitrile (30 mL) was stirred at 80° C. for 2 h under nitrogen atmosphere. After cooling down to room temperature, the resulting solution was concentrated under reduced pressure. The residue was purified by silica gel column chromatography, eluted with EA/PE (25:75). The fractions containing the desired product were combined and concentrated under reduced pressure to afford 5-(((2-methoxypyridin-3-yl)amino)methylene)-2,2-dimethyl-1,3-dioxane-4,6-dione (6.50 g, 97%) as a yellow solid.

### Step 2:8-Methoxy-1,7-naphthyridin-4-ol

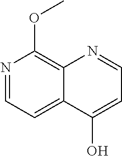

A mixture of 5-(((2-methoxypyridin-3-yl)amino)methylene)-2,2-dimethyl-1,3-dioxane-4,6-dione (1.50 g, 5.4 mmol, 1 equiv) in phenoxybenzene (38 mL) was stirred at 225° C. for 1.5 h. After cooling down to room temperature, PE was added to the reaction mixture. The precipitated solid was collected by filtration, washed with PE (30 ml×3) and then purified by reverse phase flash, eluted with ACN/H₂O (26:74) to afford 8-methoxy-1,7-naphthyridin-4-ol (600 mg, 63%) as a white solid.

### Step 3: ((3-Bromobenzyl)oxy) (tert-butyl)dimethylsilane

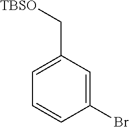 [SMILES: CC(C)(C)[Si](C)(C)OCc1cccc(Br)c1]

To a solution of tert-butyl(chloro)dimethylsilane (9.67 g, 64.16 mmol, 1.2 equiv), TEA (7.57 g, 74.85 mmol, 1.4 equiv) and DMAP (65 mg, 0.53 mmol, 0.01 equiv) in DCM (250 mL) was added (3-bromophenyl) methanol (10.00 g, 53.46 mmol, 1.0 equiv) in DCM (50 mL) at 0° C. After stirring for 16 h at room temperature, the reaction mixture was acidified to PH 4-5 with 5% HCl, diluted with water and then extracted with dichloromethane. The combined organic layers were washed with water and brine, dried over anhydrous sodium sulfate and filtered. The filtrate was concentrated under reduced pressure. The residue was purified by silica gel column chromatography, eluted with EtOAc/PE (28:72) to afford ((3-bromobenzyl)oxy) (tert-butyl)dimethylsilane (14.50 g, 90%) as an off-white oil.

### Step 4: (3-(((tert-butyl Dimethylsilyl)oxy)methyl)phenyl) boronic acid

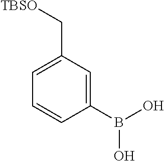 [SMILES: CC(C)(C)[Si](C)(C)OCc1cccc(B(O)O)c1]

To a solution of ((3-bromobenzyl)oxy) (tert-butyl)dimethylsilane (13.50 g, 44.81 mmol, 1.0 equiv) in THF (150 mL) was added n-BuLi (21.5 mL, 53.77 mmol, 1.2 equiv) dropwise under N₂ at −78° C. The resulting mixture was stirred at −78° C. for 1 h. Tri-tert-butyl borate (12.38 g, 53.76 mmol, 1.2 equiv) was added and then the mixture was stirred overnight at room temperature. After cooling down to 0° C., the reaction mixture was acidified to PH 4-5 with 5% H₃PO₄, diluted with water and extracted with EtOAc. The combined organic layers were washed with water and brine, dried over anhydrous sodium sulfate and filtered. The filtrate was concentrated under reduced pressure to afford (3-(((tert-butyldimethylsilyl)oxy)methyl)-phenyl) boronic acid (15.00 g, crude) as an off-white solid.

### Step 5: (R)-N-(tert-Butylsulfinyl) pivalamide

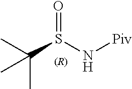

To a solution of (R)-2-methylpropane-2-sulfinamide (10.41 g, 85.89 mmol, 1.0 equiv) in THF (300 mL) was added NaH (8.59 g, 214.7 mmol, 2.5 equiv, 60%) in portions under −5° C. The reaction mixture was stirred at −5° C. for 0.5 h. A solution of trimethylacetic anhydride (17.60 g, 94.48 mmol, 1.1 equiv) in THF (200 mL) was added dropwise over 0.5 h at −5° C. After stirred at room temperature for 2 h, the reaction mixture was quenched with MeOH, followed by saturated aqueous NH₄Cl. The aqueous layer was extracted with EtOAc. The combined organic layers were washed with saturated aqueous NaHCO₃ and brine, dried over anhydrous sodium sulfate and filtered. The filtrate was concentrated under reduced pressure. The residue was purified by silica gel column chromatography, eluted with EtOAc/PE (48:52) to afford (R)-N-(tert-butylsulfinyl) pivalamide (17.00 g, 96%) as an off-white solid.

### Step 6: (S)-N-(tert-Butyl(3-(((tert-butyldimethylsilyl)oxy)methyl)phenyl) (oxo)-lambda6-sulfaneylidene) pivalamide

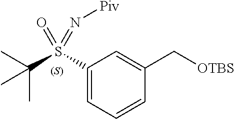

To a solution of (R)-N-(tert-butylsulfinyl) pivalamide (500 mg, 2.43 mmol, 1.0 equiv) and 4 Å MS (2.50 g) in toluene (10 mL) were added (3-(((tert-butyldimethylsilyl)oxy)methyl)-phenyl) boronic acid (1.30 g, 4.87 mmol, 2.0 equiv), Copper (II) trifluoroacetate hydrate (141 mg, 0.48 mmol, 0.2 equiv) and 2-(tert-butylperoxy)-2-methylpropane (1.07 g, 7.31 mmol, 3.0 equiv). The reaction mixture was stirred at 100° C. for 3 h under nitrogen atmosphere. After cooling down to room temperature, the reaction mixture was quenched with H₂O and extracted with EtOAc. The combined organic layers were washed with saturated sodium chloride solution, dried over anhydrous sodium sulfate and filtered. The filtrate was concentrated under reduced pressure. The residue was purified by silica gel column chromatography, eluted with EtOAc/PE (21:79) to afford(S)-N-(tert-butyl(3-(((tert-butyldimethylsilyl)oxy)methyl)phenyl) (oxo)-lambda6-sulfaneylidene) pivalamide (800 mg, 77%) as an off-white solid.

### Step 7: (S)-tert-Butyl(3-(hydroxymethyl)phenyl) (imino)-lambda6-sulfanone

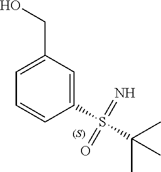

To a solution of(S)-N-(tert-butyl(3-(((tert-butyldimethylsilyl)oxy)methyl)phenyl) (oxo)-lambda6-sulfaneylidene) pivalamide (750 mg, 1.76 mmol, 1.0 equiv) in THF (5 mL) and methanol (5 mL) was added 50% KOH (10 mL). The resulting mixture was stirred for 16 h at 50° C. After cooling down to room temperature, the reaction mixture was concentrated under reduced pressure. The residue was purified by silica gel column chromatography, eluted with MeOH/DCM (8:92) to afford(S)-tert-butyl(3-(hydroxymethyl)phenyl) (imino)-lambda6-sulfanone (250 mg, 62%) as a brown solid.

### Step 8: (S)-tert-Butyl(imino) (3-(((8-methoxy-1,7-naphthyridin-4-yl)oxy)methyl)phenyl)-lambda6-sulfanone

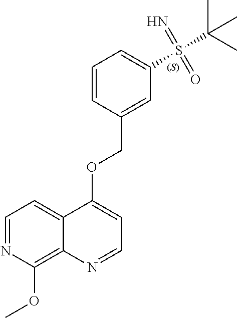

To a solution of(S)-tert-butyl(3-(hydroxymethyl)phenyl) (imino)-lambda6-sulfanone (150 mg, 0.66 mmol, 1.0 equiv) in toluene (5 mL) were added 8-methoxy-1,7-naphthyridin-4-ol (139 mg, 0.79 mmol, 1.2 equiv.) and 2-(tributyl-lambda5-phosphaneylidene) acetonitrile (530 mg, 1.32 mmol, 2.0 equiv). The resulting mixture was stirred at 130° C. for 2 h. After cooling down to room temperature, the reaction mixture was concentrated under reduced pressure. The crude product was purified by reversed phase CombiFlash, eluted with ACN/H₂O (45:55) to afford 100 mg (crude) (S)-tert-butyl(imino) (3-(((8-methoxy-1,7-naphthyridin-4-yl)oxy)methyl)phenyl)-lambda6-sulfanone.

The 100 mg (crude) (S)-tert-butyl(imino) (3-(((8-methoxy-1,7-naphthyridin-4-yl)oxy)methyl)phenyl)-lambda6-sulfanone was further purified by prep-HPLC with the following conditions (Column: XBridge Prep RP OBD C18 Column, 30*150 mm, 5 μm; Mobile Phase A: Water (10 mmol/L NH₄HCO₃), Mobile Phase B: ACN; Flow rate: 60 mL/min; Gradient (B %): 15% B to 30% B in 10 min; Wave Length: 254 nm/220 nm; RT (min): 10.82) to afford(S)-tert-butyl(imino) (3-(((8-methoxy-1,7-naphthyridin-4-yl)oxy)methyl)phenyl)-lambda6-sulfanone (51.8 mg, 20%) as an off-white solid. MS (ESI, pos. ion) m/z: 330.1 (M+1-56). ¹H NMR (400 MHZ, DMSO-d₆, ppm) δ 8.78 (d, J=5.2 Hz, 1H), 8.09 (d, J=5.6 Hz, 1H), 8.02 (t, J=1.8 Hz, 1H), 7.91-7.82 (m, 2H), 7.69 (t, J=7.6 Hz, 1H), 7.54 (d, J=6.0 Hz, 1H), 7.36 (d, J=5.2 Hz, 1H), 5.57 (s, 2H), 4.15 (s, 1H), 4.05 (s, 3H), 1.19 (s, 9H).

### Example 75

### Synthesis of(S)-imino (3-(((8-methoxy-1,7-naphthyridin-4-yl)oxy)methyl)phenyl)(methyl)-lambda6-sulfanone

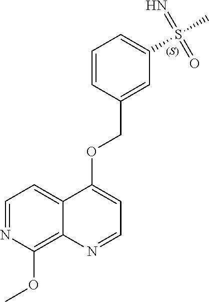

### Step 1: (S)-N-(tert-Butylsulfinyl) pivalamide

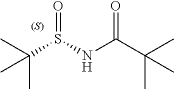

To a solution of(S)-2-methylpropane-2-sulfinamide (17.90 g, 147.65 mmol, 1.1 equiv) in THF (750 mL) was added NaH (17.93 g, 448.32 mmol, 3.3 equiv, 60%) in portions below −5° C. The reaction mixture was stirred at −5° C. for 0.5 h. A solution of trimethylacetic anhydride (25.00 g, 134.22 mmol, 1.0 equiv) in THF (350 mL) was added dropwise over 0.5 h at −5° C. After stirring for 2 h at room temperature, the reaction mixture was quenched with MeOH (20 mL) and followed by saturated aqueous NH₄Cl. The aqueous layer was extracted with EtOAc. The organic layers were combined and washed with saturated aqueous NaHCO₃ and brine, dried over anhydrous sodium sulfate and filtered. The filtrate was concentrated under reduced pressure. The residue was purified by silica gel column chromatography, eluted with EtOAc/PE (35:65) to afford(S)-N-(tert-butylsulfinyl) pivalamide (22.00 g, 79%) as an off-white solid.

### Step 2: (S)-N-(tert-Butyl(methyl) (oxo)-lambda6-sulfaneylidene) pivalamide

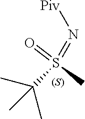

To a solution of(S)-N-(tert-butylsulfinyl) pivalamide (5.00 g, 24.35 mmol, 1.0 equiv) in dioxane (50 mL) were added 1,4,7,10,13-pentaoxacyclopentadecane (6.44 g, 29.22 mmol, 1.2 equiv) and NaH (1.95 g, 48.70 mmol, 2.0 equiv, 60%) in portions at room temperature. The reaction mixture was stirred at room temperature for 1 h. MeI (5.18 g, 36.52 mmol, 1.5 equiv) was added and then the resulting mixture was stirred at 50° C. for 24 h. After cooling down to room temperature, the reaction mixture was quenched with NH₄Cl and extracted with EtOAc. The combined organic layers were washed with brine, dried over anhydrous sodium sulfate and filtered. The filtrate was concentrated under reduced pressure. The residue was purified by silica gel column chromatography, eluted with EtOAc/PE (38:62) to afford(S)-N-(tert-butyl(methyl) (oxo)-lambda6-sulfaneylidene) pivalamide (2.75 g, 51%) as an off-white solid.

### Step 3: (R)-N-(Methylsulfinyl) pivalamide

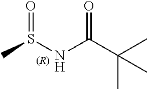

To a solution of(S)-N-(tert-butyl(methyl) (oxo)-lambda6-sulfaneylidene) pivalamide (2.75 g, 12.53 mmol, 1.0 equiv) in DCM (30 mL) was added trifluoroacetic acid (5 mL, 65.29 mmol, 5.2 equiv). After stirring at room temperature for 2 h, the reaction mixture was quenched with saturated aqueous NaHCO₃ and extracted with DCM. The combined organic layers were washed with saturated sodium chloride solution, dried over anhydrous sodium sulfate and filtered. The filtrate was concentrated under reduced pressure. The residue was purified by silica gel column chromatography, eluted with EtOAc/PE (18:82) to afford (R)-N-(methylsulfinyl) pivalamide (1.35 g, 65%) as an off-white solid.

### Step 4: (S)-N-((3-(((tert-Butyldimethylsilyl)oxy)methyl)phenyl)(methyl) (oxo)-lambda6-sulfaneylidene) pivalamide

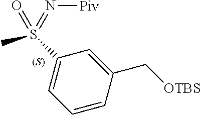

To a solution of (R)-N-(methylsulfinyl) pivalamide (1.30 g, 7.96 mmol, 1.0 equiv) and (3-(((tert-butyldimethylsilyl)oxy)methyl)phenyl) boronic acid (4.24 g, 15.93 mmol, 2.0 equiv) in toluene (20 mL) were added Copper (II) trifluoroacetate hydrate (0.49 g, 1.59 mmol, 0.2 equiv), 4 Å MS (2.5 g) and 2-(tert-butylperoxy)-2-methylpropane (3.49 g, 23.89 mmol, 3.0 equiv). The reaction mixture was stirred at 100° C. for 4 h. After cooling down to room temperature, the reaction mixture was quenched with water and extracted with EtOAc. The combined organic layers were washed with brine, dried over anhydrous sodium sulfate and filtered. The filtrate was concentrated under reduced pressure. The residue was purified by silica gel column chromatography, eluted with EtOAc/PE (48:52) to afford(S)-N-((3-(((tert-butyldimethylsilyl)-oxy)methyl)phenyl)(methyl) (oxo)-lambda6-sulfaneylidene) pivalamide (2.55 g, 81%) as an off-white solid.

### Step 5: (S)-(3-(Hydroxymethyl)phenyl) (imino)(methyl)-lambda6-sulfanone

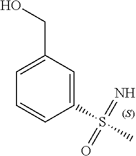

To a solution of(S)-N-((3-(((tert-butyldimethylsilyl)oxy)methyl)phenyl)(methyl) (oxo)-lambda6-sulfaneylidene) pivalamide (2.50 g, 6.51 mmol, 1.0 equiv) in THF (10 mL) and methanol (10 mL) was added 50% KOH (10 mL). The reaction mixture was stirred at 50° C. for 16 h. After cooled to room temperature, the resulting mixture was concentrated under reduced pressure. The residue was purified by silica gel column chromatography, eluted with MeOH/DCM (8:92) to afford(S)-(3-(hydroxymethyl)phenyl) (imino)(methyl)-lambda6-sulfanone (670 mg, 55%) as a white oil.

### Step 6: (S)-Imino (3-(((8-methoxy-1,7-naphthyridin-4-yl)oxy)methyl)phenyl)(methyl)-lambda6-sulfanone

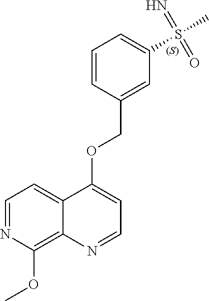

To a solution of(S)-(3-(hydroxymethyl)phenyl) (imino)(methyl)-lambda6-sulfanone (380 mg, 2.05 mmol, 1.0 equiv) in toluene (10 mL) were added 8-methoxy-1,7-naphthyridin-4-ol (469 mg, 2.66 mmol, 1.3 equiv, refer to steps 1 and 2 in example 83) and 2-(tributyl-lambda5-phosphanylidene) acetonitrile (990 mg, 4.10 mmol, 2.0 equiv). The resulting mixture was stirred at 130° C. for 3 h. After cooling down to room temperature, the resulting mixture was concentrated under reduced pressure. The crude product was purified by reverse phase Combiflash, eluted with ACN/H₂O (56:44) to afford crude product. The residue was further purified by trituration with ACN (10 mL×3) to afford(S)-imino (3-(((8-methoxy-1,7-naphthyridin-4-yl)oxy)methyl)-phenyl)(methyl)-lambda6-sulfanone (200.3 mg, 27%) as a light yellow solid. MS (ESI, pos. ion) m/z: 344.1 (M+1). ¹H NMR (400 MHZ, DMSO-d₆, ppm) δ 8.79 (d, J=5.2 Hz, 1H), 8.13 (d, J=2.0 Hz, 1H), 8.08 (d, J=5.6 Hz, 1H), 7.99-7.92 (m, 1H), 7.88-7.81 (m, 1H), 7.69 (t, J=7.6 Hz, 1H), 7.56 (d, J=6.0 Hz, 1H), 7.39 (d, J=5.2 Hz, 1H), 5.52 (s, 2H), 4.29 (s, 1H), 4.05 (s, 3H), 3.10 (s, 3H).

### Example 76

### Synthesis of (R)-tert-butyl(imino) (3-(((8-methoxy-1,7-naphthyridin-4-yl)oxy)methyl)phenyl)-lambda6-sulfanone

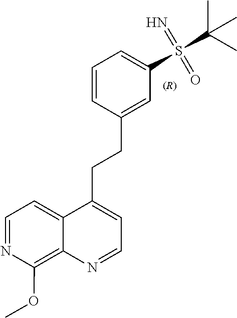

### Step 1: N-((R)-tert-Butyl(3-(((tert-butyldimethylsilyl)oxy)methyl)phenyl)oxo-lambda6-sulfanylidene)-2,2-dimethylpropanamide

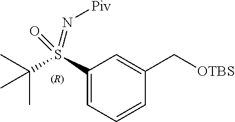

To a stirred solution of 2,2-dimethyl-N-((S)-2-methylpropane-2-sulfinyl) propanamide (500 mg, 2.44 mmol, 1.0 equiv) in toluene (5 mL) were added (3-(((tert-butyldimethylsilyl)-oxy)methyl)phenyl) boronic acid (1.30 g, 4.88 mmol, 2.0 equiv), 2-(tert-butylperoxy)-2-methylpropane (1.07 g, 7.38 mmol, 3.0 equiv), Copper (II) trifluoroacetate hydrate (141 mg, 0.48 mmol, 0.2 equiv) and 4 Å MS (1.0 g) at room temperature under nitrogen atmosphere. The resulting mixture was stirred at 100° C. for 12 h. After cooling down to room temperature, the reaction mixture was quenched with H₂O and extracted with EtOAc. The combined organic layers were washed with saturated sodium chloride solution, dried over anhydrous sodium sulfate and filtered. The filtrate was concentrated under reduced pressure. The residue was purified by silica gel column chromatography, eluted with EtOAc/PE (27:73) to afford N-((R)-tert-butyl(3-(((tert-butyldimethylsilyl)oxy)methyl)phenyl)oxo-lambda6-sulfanylidene)-2,2-dimethylpropanamide (720 mg, 45%) as a white solid.

### Step 2: (R)-tert-Butyl(3-(hydroxymethyl)phenyl) (imino)-lambda6-sulfanone

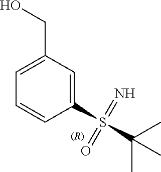

To a stirred solution of N-((R)-tert-butyl(3-(((tert-butyldimethylsilyl)oxy)methyl)-phenyl)oxo-lambda6-sulfanylidene)-2,2-dimethylpropanamide (700 mg, 1.64 mmol, 1.0 equiv) in methanol (3.5 mL) and THF (3.5 mL) was added 50% KOH (7 mL) at room temperature. The resulting mixture was stirred at 70° C. for 6 h. After cooling down to room temperature, the reaction mixture was concentrated under reduced pressure and purified by silica gel column chromatography, eluted with MeOH/DCM (8:92) to afford (R)-tert-butyl(3-(hydroxymethyl)-phenyl) (imino)-lambda6-sulfanone (315 mg, 84%) as a yellow oil.

### Step 3: (R)-tert-Butyl(imino) (3-(((8-methoxy-1,7-naphthyridin-4-yl)oxy)methyl)phenyl)-lambda6-sulfanone

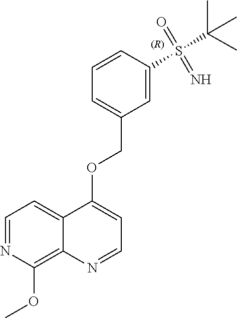

To a stirred solution of (R)-tert-butyl(3-(hydroxymethyl)phenyl) (imino)-lambda6-sulfanone (150 mg, 0.66 mmol, 1.0 equiv) in toluene (2 mL) were added 8-methoxy-1,7-naphthyridin-4-ol (116 mg, 0.66 mmol, 1.0 equiv, refer to steps 1 and 2 in example 83) and 2-(tributyl-lambda5-phosphaneylidene) acetonitrile (318 mg, 1.32 mmol, 2.0 equiv) at room temperature. The reaction mixture was stirred at 130° C. for 2 h. After cooling down to room temperature, the resulting mixture was concentrated under reduce pressure and purified by reverse phase Combiflash, eluted with ACN/H₂O (45:55) to afford 50 mg (crude) (R)-tert-butyl(imino) (3-(((8-methoxy-1,7-naphthyridin-4-yl)oxy)methyl)phenyl)-lambda6-sulfanone as a yellow solid.

The 50 mg (crude) (R)-tert-butyl(imino) (3-(((8-methoxy-1,7-naphthyridin-4-yl)oxy)methyl)phenyl)-lambda6-sulfanone was further purified by prep-HPLC with the following conditions: (Column: Xbridge Prep OBD C18 Column, 30*150 mm, 5 μm; Mobile Phase A: Water (10 mmol/L NH₄HCO₃), Mobile Phase B: ACN; Flow rate: 60 mL/min; Gradient (B %): 17% B to 38% B in 10 min; Wave Length: 254 nm/220 nm; RT1 (min): 8.51). The fractions containing desired product were concentrated under reduced pressure and lyophilized to give (R)-tert-butyl(imino) (3-(((8-methoxy-1,7-naphthyridin-4-yl)oxy)methyl)phenyl)-lambda6-sulfanone (29.5 mg, 11%) as a white solid. MS (ESI, pos. ion) m/z: 386.0 (M+1). ¹H NMR (400 MHZ, DMSO-d₆, ppm) δ 8.77 (d, J=5.2 Hz, 1H), 8.08 (d, J=5.6 Hz, 1H), 8.01 (d, J=1.6 Hz, 1H), 7.90-7.81 (m, 2H), 7.68 (t, J=7.6 Hz, 1H), 7.53 (d, J=6.0 Hz, 1H), 7.36 (d, J=5.2 Hz, 1H), 5.56 (s, 2H), 4.14 (s, 1H), 4.05 (s, 3H), 1.19 (s, 9H).

### Examples 77, 78, and 79

### Synthesis of (3-(((8-(fluoromethoxy)-1,7-naphthyridin-4-yl)oxy)methyl)phenyl) (imino)(methyl)-lambda6-sulfanone and (R)-(3-(((8-(fluoromethoxy)-1,7-naphthyridin-4-yl)oxy)methyl)phenyl) (imino)(methyl)-lambda6-sulfanone and(S)-(3-(((8-(fluoromethoxy)-1,7-naphthyridin-4-yl)oxy)methyl)phenyl) (imino)(methyl)-lambda6-sulfanone

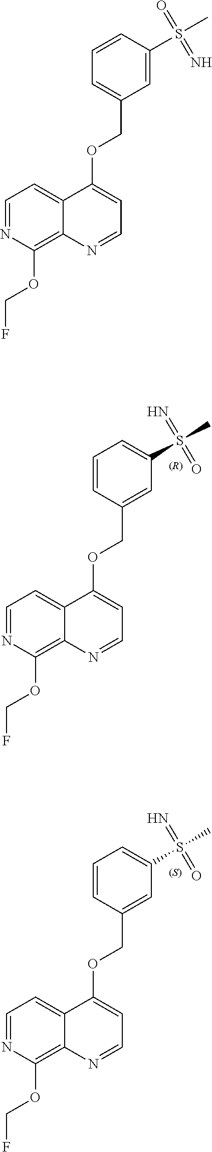

### Step 1:8-(Fluoromethoxy)-4-((3-(methylsulfanyl)phenyl) methoxy)-1,7-naphthyridine

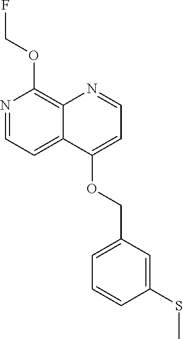 [SMILES: CSc1cccc(COc2ccnc3c(OCF)nccc23)c1]

To a mixture of 4-chloro-8-(fluoromethoxy)-1,7-naphthyridine (640 mg, 3.01 mmol, 1.0 equiv.) and Cs₂CO₃ (1.96 g, 6.02 mmol, 2.0 equiv) in DMF (10 mL) were added KF (17 mg, 0.30 mmol, 0.1 equiv) and (3-(methylsulfanyl)phenyl) methanol (696 mg, 4.52 mmol, 1.5 equiv.). The reaction mixture was stirred at 80° C. for 3 h. After cooling down to room temperature, the resulting mixture was diluted DCM and washed with H₂O and brine. The organic layer was dried over anhydrous sodium sulfate, filtered and concentrated under reduced pressure. The residue was purified by silica gel column chromatography, eluted with EtOAc/PE (38:62) to afford 8-(fluoromethoxy)-4-((3-(methylsulfanyl)phenyl) methoxy)-1,7-naphthyridine (300 mg, 30%) as an off-white solid.

### Step 2: (3-(((8-(Fluoromethoxy)-1,7-naphthyridin-4-yl)oxy)methyl)phenyl) (imino)(methyl)-lambda6-sulfanone

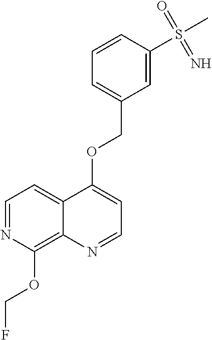 [SMILES: CS(=N)(=O)c1cccc(COc2ccnc3c(OCF)nccc23)c1]

To a stirred solution of 8-(fluoromethoxy)-4-((3-(methylsulfanyl)phenyl) methoxy)-1,7-naphthyridine (300 mg, 0.91 mmol, 1.0 equiv) in MeOH (5 mL) were added (acetyloxy) (phenyl)-lambda3-iodanyl acetate (877 mg, 2.72 mmol, 3.0 equiv) and ammonium carbamate (283 mg, 3.63 mmol, 4.0 equiv) at room temperature. The resulting mixture was stirred at room temperature for 2 h, then purified by reverse-phase CombiFlash, eluted with ACN/H₂O (33:67) to afford (3-(((8-(fluoromethoxy)-1,7-naphthyridin-4-yl)oxy)methyl)phenyl) (imino)(methyl)-lambda6-sulfanone (285 mg, 70%) as a white solid. MS (ESI, pos. ion) m/z: 362.3 (M+1). ¹H NMR (400 MHZ, DMSO-d₆, ppm) δ 8.88 (d, J=5.2 Hz, 1H), 8.19-8.12 (m, 2H), 8.00-7.93 (m, 1H), 7.93-7.83 (m, 1H), 7.77 (d, J=5.6 Hz, 1H), 7.70 (t, J=7.6 Hz, 1H), 7.45 (d, J=5.2 Hz, 1H), 6.38 (s, 1H), 6.25 (s, 1H), 5.56 (s, 2H), 4.30 (s, 1H), 3.11 (s, 3H).

¹⁹F NMR (376 MHZ, DMSO-d₆) δ-155.95 (s, 1 F)

### Step 3: (R)-(3-(((8-(fluoromethoxy)-1,7-naphthyridin-4-yl)oxy)methyl)phenyl) (imino)-(methyl)-lambda6-sulfanone and(S)-(3-(((8-(fluoromethoxy)-1,7-naphthyridin-4-yl)oxy)methyl)phenyl) (imino)(methyl)-lambda6-sulfanone

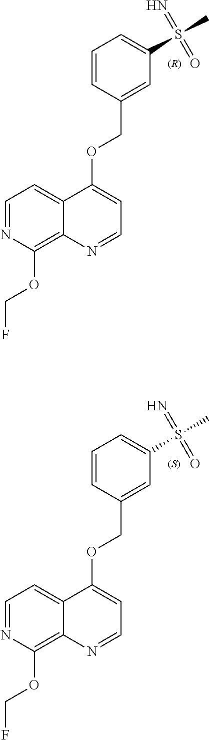

The racemate of (3-(((8-(fluoromethoxy)-1,7-naphthyridin-4-yl)oxy)methyl)phenyl)-(imino)(methyl)-lambda6-sulfanone (285 mg, 0.79 mmol) was purified by SFC with following conditions (Column: CHIRALPAK 1H, 2*25 cm, 5 μm; Mobile Phase A: Hex (0.5% 2M NH₃-MeOH)--HPLC, Mobile Phase B: MeOH: DCM=1:1-HPLC; Flow rate: 20 mL/min; Gradient (B %): isocratic 30; Wave Length: 254/220 nm; Sample Solvent: EtOH-HPLC; Injection Volume: 0.3 mL; Number Of Runs: 24) to afford two fractions.

Fraction 1: RT1 (min): 6.595; (R)-(3-(((8-(fluoromethoxy)-1,7-naphthyridin-4-yl)oxy)-methyl)phenyl) (imino)(methyl)-lambda6-sulfanone (100.5 mg, 35%, assumed absolute stereochemistry) as an off-white solid. MS (ESI, pos. ion) m/z: 362.1 (M+1). ¹H NMR (400 MHZ, DMSO-d₆, ppm) δ 8.88 (d, J=5.2 Hz, 1H), 8.19-8.12 (m, 2H), 8.00-7.93 (m, 1H), 7.93-7.83 (m, 1H), 7.77 (d, J=5.6 Hz, 1H), 7.70 (t, J=7.6 Hz, 1H), 7.45 (d, J=5.2 Hz, 1H), 6.38 (s, 1H), 6.25 (s, 1H), 5.56 (s, 2H), 4.30 (s, 1H), 3.11 (s, 3H).

¹⁹F NMR (376 MHz, DMSO-d₆) δ-155.95 (s, 1 F)

Fraction 2: RT2 (min): 7.992; (S)-(3-(((8-(fluoromethoxy)-1,7-naphthyridin-4-yl)oxy)-methyl)phenyl) (imino)(methyl)-lambda6-sulfanone (94.3 mg, 33%, assumed absolute stereochemistry) as an off-white solid. MS (ESI, pos. ion) m/z: 362.0 (M+1). ¹H NMR (400 MHz, DMSO-d₆, ppm) δ 8.88 (d, J=5.2 Hz, 1H), 8.19-8.12 (m, 2H), 8.00-7.93 (m, 1H), 7.93-7.83 (m, 1H), 7.77 (d, J=5.6 Hz, 1H), 7.70 (t, J=7.6 Hz, 1H), 7.45 (d, J=5.2 Hz, 1H), 6.38 (s, 1H), 6.25 (s, 1H), 5.56 (s, 2H), 4.30 (s, 1H), 3.11 (s, 3H).

¹⁹F NMR (376 MHZ, DMSO-d₆) δ-155.95 (s, 1 F)

### Biological Examples

### Example 1

Measurement of pNP-TMP hydrolysis by ENPP1

p-Nitrophenyl thymidine 5′-monophosphate (pNP-TMP) is a synthesized substrate for ENPP1. The ENPP1 enzyme activity assay with pNP-TMP substrate was conducted as follows:

First, in a 60 μl reaction, 7.5 ng purified ENPP1 was mixed with 1.2 μl compounds of Formula (I) (test compound) ranging from 13.7 nM to 10 μM. Incubation of ENPP1 with compounds was set at 25° C. for 10 min. Reactions with DMSO only (with ENPP1 but no compound) gave the fastest reaction (MAX Activity). For each compound dilution, wells with assay buffer (50 mM Tris -HCl, pH8.8, 250 mM NaCl, 0.1 mg/ml BSA) and test compounds but no ENPP1 were included as controls for the subtraction of test compound derived absorbance at 405 nm.

Second, after 10 minutes incubation of ENPP1 and test compound, the assay was initiated by transferring 50 μl of the above mentioned ENPP1/test compound reaction into 50 μl of 1 mM pNP-TMP in assay buffer results in a 100 μl total reaction in non-binding surface clear bottom 96 well plates. Absorbance at 405 nm was recorded immediately in kinetic mode by PerkinElmer 2300 Enspire multimode plate reader.

For each inhibitor, the specific ENPP1 activity was calculated using the following equation: ENPP1 activity (pmol/min/μg)=Adjusted Vmax (OD405 nm/min) X conversion factor (pmol/OD405 nm)/amount of enzyme (μg)

Adjusted Vmax=V₀X (Km+(S))/(S). In this assay, Km=232 μM, (S)=500 μM. Adjusted Vmax=1.464 X V₀.

V₀=(OD405 nm with ENPP1-OD405 nm ENPP1 blank)/minutes. OD405 nm was plotted, with blank subtracted, against time (minutes), the initial linear rate is V₀.

Blank subtracted, against time (minutes), the initial linear rate is V₀.

The conversion factor (pmol/OD405 nm), was determined by plotting the amount of standard, 4-Nitrophenol (Sigma -Aldrich, Catalog #241326), against absorbance at 405 nm. The slope is the conversion factor. The percent ENPP1 activity for each sample was calculated using the following equation:

$$\% enzyme activity = sample enzyme activity / MAX Activity \times 100 \% .$$

To determine the IC₅₀ for each compound, compound concentration values and percent enzyme activity values were inserted into GraphPad Prism (GraphPad Prism version 9.0 for Windows, GraphPad Software, La Jolla California USA, www.graphpad.com), and Prism's Transform analysis was used to convert the x-axis values (compound concentration) to logarithms. A sigmoidal variable slope nonlinear regression analysis was performed using the following equation: Y=Bottom+ (Top-Bottom)/(1+10\hat{ }((Log IC₅₀″X) *HillSlope)).

Ki values for each compound were calculated from the observed IC₅₀ from GraphPad analysis using the Cheng-Prusoff equation: Kᵢ=IC₅₀/(1+(S)/KM). (S) here is 500 μM and K_{M} is determined to be 232 μM.

Ki for a representative compound of Formula (I) in Compound Table 1 above is provided in Table 2 below:

TABLE 2
Ki (pNP-TMP)

| Group A (<10 nM) | Group B (10 nM | Group C (100 nM |
|---|---|---|
| Cpd # from Cpd | to <100 nM) Cpd # | to <10 μM) Cpd # |
| Table I | from Cpd Table I | from Cpd Table I |
| 1, 2a, 3, 4, 8, 9, 12, 14, | 2b, 7, 10, 13, 17a, 20, | 5, 6, 15, 19, 21, 27, |
| 16, 17b, 22, 24, 26, 28, | 23, 26, 32, 33, 34, 42, | 35, 51, 53, 54, 55, |
| 29, 30, 41, 36, 37, 38, | 52, 63, 70, 78, 76. | 56, 58, 60, 61, 62. |
| 39, 40, 43, 44, 45, 46, | | |
| 49, 50, 57, 59, 65, 67, | | |
| 68, 69, 71, 73, 75, 77, | | |
| 74, 75, 79, 84 | | |

### Example 2

Measurement of 2′3′-cGAMP hydrolysis by ENPP1

ENPP1 catalyzes the hydrolysis of 2′3′-cGAMP into 5′-AMP and 5′-GMP, and hence the ENPP1 enzyme activity with 2′3′-cGAMP as substrate was monitored by measurement of the product 5′-AMP. The AMP-Glo assay kit from Promega (catalog number V5012) was used for measurement of 5′-AMP production.

First, an ENPP1 and test compound incubation was set up in assay buffer (50 mM Tris -HCl, pH8.8, 250 mM NaCl, 0.1 mg/ml BSA) with following conditions: ENPP1 concentration: 1.25 nM; test compound concentration ranging from 137 pM to 100 nM. The incubation was carried out at 25° C. for 10 min.

Second, after the 10-minute incubation of ENPP1 and test compound, prepare on a separate plate, 15 μl of the substrate 2′3′-cGAMP at 200 μM in assay buffer. Then, 15 μl of the ENPP1/Compound incubation was transferred to the 200 μM 2′3′-cGAMP solution to initiate the reaction. The 30 μl mixture was incubated for 30 min at 25° C. In all these assays a DMSO control without compound was included which gave the maximum 5′-AMP production (MAX RLU). After 30 min the reaction was stopped by heating at 90° C. for 3 min.

Third, the Promega AMP-Glo kit was used to detect 5′-AMP production as a measurement of ENPP1 enzyme activity. Briefly, 10 μl of the above mentioned 30 μl total reaction per sample was transferred into 384 well white solid assay plate for measurement of 5′-AMP production. For each well, 10 μl of AMP-Glo Reagent I was added, mixed well, and incubated for 1 hour at 25° C. At this time AMP detection solution was prepared and 20 μl was added per well, and the resulting solution was incubated for 1 hr at 25° C. Duplicates were run for each inhibitor concentration. Luminescence signal (relative luminescence units, RLU) was recorded using a PerkinElmer 2300 Enspire multimode plate reader.

The % inhibition was calculated using the following equation: % inhibition=(MAX RLU-sample RLU)/MAX RLU X 100%.

IC₅₀ values of compounds were determined by loading compound concentration data and percent inhibition values into GraphPad Prism (GraphPad Prism version 7.0 for

Windows, GraphPad Software, La Jolla California USA, www.graphpad.com) and conducted a Sigmoidal variable slope nonlinear regression fitting.

Ki values for each compound were calculated from the observed IC₅₀ from GraphPad analysis using the Cheng-Prusoff equation: Kᵢ=IC₅₀/(1+ (S)/KM). (S) here is 100μ M and KM is 32 μm.

Ki for a representative compound of Formula (I) in Compound Table 1 above is provided in Table 3 below:

TABLE 3
Ki (2′3′-cGAMP)

| Group A (<1 nM) | Group B (1 nM to <5 |
|---|---|
| Cpd # from Cpd | nM) Cpd # from Cpd |
| Table I | Table I |
| 2a, 8, 16, 17, 17b, | 9 |
| 18, 22, 24, 25, 29, | |
| 41, 43, 47, 49, 66, | |
| 67, 69, 71, 72, 77, | |
| 79 | |

### Formulation Examples

The following are representative pharmaceutical formulations containing a compound of the present disclosure.

### Tablet Formulation

The following ingredients are mixed intimately and pressed into single scored tablets.

| | Quantity per |
|---|---|
| Ingredient | tablet mg |
| compound of this disclosure | 400 |
| cornstarch | 50 |
| croscarmellose sodium | 25 |
| lactose | 120 |
| magnesium stearate | 5 |

### Capsule Formulation

The following ingredients are mixed intimately and loaded into a hard-shell gelatin capsule.

| | Quantity per |
|---|---|
| Ingredient | capsule mg |
| compound of this disclosure | 200 |
| lactose spray dried | 148 |
| magnesium stearate | 2 |

### Injectable Formulation

Compound of the disclosure (e.g., compound 1) in 2% HPMC, 1% Tween 80 in DI water, pH 2.2 with MSA, q.s. to at least 20 mg/mL.

### Inhalation Composition

To prepare a pharmaceutical composition for inhalation delivery, 20 mg of a compound disclosed herein is mixed with 50 mg of anhydrous citric acid and 100 mL of 0.9% sodium chloride solution. The mixture is incorporated into an inhalation delivery unit, such as a nebulizer, which is suitable for inhalation administration.

### Topical Gel Composition

To prepare a pharmaceutical topical gel composition, 100 mg of a compound disclosed herein is mixed with 1.75 g of hydroxypropyl cellulose, 10 mL of propylene glycol, 10 mL of isopropyl myristate and 100 mL of purified alcohol USP. The resulting gel mixture is then incorporated into containers, such as tubes, which are suitable for topical administration.

### Ophthalmic Solution Composition

To prepare a pharmaceutical ophthalmic solution composition, 100 mg of a compound disclosed herein is mixed with 0.9 g of NaCl in 100 mL of purified water and filtered using a 0.2 micron filter. The resulting isotonic solution is then incorporated into ophthalmic delivery units, such as eye drop containers, which are suitable for ophthalmic administration.

### Nasal Spray Solution

To prepare a pharmaceutical nasal spray solution, 10 g of a compound disclosed herein is mixed with 30 mL of a 0.05M phosphate buffer solution (pH 4.4). The solution is placed in a nasal administrator designed to deliver 100 μl of spray for each application.

1. A compound of Formula (Id): 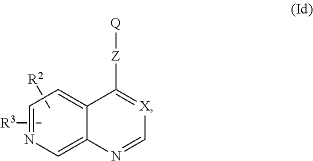
wherein:
X is:
(a) CH or CR¹; or
(b) N;
R¹ is alkyl, alkoxy, halo, haloalkyl, haloalkoxy, amino, alkylamino, dialkylamino, hydroxyalkyl, alkoxyalkyl, hydroxyalkoxy, alkoxyalkoxy, hydroxyalkylamino, alkoxyalkylamino, aminoalkyl, aminoalkoxy, aminoalkylamino, diaminoalkyl, diaminoalkoxy, diaminoalkylamino, or cyano;
R² and R³ are independently absent, alkyl, hydroxy, alkoxy, halo, haloalkyl, haloalkoxy, cyano, amino, alkylamino, dialkylamino, aminocarbonyl, alkylaminocarbonyl, dialkylaminocarbonyl, hydroxyalkyl, alkoxyalkyl, hydroxyalkoxy, alkoxyalkoxy, hydroxyalkylamino, alkoxyalkylamino, aminoalkyl, aminoalkoxy, aminoalkylamino, heterocyclyl, heterocyclyloxy, heterocyclylamino (wherein heterocyclyl either alone or part of heterocyclyloxy and heterocyclylamino is optionally substituted with one, two, or three substituents independently selected from alkyl, halo, hydroxy, alkoxy, hydroxyalkyl, alkoxyalkyl, and aminoalkyl), heterocyclylalkyl, heterocyclylalkyloxy, heterocyclylalkylamino (wherein the heterocyclyl ring in heterocyclylalkyl, heterocyclylalkyloxy, and heterocyclylalkylamino is optionally substituted with one, two, or three substituents independently selected from alkyl, halo, hydroxy, alkoxy, hydroxyalkyl, alkoxyalkyl, and aminoalkyl), cycloalkyloxy, phenyloxy, or heteroaryloxy (where phenyl in phenyloxy and heteroaryl in heteroaryloxy are optionally substituted with one, two, or three substituents independently selected from alkyl, hydroxy, alkoxy, halo, haloalkyl, haloalkoxy, and cyano);
Z is cyclylaminylene, spiro cyclylaminylene, fused cyclylaminylene, NH, N (alkyl), O, S, SO, or SO₂, wherein:
(a) when Z is cyclylaminylene, spiro cyclylaminylene, fused cyclylaminylene, then Q is -(alk) m-W, wherein each Z moiety of (a) is substituted with R⁴ and R⁵, and R⁴ and R⁵ are independently absent, alkyl, alkoxy, halo, haloalkyl, haloalkoxy, hydroxy, or cyano; and
(b) when Z is NH, N (alkyl), O, S, SO, or SO₂, then Q is -(alk¹)ₙ-Ar—W, wherein Ar is arylene or 5 or 6-membered heteroarylene, each Ar is substituted with R⁶ and R⁷, and R⁶ and R⁷ are independently absent, alkyl, alkoxy, halo, haloalkyl, haloalkoxy, hydroxy, or cyano;
alk and alk¹ are independently alkylene optionally substituted with one, two, or three halo;
m and n are independently 0 or 1; and
W is a group consisting of formula (i) or (ii): 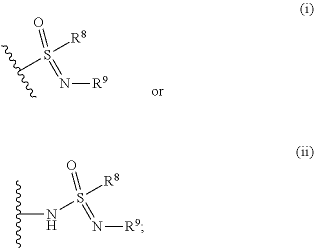
each R⁸ and R⁹ are independently hydrogen, alkyl, substituted alkyl, haloalkyl, substituted haloalkyl, cycloalkyl, substituted cycloalkyl, cycloalkylalkyl, heterocyclyl, or substituted heterocyclyl; or
a pharmaceutically acceptable salt thereof.

2. The compound of claim 1, or a pharmaceutically acceptable salt thereof, wherein X is CH or CR¹.

3. The compound of claim 1, or a pharmaceutically acceptable salt thereof, wherein X is CH.

4. The compound of claim 1, or a pharmaceutically acceptable salt thereof, wherein X is CR¹.

5. The compound of claim 1, or a pharmaceutically acceptable salt thereof, wherein X is N.

6. The compound of claim 1, or a pharmaceutically acceptable salt thereof, where Z is cyclylaminylene, spiro cyclylaminylene, or fused cyclylaminylene, wherein each Z moiety is substituted with R⁴ and R⁵.

7. The compound of claim 1, or a pharmaceutically acceptable salt thereof, wherein Z is cyclylaminylene substituted with R⁴ and R⁵.

8. The compound of claim 1, or a pharmaceutically acceptable salt thereof, wherein Z is spiro cyclylaminylene substituted with R⁴ and R⁵.

9. The compound of claim 1, or a pharmaceutically acceptable salt thereof, wherein the cyclylaminylene or spiro cyclylaminylene is attached to 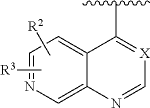
via a nitrogen ring atom, wherein the wavy line is the point of attachment.

10. The compound of claim 1, or a pharmaceutically acceptable salt thereof, wherein the cyclylaminylene, spiro cyclylaminylene, or fused cyclylaminylene of Z are selected from: 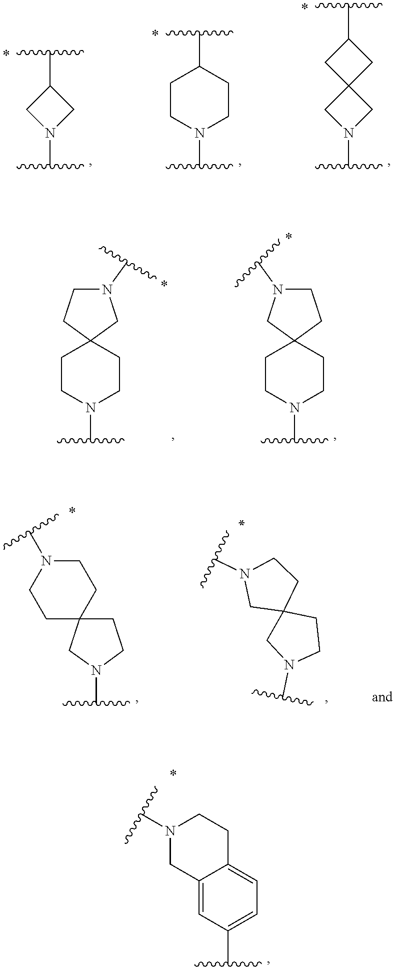
each ring being substituted with R⁴ and R⁵ and where 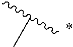
is the point of attachment to -(alk)ₘ-W and 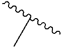
is the attachment to

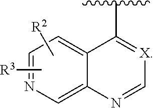

11. The compound of claim 1, or a pharmaceutically acceptable salt thereof, wherein Z is O, N (alkyl), or NH.

12. The compound of claim 1, or a pharmaceutically acceptable salt thereof, where Z is O.

13. The compound of claim 1, or a pharmaceutically acceptable salt thereof, wherein Z is NH or N (alkyl).

14. The compound of claim 1, or a pharmaceutically acceptable salt thereof, wherein m is 0 and n is 0.

15. The compound of claim 1, or a pharmaceutically acceptable salt thereof, wherein m is 1 and n is 1.

16. The compound of claim 1, a pharmaceutically acceptable salt thereof, wherein alk and alk¹ are independently methylene, ethylene, or propylene.

17. The compound of claim 1, or a pharmaceutically acceptable salt thereof, wherein alk and alk¹ are methylene.

18. The compound of claim 1, or a pharmaceutically acceptable salt thereof, wherein Ar is phenylene or 5- or 6-membered heteroarylene substituted with R⁶ and R⁷.

19. The compound of claim 1, or a pharmaceutically acceptable salt thereof, wherein Ar is phenylene substituted with R⁶ and R⁷.

20. The compound of claim 1, or a pharmaceutically acceptable salt thereof, wherein Ar is phenylene and W is attached to carbon of the phenylene ring that is para to the carbon of the phenylene ring that is attached to -(alk¹)ₙ- of -Z-(alk¹)ₙ-.

21.-54. (canceled)
